(19) United States
(12) Patent Application Publication
(10) Pub. No.: US 2022/0144853 A1
(43) Pub. Date: May 12, 2022

(54) IMIDAZOLINE DERIVATIVES AS CXCR4 MODULATORS

(71) Applicants: Université de Paris, Paris (FR); Centre National de la Recherche Scientifique, Paris (FR)

(72) Inventors: Nassima BEKADDOUR BENATIA, Angers (FR); Mathieu RODERO, Paris (FR); Jean-Philippe HERBEUVAL, Paris (FR); Nicolas PIETRANCOSTA, Montigny le Bretonneux (FR); Nikaïa SMITH, Nice (FR); Anaïs BARRÉ, Illkirch Graffenstaden (FR); Julie CASSOU, Eschau (FR); Stanislas MAYER, Eschau (FR)

(73) Assignees: Université de Paris, Paris (FR); Centre National de la Recherche Scientifique, Paris (FR)

(21) Appl. No.: 17/599,785

(22) PCT Filed: Mar. 27, 2020
(86) PCT No.: PCT/EP2020/058728

§ 371 Date: Sep. 29, 2021

(30) Foreign Application Priority Data
Mar. 29, 2019 (EP) 19305409.5

(51) Int. Cl. C07D 513/04; C07D 401/12; C07D 519/00; C07D 403/12

(52) U.S. Cl. CPC C07D 513/04; C07D 403/12; C07D 519/00; C07D 401/12

(57) ABSTRACT

The present invention provides novel compounds of formula (I) and pharmaceutical compositions containing these compounds. The compounds of formula (I) can act as CXCR4 modulators that specifically target the CXCR4 minor pocket, and they have further been found to inhibit the production of inflammatory cytokines in immune cells, which renders these compounds highly advantageous for use in therapy, particularly in the treatment or prevention of an inflammatory disorder, an autoimmune disorder, an autoinflammatory disorder, or an interferonopathy, such as, e.g., lupus erythematosus, dermatomyositis or rheumatoid arthritis.

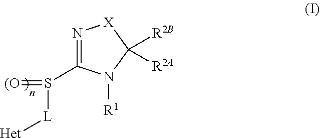

The present invention provides novel compounds of formula (I) and pharmaceutical compositions containing these compounds. The compounds of formula (I) can act as CXCR4 modulators that specifically target the CXCR4 minor pocket, and they have further been found to inhibit the production of inflammatory cytokines in immune cells, which renders these compounds highly advantageous for use in therapy, particularly in the treatment or prevention of an inflammatory disorder, an autoimmune disorder, an autoinflammatory disorder, or an interferonopathy, such as, e.g., lupus erythematosus, dermatomyositis or rheumatoid arthritis.

Disorders of the immune system are at the basis of numerous diseases that can be divided into two categories: autoinflammatory diseases that affect the innate immune system and autoimmune diseases that involve the adaptive immune system. In both cases, the immune system attacks the normal constituents of the organism considering them as foreign. It becomes pathogenic and induces lesions on a specific organ (e.g., type 1 diabetes in the pancreas or multiple sclerosis in the brain) or systemically (e.g., rheumatoid arthritis or systemic lupus erythematosus, SLE). These diseases evolve chronically, with phases of relapse and remission. At the origin of these dysfunctions, the failure of self-tolerance mechanisms is induced by multiple genetic, hormonal and environmental factors and is still largely misunderstood.

Some autoimmune diseases are rare, affecting less than one in five thousand individuals. But taken as a whole, they are common, affect mainly women and their overall prevalence is about 5 to 10%. As an example, rheumatoid arthritis is one of the most frequent with an estimated prevalence in France at 1000 to 4000 per 100 000 women (4 times less for men).

Cytokines are small proteins involved in cell signaling that orchestrate the immune response. Targeting them has therefore become a real therapeutic option for autoimmune and autoinflammatory diseases but also for chronic viral infections and inflammatory diseases such as sepsis.

Interferonopathies

Type I interferons (IFN-1) are key immune response mediators and in humans are composed of 13 IFN-alpha (IFN-α) subtypes as well as IFN-β, IFN-ε, IN-κ and IFN-ω. Type I IFNs signal through a common receptor (IFNAR) ubiquitously expressed and formed by two transmembrane proteins, IFNAR1 and IFNAR2. IFNAR engagement results in activation of the cytoplasmic kinases JAK1 and TYK2 leading to the formation of the transcription factor complex ISGF3. This complex translocates to the nucleus to promote transcription of IFN-stimulated genes (ISG). Type-I IFNs have antiproliferative and immunomodulatory effects and are essential to control viral infection and spread. However, sustained overproduction of IFN-I can be deleterious for the host. The negative impact of IFN-1 is well illustrated by a class of disorders collectively termed type 1 interferonopathies (Gitiaux C et al., Arthritis Rheumatol, 2018, 70, 134-145; Melki I et al., J Allergy Clin Immunol, 2017, 140, 543-552 e545; Rice G I et al, J Clin Immunol, 2017, 37, 123-132; Rodero M P et al, J Exp Med, 2017, 214, 1547-1555; Rodero M P et al., Nature communications, 2017, 8, 2176), which include rare monogenic diseases and complex autoinflammatory/autoimmune diseases such as systemic lupus erythematous (SLE).

Autoinflammation and Autoimmunity Triggered by Type I Interferon

The type I interferonopathies comprise a growing number of genetically determined disorders that are primarily caused by perturbations of the innate immune system. The term type I interferonopathy was coined in recognition of an abnormal upregulation of type I IFN as a unifying phenotype of this novel group of diseases (Crow Y J, Curr Opin Immunol, 2015, 32, 7-12). Despite a remarkable phenotypic heterogeneity, type I interferonopathies are commonly characterized by systemic autoinflammation and varying degrees of autoimmunity or immunodeficiency. Based on the currently identified molecular defects, a pathogenic type I IFN response can result from (a) abnormal accumulation of or abnormal chemical modification of endogenous nucleic acids, (b) enhanced sensitivity or ligand-independent activation of nucleic acid sensors or of downstream components of type I IFN signaling pathways, (c) impaired negative regulation of nucleic acid-induced type I IFN signaling, or (d) defects in pathways that modulate type I IFN responses independent of nucleic acid sensing (Lee-Kirsch M A, Annu Rev Med, 2017, 68, 297-315).

Type I interferonopathies include, for example, Aicardi-Goutières syndrome (AGS), retinal vasculopathy with cerebral leukodystrophy (RVCL), familial chilblain lupus (CHBL), systemic lupus erythematosus (SLE), STING-associated vasculopathy with onset in infancy (SAVI), Singleton-Merten syndrome (SGMRT), spondyloenchondrodysplasia (SPENCD), ISG15 deficiency, proteasome-associated autoinflammatory syndrome, and deficiency of adenosine deaminase 2.

The development of therapies aiming to inhibit type I IFN production in autoimmune diseases has been stimulated by the observation that type I IFNAR knock-out murine lupus models have a reduced disease activity. Although upregulation of IFNs in SLE has been detected for a long time, however, the anti-IFN therapy developed very slowly (Felten R et al., Autoimmunity reviews, 2018, 17, 781-790). It is not only due to no effective approach to block IFN but also the high risk to induce infection or tumor induced by anti-IFN therapy (Crow M K, Rheumatic diseases clinics of North America, 2010, 36, 173-186). There are possible methods to downregulate the IFN pathway in SLE, but a more personalized approach to modulate the type I IFN system in order to reduce the risk for increased frequency and severity of infectious diseases would be a major therapeutic leap forward for this vulnerable group of patients. At this moment, a number of clinical trials are in progress. Given promising results, the efficacy of sifalimumab, an IFN-α monoclonal antibody, is now being investigated in phase II clinical trials. Based on preliminary results, experimental group has an obvious improvement compared with placebo group and a single injection of an anti-IFN-α antibody could give a sustained neutralization of the IFN signature. In this study, researchers found that sifalimumab suppresses IFN-α level not only in whole blood but also in skin tissue of SLE. So far, no increase in serious viral infections has been reported among anti-IFN-α-treated patients, which could be due to the fact that, besides IFN-α, several other type I IFNs exist with strong antiviral activity.

Autoinflammatory Diseases

Autoinflammatory diseases are conditions where inflammatory cytokines are involved in the pathogenesis. They are characterized by immune activation, infiltration and abnormal cytokine production. They include conditions such as: rheumatologic inflammatory diseases, skin inflammatory diseases, lung inflammatory diseases, muscle inflammatory diseases, bowel inflammatory diseases, brain inflammatory diseases and autoimmune diseases.

Among this large panel of diseases, rheumatoid arthritis (RA) is a long-term autoimmune disorder that primarily affects joints (Smolen J S et al., The Lancet, 2016, 388, 2023-2038). It typically results in warm, swollen, and painful joints. Pain and stiffness often worsen following rest. Most commonly, the wrist and hands are involved, with the same joints typically involved on both sides of the body (https://www.niams.nih.gov/health-topics/rheumatoid-arthritis). The disease may also affect other parts of the body (Smolen J S et al., The Lancet, 2016, 388, 2023-2038). This may result in a low red blood cell count, and inflammation around the heart. Fever and low energy may also be present. Often, symptoms come on gradually over weeks to months. While the cause of rheumatoid arthritis is not clear, it is believed to involve a combination of genetic and environmental factors (https://www.niams.nih.gov/health-topics/rheumatoid-arthritis). The underlying mechanism involves the body's immune system attacking the joints (Smolen J S et al., The Lancet, 2016, 388, 2023-2038). This results in inflammation and thickening of the joint capsule (Smolen J S et al., The Lancet, 2016, 388, 2023-2038). It also affects the underlying bone and cartilage (Smolen J S et al., The Lancet, 2016, 388, 2023-2038). The diagnosis is made mostly on the basis of a person's signs and symptoms. X-rays and laboratory testing may support a diagnosis or exclude other diseases with similar symptoms (Smolen J S et al., The Lancet, 2016, 388, 2023-2038). Other diseases that may present similarly include systemic lupus erythematosus, psoriatic arthritis, and fibromyalgia among others.

The goals of treatment are to reduce pain, decrease inflammation, and improve a person's overall functioning. This may be helped by balancing rest and exercise, the use of splints and braces, or the use of assistive devices. Pain medications, steroids, and NSAIDs are frequently used to help with symptoms. Disease-modifying antirheumatic drugs (DMARDs), such as hydroxychloroquine and methotrexate, may be used to try to slow the progression of disease. Biological DMARDs may be used when disease does not respond to other treatments. However, they may have a greater rate of adverse effects. Surgery to repair, replace, or fuse joints may help in certain situations.

Inflammatory Diseases

While autoimmune and autoinflammatory diseases evolve chronically, some conditions can lead to an acute immune disorder. Indeed, a sudden excessive and uncontrolled release of pro-inflammatory cytokines, also called cytokine storm, has been observed in graft-versus-host disease, multiple sclerosis, pancreatitis, multiple organ dysfunction syndrome, viral diseases, bacterial infections, hemophagocytic lymphohistiocytosis, and sepsis (Gerlach H, F1000Res, 2016, 5, 2909; Tisoncik J R et al., Microbiol Mol Biol Rev, 2012, 76(1), 16-32). In these conditions, a dysregulated immune response and subsequent hyperinflammation may lead to multiple organ failure that can be fatal.

Sepsis is a systemic inflammatory response to infection with highly variable clinical manifestations (Angus D C et al., N Engl J Med, 2013, 369(9), 840-851). Acute organ dysfunction commonly affects the respiratory and cardiovascular system with acute respiratory distress syndrome (ARDS) and hypotension or elevated serum lactate level. The brain and kidneys are also often affected leading to obtundation, delirium, polyneuropathy, myopathy or acute kidney injuries (Angus D C et al, N Engl J Med, 2013, 369(9), 840-851).

Treatment of sepsis consists in 2 phases. The initial management within the first 6 hours after the patient's presentation consists in providing cardiorespiratory resuscitation (fluids, vasopressors, oxygen therapy and mechanical ventilation) and controlling the infection (antibiotics). After these first 6 hours, attention focuses on supporting organ functions and avoiding complications. In this second part, immunomodulatory therapy such as hydrocortisone can be administered (Angus D C et al., N Engl J Med, 2013, 369(9), 840-851).

Despite substantial advances in modern intensive care, mortality in sepsis patients is still close to 20 to 30%. Patients who survive sepsis remain at risk for death in the following months and years and often have impaired physical or neurocognitive functioning, mood disorders and low quality of life (Angus D C et al., N Engl J Med, 2013, 369(9), 840-851). Therefore, new therapeutic strategies are urgently needed.

CXCR4 as Therapeutic Target

CXCR4 is a well-known chemokine receptor described for its role in cell migration (chemotaxis). CXCR4 expression has been reported in most hematopoietic cell types, including neutrophils, monocytes, B and T lymphocytes, CD34+ progenitor cells, immature and mature dendritic cells, and platelets. It is also highly expressed in vascular endothelial cells, neurons, microglia, astrocytes and several types of cancer cells. Upon injury, the blockade of the interaction between CXCR4 and its ligand CXCL12 or SDF1-α enhances progenitor cell mobilization from the bone marrow to the periphery. Similarly, CXCR4 influences trafficking of other immune cells, but also CXCR4-positive cancer cells. Furthermore, CXCR4 and CCR5 are coreceptors involved in Human immunodeficiency Virus (HIV) entry into CD4+ T cells in humans. Based on these functions, CXCR4 has been widely studied by the pharmaceutical industries. For example, the CXCR4 antagonist AMD3100 or plerixafor is clinically approved for the mobilization of hematopoietic progenitor cells for autologous transplantations in patients with lymphoma and multiple myeloma. Antagonists of CXCR4 are also actively developed to prevent the migration of CXCR4-expressing cancer cells either to prevent metastasis of solid tumors or the homing of leukemic cells in the bone marrow which is associated with drug resistance.

Numerous CXCR4 ligands have been described including pyridines, quinolones, peptides or polyazamacrocycles with a large range of affinities (Debnath B et al., Theranostics, 2013, 3, 47-75). CXCR4 is overexpressed by activated immune cells in autoimmune and autoinflammatory disease patients (Wang A et al., Arthritis and Rheumatism, 2010, 62, 3436-3446). It has further been demonstrated that engagement of CXCR4 by natural amine and the synthetic mimic of histamine (clobenpropit) strongly inhibits viral-induced production of inflammatory cytokines on primary human peripheral Dendritic Cells (pDC) (Smith N et al., Nature communications, 2017, 8, 14253; WO 2017/216368). In order to identify synthetic compounds with similar properties, known CXCR4 ligands with similar structures were searched. Excitingly, the first co-crystallized structure of CXCR4 was achieved with a small compound called IT1t showing a strong structural homology with clobenpropit (Wu B et al, Science, 2010, 330, 1066-1071). IT1t binds to an allosteric deep pocket that appeared to be distinct from the FDA approved CXCR4 ligand AMD3100 (plerixafor) binding site (Rosenkilde M M et at, J Biol Chem, 2007, 282, 27354-27365; Rosenkilde M M et al, J Biol Chem, 2004, 279, 3033-3041). These pockets were called major for the AMD3100 binding site and minor for IT1t (Wu B et al, Science, 2010, 330, 1066-1071) opening the possibility for distinct biological activity. A combination of structure- and ligand-based virtual screening of the “IT1t pocket” (Mishra R K et at, Scientific reports, 2016, 6, 30155) identified a set of small molecules with agonist or antagonist properties towards CXCL12 demonstrating for the first time the functionality of this pocket. The structural similarity between IT1t and clobenpropit (CB) and molecular modeling prediction support the idea of a common binding site. It has been demonstrated that, as observed with CB, IT1t also controls inflammation in vitro (production of interferons by dendritic cells, NK cells and monocytes) as well as in vivo in models of rheumatoid arthritis and systemic lupus erythematosus (WO 2017/216368). This validates the CXCR4 minor pocket (IT1t binding pocket) as a tool to prevent the production of inflammatory cytokines in contrast to the CXCR4 major pocket of AMD3100 involved in cell migration (Rosenkilde M M et al., J Biol Chem, 2007, 282, 27354-27365; Wu B et at, Science, 2010, 330, 1066-1071). This work therefore clearly demonstrates that CXCR4 minor pocket-targeting molecules constitute a promising therapeutic strategy for inflammatory, autoimmune and autoinflammatory diseases as well as type I interferonopathies.

While various CXCR4 ligands have been reported in the literature (Debnath B et al., Theranostics, 2013, 3, 47-75; Thoma G et al., J Med Chem, 2008, 51(24), 7915-20; Wu B et al., Science, 2010, 330, 1066-1071; Rosenkilde M M et al., J Biol Chem, 2004, 279, 3033-3041; Rosenkilde M M et al., J Biol Chem, 2007, 282, 27354-27365; Mishra R K et al, Scientific reports, 2016, 6, 30155; Mona C E et al, Org Biomol Chem, 2016, 14(43), 10298-10311; Bai R et at, Eur J Med Chem, 2017, 126, 464-475; Mosley C A et al, Expert Opin Ther Pat, 2009, 19(1), 23-38; Smith N et at, Nature communications, 2017, 8, 14253; WO 2017/216368; EP-A-1 431 290), none of them has been shown to inhibit the production of inflammatory cytokines. Furthermore, long term inhibition of the CXCR4-CXCL12 signaling pathway can be highly toxic in vivo. Indeed, experiments with genetically modified animals have indicated that this pathway is essential for B lymphocyte development, maintenance of the hematopoietic stem cell pool in the bone marrow stromal cell niche, cardiac vascular formation, vascularization of the gastrointestinal tract, branching morphology in the pancreas, and cerebellar formation (Tsuchiya A et al., Dig Dis Sci, 2012, 57(11), 2892-2900). Chronic inhibition of the CXCR4-CXCL12 pathway has therefore a high risk of cardio-toxicity, muscle regeneration, neuro-protection or embryonic development disorders as well as increased risk of liver damages (Tsuchiya A et al., Dig Dis Sci, 2012, 57(11), 2892-2900; Li M et al, Trends Neurosci, 2012, 35(10), 619-628; Odemis V et al., Mol Cell Neurosci, 2005, 30(4), 494-505; Cash-Padgett T et al., Neurosci Res, 2016, 105, 75-79). This explains why, although current CXCR4 antagonists can be used via an acute or a chronic low dose administration, long-term high dose administrations have been avoided so far. It could also be extremely detrimental to use a treatment inducing the migration of immune cells, such as monocytes, from the bone marrow to the blood in a context of inflammatory, autoimmune or autoinflammatory disorder where these cells are responsible for the pathology. There is therefore still an unmet need for novel and improved CXCR4 modulators, particularly CXCR4 minor pocket-targeting molecules that block the pathogenic production of inflammatory cytokines while having minimal impact on the CXCR4-CXCL12 signaling, for the therapeutic intervention in inflammatory disorders, autoimmune disorders, autoinflammatory disorders, and interferonopathies.

U.S. Pat. No. 4,349,674 discloses certain quinoxalinyl esters of carbamimidothioic acids. All of the compounds described in this document, however, are based on a quinoxalin-2-yl carbamimidothioate scaffold, in which the quinoxaline group is attached via its 2-position to the sulfur atom of the carbamimidothioate moiety. The compounds of formula (I) according to the present invention, as defined herein below, do not embrace such compounds having a quinoxalin-2-yl group as disclosed in U.S. Pat. No. 4,349,674.

FR 2 201 094 relates to certain 2-[2-(2-methyl-5-nitro-1-imidazolyl)ethyl]isothiourea derivatives, all of which contain a 2-methyl-5-nitro-imidazol-1-yl group attached to a thioethyl moiety. The compounds of formula (I) according to the present invention, however, do not encompass such compounds having a 2-methyl-5-nitro-imidazol-1-yl group as disclosed in FR 2 201 094. Horiuchi J et al., Chem Pharm Bull, 1989, 37(4), 1080-1084 describes the synthesis of certain isothiourea derivatives, including the compound 2-imidazolin-2-yl 2-quinolylmethyl sulfide dihydrochloride (12i). The compounds of formula (I) according to the invention, however, do not embrace such compounds having a quinolin-2-yl group as disclosed in Horiuchi J et al. U.S. Pat. No. 4,205,071 discloses specific isothiourea derivatives containing a heterocyclic group which is attached via a ring nitrogen atom and which is selected from pyrrolidine, piperidine, morpholine, 4-(lower alkyl)-piperazine and hexahydro-1H-azepine. The compounds of formula (I) according to the invention, however, do not encompass such compounds having any of the aforementioned heterocyclic groups attached via a ring nitrogen atom as taught in U.S. Pat. No. 4,205,071.

GB 847,701 relates to certain thioether compounds, all of which contain an unsubstituted or substituted pyridine-N-oxide or quinoline-N-oxide group, which is attached via the 2-position to a sulfur atom of the respective thioether compound, wherein the substituents on the pyridine-N-oxide or quinoline-N-oxide group are alkyl or alkoxy groups containing not more than three carbon atoms, or halogen atoms. In contrast thereto, the compounds of formula (I) according to the invention do not embrace any compounds having such pyridine-N-oxide or quinoline-N-oxide groups attached via the 2-position, as disclosed in GB 847,701.

In the context of the present invention, it has surprisingly been found that the compounds of formula (I) as provided herein are potent inhibitors of the production of interferons and inflammatory cytokines by specifically targeting the CXCR4 minor pocket (IT1t binding pocket) while showing minimal to undetectable impact on the CXCR4-CXCL12 signaling pathway, which renders these compounds highly advantageous for use in therapy, particularly in the treatment or prevention of an inflammatory disorder, an autoimmune disorder, an autoinflammatory disorder, or an interferonopathy, such as, e.g., lupus erythematosus, dermatomyositis or rheumatoid arthritis.

Accordingly, the present invention relates to a compound of the following formula (I)

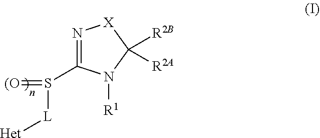

or a pharmaceutically acceptable salt or solvate thereof.

In formula (I), the group X is selected from

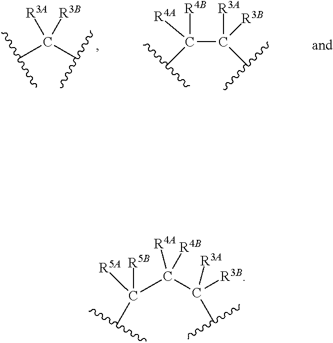

R¹ is selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C₁₋₅ alkyl), and the alkyl moiety in said —COO(C₁₋₅ alkyl) are each optionally substituted with one or more groups R^{Alk}, and further wherein said carbocyclyl and said heterocyclyl are each optionally substituted with one or more groups R^{Cyc}.

R^{2A}, R^{2B}, R^{3A}, R^{3B}, R^{4A}, R^{4B}, R^{5A} and R^{5B} are each independently a group -L²⁰-R²⁰, or alternatively:
R^{2A} and R^{3A} may be mutually joined to form, together with the carbon atoms that they are attached to, a cycloalkyl or heterocycloalkyl, wherein said cycloalkyl or said heterocycloalkyl is optionally substituted with one or more groups R⁶, and/or
R^{3A} and R^{4A} may be mutually joined to form, together with the carbon atoms that they are attached to, a cycloalkyl or heterocycloalkyl, wherein said cycloalkyl or said heterocycloalkyl is optionally substituted with one or more groups R⁶, and/or
R^{4A} and R^{5A} may be mutually joined to form, together with the carbon atoms that they are attached to, a cycloalkyl or heterocycloalkyl, wherein said cycloalkyl or said heterocycloalkyl is optionally substituted with one or more groups R⁶; and
if R^{2A} is not mutually joined with R^{3A}, then R^{2A} and R^{2B} may be mutually joined to form, together with the carbon atom that they are attached to, a cycloalkyl or heterocycloalkyl, wherein said cycloalkyl or said heterocycloalkyl is optionally substituted with one or more groups R⁶, and/or
if R^{3A} is not mutually joined with R^{2A} or R^{4A}, then R^{3A} and R^{3B} may be mutually joined to form, together with the carbon atom that they are attached to, a cycloalkyl or heterocycloalkyl, wherein said cycloalkyl or said heterocycloalkyl is optionally substituted with one or more groups R⁶, and/or
if R^{4A} is not mutually joined with R^{3A} or R^{5A}, then R^{4A} and R^{4B} may be mutually joined to form, together with the carbon atom that they are attached to, a cycloalkyl or heterocycloalkyl, wherein said cycloalkyl or said heterocycloalkyl is optionally substituted with one or more groups R⁶, and/or
if R^{5A} is not mutually joined with R^{4A}, then R^{5A} and R^{5B} may be mutually joined to form, together with the carbon atom that they are attached to, a cycloalkyl or heterocycloalkyl, wherein said cycloalkyl or said heterocycloalkyl is optionally substituted with one or more groups R⁶; and
if R^{2B} is not mutually joined with R^{2A}, and if R^{4B} is not mutually joined with R^{4A}, then R^{2B} and R^{4B} may be mutually joined to form a C₁₋₃ alkylene, wherein said alkylene is optionally substituted with one or more groups R⁶, and wherein one —CH₂— unit comprised in said alkylene is optionally replaced by a group selected from —O—, —NH—, —N(C₁₋₅ alkyl)-, —CO—, —S—, —SO—, and —SO₂—, or
if R^{2B} is not mutually joined with R^{2A}, and if R^{5B} is not mutually joined with R^{5A}, then R^{2B} and R^{5B} may be mutually joined to form a C₁₋₃ alkylene, wherein said alkylene is optionally substituted with one or more groups R⁶, and wherein one —CH₂— unit comprised in said alkylene is optionally replaced by a group selected from —O—, —NH—, —N(C₁₋₅ alkyl)-, —CO—, —S—, —SO—, and —SO₂—, or
if R³ is not mutually joined with R^{3A}, and if R^{5B} is not mutually joined with R^{5A}, then R^{3B} and R^{5B} may be mutually joined to form a C₁₋₃ alkylene, wherein said alkylene is optionally substituted with one or more groups R⁶, and wherein one —CH₂— unit comprised in said alkylene is optionally replaced by a group selected from —O—, —NH—, —N(C₁₋₅ alkyl)-, —CO—, —S—, —SO—, and —SO₂—; and
any remaining groups among R^{2A}, R^{2B}, R^{3A}, R^{3B}, R^{4A}, R^{4B}, R^{5A} and R^{5B}, which are not mutually joined, are each independently a group -L²⁰-R²⁰.

Each R⁶ is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{AC}-R^{AC}.

Each L²⁰ is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —O(C₁₋₅ haloalkyl), —CN, —OR²¹, —NR²¹R²¹, —NR²¹OR²¹, —COR²¹, —COOR²¹, —OCOR²¹, —CONR²¹R²¹, —NR²¹COR²¹, —NR²¹COOR²¹, —OCONR²¹R²¹, —SR²¹, —SOR²¹, —SO₂R²¹, —SO₂NR²¹R²¹, —NR²¹SO₂R²¹, —SO₃R²¹ and —NO₂, and further wherein one or more —CH₂— units comprised in said alkylene, said alkenylene or said alkynylene are each optionally replaced by a group independently selected from —O—, —NR²¹—, —CO—, —S—, —SO—, and —SO₂—.

Each R²⁰ is independently selected from hydrogen, CO₅ alkyl, C₂₋₅ alkenyl, C₂₋₅ alkynyl, halogen, C₁₋₅ haloalkyl, —O(C₁₋₅ haloalkyl), —ON, —OR²², —NR²²R²², —NR²²OR²², —COR²², —COOR²², —OCOR²², —CONR²²R²², —NR²COR²², —NR²²COOR²², —OCONR²²R²², —SR²², —SOR²², —SO₂R²², —SO₂NR²²R²², —NR²²SO₂R²², —SO₃R²², —NO₂, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc}.

Each R²¹ and each R²² is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc}.

Each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{AC}-R^{AC}.

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —ON, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{AC}-R^{AC}.

Each L^{AC} is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{AC} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

n is 0, 1 or 2.

L is a covalent bond or C₁₋₅ alkylene, wherein said alkylene is optionally substituted with one or more groups R^{L}, and further wherein one or more —CH₂— units comprised in said alkylene are each optionally replaced by a group independently selected from cycloalkylene and heterocycloalkylene.

Each R^{L} is independently selected from —OH, —O(C₁₋₅ alkyl), ═O, —SH, —S(C₁₋₅ alkyl), —NH₂, —NH(C₁₋₅ alkyl), —N(C₁₋₅ alkyl)(C₁₋₅ alkyl), halogen, —CF₃, —CN, cycloalkyl, and heterocycloalkyl.

The group Het is a cyclic group selected from any one of the following groups:

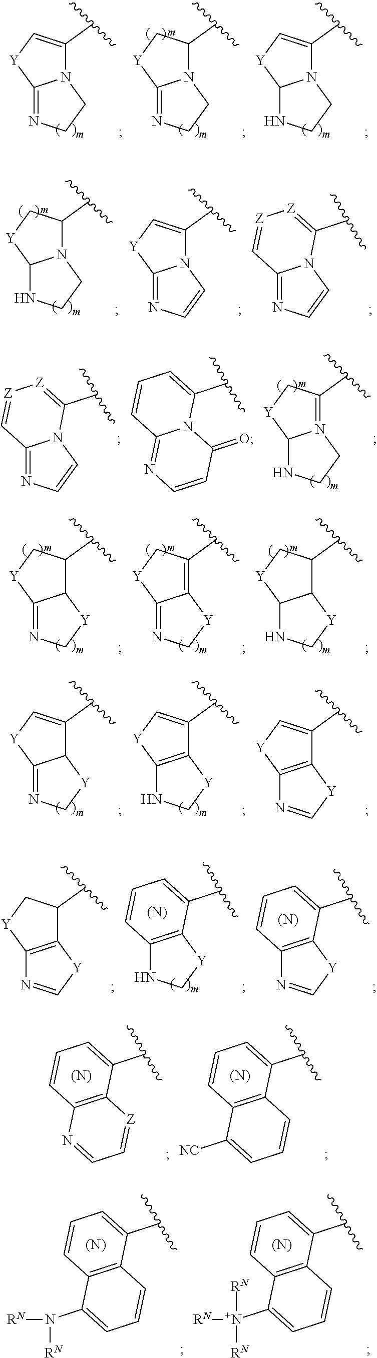

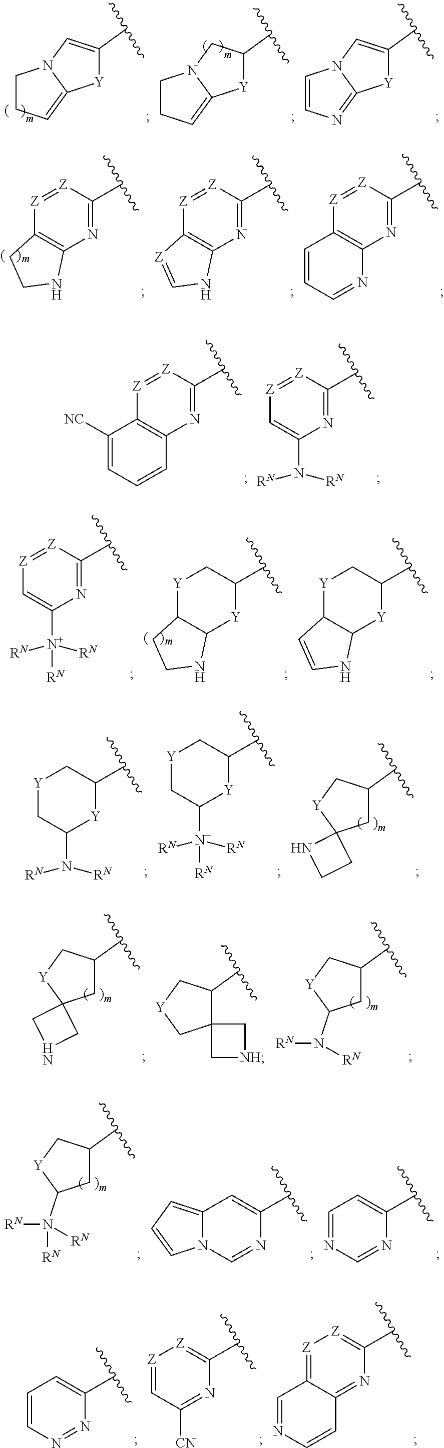

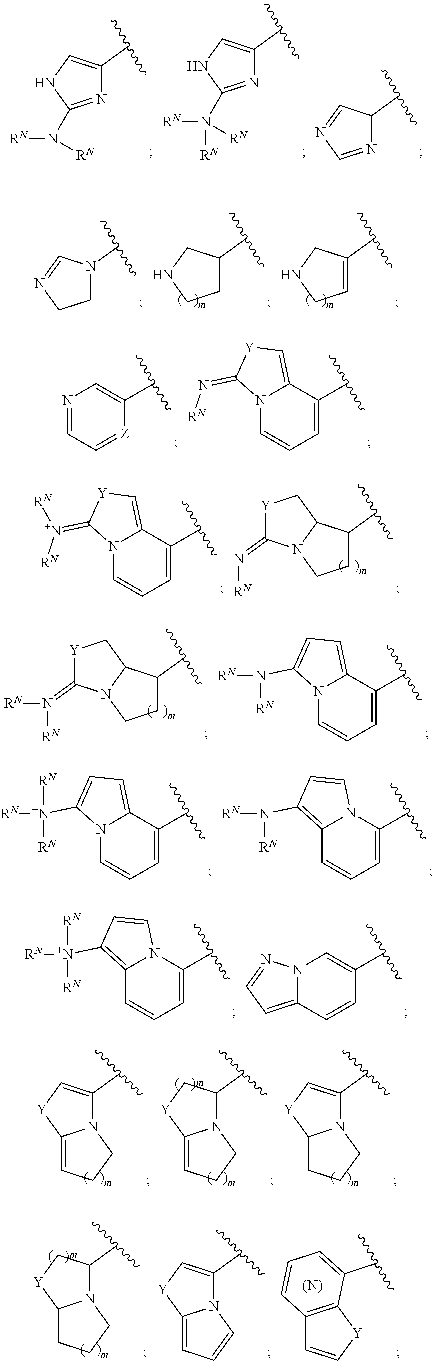

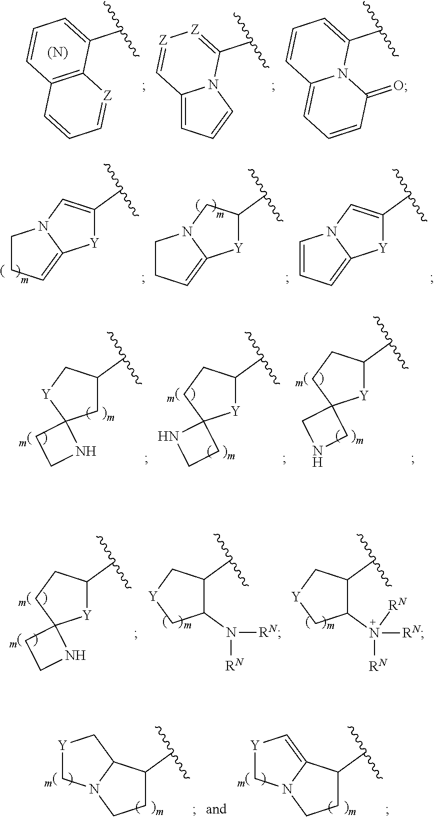

wherein each of the above-depicted groups is optionally substituted with one or more groups R^{Het};
wherein each m is independently 1, 2 or 3;
wherein each ring atom Y is independently selected from S, O, SO₂, NH and CH₂;
wherein each ring atom Z is independently C or N; and
wherein the symbol “(N)” depicted inside a ring indicates that 0, 1, 2 or 3 ring atom(s) of the respective ring is/are nitrogen ring atom(s).

Each R^{N} is independently selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C₁₋₅ alkyl), and the alkyl moiety in said —COO(C₁₋₅ alkyl) are each optionally substituted with one or more groups R^{Alk}, wherein said carbocyclyl and said heterocyclyl are each optionally substituted with one or more groups R^{Cyc}, and further wherein any two groups R^{N} that are attached to the same nitrogen atom may also be mutually joined to form, together with the nitrogen atom that they are attached to, a heterocyclyl which is optionally substituted with one or more groups R^{Cyc}.

Each R^{Het} is independently a group -L^{H1}-R^{H1}; any two groups R^{Het}, which are attached to the same carbon ring atom of Het, may also be mutually joined to form, together with the carbon atom that they are attached to, a cycloalkyl or a heterocycloalkyl, wherein said cycloalkyl or said heterocycloalkyl is optionally substituted with one or more groups R^{Cyc}; and any two groups R^{Het}, which are attached to different ring atoms of Het, may also be mutually joined to form a C₁₋₅ alkylene, wherein said alkylene is optionally substituted with one or more groups R^{Cyc}, and wherein one —CH₂— unit comprised in said alkylene is optionally replaced by a group selected from —O—, —NH—, —N(C₁₋₅ alkyl)-, —CO—, —S—, —SO—, and —SO₂—.

Each L^{H1} is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —O(C₁₋₅ haloalkyl), —ON, —OR^{H2}, —NR^{H2}R^{H2}, —N⁺R^{H2}R^{H2}R^{H2}, —NR^{H2}OR^{H2}, —COR^{H2}, —COOR^{H2}, —OCOR^{H2}, —CONR^{H2}R^{H2}, —NR^{H2}COR^{H2}, —NR^{H2}COOR^{H2}, —OCONR^{H2}R^{H2}, —SR^{H2}, —SOR^{H2}, —SO₂R^{H2}, —SO₂NR^{H2}R^{H2}, —NR^{H2}SO₂R^{H2}, —SO₃R^{H2}, and —NO₂, and further wherein one or more —CH₂— units comprised in said alkylene, said alkenylene or said alkynylene are each optionally replaced by a group independently selected from —O—, —NR^{H2}—, —CO—, —S—, —SO—, and —SO₂—.

Each R^{H1} is independently selected from C₁₋₅ alkyl, C₂₋₅ alkenyl, C₂₋₅ alkynyl, halogen, C₁₋₅ haloalkyl, —O(C₁₋₅ haloalkyl), —CN, —OR^{H3}, —NR^{H3}R^{H3}, —N⁺R^{H3}R^{H3}R^{H3}, —NR^{H3}OR^{H3}, —COR^{H3}, —COOR^{H3}, —OCOR^{H3}, —CONR^{H3}R^{H3}, —NR^{H3}COR^{H3}, —NR^{H3}COOR^{H3}, —OCONR^{H3}R^{H3}, —SR^{H3}, —SOR^{H3}, —SO₂R^{H3}, —SO₂NR^{H3}R^{H3}, —NR^{H3}SO₂R^{H3}, —SO₃R^{H3}, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc}.

Each R^{H2} and each R^{H3} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc}.

Furthermore, in accordance with the present invention, the following compounds are excluded from formula (I):
3-((4,5-dihydro-1H-imidazol-2-ylthio)methyl)-2,3,5,6-tetrahydroimidazo[2,1-b]thiazol-3-ol;
2-(4,5-dihydro-1H-imidazol-2-ylthio)-2,3,5,6-tetrahydroimidazo[2,1-b]thiazole;
3-(4,5-dihydro-1H-imidazol-2-ylthio)-2,3,5,6-tetrahydroimidazo[2,1-b]thiazole;
2-(4,5-dihydro-1H-imidazol-2-ylthio)-1-(2-mercapto-4,5-dihydro-1H-imidazol-1-yl)ethanone;
5-chloro-6-((4,5-dihydro-1H-imidazol-2-ylthio)methyl)pyrimidine-2,4-diol;
5-methyl-6-((4,5-dihydro-1H-imidazol-2-ylthio)methyl)pyrimidine-2,4-diol;
6-(4,5-dihydro-1H-imidazol-2-ylthio)-9H-purine;
2-chloro-6-(4,5-dihydro-1-imidazol-2-ylthio)-9H-purine;
2-amino-6-(4,5-dihydro-1H-imidazol-2-ylthio)-9H-purine;
8-isopropoxy-7-(1-methyl-1H-imidazol-2-ylthio)-5-(1-methyl-4,5-dihydro-1H-imidazol-2-ylthio)quinoline; and
2-(4,5-dihydro-1H-imidazol-2-ylthio)-1-(pyridin-3-yl)ethanone.

The present invention also relates to a pharmaceutical composition comprising a compound of formula (I), or a pharmaceutically acceptable salt or solvate thereof, in combination with a pharmaceutically acceptable excipient. Accordingly, the invention relates to a compound of formula (I) or a pharmaceutically acceptable salt or solvate thereof, or a pharmaceutical composition comprising any of the aforementioned entities and a pharmaceutically acceptable excipient, for use as a medicament.

The invention further relates to a compound of formula (I) or a pharmaceutically acceptable salt or solvate thereof, or a pharmaceutical composition comprising any of the aforementioned entities and a pharmaceutically acceptable excipient, for use in the treatment or prevention of an inflammatory disorder, an autoimmune disorder, an autoinflammatory disorder, or an interferonopathy.

Moreover, the present invention relates to the use of a compound of formula (I) or a pharmaceutically acceptable salt or solvate thereof in the preparation of a medicament for the treatment or prevention of an inflammatory disorder, an autoimmune disorder, an autoinflammatory disorder, or an interferonopathy.

The invention likewise relates to a method of treating or preventing an inflammatory disorder, an autoimmune disorder, an auto inflammatory disorder, or an interferonopathy, the method comprising administering a compound of formula (I) or a pharmaceutically acceptable salt or solvate thereof, or a pharmaceutical composition comprising any of the aforementioned entities in combination with a pharmaceutically acceptable excipient, to a subject (preferably a human) in need thereof. It will be understood that a therapeutically effective amount of the compound of formula (I) or the pharmaceutically acceptable salt or solvate thereof (or of the pharmaceutical composition) is to be administered in accordance with this method.

The diseases/disorders to be treated or prevented in accordance with the present invention, i.e. the inflammatory disorders, autoimmune disorders, autoinflammatory disorders and interferonopathies, include in particular a rheumatologic inflammatory disorder, a skin inflammatory disorder, a lung inflammatory disorder, a muscle inflammatory disorder, a bowel inflammatory disorder, a brain inflammatory disorder, an autoimmune disorder, an autoinflammatory disorder, or a type I interferonopathy.

The interferonopathy (or type I interferonopathy) to be treated or prevented in accordance with the invention is preferably selected from Aicardi-Goutières syndrome, familial chilblain lupus, Singleton-Merten syndrome, proteasome-associated autoinflammatory syndrome, deficiency of adenosine deaminase 2, retinal vasculopathy with cerebral leukodystrophy, STING-associated vasculopathy with onset in infancy, spondyloenchondrodysplasia (e.g., spondyloenchondrodysplasia with immune dysregulation), systemic lupus erythematosus, ISG15 deficiency, or an interferonopathy associated with genetic dysfunction (e.g., an interferonopathy associated with DNASEII deficiency, proteasome deficiency (CANDLE/PRAAS), TREX1 deficiency, IFIH1 gain of function (GOF), STING GOF, DDX58 GOF, CECR1 deficiency, ADAR1 deficiency, RNASEH2 deficiency, RNASET2 deficiency, DNASE1L3 deficiency, complement deficiency (C1Q, C3 and/or C4), ACP5 deficiency, or SAMHD1 deficiency).

The inflammatory disorder, autoimmune disorder or autoinflammatory disorder is preferably selected from familial Mediterranean fever, TNF receptor associated periodic fever syndrome, periodic fever, aphthous stomatitis, pharyngitis, cervical adenitis, pyogenic arthritis, pyoderma gangrenosum, acne, Blau syndrome, neonatal onset multisystem inflammatory disease, familial cold autoinflammatory syndrome, hyperimmunoglobulinemia D with periodic fever syndrome, Muckle-Wells syndrome, chronic infantile neurological cutaneous and articular syndrome, deficiency of interleukin-1 receptor antagonist, haploinsufficiency of A20, deficiency of IL-36 receptor antagonist, CARD14-mediated psoriasis, inflammatory bowel disease (e.g., early-onset inflammatory bowel disease), PLCG2-associated autoinflammation, antibody deficiency and immune dysregulation, an inflammatory disorder associated with genetic dysfunction (e.g., an inflammatory disorder associated with MEFV deficiency, MEFV gain of function (GOF), MFV deficiency, TNFRSF1A GOF, NOD2 GOF, NLRP3 GOF, PSTPIP1 GOF, A20 LOF, IL36RN deficiency, CARD14 GOF, NLRC4 GOF, IL10 RA/RB deficiency, IL-10 deficiency, NOD2 GOF, or PLCG2 GOF), rheumatoid arthritis, spondyloarthritis, osteoarthritis, gout, idiopathic juvenile arthritis, psoriatic arthritis, eczema, psoriasis, scleroderma, systemic lupus erythematosus, Sjögren's syndrome, dermatomyositis, overlapping myositis, mixed connective tissue disease, undifferentiated connective tissue disease, chronic obstructive pulmonary disease, bowel inflammation, Crohn disease, Behçet's disease, ulcerative colitis, sepsis, macrophages activation syndrome, acute respiratory distress syndrome, type II diabetes, asthma, chronic wounds, autism, multiple sclerosis, Alzheimer's disease, Parkinson's disease, chronic inflammatory demyelinating polyneuropathy, juvenile dermatomyositis, or an inflammatory complication associated with a viral infection (e.g., an inflammatory complication associated with Ebola, dengue fever, measles, or meningitis).

Accordingly, the present invention relates, in particular, to the compound of formula (I) or a pharmaceutically acceptable salt or solvate thereof for use in treating or preventing any of the following diseases/disorders: a rheumatologic inflammatory disorder, a skin inflammatory disorder, a lung inflammatory disorder, a muscle inflammatory disorder, a bowel inflammatory disorder, a brain inflammatory disorder, an autoinflammatory disorder, an autoimmune disorder, a type I interferonopathy, Aicardi-Goutières syndrome, familial chilblain lupus, Singleton-Merten syndrome, proteasome-associated autoinflammatory syndrome, deficiency of adenosine deaminase 2, retinal vasculopathy with cerebral leukodystrophy, STING-associated vasculopathy with onset in infancy, spondyloenchondrodysplasia (e.g., spondyloenchondrodysplasia with immune dysregulation), ISG15 deficiency, an interferonopathy associated with genetic dysfunction (e.g., an interferonopathy associated with DNASEII deficiency, proteasome deficiency (CANDLE/PRAAS), TREX1 deficiency, IFIH1 gain of function (GOF), STING GOF, DDX58 GOF, CECR1 deficiency, ADAR1 deficiency, RNASEH2 deficiency, RNASET2 deficiency, DNASE1L3 deficiency, complement deficiency (C1Q, C3 and/or C4), ACP5 deficiency, or SAMHD1 deficiency), familial Mediterranean fever, TNF receptor associated periodic fever syndrome, periodic fever, aphthous stomatitis, pharyngitis, cervical adenitis, pyogenic arthritis, pyoderma gangrenosum, acne, Blau syndrome, neonatal onset multisystem inflammatory disease, familial cold autoinflammatory syndrome, hyperimmunoglobulinemia D with periodic fever syndrome, Muckle-Wells syndrome, chronic infantile neurological cutaneous and articular syndrome, deficiency of interleukin-1 receptor antagonist, haploinsufficiency of A20, deficiency of IL-36 receptor antagonist, CARD14-mediated psoriasis, inflammatory bowel disease (e.g., early-onset inflammatory bowel disease), PLCG2-associated autoinflammation, antibody deficiency and immune dysregulation, an inflammatory disorder associated with genetic dysfunction (e.g., an inflammatory disorder associated with MEFV deficiency, MEFV gain of function (GOF), MFV deficiency, TNFRSF1A GOF, NOD2 GOF, NLRP3 GOF, PSTPIP1 GOF, A20 LOF, IL36RN deficiency, CARD14 GOF, NLRC4 GOF, IL10 RA/RB deficiency, IL-10 deficiency, NOD2 GOF, or PLCG2 GOF), rheumatoid arthritis, spondyloarthritis, osteoarthritis, gout, idiopathic juvenile arthritis, psoriatic arthritis, eczema, psoriasis, scleroderma, systemic lupus erythematosus, Sjögren's syndrome, dermatomyositis, overlapping myositis, mixed connective tissue disease, undifferentiated connective tissue disease, chronic obstructive pulmonary disease, bowel inflammation, Crohn disease, Behçet's disease, ulcerative colitis, sepsis, macrophages activation syndrome, acute respiratory distress syndrome, type II diabetes, asthma, chronic wounds, autism, multiple sclerosis, Alzheimer's disease, Parkinson's disease, chronic inflammatory demyelinating polyneuropathy, juvenile dermatomyositis, or an inflammatory complication associated with a viral infection (e.g., an inflammatory complication associated with Ebola, dengue fever, measles, or meningitis).

Preferably, the invention relates to the compound of formula (I) or a pharmaceutically acceptable salt or solvate thereof for use in treating or preventing any of the following diseases/disorders: Aicardi-Goutières syndrome, familial chilblain lupus, Singleton-Merten syndrome, proteasome-associated autoinflammatory syndrome, deficiency of adenosine deaminase 2, retinal vasculopathy with cerebral leukodystrophy, STING-associated vasculopathy with onset in infancy, spondyloenchondrodysplasia (e.g., spondyloenchondrodysplasia with immune dysregulation), ISG15 deficiency, an interferonopathy associated with genetic dysfunction (e.g., an interferonopathy associated with DNASEII deficiency, proteasome deficiency (CANDLE/PRAAS), TREX1 deficiency, IFIH1 gain of function (GOF), STING GOF, DDX58 GOF, CECR1 deficiency, ADAR1 deficiency, RNASEH2 deficiency, RNASET2 deficiency, DNASE1L3 deficiency, complement deficiency (C1Q, C3 and/or C4), ACP5 deficiency, or SAMHD1 deficiency), familial Mediterranean fever, TNF receptor associated periodic fever syndrome, periodic fever, aphthous stomatitis, pharyngitis, cervical adenitis, pyogenic arthritis, pyoderma gangrenosum, acne, Blau syndrome, neonatal onset multisystem inflammatory disease, familial cold autoinflammatory syndrome, hyperimunoglobulinemia D with periodic fever syndrome, Muckle-Wells syndrome, chronic infantile neurological cutaneous and articular syndrome, deficiency of interleukin-1 receptor antagonist, haploinsufficiency of A20, deficiency of IL-36 receptor antagonist, CARD14-mediated psoriasis, inflammatory bowel disease (e.g., early-onset inflammatory bowel disease), PLCG2-associated autoinflammation, antibody deficiency and immune dysregulation, an inflammatory disorder associated with genetic dysfunction (e.g., an inflammatory disorder associated with MEFV deficiency, MEFV gain of function (GOF), MFV deficiency, TNFRSF1A GOF, NOD2 GOF, NLRP3 GOF, PSTPIP1 GOF, A20 LOF, IL36RN deficiency, CARD14 GOF, NLRC4 GOF, IL10 RA/RB deficiency, IL-10 deficiency, NOD2 GOF, or PLCG2 GOF), rheumatoid arthritis, spondyloarthritis, osteoarthritis, gout, idiopathic juvenile arthritis, psoriatic arthritis, eczema, psoriasis, scleroderma, systemic lupus erythematosus, Sjögren's syndrome, dermatomyositis, overlapping myositis, mixed connective tissue disease, undifferentiated connective tissue disease, chronic obstructive pulmonary disease, bowel inflammation, Crohn disease, Behçet's disease, ulcerative colitis, sepsis, macrophages activation syndrome, acute respiratory distress syndrome, type II diabetes, asthma, chronic wounds, autism, multiple sclerosis, Alzheimer's disease, Parkinson's disease, chronic inflammatory demyelinating polyneuropathy, juvenile dermatomyositis, or an inflammatory complication associated with a viral infection (e.g., an inflammatory complication associated with Ebola, dengue fever, measles, or meningitis).

More preferably, the present invention relates to the compound of formula (I) or a pharmaceutically acceptable salt or solvate thereof for use in treating or preventing rheumatoid arthritis, dermatomyositis (e.g., juvenile dermatomyositis), or systemic lupus erythematosus.

The present invention furthermore relates to the use of a compound of formula (I) or a pharmaceutically acceptable salt or solvate thereof as a C—X—C chemokine receptor type 4 (CXCR4) modulator in research, particularly as a research tool compound for modulating CXCR4. Accordingly, the invention refers to the in vitro use of a compound of formula (I) or a pharmaceutically acceptable salt or solvate thereof as a CXCR4 modulator and, in particular, to the in vitro use of a compound of formula (I) or a pharmaceutically acceptable salt or solvate thereof as a research tool compound acting as a CXCR4 modulator. The invention likewise relates to a method, particularly an in vitro method, of modulating CXCR4, the method comprising the application of a compound of formula (I) or a pharmaceutically acceptable salt or solvate thereof. The invention further relates to a method of modulating CXCR4, the method comprising applying a compound of formula (I) or a pharmaceutically acceptable salt or solvate thereof to a test sample (e.g., a biological sample) or a test animal (i.e., a non-human test animal). The invention also refers to a method, particularly an in vitro method, of modulating CXCR4 in a sample (e.g., a biological sample), the method comprising applying a compound of formula (I) or a pharmaceutically acceptable salt or solvate thereof to said sample. The present invention further provides a method of modulating CXCR4, the method comprising contacting a test sample (e.g., a biological sample) or a test animal (i.e., a non-human test animal) with a compound of formula (I) or a pharmaceutically acceptable salt or solvate thereof. The terms “sample”, “test sample” and “biological sample” include, without being limited thereto: a cell, a cell culture or a cellular or subcellular extract; biopsied material obtained from an animal (e.g., a human), or an extract thereof; or blood, serum, plasma, saliva, urine, feces, or any other body fluid, or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The compound of formula (I) as well as the pharmaceutically acceptable salts and solvates thereof will be described in more detail in the following.

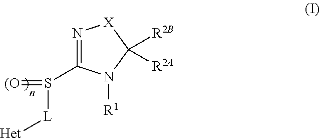

In formula (I), the group X is selected from

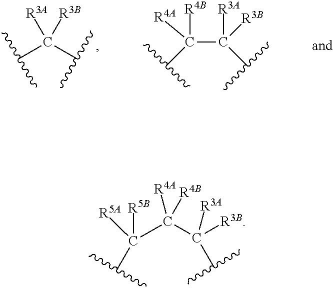

Accordingly, depending on the meaning of X, the compound of formula (I) may have any of the following structures:

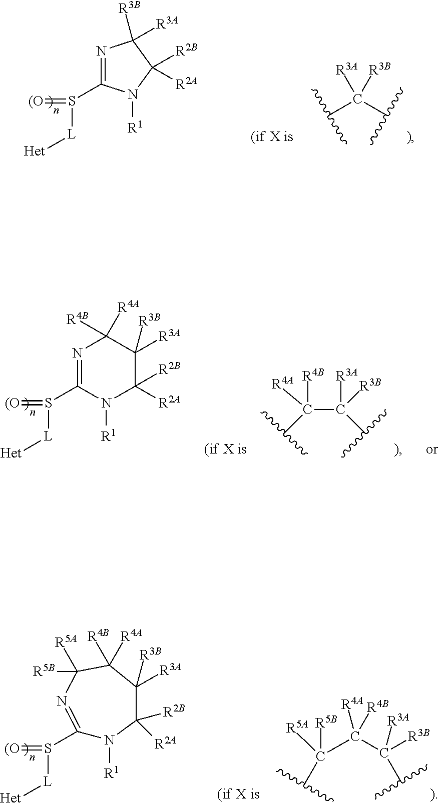

Preferably, X is

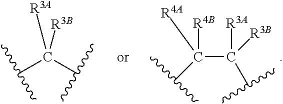

More preferably, X is

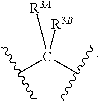

R¹ is selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C %₅ alkyl), and the alkyl moiety in said —COO(C₁₋₅ alkyl) are each optionally substituted with one or more (e.g., one, two or three) groups R^{Alk}, and further wherein said carbocyclyl and said heterocyclyl are each optionally substituted with one or more (e.g., one, two or three) groups R^{Cyc}.

Preferably, R¹ is selected from hydrogen, C₁₋₅ alkyl, —CO(C₁₋₅ alkyl), carbocyclyl, and heterocyclyl, wherein said alkyl and the alkyl moiety in said —CO(C₁₋₅ alkyl) are each optionally substituted with one or more (e.g., one, two or three) groups R^{Alk}, and further wherein said carbocyclyl and said heterocyclyl are each optionally substituted with one or more (e.g., one, two or three) groups R^{Cyc}. More preferably, R¹ is selected from hydrogen, C₁₋₅ alkyl, and cycloalkyl (e.g., cyclopropyl, cyclopentyl, or cyclohexyl), wherein said cycloalkyl is optionally substituted with one or more groups R^{Cyc}. Even more preferably, R¹ is hydrogen, C₁₋₅ alkyl (e.g., methyl or ethyl), or cyclohexyl. Yet even more preferably, R¹ is hydrogen or methyl. Yet even more preferably, R¹ is hydrogen.

R^{2A}, R^{2B}, R^{3A}, R^{3B}, R^{4A}, R^{4B}, R^{5A} and R^{5B} are each independently a group -L²⁰-R²⁰, or alternatively:
R^{2A} and R^{3A} may be mutually joined to form, together with the carbon atoms that they are attached to, a cycloalkyl or heterocycloalkyl, wherein said cycloalkyl or said heterocycloalkyl is optionally substituted with one or more (e.g., one, two or three) groups R⁶, and/or
R^{3A} and R^{4A} may be mutually joined to form, together with the carbon atoms that they are attached to, a cycloalkyl or heterocycloalkyl, wherein said cycloalkyl or said heterocycloalkyl is optionally substituted with one or more (e.g., one, two or three) groups R⁶, and/or
R^{4A} and R^{5A} may be mutually joined to form, together with the carbon atoms that they are attached to, a cycloalkyl or heterocycloalkyl, wherein said cycloalkyl or said heterocycloalkyl is optionally substituted with one or more (e.g., one, two or three) groups R⁶; and
if R^{2A} is not mutually joined with R^{3A}, then R^{2A} and R^{2B} may be mutually joined to form, together with the carbon atom that they are attached to, a cycloalkyl or heterocycloalkyl, wherein said cycloalkyl or said heterocycloalkyl is optionally substituted with one or more (e.g., one, two or three) groups R⁶, and/or
if R^{3A} is not mutually joined with R^{2A} or R^{4A}, then R^{3A} and R^{3B} may be mutually joined to form, together with the carbon atom that they are attached to, a cycloalkyl or heterocycloalkyl, wherein said cycloalkyl or said heterocycloalkyl is optionally substituted with one or more (e.g., one, two or three) groups R⁶, and/or
if R^{4A} is not mutually joined with R^{3A} or R^{5A}, then R^{4A} and R^{4B} may be mutually joined to form, together with the carbon atom that they are attached to, a cycloalkyl or heterocycloalkyl, wherein said cycloalkyl or said heterocycloalkyl is optionally substituted with one or more (e.g., one, two or three) groups R⁶, and/or
if R^{5A} is not mutually joined with R^{4A}, then R^{5A} and R^{5B} may be mutually joined to form, together with the carbon atom that they are attached to, a cycloalkyl or heterocycloalkyl, wherein said cycloalkyl or said heterocycloalkyl is optionally substituted with one or more (e.g., one, two or three) groups R⁶; and
if R^{2B} is not mutually joined with R^{2A}, and if R^{4B} is not mutually joined with R^{4A}, then R^{2B} and R^{4B} may be mutually joined to form a C₁₋₃ alkylene, wherein said alkylene is optionally substituted with one or more (e.g., one, two or three) groups R⁶, and wherein one —CH₂— unit comprised in said alkylene is optionally replaced by a group selected from —O—, —NH—, —N(C₁₋₅ alkyl)-, —CO—, —S—, —SO—, and —SO₂—, or
if R^{2B} is not mutually joined with R^{2A}, and if R^{5B} is not mutually joined with R^{5A}, then R^{2B} and R^{5B} may be mutually joined to form a C₁₋₃ alkylene, wherein said alkylene is optionally substituted with one or more (e.g., one, two or three) groups R⁶, and wherein one —CH₂— unit comprised in said alkylene is optionally replaced by a group selected from —O—, —NH—, —N(C₁₋₅ alkyl)-, —CO—, —S—, —SO—, and —SO₂—, or
if R^{3B} is not mutually joined with R^{3A}, and if R^{5B} is not mutually joined with R^{5A}, then R^{3B} and R^{5B} may be mutually joined to form a C₁₋₃ alkylene, wherein said alkylene is optionally substituted with one or more (e.g., one, two or three) groups R⁶, and wherein one —CH₂— unit comprised in said alkylene is optionally replaced by a group selected from —O—, —NH—, —N(C₁₋₅ alkyl)-, —CO—, —S—, —SO—, and —SO₂—; and
any remaining groups among R^{2A}, R^{2B}, R^{3A}, R^{3B}, R^{4A}, R^{4B}, R^{5A} and R^{5B}, which are not mutually joined, are each independently a group -L²⁰-R²⁰.

It will be understood that if R^{2A} and R^{3A} are mutually joined (as described above), the ring formed from R^{2A} and R^{3A} will be fused to the heterocycle containing the ring atom N(—R¹). The two carbon atoms carrying R^{2A} and R^{3A}, respectively, will thus be common to (i.e., shared by) the ring formed from R^{2A} and R^{3A} and the heterocycle containing the ring atom N(—R¹).

Thus, for example, if X is

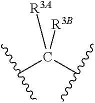

and if R^{2A} and R^{3A} are mutually joined to form a cyclohexyl, then the resulting compound will have the following structure:

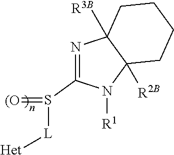

The two stereocenters of the cyclohexyl moiety in the above-depicted compound may each independently have the R-configuration or the S-configuration, and preferably have the following absolute configuration:

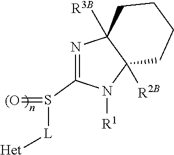

The cycloalkyl or heterocycloalkyl ring formed from R^{2A} and R^{3A} may be, e.g., a monocyclic ring (e.g., a cyclohexyl, as in the example illustrated above), a bridged polycyclic ring (e.g., a bridged bicyclic ring, such as a norbornanyl, a quinuclidinyl or a nortropanyl), or a fused polycyclic ring (e.g., a fused bicyclic ring, such as a decalinyl or a decahydroquinolinyl).

For example, if X is

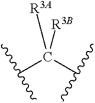

and if R^{2A} and R^{3A} are mutually joined to form a norbornanyl, then the resulting compound will have the following structure:

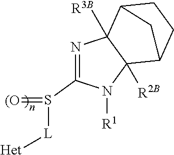

Moreover, it will be understood that if R^{2A} and R^{3A} are mutually joined and if additionally R^{3A} and R^{4A} are mutually joined (as described above), the ring formed from R^{2A} and R^{3A} can either be fused to, or separate from, the ring formed from R^{3A} and R^{4A}.

Thus, for example, if X is

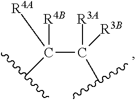

if R^{2A} and R^{3A} are mutually joined to form a cyclohexyl, and if R^{3A} and R^{4A} are mutually joined to form a cyclopentyl, then the resulting compound can have any of the following structures:

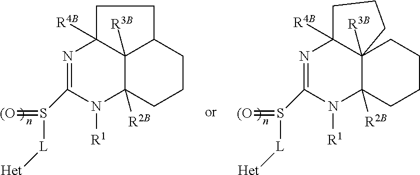

Likewise, it will be understood that if R^{3A} and R^{4A} are mutually joined and additionally R^{4A} and R^{5A} are mutually joined (as described above), then the respective rings (i.e., the ring formed from R^{3A} and R^{4A} and the ring formed from R^{4A} and R^{5A}) can either be fused or separate.

It will further be understood that if R^{2A} and R^{2B} are mutually joined (as described above), then the ring formed from R^{2A} and R^{2B} will form a spiro ring system with the heterocycle containing the ring atom N(—R¹). The ring formed from R^{2A} and R^{2B} and the heterocycle containing the ring atom N(—R¹) will thus have one ring atom in common, i.e. the carbon ring atom carrying R^{2A} and R^{2B}.

For example, if X is

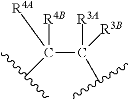

and if R^{3A} and R^{3B} are mutually joined to form a cyclopropyl, then the resulting compound will have the following structure:

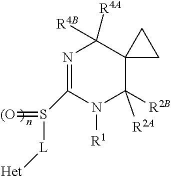

It will further be understood that if R^{2B} and R^{4B} are mutually joined to form a C₁₋₃ alkylene (which is optionally substituted with one or more R⁶, and wherein one —CH₂— unit is optionally replaced by —O—, —NH—, etc., as described above), then this alkylene group will form a bridge over the corresponding ring carbon atoms carrying R^{2B} and R^{4B}.

Thus, for example, if X is

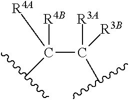

and if R^{2B} and R^{4B} are mutually joined to form a methylene (—CH₂—), then the resulting compound will have the following structure:

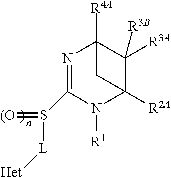

Likewise, if R^{2B} and R^{5B} are mutually joined to form a C₁₋₃ alkylene, then this alkylene group will form a bridge over the corresponding ring carbon atoms carrying R^{2B} and R^{5B}. If R^{3B} and R^{5B} are mutually joined to form a C₁₋₃ alkylene, then this alkylene group will form a bridge over the corresponding ring carbon atoms carrying R^{3B} and R^{5B}.

For example, if X is

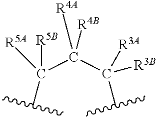

and if R^{2B} and R^{5B} are mutually joined to form a group —CH₂CH₂—, then the resulting compound will have the following structure:

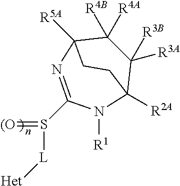

Moreover, it will be understood that the groups R^{4A}, R^{4B}, R^{5A} and R^{5B} can be present or absent, depending on the meaning of the group X, and that the above definitions of these groups (R^{4A}, R^{4B}, R^{5A} and R^{5B}) are relevant only if the respective groups are present. Therefore, unless X is limited to a moiety that requires the presence of R^{4A} and R^{4B} or of R^{4A}, R^{4B}, R^{6A} and R^{5B}, any reference to any of the groups “R^{4A}”, “R^{4B}”, “R^{5A}” and “R^{5B}” can also be expressed as “R^{4A} (if present)”, “R^{4B} (if present)”, “R^{5A} (if present)” and “R^{5B} (if present)”, respectively. The term “if present” is omitted herein solely for ease of legibility.

As described above, the groups R^{2A} and R^{3A}, the groups R^{3A} and R^{4A}, and/or the groups R^{4A} and R^{5A} may be mutually joined to form, together with the respective carbon atoms that they are attached to, a cycloalkyl or heterocycloalkyl, wherein said cycloalkyl or said heterocycloalkyl is optionally substituted with one or more groups R⁶. It is preferred that the cycloalkyl or heterocycloalkyl which is formed from any of the aforementioned pairs of groups (i.e., from R^{2A} and R^{3A}, from R^{3A} and R^{4A}, and/or from R^{4A} and R^{5A}), and which is optionally substituted with one or more R⁶, has 5 to 14 ring members, more preferably 5 to 10 ring members. Moreover, it is preferred that said cycloalkyl or said heterocycloalkyl is monocyclic, bridged polycyclic (e.g., bridged bicyclic), or fused polycyclic (e.g., fused bicyclic), more preferably said cycloalkyl or said heterocycloalkyl is monocyclic or bridged bicyclic. It is particularly preferred that the cycloalkyl which is formed from any of the aforementioned pairs of groups (i.e., from R^{2A} and R^{3A}, from R^{3A} and R^{4A}, and/or from R^{4A} and R^{5A} optionally substituted with one or more R⁶, is a monocyclic C₅₋₇ cycloalkyl (e.g., cyclopentyl or cyclohexyl) or a bicyclic bridged C₇₋₁₀ cycloalkyl (e.g., norbornanyl or adamantyl). It is further particularly preferred that the heterocycloalkyl which is formed from any of the aforementioned pairs of groups, and which is optionally substituted with one or more R⁶, is a monocyclic 5 to 7-membered heterocycloalkyl (e.g., piperidinyl) or a bicyclic bridged 7 to 10-membered heterocycloalkyl (e.g., quinuclidinyl or nortropanyl).

As also described above, the groups R^{2A} and R^{2B}, the groups R^{3A} and R^{3B}, the groups R^{4A} and R^{4B}, and/or the groups R^{5A} and R^{5B}, may be mutually joined to form, together with the respective carbon atom that they are attached to, a cycloalkyl or heterocycloalkyl, wherein said cycloalkyl or said heterocycloalkyl is optionally substituted with one or more groups R⁶. It is preferred that the cycloalkyl or heterocycloalkyl which is formed from any of the aforementioned pairs of groups (i.e., from R^{2A} and R^{2B}, from R^{3A} and R^{3B}, from R^{4A} and R^{4B}, and/or from R^{5A} and R^{5B}), and which is optionally substituted with one or more R⁶, has 3 to 8 ring members, more preferably 3, 4, 5 or 6 ring members. Moreover, it is preferred that said cycloalkyl or said heterocycloalkyl is monocyclic. Accordingly, it is particularly preferred that the cycloalkyl which is formed from any of the aforementioned pairs of groups (i.e., from R^{2A} and R^{2B}, from R^{3A} and R^{3B}, from R^{4A} and R^{4B}, and/or from R^{5A} and R^{5B}), and which is optionally substituted with one or more R⁶, is a monocyclic C₃₋₈ cycloalkyl, more preferably a monocyclic C₃₋₅ cycloalkyl (e.g., cyclopropyl). It is furthermore particularly preferred that the heterocycloalkyl which is formed from any of the aforementioned pairs of groups, and which is optionally substituted with one or more R⁶, is a monocyclic 3 to 8-membered heterocycloalkyl, more preferably a monocyclic 4 to 6-membered heterocycloalkyl (e.g., tetrahydrofuranyl).

As described above, the groups R^{2B} and R^{4B}, or the groups R^{2B} and R^{5B}, or the groups R^{3B} and R^{5B} may be mutually joined to form a C₁₋₃ alkylene, wherein said alkylene is optionally substituted with one or more (e.g., one, two or three) groups R⁶, and wherein one —CH₂— unit comprised in said alkylene is optionally replaced by a group selected from —O—, —NH—, —N(C₁₋₅ alkyl)-, —CO—, —S—, —SO—, and —SO₂—. It is preferred that said alkylene is not substituted with any groups R⁶, and that one —CH₂— unit comprised in said alkylene is optionally replaced by a group selected from —O—, —NH—, —N(C₁₋₅ alkyl)-, —CO—, and —S—, more preferably from —O—, —NH—, and —N(C₁₋₅ alkyl)-. Moreover, said C₁₋₃ alkylene (i.e., the C₁₋₃ alkylene formed from R^{2B} and R^{4B}, or from R^{2B} and R^{5B}, or from R^{3B} and R^{5B}) is preferably selected from —CH₂—, —CH₂CH₂— and —C_{H}C₂CH₂—. More preferably, said alkylene is —CH₂— or —CH₂CH₂—.

Each R⁶ is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C %₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂(C₁₋₅ alkyl), —SO₂—(C₁₋₅ alkyl), —SO—(C₁₋₅ alkyl), aryl, heteroaryl, cycloalkyl, heterocycloalkyl, and -L^{AC}-R^{AC}.

Preferably, each R⁶ is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), and —SO—(C₁₋₅ alkyl). More preferably, each R⁶ is independently selected from C₁₋₅ alkyl, —OH, —O(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), and —CN.

Each L²⁰ is independently selected from a bond, C₁₋₅ alkylene, C₂₋₅ alkenylene, and C₂₋₅ alkynylene, wherein said alkylene, said alkenylene and said alkynylene are each optionally substituted with one or more (e.g., one, two, or three) groups independently selected from halogen, C₁₋₅ haloalkyl, —O(C₁₋₅ haloalkyl), —CN, —R²¹, —NR²¹R²¹, —NR²¹R²¹, —COR²¹, —COR²¹, —OCR²¹, —CONR²¹R²¹, —NR²¹COR²¹, —NR¹COOR²¹, —OCONR²¹R²¹, —SR²¹, —SR²¹, —SO₂R²¹, —SO₂NR²¹R²¹, —NR²¹SO₂R²¹, —SO₃R²¹, and —NO₂, and further wherein one or more (e.g., one, two, or three) —CH₂— units comprised in said alkylene, said alkenylene or said alkynylene are each optionally replaced by a group independently selected from —O—, —NR²¹—, —CO—, —S—, —SO—, and —SO₂—.

Preferably, each L²⁰ is independently selected from a bond and C₁₋₅ alkylene, wherein said alkylene is optionally substituted with one or more (e.g., one, two, or three) groups independently selected from halogen, C₁₋₅ haloalkyl, —O(C₁₋₅ haloalkyl), —CN, —OR²¹, —NR²¹R²¹, —NR²¹OR²¹, —COR²¹, —COOR²¹, —OCR²¹, —CONR²¹R²¹, —NR²¹COR²¹, —NR²¹COOR²¹, —OCONR²¹R²¹, —SR²¹, —SOR²¹, —SO₂R²¹, —SO₂NR²¹R²¹, —NR²¹R²¹, —SO₃R²¹, and —NO₂, and further wherein one or more (e.g., one, two, or three) —CH₂— units comprised in said alkylene are each optionally replaced by a group independently selected from —O—, —NR²¹—, —CO—, —S—, —SO—, and —SO₂—. More preferably, each L²⁰ is independently selected from a bond and C₁₋₅ alkylene, wherein said alkylene is optionally substituted with one or more groups independently selected from halogen, C₁₋₅ haloalkyl, —O(C₁₋₅ haloalkyl), —CN, —OH, —O(C₁₋₅ alkyl), —NH₂, —NH(C₁₋₅ alkyl), and —N(C₁₋₅ alkyl)(C₁₋₅ alkyl), and further wherein one or two —CH₂— units comprised in said alkylene is/are each optionally replaced by a group independently selected from —O—, —NH—, —N(C₁₋₅ alkyl)-, —CO—, and —SO₂—. Even more preferably, each L²⁰ is independently selected from a bond and C₁₋₅ alkylene. Yet even more preferably, each L²⁰ is independently selected from a bond, methylene, ethylene, and propylene. Still more preferably, L²⁰ is a bond.

Each R²⁰ is independently selected from hydrogen, C₁₋₅ alkyl, C₂₋₅ alkenyl, C₂₋₅ alkynyl, halogen, C₁₋₅ haloalkyl, —(C₁₋₅ haloalkyl), —ON, —OR²², —NR²²R²², —NR²²OR²², —CR²², —COOR²², —OCOR²², —CONR²²R²², —NR²²COR²², —NR²²COOR²², —OCONR²²R²², —SR²², —SOR²², —SO₂R²², —SO₂NR²²R²², —NR²²SO₂R²², —SO₃R²², —NO₂, carbocyclyl, and heterocyclyl, wherein said alkyl, said alkenyl and said alkynyl are each optionally substituted with one or more (e.g., one, two or three) groups R^{Alk}, and further wherein said carbocyclyl and said heterocyclyl are each optionally substituted with one or more (e.g., one, two or three) groups R^{Cyc}.

Preferably, each R²⁰ is independently selected from hydrogen, C₁₋₅ alkyl, C₂₋₅ alkenyl, C₂₋₅ alkynyl, halogen, C₁₋₅ haloalkyl, —O(C₁₋₅ haloalkyl), —ON, —OR²², —NR²²R²², —COR²², —COOR²², —OOR²², —CONR²²R²², —NR²²R²², —SR²², —SR²², —SO₂R²², —SO₂NR²²R²², —NR²²SO₂R²², cycloalkyl, aryl, heterocycloalkyl, and heteroaryl, wherein said alkyl, said alkenyl and said alkynyl are each optionally substituted with one or more (e.g., one, two or three) groups R^{Alk}, and further wherein said cycloalkyl, said aryl, said heterocycloalkyl and said heteroaryl are each optionally substituted with one or more (e.g., one, two or three) groups R^{Cyc}. More preferably, each R²⁰ is independently selected from hydrogen, C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —O(C₁₋₅ alkylene)-OH, —O(C₁₋₅ alkylene)-O(C₁₋₅ alkyl), —NH₂, —NH(C₁₋₅ alkyl), —N(C₁₋₅ alkyl)(C₁₋₅ alkyl), —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —SH, —S(C₁₋₅ alkyl), —SO—(C₁₋₅ alkyl), —SO₂—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}. Even more preferably, each R²⁰ is independently selected from hydrogen, C₁₋₅ alkyl (e.g., butyl or pentyl), halogen, C₁₋₅ haloalkyl (e.g., —CF₃), —O(C₁₋₅ haloalkyl), —ON, —OH, —O(C₁₋₅ alkyl), —NH₂, —NH(C₁₋₅ alkyl), —N(C₁₋₅ alkyl)(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}. Yet even more preferably, each R²⁰ is independently selected from hydrogen, butyl, pentyl, cycloalkyl, aryl, heterocycloalkyl, and heteroaryl, wherein said cycloalkyl, said aryl, said heterocycloalkyl and said heteroaryl are each optionally substituted with one or more groups R^{Cyc}. Still more preferably, each R²⁰ is independently hydrogen, aryl, or heteroaryl, wherein said aryl and said heteroaryl are each optionally substituted with one or more groups R^{Cyc}.

In accordance with the above definitions of L²⁰ and R²⁰, it is particularly preferred that each group -L²⁰-R²⁰ is independently selected from hydrogen, C₁₋₅ alkyl, C₂₋₅ alkenyl, C₂₋₅ alkynyl, halogen, C₁₋₅ haloalkyl, —(C₁₋₅ alkylene)-O(C₁₋₅ haloalkyl), —(C₀₋₅ alkylene)-CN, —(C₀₋₅ alkylene)-OH, —(C₀₋₅ alkylene)-O(C₁₋₅ alkyl), —(C₀₋₅ alkylene)-O(C₁₋₅ alkylene)-OH, —(C₀₋₅ alkylene)-O(C₁₋₅ alkylene)-O(C₁₋₅ alkyl), —(C₀₋₅ alkylene)-NH₂, —(C₀₋₅ alkylene)-NH(C₁₋₅ alkyl), —(C₀₋₅ alkylene)-N(C₁₋₅ alkyl)(C₁₋₅ alkyl), —(C₀₋₅ alkylene)-CHO, —(C₀₋₅ alkylene)-CO(C₁₋₅ alkyl), —(C₀₋₅ alkylene)-COOH, —(C₀₋₅ alkylene)-COO(C₁₋₅ alkyl), —(C₀₋₅ alkylene)-O—CO(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₀₋₅ alkylene)-NH—CO(C₁₋₅ alkyl), —(C₀₋₅ alkylene)-N(C₁₋₅ alkyl)-CO(C₁₋₅ alkyl), —(C₀₋₅ alkylene)-SH, —(C₀₋₅ alkylene)-S(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₀₋₅ alkylene)-NH—SO₂—(C₁₋₅ alkyl), —(C₀₋₅ alkylene)-N(C₁₋₅ alkyl)-SO₂—(C₁₋₅ alkyl), —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein the cycloalkyl moiety in said —(C₀₋₅ alkylene)-cycloalkyl, the aryl moiety in said —(C₀₋₅ alkylene)-aryl, the heterocycloalkyl moiety in said —(C₁₋₅ alkylene)-heterocycloalkyl, and the heteroaryl moiety in said —(C₀₋₅ alkylene)-heteroaryl are each optionally substituted with one or more groups R^{Cyc}. Even more preferably, each group -L²⁰-R²⁰ is independently selected from hydrogen, C₁₋₅ alkyl (e.g., butyl or pentyl), halogen, C₁₋₅ haloalkyl (e.g., —CF₃), —(C₀₋₃ alkylene)-O(C₁₋₅ haloalkyl) (e.g., —OCF₃), —(C₀₋₃ alkylene)-CN, —(C₀₋₃ alkylene)-OH, —(C₀₋₃ alkylene)-O(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-cycloalkyl, —(C₀₋₃ alkylene)-aryl, —(C₀₋₃ alkylene)-heterocycloalkyl, and —(C₀₋₃ alkylene)-heteroaryl, wherein the cycloalkyl moiety in said —(C₀₋₃ alkylene)-cycloalkyl, the aryl moiety in said —(C₀₋₃ alkylene)-aryl, the heterocycloalkyl moiety in said —(C₀₋₃ alkylene)-heterocycloalkyl, and the heteroaryl moiety in said —(C₀₋₃ alkylene)-heteroaryl are each optionally substituted with one or more groups R^{Cyc}. Yet even more preferably, each group -L²⁰-R²⁰ is independently selected from hydrogen, butyl, pentyl, —(C₀₋₃ alkylene)-cycloalkyl, —(C₀₋₃ alkylene)-aryl, —(C₀₋₃ alkylene)-heterocycloalkyl, and —(C₀₋₃ alkylene)-heteroaryl, wherein the cycloalkyl moiety in said —(C₀₋₃ alkylene)-cycloalkyl, the aryl moiety in said —(C₀₋₃ alkylene)-aryl, the heterocycloalkyl moiety in said —(C₀₋₃ alkylene)-heterocycloalkyl, and the heteroaryl moiety in said —(C₀₋₃ alkylene)-heteroaryl are each optionally substituted with one or more groups R^{Cyc}. Still more preferably, each group -L²⁰-R²⁰ is independently selected from hydrogen, —(C₀₋₃ alkylene)-aryl and —(C₀₋₃ alkylene)-heteroaryl, wherein the aryl moiety in said —(C₀₋₃ alkylene)-aryl and the heteroaryl moiety in said —(C₀₋₃ alkylene)-heteroaryl are each optionally substituted with one or more groups R^{Cyc}. In relation to each one of the aforementioned preferred definitions of the group -L²⁰-R²⁰, it is furthermore preferred that at most one or two groups -L²⁰-R²⁰ is/are different from hydrogen, and it is even more preferred that at most one group -L²⁰-R²⁰ is different from hydrogen (while any other groups -L²⁰-R²⁰, which may be present in the compound of formula (I), are hydrogen). Specific examples of preferred groups -L²⁰-R²⁰ include, in particular, n-butyl, cyclohexyl, —(C₀₋₃ alkylene)-phenyl (e.g., phenyl or benzyl), —(C₀₋₃ alkylene)-phenyl-halogen (e.g., 4-chlorophenyl or 4-chlorobenzyl), or —(C₀₋₃ alkylene)-imidazolyl (e.g., 3-(imidazol-5-yl)propyl).

Each R²¹ and each R²² is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more (e.g., one, two or three) groups R^{Alk}, and further wherein said carbocyclyl and said heterocyclyl are each optionally substituted with one or more (e.g., one, two or three) groups R^{Cyc}.

Preferably, each R²¹ and each R²² is independently selected from hydrogen and C₁₋₅ alkyl, wherein said alkyl is optionally substituted with one or more (e.g., one, two or three) groups R^{Alk}. More preferably, each R²¹ and each R²² is independently selected from hydrogen and C₁₋₅ alkyl (e.g., methyl or ethyl).

Each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{AC}-R^{AC}.

Preferably, each R^{Alk} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —ON, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl More preferably, each R^{Alk} is independently selected from —OH, —O(C₁₋₅ alkyl), —NH₂, —NH(C₁₋₅ alkyl), —N(C₁₋₅ alkyl)(C₁₋₅ alkyl), halogen, C₁-5 haloalkyl, —O(C₁₋₅ haloalkyl), and —CN.

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{AC}-R^{AC}.

Preferably, each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl. More preferably, each R^{Cyc} is independently selected from C₁₋₅ alkyl, —OH, —O(C₁₋₅ alkyl), —NH₂, —NH(C₁₋₅ alkyl), —N(C₁₋₅ alkyl)(C₁₋₅ alkyl), halogen, C₁₋₅ haloalkyl, —O(C₁₋₅ haloalkyl), and —CN.

Each L^{AC} is independently selected from a bond, C₁₋₅ alkylene, C₂₋₅ alkenylene, and C₂₋₅ alkynylene, wherein said alkylene, said alkenylene and said alkynylene are each optionally substituted with one or more (e.g., one, two, or three) groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more (e.g., one, two, or three) —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{AC} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COO H, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more (e.g., one, two or three) groups independently selected from C₁₋₅ alkyl, C₂₋₅ alkenyl, C₂₋₅ alkynyl, halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

n is 0, 1 or 2. Preferably, n is 0.

It will be understood that the variable n indicates the number of ═O groups attached to the sulfur atom in the corresponding group —S(═O)ₙ— in the compound of formula (I). Thus, if n is 0, then the group —S(═O)ₙ— is a group —S—. If n is 1, then the group —S(═O)ₙ— is a group —SO—. If n is 2, then the group —S(═O)ₙ— is a group —SO₂—.

L is a covalent bond or C₁₋₅ alkylene, wherein said C₁₋₅ alkylene is optionally substituted with one or more (e.g., one, two or three) groups R^{L}, and further wherein one or more (e.g., one or two) —CH₂— units comprised in said alkylene are each optionally replaced by a group independently selected from cycloalkylene and heterocycloalkylene.

It is preferred that said cycloalkylene (which may replace a —CH₂— unit in the alkylene in group L) is a C₃₋₅ cycloalkylene, more preferably a cyclopropylene. It is furthermore preferred that said cycloalkylene (including said C₃₋₅ cycloalkylene or said cyclopropylene) is attached via the same ring carbon atom to the remainder of the compound (i.e., that said cycloalkylene is a cycloalkan-1,1-diyl group). Moreover, it is preferred that said heterocycloalkylene (which may replace a —CH₂— unit in the alkylene in group L) is a heterocycloalkylene having 3 to 5 ring members, more preferably a heterocycloalkylene having 3 to 5 ring members wherein 1 ring member is a heteroatom selected from O, S and N (and the remaining ring members are carbon atoms), such as, e.g., oxetanylene. It is further preferred that said heterocycloalkylene is attached via the same ring carbon atom to the remainder of the compound (as in, e.g., oxetan-3,3-diyl). It will be understood that if L is methylene in which one —CH₂— unit is replaced, e.g., by cyclopropan-1,1-diyl, then the resulting group L is cyclopropan-1,1-diyl.

Preferably, L is a covalent bond or C₁₋₃ alkylene (e.g., —CH₂—, —CH₂CH₂— or —CH₂CH₂CH—), wherein said C₁₋₃ alkylene is optionally substituted with one or more (e.g., one or two) groups R^{L}, and further wherein one —CH₂— unit comprised in said C₁₋₃ alkylene is optionally replaced by cycloalkylene or heterocycloalkylene. More preferably, L is a covalent bond, methylene, ethylene, cycloalkylene (e.g., cyclopropan-1,1-diyl), or heterocycloalkylene (e.g., oxetan-3,3-diyl), wherein said methylene or said ethylene is optionally substituted with one R^{L}(e.g., with ═O). Even more preferably, L is a covalent bond, —CH₂—, or —C(═O)CH₂—, wherein said —C(═O)CH₂— is attached via its C(═O) carbon atom to the group Het and via its CH₂ carbon atom to the group —S(═O)ₙ— in formula (I). Yet even more preferably, L is a covalent bond or —CH₂—. Still more preferably, L is —CH₂—.

Each R^{L} is independently selected from —OH, —O(C₁₋₅ alkyl), ═O, —SH, —S(C₁₋₅ alkyl), —NH₂, —NH(C₁₋₅ alkyl), —N(C₁₋₅ alkyl)(C₁₋₅ alkyl), halogen, —CF₃, —CN, cycloalkyl, and heterocycloalkyl.

Preferably, each R^{L} is independently selected from —OH, —O(C₁₋₅ alkyl) and ═O. In particular, R^{L} may, e.g., be ═O which is attached to the carbon atom of L that also carries the group Het.

The group et is cyclic group selected from any one of the following groups:

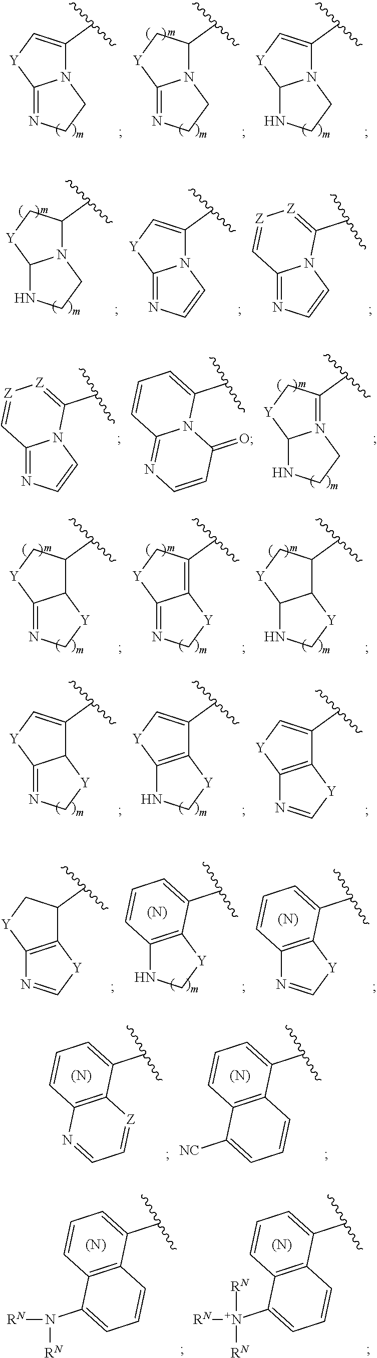

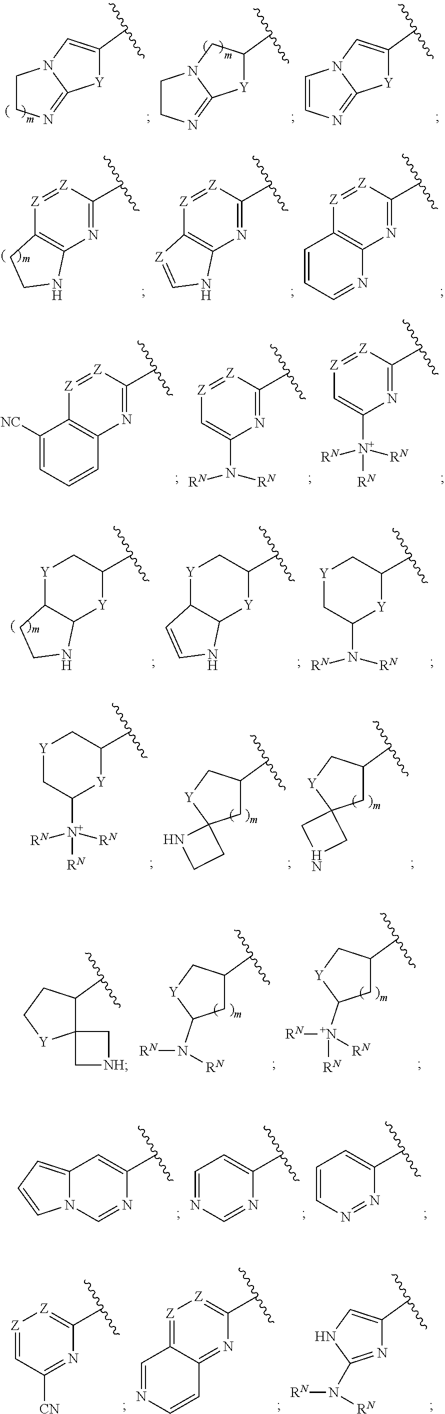

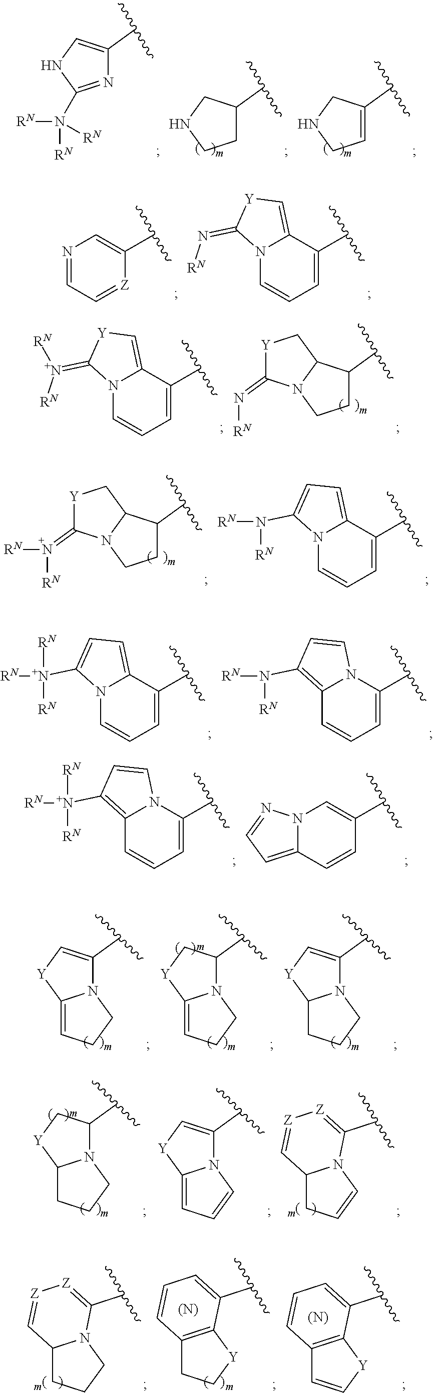

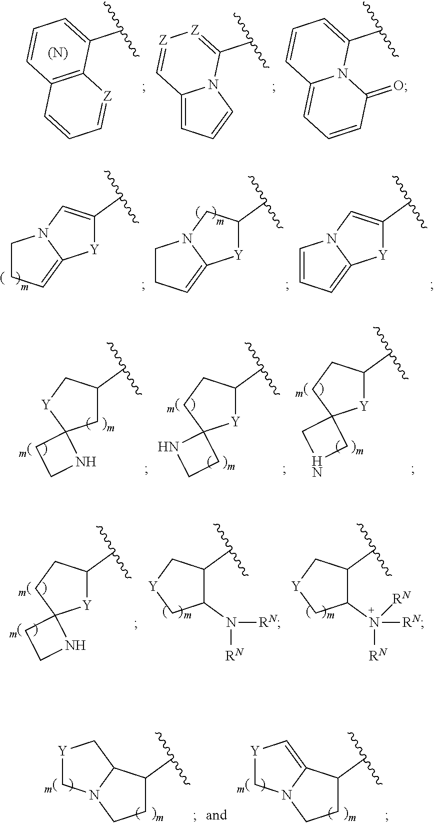

wherein each of to above-depicted groups is optionally substituted with one or more (e.g., one, to or three) groups R^{Het};
wherein each is independently 1, 2 or 3;
wherein each ring atom is independently selected from, O, SO₂, and CH₂;
wherein each ring atom is independently C or N; and
wherein to symbol “(N)” depicted inside a ring (e.g., as in

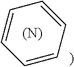

indicates that 0, 1, 2 or 3 ring atom(s) of to respective ring is/are nitrogen ring atom(s).

It will be understood that the variable m indicates the number of the respective ring atoms.

Thus, for example, if m in the group

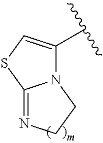

is 1, 2 or 3, then the corresponding group will have the following structure:

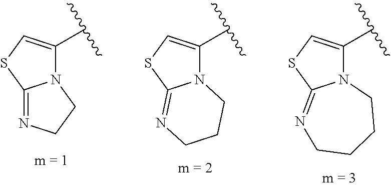 [SMILES: *C1=CSC2=NCCN12 ; *C1=CSC2=NCCCN12 ; *C1=CSC2=NCCCCN12]

Furthermore, as described above, the value of each m is selected independently (i.e., independently from the value of any other m). Consequently, if a group Het contains more than one variable m, the respective variables m may have the same value or different values.

Preferably, each m is independently 1 or 2.

As explained above, the symbol “(N)” inside a ring, as in

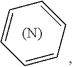

indicates that 0, 1, 2 or 3 ring atom(s) of the respective ring is/are nitrogen ring atom(s). The remaining ring atoms are carbon ring atoms. Thus, a ring

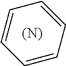

(which may form part of a ring system) may be a phenyl ring, a pyridine ring, a diazine ring, or a triazine ring.

Where the symbol “(N)” is depicted inside a ring, it is preferred that 0, 1 or 2 ring atom(s) of the respective ring is/are nitrogen ring atom(s).

The group Het may be selected, in particular, from any one of the following groups:

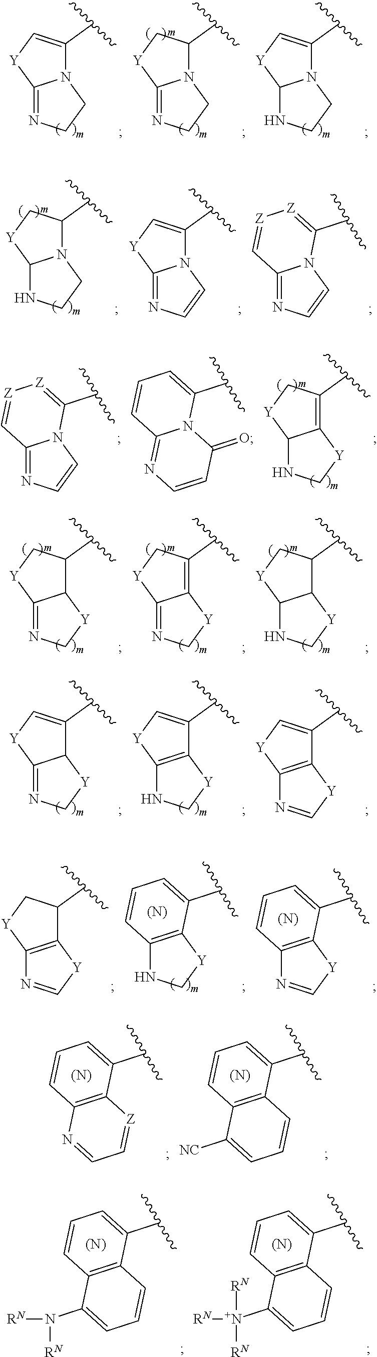

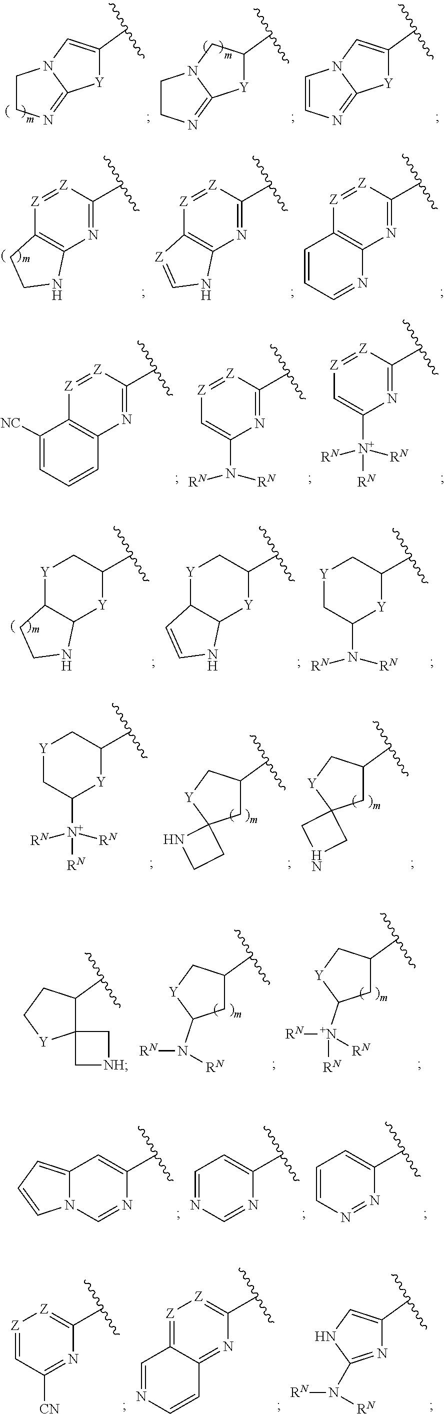

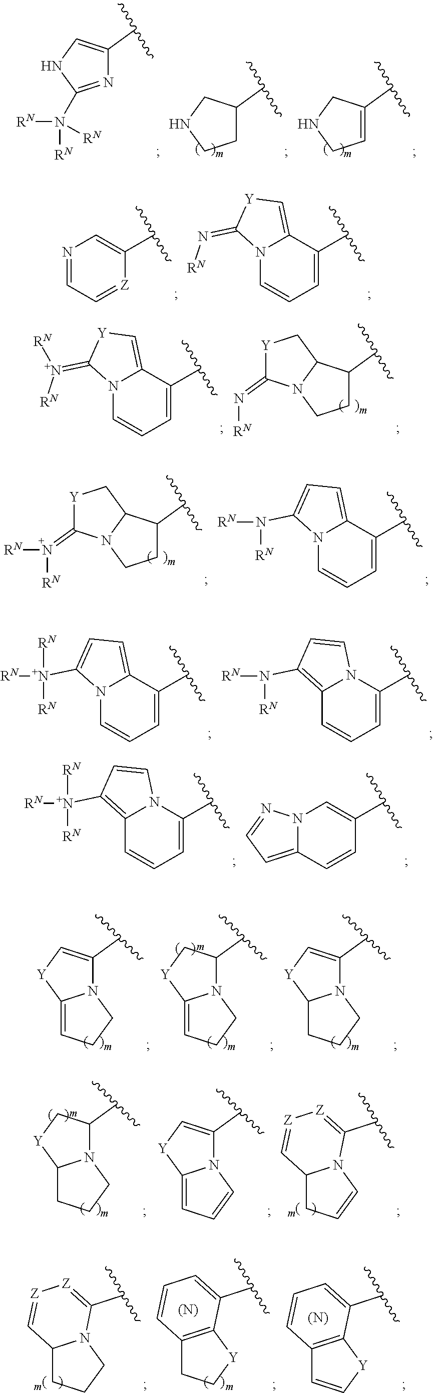

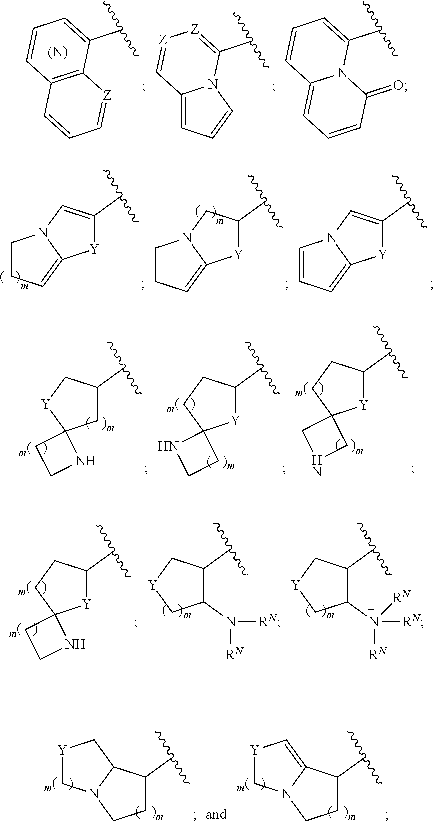

wherein each of the above-depicted groups is optionally substituted with one or more R^{Het};
wherein each m is independently 1, 2 or 3;
wherein each ring atom Y is independently selected from S, O, SO₂, NH and CH₂;
wherein each ring atom Z is independently C or N; and
wherein the symbol “(N)” depicted inside a ring indicates that 0, 1, 2 or 3 ring atom(s) of the respective ring is/are nitrogen ring atom(s).

It is preferred that the group Het is a cyclic group selected from any one of the following groups:

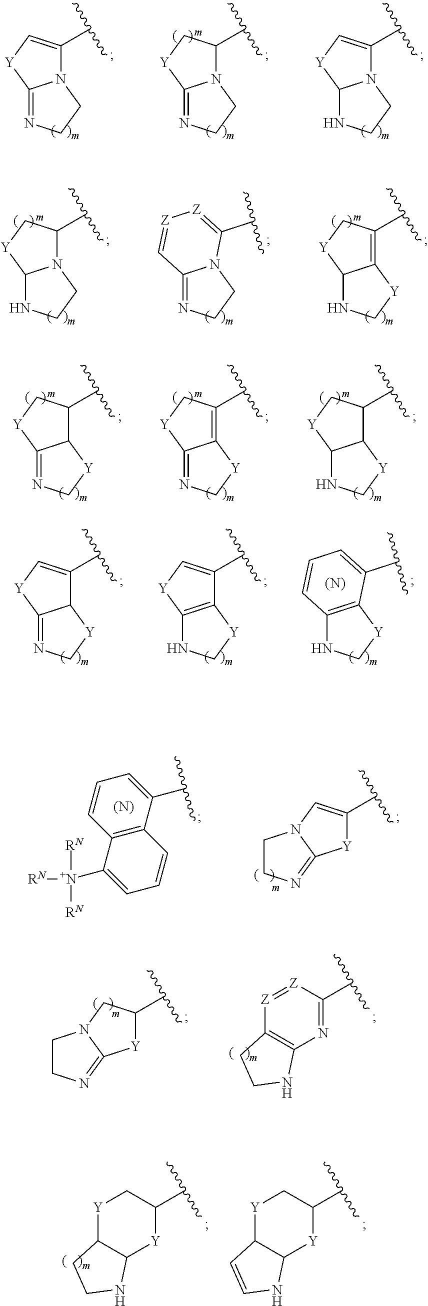

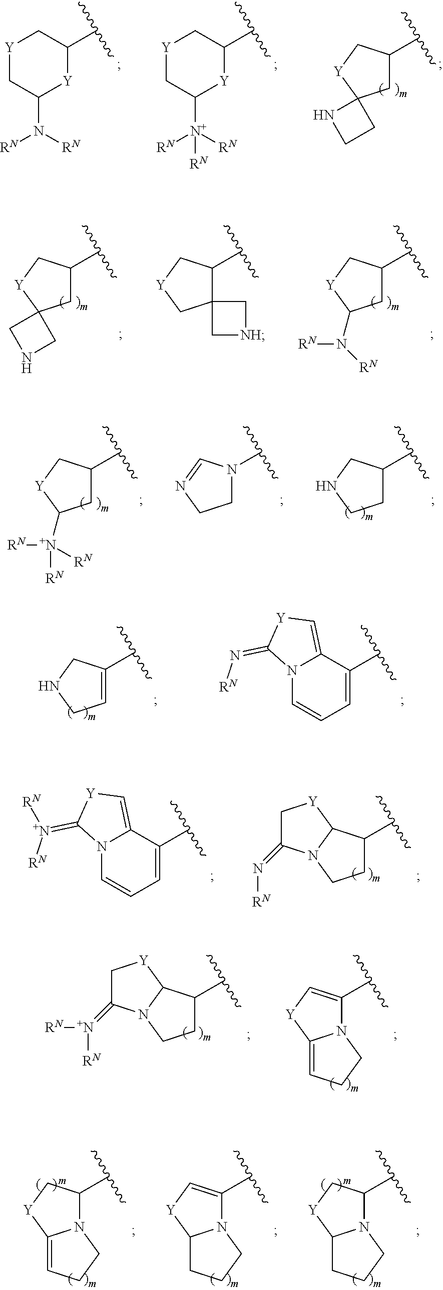

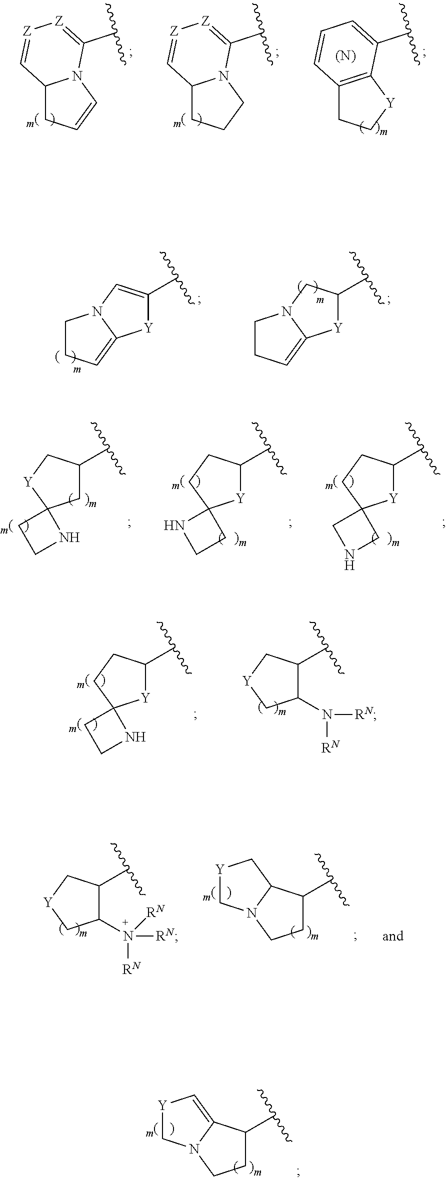

wherein each of the above-depicted groups is optionally substituted with one or more (e.g., one, to or three) groups R^{Het};
wherein each m is independently 1, 2 or 3;
wherein each ring atom Y is independently selected from S, O, SO₂, NH and CH₂;
wherein each ring atom Z is independently C or N; and
wherein the symbol “(N)” depicted inside a ring indicates that 0, 1, 2 or 3 ring atom(s) of the respective ring is/are nitrogen ring atom(s).

More preferably, Het is a group

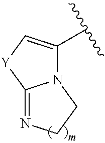

which is optionally substituted with one or more groups R^{Het}, wherein m is 1 or 2, and wherein Y is S, O or SO₂.

Even more preferably, Het is a group

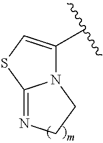

which is optionally substituted with one or more groups R^{Het}, and wherein m is 1 or 2.

Yet even more preferably, Het is a group

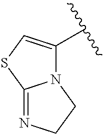 [SMILES: *C1=CSC2=NCCN12]

which is optionally substituted with one or more groups R^{Het}.

For example, Het may be any one of the following groups:

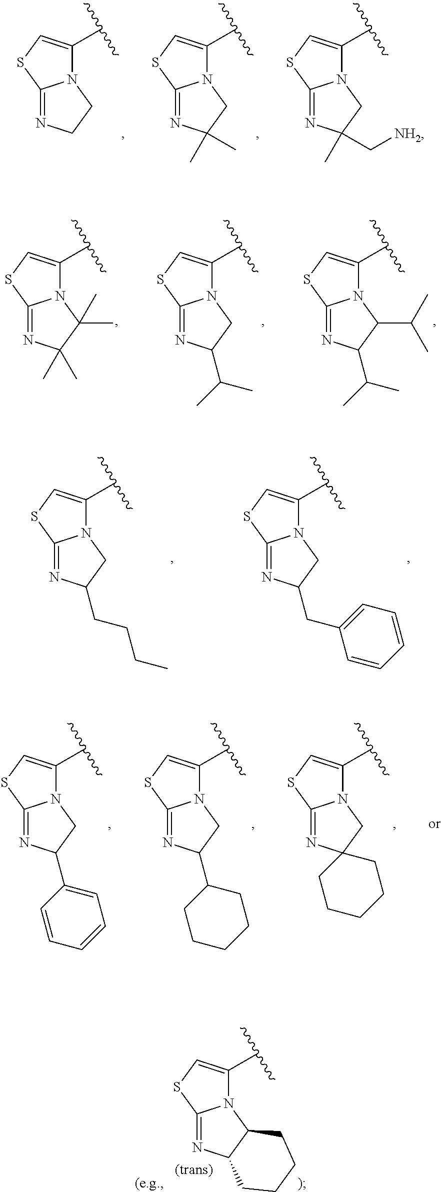

wherein each of to above-depicted groups is optionally further substituted with one or more groups R^{Het} (preferably, each of these groups is not further substituted with R^{Het}).

Moreover, in accordance with the above definitions of L an Het, it is particularly preferred that the moiety -L-Het is selected from any one of the following groups:

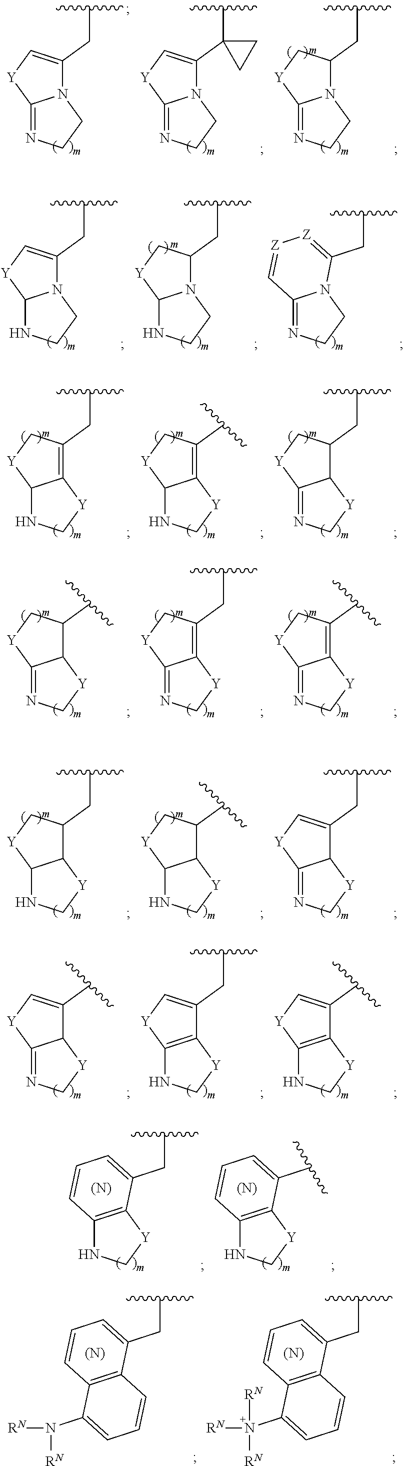

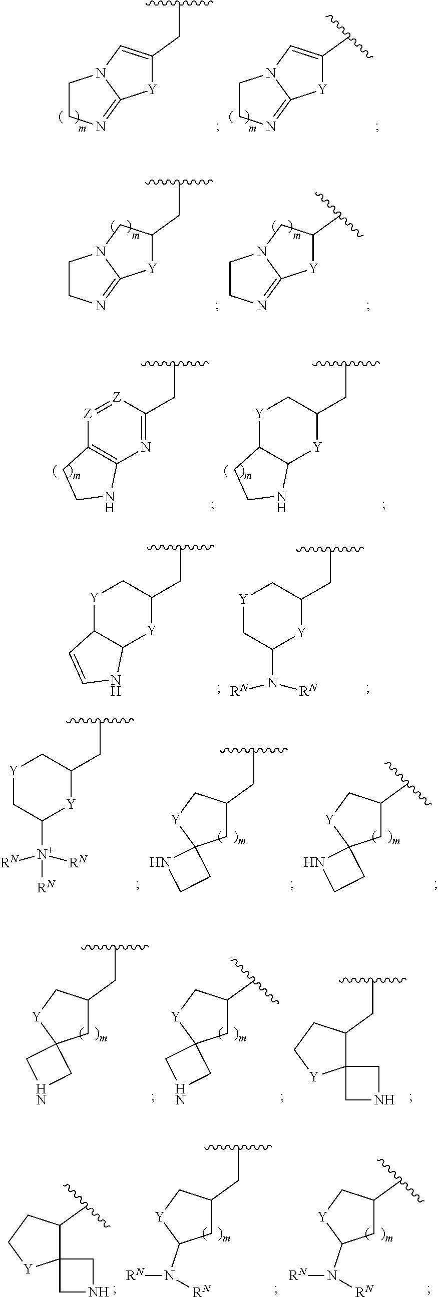

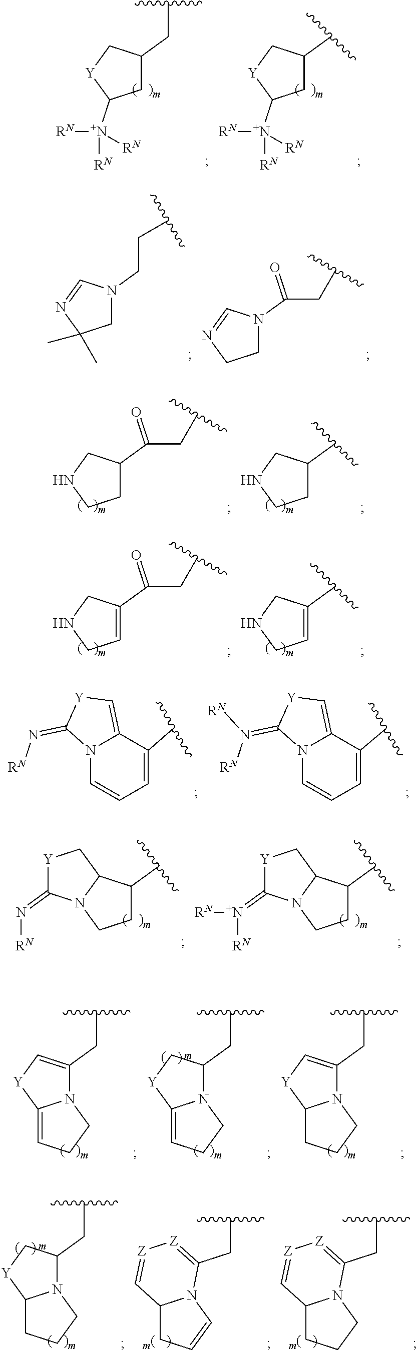

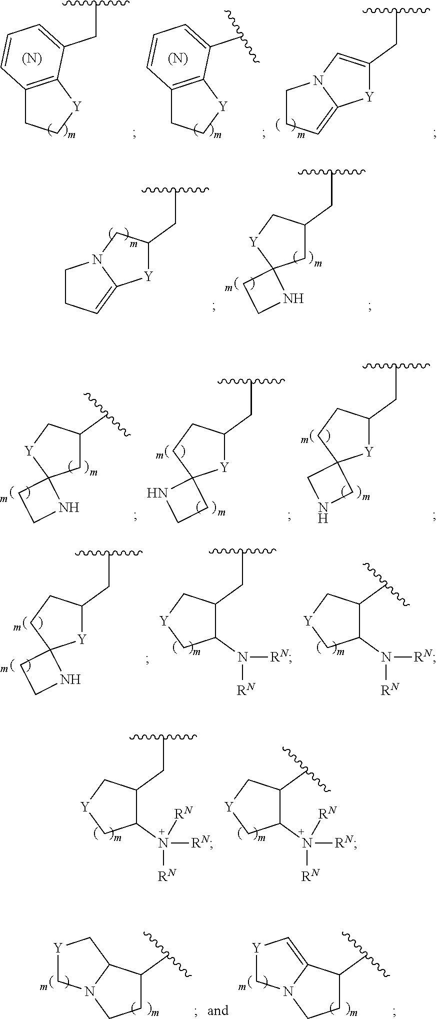

wherein the cyclic moiety (Het) in each of the above-depicted groups is optionally substituted with one or more (e.g., one, two or three) groups R^{Het};
wherein each m is independently 1, 2 or 3;
wherein each ring atom Y is independently selected from S, O, SO₂, NH and CH₂;
wherein each ring atom Z is independently C or N; and
wherein the symbol “(N)” depicted inside a ring indicates that 0, 1, 2 or 3 ring atom(s) of the respective ring is/are nitrogen ring atom(s).

Even more preferably, the moiety -L-Het is selected from any one of the following groups:

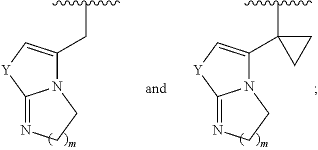

wherein the bicyclic moiety (Het) in each of the above-depicted groups is optionally substituted with one or more groups R^{Het}, wherein each m is independently 1 or 2, and wherein each Y is independently S, O or SO₂.

Yet even more preferably, the moiety -L-Het is a group

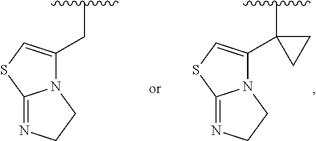 [SMILES: *CC1=CSC2=NCCN12 ; *C1(C2=CSC3=NCCN23)CC1]

which is optionally substituted with one or more groups R^{Het}.

Still more preferably, the moiety -L-Het is a group

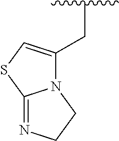 [SMILES: *CC1=CSC2=NCCN12]

which is optionally substituted with one or more groups R^{Het}.

Each R^{N} is independently selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C₁₋₅ alkyl), and the alkyl moiety in said —COO(C₁₋₅ alkyl) are each optionally substituted with one or more (e.g., one, two, or three) groups R^{Alk}, and wherein said carbocyclyl and said heterocyclyl are each optionally substituted with one or more (e.g., one, two, or three) groups R^{Cyc}; and any two groups R^{N} that are attached to the same nitrogen atom may also be mutually joined to form, together with the nitrogen atom that they are attached to, a heterocyclyl which is optionally substituted with one or more (e.g., one, two, or three) groups R^{Cyc}.

Preferably, each R^{N} is independently selected from hydrogen, C₁₋₅ alkyl, —O(C₁₋₅ alkyl), and —CO(C₁₋₅ alkyl), wherein said alkyl, the alkyl moiety in said —O(C₁₋₅ alkyl), and the alkyl moiety in said —CO(C₁₋₅ alkyl) are each optionally substituted with one or more groups R^{Alk}; and any two groups R^{N} that are attached to the same nitrogen atom may also be mutually joined to form, together with the nitrogen atom that they are attached to, a heterocyclyl which is optionally substituted with one or more groups R^{Cyc}. More preferably, each R^{N} is independently selected from hydrogen, C₁₋₅ alkyl, —O(C₁₋₅ alkyl), and —CO(C₁₋₅ alkyl), wherein said alkyl, the alkyl moiety in said —O(C₁₋₅ alkyl), and the alkyl moiety in said —CO(C₁₋₅ alkyl) are each optionally substituted with one or more groups R^{Alk}. Even more preferably, each R^{N} is independently selected from hydrogen, C₁₋₅ alkyl, —O(C₁₋₅ alkyl), and —CO(C₁₋₅ alkyl). Yet even more preferably, each R^{N} is independently selected from hydrogen and C₁₋₅ alkyl (e.g., methyl or ethyl).

Each R^{Het} is independently a group -L^{H1}-R^{H1}; any two groups R^{Het}, which are attached to the same carbon ring atom of Het, may also be mutually joined to form, together with the carbon atom that they are attached to, a cycloalkyl or a heterocycloalkyl, wherein said cycloalkyl or said heterocycloalkyl is optionally substituted with one or more (e.g., one, two, or three) groups R^{Cyc}; and any two groups R^{Het}, which are attached to different ring atoms of Het, may also be mutually joined to form a C₁₋₅ alkylene, wherein said alkylene is optionally substituted with one or more (e.g., one, two or three) groups R^{Cyc}, and wherein one —CH₂— unit comprised in said alkylene is optionally replaced by a group selected from —O—, —NH—, —N(C₁₋₅ alkyl)-, —CO—, —S—, —SO—, and —SO₂—.

It will be understood that an optional substituent R^{Het}, if present, may be attached to any carbon ring atom or any nitrogen ring atom of the corresponding group Het, which carbon or nitrogen ring atom would otherwise (i.e., without R^{Het}) carry a hydrogen atom. Likewise, if two groups R^{Het} (which are attached to the same ring atom of the group Het) are mutually joined to form a cycloalkyl or heterocycloalkyl (as described above), these groups R^{Het} may be attached to any carbon ring atom of Het which would otherwise (i.e., without the two groups R^{Het}) carry two hydrogen atoms. Moreover, if two groups R^{Het} (which are attached to different ring atoms of Het) are mutually joined to form a C₁₋₅ alkylene (as described above), these groups R^{Het} may be attached to any carbon ring atom or any nitrogen ring atom of the corresponding group Het, which carbon or nitrogen ring atom would otherwise (i.e., without R^{Het}) carry a hydrogen atom.

As described above, any two groups R^{Het}, which are attached to the same carbon ring atom of Het, may be mutually joined to form, together with that carbon ring atom, a cycloalkyl or a heterocycloalkyl (which is optionally substituted with one or more groups R^{Cyc}). It is preferred that the cycloalkyl or heterocycloalkyl which is formed from such two groups R^{Het}, and which is optionally substituted with one or more groups R^{Cyc}, has 3 to 14 ring members, more preferably 3 to 10 (i.e., 3, 4, 5, 6, 7, 8, 9 or 10) ring members. Moreover, it is preferred that said cycloalkyl or said heterocycloalkyl is monocyclic, bridged polycyclic (e.g., bridged bicyclic), or fused polycyclic (e.g., fused bicyclic); more preferably, said cycloalkyl or said heterocycloalkyl is monocyclic or bridged bicyclic. It is particularly preferred that the cycloalkyl which is formed from two groups R^{Het}, and which is optionally substituted with one or more groups R^{Cyc}, is a monocyclic C₃₋₇ cycloalkyl (e.g., cyclopropyl, cyclobutyl, cyclopentyl, cyclohexyl, or cycloheptyl) or a bicyclic bridged C₇₋₁₀ cycloalkyl (e.g., norbornanyl or adamantyl). It is furthermore particularly preferred that the heterocycloalkyl which is formed from two groups R^{Het}, and which is optionally substituted with one or more groups R^{Cyc}, is a monocyclic 3 to 7-membered heterocycloalkyl (e.g., azetidinyl, oxetanyl, thietanyl, pyrrolidinyl, tetrahydrofuranyl, tetrahydrothiophenyl, piperidinyl, tetrahydropyranyl, or thianyl) or a bicyclic bridged 7 to 10-membered heterocycloalkyl (e.g., quinuclidinyl or nortropanyl).

As also described above, any two groups R^{Het}, which are attached to different ring atoms of Het, may be mutually joined to form a C₁₋₅ alkylene (e.g., a C₁₋₃ alkylene), wherein said alkylene is optionally substituted with one or more (e.g., one, two or three) groups R^{Cyc}, and wherein one —CH₂— unit comprised in said alkylene is optionally replaced by a group selected from —O—, —NH—, —N(C₁₋₅ alkyl)-, —CO—, —S—, —SO—, and —SO₂—. It is preferred that said alkylene is optionally substituted with one or two groups R^{Cyc}, and it is furthermore preferred that one —CH₂— unit comprised in said alkylene is optionally replaced by a group selected from —O—, —NH—, —N(C₁₋₅ alkyl)-, —CO—, and —S—, more preferably from —O—, —NH—, and —N(C₁₋₅ alkyl)-. Moreover, said C₁₋₅ alkylene is preferably selected from —CH₂—, —CH₂CH₂—, —CH₂CH₂CH₂—, —CH₂CH₂CH₂CH₂— and —CH₂C₂CH₂CH₂CH₂—. In particular, it is preferred that said alkylene is a C₁₋₃ alkylene (more preferably —CH₂— or —CH₂CH₂—), and that the two ring atoms of Het, which carry the two groups R^{Het}, that are mutually joined to form the alkylene, are non-adjacent ring atoms of Het; accordingly, it is preferred that there is at least one other ring atom (e.g., one, two or three other ring atoms) in between the two ring atoms of Het which carry the two mutually joined groups R^{Het}, However, if two groups R^{Het}, which are attached to adjacent ring atoms of Het, are mutually joined to form an alkylene (which is optionally substituted with one or more R^{Cyc}, and wherein one —CH₂— unit comprised in the alkylene is optionally replaced, as defined above), it is preferred that said alkylene is a C₃₋₅ alkylene, such as, e.g., —CH₂CH₂CH₂—, —CH₂CH₂CH₂CH₂— or —CH₂CH₂CH₂CH₂CH₂—.

Unless defined otherwise, it is preferred that each R^{Het} is independently a group -L^{H1}-R^{H1} (i.e., that no groups R^{Het} are mutually joined).

Each L^{H1} is independently selected from a bond, C₁₋₅ alkylene, C₂₋₅ alkenylene, and C₂₋₅ alkynylene, wherein said alkylene, said alkenylene and said alkynylene are each optionally substituted with one or more (e.g., one, two, or three) groups independently selected from halogen, C₁₋₅ haloalkyl, —O(C₁₋₅ haloalkyl), —ON, —OR^{H2}, —NR^{H2}R^{H2}, —N⁺R^{H2}R^{H2}R^{H2}, —NR^{H2}OR^{H2}, —COR^{H2}, —COOR^{H2}, —OCOR^{H2}, —CONR^{H2}R^{H2}, —NR^{H2}COR^{H2}, —NR^{H2}COOR^{H2}, —OCONR^{H2}R^{H2}, —SR^{H2}, —SOR^{H2}, —SO₂R^{H2}, —SO₂NR^{H2}R^{H2}, —NR^{H2}SO₂R^{H2}, —SOR², and —NO₂, and further wherein one or more (e.g., one or two) —CH₂— units comprised in said alkylene, said alkenylene or said alkynylene are each optionally replaced by a group independently selected from —O—, —NR^{H2}—, —CO—, —S—, —SO—, and —SO₂—.

Preferably, each L^{H1} is independently selected from a bond and C₁₋₅ alkylene, wherein said alkylene is optionally substituted with one or more (e.g., one, two, or three) groups independently selected from halogen, C₁₋₅ haloalkyl, —O(C₁₋₅ haloalkyl), —CN, —OR^{H2}, —NR^{H2}R^{H2}, —N⁺R^{H2}R^{H2}R^{H2}, —NR^{H2}OR^{H2}, —COR^{H2}, —COOR^{H2}, —OCOR^{H2}, —CONR^{H2}R^{H2}, —NR^{H2}COR^{H2}, —NR^{H2}COOR^{H2}, —OCONR^{H2}R^{H2}, —SR^{H2}, —SOR^{H2}, —SO₂R^{H2}, —SO₂NR^{H2}R^{H2}, —NR^{H2}SO₂R^{H2}, —SO₃R^{H2}, and —NO₂, and further wherein one or more (e.g., one, two, or three) —CH₂— units comprised in said alkylene are each optionally replaced by a group independently selected from —O—, —NR^{H2}—, —CO—, —S—, —SO—, and —SO₂—. More preferably, each L^{H1} is independently selected from a bond and C₁₋₅ alkylene, wherein said alkylene is optionally substituted with one or more groups independently selected from halogen, C₁₋₅ haloalkyl, —O(C₁₋₅ haloalkyl), —CN, —OH, —O(C₁₋₅ alkyl), —NH₂, —NH(C₁₋₅ alkyl), and —N(C₁₋₅ alkyl)(C₁₋₅ alkyl), and further wherein one or two —CH₂— units comprised in said alkylene is/are each optionally replaced by a group independently selected from —O—, —NH—, —N(C₁₋₅ alkyl)-, —CO—, and —SO₂—. Even more preferably, each L^{H1} is independently selected from a bond and C₁₋₅ alkylene (e.g., methylene or ethylene). Yet even more preferably, L^{H1} is a bond.

Each R^{H1} is independently selected from C₁₋₅ alkyl, C₂₋₅ alkenyl, C₂₋₅ alkynyl, halogen, C₁₋₅ haloalkyl, —O(C₁₋₅ haloalkyl), —ON, —OR^{H3}, —NR^{H3}R^{H3}, —NR^{H3}R^{H3}R^{H3}, —NR^{H3}OR^{H3}, —COR^{H3}, —COOR^{H3}, —OCOR^{H3}, —CONR^{H3}R^{H3}, —NR^{H3}COR^{H3}, —NR^{H3}COOR^{H3}, —OCONR^{H3}R^{H3}, —SR^{H3}, —SOR^{H3}, —SO₂R^{H3}, —SO₂NR^{H3}R^{H3}, —NR^{H3}SO₂R^{H3}, —SO₃R^{H3}, carbocyclyl, and heterocyclyl, wherein said alkyl, said alkenyl and said alkynyl are each optionally substituted with one or more (e.g., one, two, or three) groups R^{Alk}, and further wherein said carbocyclyl and said heterocyclyl are each optionally substituted with one or more (e.g., one, two, or three) groups R^{Cyc}.

Preferably, each R^{H1} is independently selected from C₁₋₅ alkyl, C₂₋₅ alkenyl, C₂₋₅ alkynyl, halogen, C₁₋₅ haloalkyl, —O(C₁₋₅ haloalkyl), —CN, —OR^{H3}, —NR^{H3}R^{H3}, —N⁺R^{H3}R^{H3}R^{H3}, —COR^{H3}, —COOR^{H3}, —OCOR^{H3}, —CONR^{H3}R^{H3}, —NR^{H3}COR^{H3}, —SR^{H3}, —SOR^{H3}, —SO₂R^{H3}, —SO₂NR^{H3}R^{H3}, —NR^{H3}SO₂R^{H3}, cycloalkyl, aryl, heterocycloalkyl, and heteroaryl, wherein said alkyl, said alkenyl and said alkynyl are each optionally substituted with one or more (e.g., one, two or three) groups R^{Alk}, and further wherein said cycloalkyl, said aryl, said heterocycloalkyl and said heteroaryl are each optionally substituted with one or more (e.g., one, two or three) groups R^{Cyc} More preferably, each R^{H1} is independently selected from C₁₋₅ alkyl, C₂₋₅ alkenyl, C₂₋₅ alkynyl, halogen, C₁₋₅ haloalkyl, —O(C₁₋₅ haloalkyl), —CN, —OH, —O(C₁₋₅ alkyl), —O(C₁₋₅ alkylene)-OH, —O(C₁₋₅ alkylene)-O(C₁₋₅ alkyl), —NH₂, —NH(C₁₋₅ alkyl), —N(C₁₋₅ alkyl)(C₁₋₅ alkyl), —CHO, —CO(C₁₋₅ alkyl), —COO H, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —SH, —S(C₁₋₅ alkyl), —SO—(C₁₋₅ alkyl), —SO₂—(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}. Even more preferably, each R^{H1} is independently selected from C₁₋₅ alkyl (e.g., methyl, ethyl, propyl, or butyl), halogen, C₁₋₅ haloalkyl (e.g., —CF₃), —O(C₁₋₅ haloalkyl), —CN, —OH, —O(C₁₋₅ alkyl), —NH₂, —NH(C₁₋₅ alkyl), —N(C₁₋₅ alkyl)(C₁₋₅ alkyl), cycloalkyl, aryl, heterocycloalkyl, and heteroaryl, wherein said cycloalkyl, said aryl, said heterocycloalkyl and said heteroaryl are each optionally substituted with one or more groups R^{Cyc}.

In accordance with the above definitions of L^{H1} and R^{H1}, it is particularly preferred that each group -L^{H1}-R^{H1} is independently selected from C₁₋₅ alkyl, C₂₋₅ alkenyl, C₂₋₅ alkynyl, halogen, C₁₋₅ haloalkyl, —(C₀₋₅ alkylene)-O(C₁₋₅ haloalkyl), —(C₀₋₅ alkylene)-CN, —(C₀₋₅ alkylene)-OH, —(C₀₋₅ alkylene)-O(C₁₋₅ alkyl), —(C₀₋₅ alkylene)-O(C₁₋₅ alkylene)-OH, —(C₀₋₅ alkylene)-O(C₁₋₅ alkylene)-O(C₁₋₅ alkyl), —(C₀₋₅ alkylene)-NH₂, —(C₁₋₅ alkylene)-NH(C₁₋₅ alkyl), —(C₁₋₅ alkylene)-N(C₁₋₅ alkyl)(C₁₋₅ alkyl), —(C₀₋₅ alkylene)-CHO, —(C₀₋₅ alkylene)-CO(C₁₋₅ alkyl), —(C₀₋₅ alkylene)-COOH, —(C₀₋₅ alkylene)-COO(C₁₋₅ alkyl), —(C₀₋₅ alkylene)-O—CO(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅alkyl), —(C₀₋₅ alkylene)-NH—CO(C₁₋₅ alkyl), —(C₀₋₅ alkylene)-N(C₁₋₅ alkyl)-CO(C₁₋₅ alkyl), —(C₀₋₅ alkylene)-SH, —(C₀₋₅ alkylene)-S(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-SO₂—NH₂, —(C₀₋₅ alkylene)-SO₂—NH(C₁₋₅ alkyl), —(C₁₋₅ alkylene)-SO₂—N(C₁₋₅ alkyl)(C₁₋₅ alkyl), —(C₀₋₅ alkylene)-NH—SO₂—(C₁₋₅ alkyl), —(C₀₋₅ alkylene)-N(C₁₋₅ alkyl)-SO₂—(C₁₋₅ alkyl), —(C₀₋₅ alkylene)-cycloalkyl, —(C₀₋₅ alkylene)-aryl (e.g., phenyl or benzyl), —(C₀₋₅ alkylene)-heterocycloalkyl, and —(C₀₋₅ alkylene)-heteroaryl, wherein the cycloalkyl moiety in said —(C₀₋₅ alkylene)-cycloalkyl, the aryl moiety in said —(C₀₋₅ alkylene)-aryl, the heterocycloalkyl moiety in said —(C₀₋₅ alkylene)-heterocycloalkyl, and the heteroaryl moiety in said —(C₀₋₅ alkylene)-heteroaryl are each optionally substituted with one or more groups R^{Cyc}.

If a group -L^{H1}-R^{H1} is attached to a carbon ring atom of Het, which carbon ring atom is adjacent to the ring atom through which Het is attached to the group L, then this group -L^{H1}-R^{H1} may be, in particular, C₁₋₅ alkyl (e.g., methyl, ethyl, or isopropyl), cycloalkyl (e.g., cyclopropyl), or halogen (e.g., —I).

Each R^{H2} and each R^{H3} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more (e.g., one, two, or three) groups R^{Alk}, and further wherein said carbocyclyl and said heterocyclyl are each optionally substituted with one or more (e.g., one, two, or three) groups R^{Cyc}.

Preferably, each R^{H2} and each R^{H3} is independently selected from hydrogen and C₁₋₅ alkyl, wherein said alkyl is optionally substituted with one or more (e.g., one, two or three) groups R^{Alk}. More preferably, each R^{H2} and each R^{H3} is independently selected from hydrogen and C₁₋₅ alkyl (e.g., methyl or ethyl).

Furthermore, in accordance with the present invention, the following compounds are excluded from formula (I):
3-((4,5-dihydro-1H-imidazol-2-ylthio)methyl)-2,3,5,6-tetrahydroimidazo[2,1-b]thiazol-3-ol;
2-(4,5-dihydro-1H-imidazol-2-ylthio)-2,3,5,6-tetrahydroimidazo[2,1-b]thiazole;
3-(4,5-dihydro-1H-imidazol-2-ylthio)-2,3,5,6-tetrahydroimidazo[2,1-b]thiazole;
2-(4,5-dihydro-1H-imidazol-2-ylthio)-1-(2-mercapto-4,5-dihydro-1H-imidazol-1-yl)ethanone;
5-chloro-6-((4,5-dihydro-1H-imidazol-2-ylthio)methyl)pyrimidine-2,4-diol;
5-methyl-6-((4,5-dihydro-1H-imidazol-2-ylthio)methyl)pyrimidine-2,4-diol;
6-(4,5-dihydro-1H-imidazol-2-ylthio)-9H-purine;
2-chloro-6-(4,5-dihydro-1H-imidazol-2-ylthio)-9H-purine;
2-amino-6-(4,5-dihydro-1H-imidazol-2-ylthio)-9H-purine;
8-isopropoxy-7-(1-methyl-1H-imidazol-2-ylthio)-5-(1-methyl-4,5-dihydro-1H-imidazol-2-ylthio)quinoline; and
2-(4,5-dihydro-1H-imidazol-2-ylthio)-1-(pyridin-3-yl)ethanone.

Accordingly, the present invention does not relate to the compounds listed in the preceding paragraph or pharmaceutically acceptable salts or solvates thereof.

In accordance with the definition of formula (I), the present invention does not relate to the following compounds or pharmaceutically acceptable salts or solvates thereof either, which are not encompassed by formula (I):
4-(2-(4,5-dihydro-1H-imidazol-2-ylthio)ethyl)morpholine;
1-(2-(4,5-dihydro-1H-imidazol-2-ylthio)ethyl)piperidine;
1-(2-(4-phenyl-4,5-dihydro-1H-imidazol-2-ylthio)ethyl)piperidine;
1-(2-(4,5-dihydro-1H-imidazol-2-ylthio)ethyl)-4-methylpiperazine;
1-(2-(4,5-dihydro-1H-imidazol-2-ylthio)ethyl)azepane;
2-(2-(2-methyl-5-nitro-1H-imidazol-1-yl)ethylthio)-1,4,5,6-tetrahydropyrimidine;
1-(4-methylpiperazin-1-yl)-2-(1,4,5,6-tetrahydropyrimidin-2-ylthio)ethanone;
1-(4-methylpiperazin-1-yl)-2-(1,4,5,6-tetrahydropyrimidin-2-ylthio)propan-1-one;
1-(4-methylpiperazin-1-yl)-2-(4,5,6,7-tetrahydro-1H-1,3-diazepin-2-ylthio)ethanone; and
1-(4-methylpiperazin-1-yl)-2-(4,5,6,7-tetrahydro-1H-1,3-diazepin-2-ylthio)propan-1-one.

Moreover, it is preferred that any one or more (particularly all) of the following conditions apply to formula (I):
if Het is a group

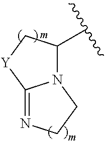

which is optionally substituted with one or more groups R^{Het}, wherein Y is S and each m is 1, if L is —CH₂—, if X is

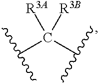

and if R¹, R^{2A}, R^{2B}, R^{3A} and R^{3B} are each hydrogen, then R^{Het} is not —OH;
if Het is a group

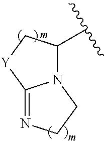

which is optionally substituted with one or more groups R^{Het}, wherein Y is S and each m is 1, if X is

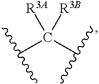

and if R¹, R^{2A}, R^{2B}, R^{3A} and R^{3B} are each hydrogen, then L is not a covalent bond;
if Het is a group

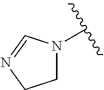 [SMILES: *N1C=NCC1]

which is optionally substituted with one or more groups R^{Het}, if X is

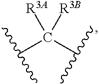

and if R¹, R^{2A}, R^{2B}, R^{3A} and R^{3B} are each hydrogen, then R^{L} is not ═O;
if Het is a group

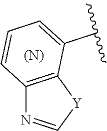

which is optionally substituted with one or more groups R^{Het}, wherein Y is NH, if X is

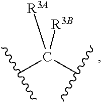

and if R¹, R^{2A}, R^{2B}, R^{3A} and R^{3B} are each hydrogen, then L is not a covalent bond;
if Het is a group

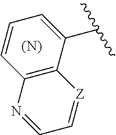

which is optionally substituted with one or more groups R^{Het}, wherein Z is C and the ring marked with the symbol “(N)” contains 0 nitrogen ring atoms, if X is

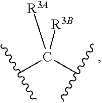

if R¹ is methyl, and if R^{2A}, R^{2B}, R^{3A} and R^{3B} are each hydrogen, then L is not a covalent bond;
if Het is a group

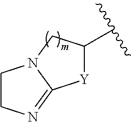

which is optionally substituted with one or more groups R^{Het}, wherein Y is S and m is 1, if X is

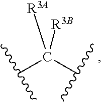

and if R¹, R^{2A}, R^{2B}, R^{3A} and R^{3B} are each hydrogen, then L is not a covalent bond;
if Het is a group

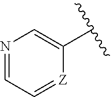

which is optionally substituted with one or more groups R^{Het}, wherein Z is C, if X is

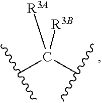

and if R¹, R^{2A}, R^{2B}, R^{3A} and R^{3B} are each hydrogen, then R^{L} is not ═O;
if Het is a group

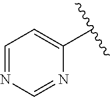 [SMILES: *c1ccncn1]

which is optionally substituted with one or more groups R^{Het}, if X is

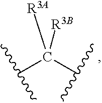

and if R¹, R^{2A}, R^{2B}, R^{3A} and R^{3B} are each hydrogen, then L is not —CH₂—.

It is particularly preferred that the compound of formula (I) is one of the specific compounds of formula (I) described in the examples section of this specification, including any one of Examples 1 to 70 described further below, either in non-salt form or as a pharmaceutically acceptable salt or solvate of the respective compound.

Accordingly, it is particularly preferred that the compound of formula (I) is selected from:
3-(((3a,4,5,6,7,7a-hexahydro-1H-benzo[d]imidazol-2-yl)thio)methyl)-6,6-dimethyl-5,6-dihydroimidazo[2,1-b]thiazole;
trans-3-(((3a,4,5,6,7,7a-hexahydro-1H-benzo[d]imidazol-2-yl)thio)methyl)-6,6-dimethyl-5,6-dihydroimidazo[2,1-b]thiazole;
cis-3-(((3a,4,5,6,7,7a-hexahydro-1H-benzo[d]imidazol-2-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-6,6-dimethyl-5,6-dihydroimidazo[2,1-b]thiazole;
3-((((3aR,7aR)-3a,4,5,6,7,7a-hexahydro-1H-benzo[d]imidazol-2-yl)thio)methyl)-6,6-dimethyl-5,6-dihydroimidazo[2,1-b]thiazole;
3-((((3aS,7aS)-3a,4,5,6,7,7a-hexahydro-1H-benzo[d]imidazol-2-yl)thio)methyl)-6,6-dimethyl-5,6-dihydroimidazo[2,1-b]thiazole;
3-((((3aR,7aS)-3a,4,5,6,7,7a-hexahydro-1H-benzo[d]imidazol-2-yl)thio)methyl)-6,6-dimethyl-5,6-dihydroimidazo[2,1-b]thiazole;
3-((((3aS,7aR)-3a,4,5,6,7,7a-hexahydro-1H-benzo[d]imidazol-2-yl)thio)methyl)-6,6-dimethyl-5,6-dihydroimidazo[2,1-b]thiazole;
3-(((4,5-dihydro-1H-imidazol-2-yl)thio)methyl)-5,6-dihydroimidazo[2,1-b]thiazole;
3-(((4,4-dimethyl-4,5-dihydro-1H-imidazol-2-yl)thio)methyl)-4a,5,6,7,8,8a-hexahydrobenzo[4,5]imidazo[2,1-b]thiazole;
trans-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
3-(((4,5-dihydro-1H-imidazol-2-yl)thio)methyl)-6,6-dimethyl-5,6-dihydroimidazo[2,1-b]thiazole; 6,6-dimethyl-3-(((1,4,5,6-tetrahydropyrimidin-2-yl)thio)methyl)-5,6-dihydroimidazo[2,1-b]thiazole;
cis-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
(4aR,8aS)-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
(4aR,8aR)-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
(4aS,8aR)-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
(4aS,8aS)-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
6,6-dimethyl-3-(((1-methyl-4,5-dihydro-1H-imidazol-2-yl)thio)methyl)-5,6-dihydroimidazo[2,1-b]thiazole;
6,6-dimethyl-3-(((4,5,6,7-tetrahydro-1H-1,3-diazepin-2-yl)thio)methyl)-5,6-dihydroimidazo[2,1-b]thiazole;
3-(((5-butyl-4,5-dihydro-1H-imidazol-2-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-6,7-dihydro-5H-thiazolo[3,2-a]pyrimidine;
3-(((5,5-dimethyl-4,5-dihydro-1H-imidazol-2-yl)thio)methyl)-5,6,7,8-tetrahydrothiazolo[3,2-a][1,3]diazepine;
6,6-dimethyl-3-(((4,4,5,5-tetramethyl-4,5-dihydro-1H-imidazol-2-yl)thio)methyl)-5,6-dihydroimidazo[2,1-b]thiazole;
3-(((5-benzyl-4,5-dihydro-1H-imidazol-2-yl)thio)methyl)-6,6-dimethyl-5,6-dihydroimidazo[2,1-b]thiazole;
6-benzyl-3-(((5,5-dimethyl-4,5-dihydro-1H-imidazol-2-yl)thio)methyl)-5,6-dihydroimidazo[2,1-b]thiazole;
6-butyl-3-(((4,4-dimethyl-4,5-dihydro-1H-imidazol-2-yl)thio)methyl)-5,6-dihydroimidazo[2,1-b]thiazole;
3-(((5,5-dimethyl-4,5-dihydro-1H-imidazol-2-yl)thio)methyl)-5,5,6,6-tetramethyl-5,6-dihydroinidazo[2,1-b]thiazole;
3-(((5,5-dimethyl-4,5-dihydro-1H-imidazol-2-yl)thio)methyl)-5,6-dihydroimidazo[2,1-b]thiazole;
3-(((2,4-diazabicyclo[3.3.1]non-2-en-3-yl)thio)methyl)-6,6-dimethyl-5,6-dihydroimidazo[2,1-b]thiazole;
cis-3-(((2,4-diazabicyclo[3.3.1]non-2-en-3-yl)thio)methyl)-6,6-dimethyl-5,6-dihydroimidazo[2,1-b]thiazole;
trans-3-(((2,4-diazabicyclo[3.3.1]non-2-en-3-yl)thio)methyl)-6,6-dimethyl-5,6-dihydroimidazo[2,1-b]thiazole;
3-((((1R,5S)-2,4-diazabicyclo[3.3.1]non-2-en-3-yl)thio)methyl)-6,6-dimethyl-5,6-dihydroimidazo[2,1-b]thiazole;
3-((((1S,5R)-2,4-diazabicyclo[3.3.1]non-2-en-3-yl)thio)methyl)-6,6-dimethyl-5,6-dihydroimidazo[2,1-b]thiazole;
3-((((1R,5R)-2,4-diazabicyclo[3.3.1]non-2-en-3-yl)thio)methyl)-6,6-dimethyl-5,6-dihydroimidazo[2,1-b]thiazole;
3-((((1S,5S)-2,4-diazabicyclo[3.3.1]non-2-en-3-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-6,7,8,9-tetrahydro-5H-5,9-methanothiazolo[3,2-a][1,3]diazocine;
cis-3-(((5,5-dimethyl-4,5-dihydro-1H-imidazol-2-yl)thio)methyl)-6,7,8,9-tetrahydro-5H-5,9-methanothiazolo[3,2-a][1,3]diazocine;
trans-3-(((5,5-dimethyl-4,5-dihydro-1H-imidazol-2-yl)thio)methyl)-6,7,8,9-tetrahydro-5H-5,9-methanothiazolo[3,2-a][1,3]diazocine;
3-(((5,5-dimethyl-4,5-dihydro-1H-imidazol-2-yl)thio)methyl)-(5S,9R)-6,7,8,9-tetrahydro-5H-5,9-methanothiazolo[3,2-a][1,3]diazocine;
3-(((5,5-dimethyl-4,5-dihydro-1H-imidazol-2-yl)thio)methyl)-(5R,9S)-6,7,8,9-tetrahydro-5H-5,9-methanothiazolo[3,2-a][1,3]diazocine;
3-(((5,5-dimethyl-4,5-dihydro-1H-imidazol-2-yl)thio)methyl)-(5S,9S)-6,7,8,9-tetrahydro-5H-5,9-methanothiazolo[3,2-a][1,3]diazocine;
3-(((5,5-dimethyl-4,5-dihydro-1H-imidazol-2-yl)thio)methyl)-(5R,9R)-6,7,8,9-tetrahydro-5H-5,9-methanothiazolo[3,2-a][1,3]diazocine;
3-(((4,6-diazaspiro[2.4]hept-5-en-5-yl)thio)methyl)-6,6-dimethyl-5,6-dihydroimidazo[2,1-b]thiazole;
2-(((6,6-dimethyl-5,6-dihydroimidazo[2,1-b]thiazol-3-yl)methyl)thio)-3a,4,6,6a-tetrahydro-1H-furo[3,4-d]imidazole;
cis-2-(((6,6-dimethyl-5,6-dihydroimidazo[2,1-b]thiazol-3-yl)methyl)thio)-3a,4,6,6a-tetrahydro-1H-furo[3,4-d]imidazole;
trans-2-(((6,6-dimethyl-5,6-dihydroimidazo[2,1-b]thiazol-3-yl)methyl)thio)-3a,4,6,6a-tetrahydro-1H-furo[3,4-d]imidazole;
(3aR,6aS)-2-(((6,6-dimethyl-5,6-dihydroimidazo[2,1-b]thiazol-3-yl)methyl)thio)-3a,4,6,6a-tetrahydro-1H-furo[3,4-d]imidazole;
(3aS,6aR)-2-(((6,6-dimethyl-5,6-dihydroimidazo[2,1-b]thiazol-3-yl)methyl)thio)-3a,4,6,6a-tetrahydro-1H-furo[3,4-d]imidazole;
(3aR,6aR)-2-(((6,6-dimethyl-5,6-dihydroimidazo[2,1-b]thiazol-3-yl)methyl)thio)-3a,4,6,6a-tetrahydro-1H-furo[3,4-d]imidazole;
(3aS,6aS)-2-(((6,6-dimethyl-5,6-dihydroimidazo[2,1-b]thiazol-3-yl)methyl)thio)-3a,4,6,6a-tetrahydro-1H-furo[3,4-d]imidazole;
3-(((4,5-dihydro-1H-imidazol-2-yl)thio)methyl)-6,7-dihydro-5H-thiazolo[3,2-a]pyrimidine;
3-(((4,5-dihydro-1H-imidazol-2-yl)thio)methyl)-5,6,7,8-tetrahydrothiazolo[3,2-a][1,3]diazepine;
3-(((5,5-dimethyl-1,4,5,6-tetrahydropyrimidin-2-yl)thio)methyl)-6,6-dimethyl-5,6-dihydroimidazo[2,1-b]thiazole;
3-(((1,4,5,6-tetrahydropyrimidin-2-yl)thio)methyl)-6,7-dihydro-5H-thiazolo[3,2-a]pyrimidine;
3-(((5-butyl-4,5-dihydro-1H-imidazol-2-yl)thio)methyl)-5,6-dihydroimidazo[2,1-b]thiazole;
3-(3-((5,5-dimethyl-4,5-dihydro-1H-imidazol-2-yl)thio)propyl)pyridine;
3-(((5,5-dimethyl-4,5-dihydro-1H-imidazol-2-yl)thio)methyl)pyridine;
3′-(((5,5-dimethyl-4,5-dihydro-1H-imidazol-2-yl)thio)methyl)-5′H-spiro[cyclopropane-1,6′-imidazo[2,1-b]thiazole];
3-(((5,5-dimethyl-4,5-dihydro-1H-imidazol-2-yl)thio)methyl)-6,6-dimethyl-6,7-dihydro-5H-thiazolo[3,2-a]pyrimidine;
3-(((1,4,5,6-tetrahydropyrimidin-2-yl)thio)methyl)-5,6-dihydroimidazo[2,1-b]thiazole;
3-(((3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
cis-3-((((cis)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
cis-3-((((trans)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
trans-3-((((cis)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
trans-3-((((trans)-3a,4,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aR)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aS)-3-((((3aR,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aR)-3-((((3aR,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aR)-3-((((3aR,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aR)-3-((((3aS,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aS)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aS)-3-((((3aR,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aS)-3-((((3aS,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aR)-3-((((3aR,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aR)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aR)-3-((((3aR,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aR)-3-((((3aS,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aS)-3-((((3aR,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aS)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aS)-3-((((3aR,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aS)-3-((((3aS,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
3-(((1,4,5,6-tetrahydropyrimidin-2-yl)thio)methyl)-5,6,7,8-tetrahydrothiazolo[3,2-a][1,3]diazepine;
1-(3-((5,5-dimethyl-4,5-dihydro-1H-imidazol-2-yl)thio)propyl)-1H-imidazole;
3-(((1,3-diazaspiro[4.5]dec-2-en-2-yl)thio)methyl)-6,6-dimethyl-5,6-dihydroimidazo[2,1-b]thiazole;
6,6-dimethyl-3-(((1,4,4a,5,6,7,8,8a-octahydroquinazolin-2-yl)thio)methyl)-5,6-dihydroimidazo[2,1-b]thiazole;
trans-6,6-dimethyl-3-(((1,4,4a,5,6,7,8,8a-octahydroquinazolin-2-yl)thio)methyl)-5,6-dihydroimidazo[2,1-b]thiazole;
cis-6,6-dimethyl-3-(((1,4,4a,5,6,7,8,8a-octahydroquinazolin-2-yl)thio)methyl)-5,6-dihydroimidazo[2,1-b]thiazole;
6,6-dimethyl-3-((((4aR,8aR)-1,4,4a,5,6,7,8,8a-octahydroquinazolin-2-yl)thio)methyl)-5,6-dihydroimidazo[2,1-b]thiazole;
6,6-dimethyl-3-((((4aS,8aS)-1,4,4a,5,6,7,8,8a-octahydroquinazolin-2-yl)thio)methyl)-5,6-dihydroimidazo[2,1-b]thiazole;
6,6-dimethyl-3-((((4aR,8aS)-1,4,4a,5,6,7,8,8a-octahydroquinazolin-2-yl)thio)methyl)-5,6-dihydroimidazo[2,1-b]thiazole;
6,6-dimethyl-3-((((4aS,8aR)-1,4,4a,5,6,7,8,8a-octahydroquinazolin-2-yl)thio)methyl)-5,6-dihydroimidazo[2,1-b]thiazole;
5-(2-((5,5-dimethyl-4,5-dihydro-1H-imidazol-2-yl)thio)ethyl)quinoline;
3′-(((5,5-dimethyl-4,5-dihydro-1H-imidazol-2-yl)thio)methyl)-5′H-spiro[cyclohexane-1,6′-imidazo[2,1-b]thiazole];
3-(((5,5-dimethyl-4,5-dihydro-1H-imidazol-2-yl)thio)methyl)-5a,6,7,8,9,9a-hexahydro-5H-thiazolo[2,3-b]quinazoline;
trans-3-(((5,5-dimethyl-4,5-dihydro-1H-imidazol-2-yl)thio)methyl)-5a,6,7,8,9,9a-hexahydro-5H-thiazolo[2,3-b]quinazoline;
cis-3-(((5,5-dimethyl-4,5-dihydro-1H-imidazol-2-yl)thio)methyl)-5a,6,7,8,9,9a-hexahydro-5H-thiazolo[2,3-b]quinazoline;
3-(((5,5-dimethyl-4,5-dihydro-1H-imidazol-2-yl)thio)methyl)-(5aR,9aR)-5a,6,7,8,9,9a-hexahydro-5H-thiazolo[2,3-b]quinazoline;
3-(((5,5-dimethyl-4,5-dihydro-1H-imidazol-2-yl)thio)methyl)-(5aS,9aS)-5a,6,7,8,9,9a-hexahydro-5H-thiazolo[2,3-b]quinazoline;
3-(((5,5-dimethyl-4,5-dihydro-1H-imidazol-2-yl)thio)methyl)-(5aR,9aS)-5a,6,7,8,9,9a-hexahydro-5H-thiazolo[2,3-b]quinazoline;
3-(((5,5-dimethyl-4,5-dihydro-1H-imidazol-2-yl)thio)methyl)-(5aS,9aR)-5a,6,7,8,9,9a-hexahydro-5H-thiazolo[2,3-b]quinazoline;
3-(((3a,4,5,6,7,7a-hexahydro-1H-benzo[d]imidazol-2-yl)thio)methyl)-5,6-dihydroimidazo[2,1-b]thiazole;
trans-3-(((3a,4,5,6,7,7a-hexahydro-1H-benzo[d]imidazol-2-yl)thio)methyl)-5,6-dihydroimidazo[2,1-b]thiazole;
cis-3-(((3a,4,5,6,7,7a-hexahydro-1H-benzo[d]imidazol-2-yl)thio)methyl)-5,6-dihydroimidazo[2,1-b]thiazole;
3-((((3aR,7aR)-3a,4,5,6,7,7a-hexahydro-1H-benzo[d]imidazol-2-yl)thio)methyl)-5,6-dihydroimidazo[2,1-b]thiazole;
3-((((3aS,7aS)-3a,4,5,6,7,7a-hexahydro-1H-benzo[d]imidazol-2-yl)thio)methyl)-5,6-dihydroimidazo[2,1-b]thiazole;
3-((((3aR,7aS)-3a,4,5,6,7,7a-hexahydro-1H-benzo[d]imidazol-2-yl)thio)methyl)-5,6-dihydroimidazo[2,1-b]thiazole;
3-((((3aS,7aR)-3a,4,5,6,7,7a-hexahydro-1H-benzo[d]imidazol-2-yl)thio)methyl)-5,6-dihydroimidazo[2,1-b]thiazole;
3-(((3a,4,5,6,7,7a-hexahydro-1H-benzo[d]imidazol-2-yl)thio)methyl)-6,7-dihydro-5H-thiazolo[3,2-a]pyrimidine;
trans-3-(((3a,4,5,6,7,7a-hexahydro-1H-benzo[d]imidazol-2-yl)thio)methyl)-6,7-dihydro-5H-thiazolo[3,2-a]pyrimidine;
cis-3-(((3a,4,5,6,7,7a-hexahydro-1H-benzo[d]imidazol-2-yl)thio)methyl)-6,7-dihydro-5H-thiazolo[3,2-a]pyrimidine;
3-((((3aR,7aR)-3a,5,6,7,7a-hexahydro-1H-benzo[d]imidazol-2-yl)thio)methy)-6,7-dihydro-5H-thiazolo[3,2-a]pyrimidine;
3-((((3aS,7aS)-3a,4,5,6,7,7a-hexahydro-1H-benzo[d]imidazol-2-yl)thio)methyl)-6,7-dihydro-5H-thiazolo[3,2-a]pyrimidine;
3-((((3aR,7aS)-3a,4,5,6,7,7a-hexahydro-1H-benzo[d]imidazol-2-yl)thio)methyl)-6,7-dihydro-5H-thiazolo[3,2-a]pyrimidine;
3-((((3aS,7aR)-3a,4,5,6,7,7a-hexahydro-1H-benzo[d]imidazol-2-yl)thio)methyl)-6,7-dihydro-5H-thiazolo[3,2-a]pyrimidine;
3-(((3a,4,5,6,7,7a-hexahydro-1H-benzo[d]imidazo-2-yl)thio)methyl)-5,6,7,8-tetrahydrothiazolo[3,2-a][1,3]diazepine;
trans-3-(((3a,5,6,7,7a-hexahydro-1H-benzo[d]imidazol-2-yl)thio)methyl)-5,6,7,8-tetrahydrothiazolo[3,2-a][1,3]diazepine;
cis-3-(((3a,4,5,6,7,7a-hexahydro-1H-benzo[d]imidazol-2-yl)thio)methyl)-5,6,7,8-tetrahydrothiazolo[3,2-a][1,3]diazepine;
3-((((3aR,7aR)-3a,4,5,6,7,7a-hexahydro-1H-benzo[d]imidazol-2-yl)thio)methyl)-5,6,7,8-tetrahydrothiazolo[3,2-a][1,3]diazepine;
3-((((3aS,7aS)-3a,4,5,6,7,7-hexahydro-1H-benzo[d]imidazol-2-yl)thio)methyl)-5,6,7,8-tetrahydrothiazolo[3,2-a][1,3]diazepine;
3-((((3aR,7aS)-3a,4,5,6,7,7a-hexahydro-1H-benzo[d]imidazol-2-yl)thio)methyl)-5,6,7,8-tetrahydrothiazolo[3,2-a][1.3]diazepine;
3-((((3aS,7aR)-3a,4,5,6,7,7a-hexahydro-1H-benzo[d]imidazol-2-yl)thio)methyl)-5,6,7,8-tetrahydrothiazolo[3,2-a][1,3]diazepine;
1-(2-((5,5-dimethyl-4,5-dihydro-1H-imidazol-2-yl)thio)ethyl)-1H-imidazole;
3-(((5-benzyl-4,5-dihydro-1H-imidazol-2-yl)thio)methyl)-6,7-dihydro-5H-thiazolo[3,2-a]pyrimidine;
3-(((5-isopropyl-4,5-dihydro-1H-imidazol-2-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-6-phenyl-5,6-dihydroimidazo[2,1-b]thiazole;
6-benzyl-3-(((5-butyl-4,5-dihydro-1H-imidazol-2-yl)thio)methyl)-5,6-dihydroimidazo[2,1-b]thiazole;
3-(((5,5-dimethyl-4,5-dihydro-1H-imidazol-2-yl)thio)methyl)-6-isopropyl-5,6-dihydroimidazo[2,1-b]thiazole;
6,6-dimethyl-3-(((4-phenyl-4,5-dihydro-1H-imidazol-2-yl)thio)methyl)-5,6-dihydroimidazo[2,1-b]thiazole;
3-(((5-butyl-4,5-dihydro-1H-imidazol-2-yl)thio)methyl)-4a,5,6,7,8,8a-hexahydrobenzo[4,5]imidazo[2,1-b]thiazole;
trans-3-(((5-butyl-4,5-dihydro-1H-imidazol-2-yl)thio)methyl)-4a,5,6,7,8,8a-hexahydrobenzo[4,5]imidazo[2,1-b]thiazole;
cis-3-(((5-butyl-4,5-dihydro-1H-imidazol-2-yl)thio)methyl)-4a,5,6,7,8,8a-hexahydrobenzo[4,5]imidazo[2,1-b]thiazole;
3-(((5-butyl-4,5-dihydro-1H-imidazol-2-yl)thio)methyl)-(4aR,8aR)-4a,5,6,7,8,8a-hexahydrobenzo[4,5]imidazo[2,1-b]thiazole;
3-(((5-butyl-4,5-dihydro-1H-imidazol-2-yl)thio)methyl)-(4aS,8aS)-4a,5,6,7,8,8a-hexahydrobenzo[4,5]imidazo[2,1-b]thiazole;
3-(((5-butyl-4,5-dihydro-1H-imidazol-2-yl)thio)methyl)-(4aR,8aS)-4a,5,6,7,8,8a-hexahydrobenzo[4,5]imidazo[2,1-b]thiazole;
3-(((5-butyl-4,5-dihydro-1H-imidazol-2-yl)thio)methyl)-(4aS,8aR)-4a,5,6,7,8,8a-hexahydrobenzo[4,5]imidazo[2,1-b]thiazole;
3-(((5-benzyl-4,5-dihydro-1H-imidazol-2-yl)thio)methyl)-4a,5,6,7,8,8a-hexahydrobenzo[4,5]imidazo[2,1-b]thiazole;
trans-3-(((5-benzyl-4,5-dihydro-1H-imidazol-2-yl)thio)methyl)-4a,5,6,7,8,8a-hexahydrobenzo[4,5]imidazo[2,1-b]thiazole;
cis-3-(((5-benzyl-4,5-dihydro-1H-imidazol-2-yl)thio)methyl)-4a,5,6,7,8,8a-hexahydrobenzo[4,5]imidazo[2,1-b]thiazole;
3-(((5-benzyl-4,5-dihydro-1H-imidazol-2-yl)thio)methyl)-(4aS,8aR)-4a,5,6,7,8,8a-hexahydrobenzo[4,5]imidazo[2,1-b]thiazole;
3-(((5-benzyl-4,5-dihydro-1H-imidazol-2-yl)thio)methyl)-(4aR,8aS)-4a,5,6,7,8,8a-hexahydrobenzo[4,5]imidazo[2,1-b]thiazole;
3-(((5-benzyl-4,5-dihydro-1H-imidazol-2-yl)thio)methyl)-(4aR,8aR)-4a,5,6,7,8,8a-hexahydrobenzo[4,5]imidazo[2,1-b]thiazole;
3-(((5-benzyl-4,5-dihydro-1H-imidazol-2-yl)thio)methyl)-(4aS,8aS)-4a,5,6,7,8,8a-hexahydrobenzo[4,5]imidazo[2,1-b]thiazole;
3-(((3a,4,5,6,7,7a-hexahydro-1H-benzo[d]imidazol-2-yl)thio)methyl)-4a,5,6,7,8,8a-hexahydrobenzo[4,5]imidazo[2,1-b]thiazole;
trans-3-((((3aS,7aS)-3a,4,5,6,7,7a-hexahydro-1H-benzo[d]imidazol-2-yl)thio)methyl)-4a,5,6,7,8,8a-hexahydrobenzo[4,5]imidazo[2,1-b]thiazole;
cis-3-((((3aS,7aS)-3a,4,5,6,7,7a-hexahydro-1H-benzo[d]imidazol-2-yl)thio)methyl)-4a,5,6,7,8,8a-hexahydrobenzo[4,5]imidazo[2,1-b]thiazole;
3-((((3aS,7aS)-3a,4,5,6,7,7a-hexahydro-1H-benzo[d]imidazol-2-yl)thio)methyl)-(4aR,8aS)-4a,5,6,7,8,8a-hexahydrobenzo[4,5]imidazo[2,1-b]thiazole;
3-((((3aS,7aS)-3a,4,5,6,7,7a-hexahydro-1H-benzo[d]imidazol-2-yl)thio)methyl)-(4aS,8aR)-4a,5,6,7,8,8a-hexahydrobenzo[4,5]imidazo[2,1-b]thiazole;
3-((((3aS,7aS)-3a,4,5,6,7,7a-hexahydro-1H-benzo[d]imidazol-2-yl)thio)methyl)-(4aR,8aR)-4a,5,6,7,8,8a-hexahydrobenzo[4,5]imidazo[2,1-b]thiazole;
3-((((3aS,7aS)-3a,4,5,6,7,7a-hexahydro-1H-benzo[d]imidazol-2-yl)thio)methyl)-(4aS,8aS)-4a,5,6,7,8,8a-hexahydrobenzo[4,5]imidazo[2,1-b]thiazole;
3-((((3aR,7aR)-3a,4,5,6,7,7a-hexahydro-1H-benzo[d]imidazol-2-yl)thio)methyl)-(4aR,8aS)-4a,5,6,7,8,8a-hexahydrobenzo[4,5]imidazo[2,1-b]thiazole;
3-((((3aR,7aR)-3a,4,5,6,7,7a-hexahydro-1H-benzo[d]imidazol-2-yl)thio)methyl)-(4aS,8aR)-4a,5,6,7,8,8a-hexahydrobenzo[4,5]imidazo[2,1-b]thiazole;
3-((((3aR,7aR)-3a,4,5,6,7,7a-hexahydro-1H-benzo[d]imidazol-2-yl)thio)methyl)-(4aR,8aR)-4a,5,6,7,8,8a-hexahydrobenzo[4,5]imidazo[2,1-b]thiazole;
3-((((3aR,7aR)-3a,4,5,6,7,7a-hexahydro-1H-benzo[d]imidazol-2-yl)thio)methyl)-(4aS,8aS)-4a,5,6,7,8,8a-hexahydrobenzo[4,5]imidazo[2,1-b]thiazole;
3-((((3aR,7aS)-3a,4,5,6,7,7a-hexahydro-1H-benzo[d]imidazol-2-yl)thio)methyl)-(4aR,8aS)-4a,5,6,7,8,8a-hexahydrobenzo[4,5]imidazo[2,1-b]thiazole;
3-((((3aR,7aS)-3a,4,5,6,7,7a-hexahydro-1H-benzo[d]imidazol-2-yl)thio)methyl)-(4aS,8aR)-4a,5,6,7,8,8a-hexahydrobenzo[4,5]imidazo[2,1-b]thiazole;
3-((((3aR,7aS)-3a,4,5,6,7,7a-hexahydro-1H-benzo[d]imidazol-2-yl)thio)methyl)-(4aR,8aR)-4a,5,6,7,8,8a-hexahydrobenzo[4,5]imidazo[2,1-b]thiazole;
3-((((3aR,7aS)-3a,4,5,6,7,7a-hexahydro-1H-benzo[d]imidazol-2-yl)thio)methyl)-(4aS,8aS)-4a,5,6,7,8,8a-hexahydrobenzo[4,5]imidazo[2,1-b]thiazole;
3-((((3aS,7aR)-3a,4,5,6,7,7a-hexahydro-1H-benzo[d]imidazol-2-yl)thio)methyl)-(4aR,8aS)-4a,5,6,7,8,8a-hexahydrobenzo[4,5]imidazo[2,1-b]thiazole;
3-((((3aS,7aR)-3a,4,5,6,7,7a-hexahydro-1H-benzo[d]imidazol-2-yl)thio)methyl)-(4aS,8aR)-4a,5,6,7,8,8a-hexahydrobenzo[4,5]imidazo[2,1-b]thiazole;
3-((((3aS,7aR)-3a,4,5,6,7,7a-hexahydro-1H-benzo[d]imidazol-2-yl)thio)methyl)-(4aR,8aR)-4a,5,6,7,8,8a-hexahydrobenzo[4,5]imidazo[2,1-b]thiazole;
3-((((3aS,7aR)-3a,4,5,6,7,7a-hexahydro-1H-benzo[d]imidazol-2-yl)thio)methyl)-(4aS,8aS)-4a,5,6,7,8,8a-hexahydrobenzo[4,5]imidazo[2,1-b]thiazole;
6,6-dimethyl-3-(((1-methyl-1,4,5,6-tetrahydropyrimidin-2-yl)thio)methyl)-5,6-dihydroimidazo[2,1-b]thiazole;
3-(((4,5-diisopropyl-4,5-dihydro-1H-imidazol-2-yl)thio)methyl)-6,6-dimethyl-5,6-dihydroimidazo[2,1-b]thiazole;
trans-3-(((4,5-diisopropyl-4,5-dihydro-1H-imidazol-2-yl)thio)methyl)-6,6-dimethyl-5,6-dihydroimidazo[2,1-b]thiazole;
cis-3-(((4,5-diisopropyl-4,5-dihydro-1H-imidazol-2-yl)thio)methyl)-6,6-dimethyl-5,6-dihydroimidazo[2,1-b]thiazole;
3-((((4S,5S)-4,5-diisopropyl-4,5-dihydro-1H-imidazol-2-yl)thio)methyl)-6,6-dimethyl-5,6-dihydroimidazo[2,1-b]thiazole;
3-((((4R,5R)-4,5-diisopropyl-4,5-dihydro-1H-imidazol-2-yl)thio)methyl)-6,6-dimethyl-5,6-dihydroimidazo[2,1-b]thiazole;
3-((((4S,5R)-4,5-diisopropyl-4,5-dihydro-1H-imidazol-2-yl)thio)methyl)-6,6-dimethyl-5,6-dihydroimidazo[2,1-b]thiazole;
3-((((4R,5S)-4,5-diisopropyl-4,5-dihydro-1H-imidazol-2-yl)thio)methyl)-6,6-dimethyl-5,6-dihydroimidazo[2,1-b]thiazole;
3-(((4,5-dihydro-1H-imidazol-2-yl)thio)methyl)-4a,5,6,7,8,8a-hexahydrobenzo[4,5]imidazo[2,1-b]thiazole;
trans-3-(((4,5-dihydro-1H-imidazol-2-yl)thio)methyl)-4a,5,6,7,8,8a-hexahydrobenzo[4,5]imidazo[2,1-b]thiazole;
cis-3-(((4,5-dihydro-1H-imidazol-2-yl)thio)methyl)-4a,5,6,7,8,8a-hexahydrobenzo[4,5]imidazo[2,1-b]thiazole;
3-(((4,5-dihydro-1H-imidazol-2-yl)thio)methyl)-(4aR,8aS)-4a,5,6,7,8,8a-hexahydrobenzo[4,5]imidazo[2,1-b]thiazole;
3-(((4,5-dihydro-1H-imidazol-2-yl)thio)methyl)-(4aS,8aR)-4a,5,6,7,8,8a-hexahydrobenzo[4,5]imidazo[2,1-b]thiazole;
3-(((4,5-dihydro-1H-imidazol-2-yl)thio)methyl)-(4aR,8aR)-4a,5,6,7,8,8a-hexahydrobenzo[4,5]imidazo[2,1-b]thiazole;
3-(((4,5-dihydro-1H-imidazol-2-yl)thio)methyl)-(4aS,8aS)-4a,5,6,7,8,8a-hexahydrobenzo[4,5]imidazo[2,1-b]thiazole;
6-benzyl-3-(((5-benzyl-4,5-dihydro-1H-imidazol-2-yl)thio)methyl)-5,6-dihydroimidazo[2,1-b]thiazole;
6-benzyl-3-(((3a,4,5,6,7,7a-hexahydro-1H-benzo[d]imidazol-2-yl)thio)methyl)-5,6-dihydroimidazo[2,1-b]thiazole;
trans-6-benzyl-3-(((3a,4,5,6,7,7a-hexahydro-1H-benzo[d]imidazol-2-yl)thio)methyl)-5,6-dihydroimidazo[2,1-b]thiazole;
cis-6-benzyl-3-(((3a,4,5,6,7,7a-hexahydro-1H-benzo[d]imidazol-2-yl)thio)methyl)-5,6-dihydroimidazo[2,1-b]thiazole;
6-benzyl-3-((((3aR,7aR)-3a,4,5,6,7,7a-hexahydro-1H-benzo[d]imidazol-2-yl)thio)methyl)-5,6-dihydroimidazo[2,1-b]thiazole;
6-benzyl-3-((((3aS,7aS)-3a,4,5,6,7,7a-hexahydro-1H-benzo[d]imidazol-2-yl)thio)methyl)-5,6-dihydroimidazo[2,1-b]thiazole;
6-benzyl-3-((((3aR,7aS)-3a,4,5,6,7,7a-hexahydro-1H-benzo[d]imidazol-2-yl)thio)methyl)-5,6-dihydroimidazo[2,1-b]thiazole;
6-benzyl-3-((((3aS,7aR)-3a,4,5,6,7,7a-hexahydro-1H-benzo[d]imidazol-2-yl)thio)methyl)-5,6-dihydroimidazo[2,1-b]thiazole;
3-(((5,5-dimethyl-4,5-dihydro-1H-imidazol-2-yl)thio)methyl)-5,6-diisopropyl-5,6-dihydroimidazo[2,1-b]thiazole;
trans-3-(((5,5-dimethyl-4,5-dihydro-1H-imidazol-2-yl)thio)methyl)-5,6-diisopropyl-5,6-dihydroimidazo[2,1-b]thiazole;
cis-3-(((5,5-dimethyl-4,5-dihydro-1H-imidazol-2-yl)thio)methyl)-5,6-diisopropyl-5,6-dihydroimidazo[2,1-b]thiazole;
3-(((5,5-dimethyl-4,5-dihydro-1H-imidazol-2-yl)thio)methyl)-(5R,6R)-5,6-diisopropyl-5,6-dihydroimidazo[2,1-b]thiazole;
3-(((5,5-dimethyl-4,5-dihydro-1H-imidazol-2-yl)thio)methyl)-(5S,6S)-5,6-diisopropyl-5,6-dihydroimidazo[2,1-b]thiazole;
3-(((5,5-dimethyl-4,5-dihydro-1H-imidazol-2-yl)thio)methyl)-(5R,6S)-5,6-diisopropyl-5,6-dihydroimidazo[2,1-b]thiazole;
3-(((5,5-dimethyl-4,5-dihydro-1H-imidazol-2-yl)thio)methyl)-(5S,6R)-5,6-diisopropyl-5,6-dihydroimidazo[2,1-b]thiazole;
3-(((1-methyl-4,5-dihydro-1H-imidazol-2-yl)thio)methyl)-4a,5,6,7,8,8a-hexahydrobenzo[4,5]imidazo[2,1-b]thiazole;
trans-3-(((1-methyl-4,5-dihydro-1H-imidazol-2-yl)thio)methyl)-4a,5,6,7,8,8a-hexahydrobenzo[4,5]imidazo[2,1-b]thiazole;
cis-3-(((1-methyl-4,5-dihydro-1H-imidazol-2-yl)thio)methyl)-4a,5,6,7,8,8a-hexahydrobenzo[4,5]imidazo[2,1-b]thiazole;
3-(((1-methyl-4,5-dihydro-1H-imidazol-2-yl)thio)methyl)-(4aR,8aR)-4a,5,6,7,8,8a-hexahydrobenzo[4,5]imidazo[2,1-b]thiazole;
3-(((1-methyl-4,5-dihydro-1H-imidazol-2-yl)thio)methyl)-(4aS,8aS)-4a,5,6,7,8,8a-hexahydrobenzo[4,5]imidazo[2,1-b]thiazole;
3-(((1-methyl-4,5-dihydro-1H-imidazol-2-yl)thio)methyl)-(4aR,8aS)-4a,5,6,7,8,8a-hexahydrobenzo[4,5]imidazo[2,1-b]thiazole;
3-(((1-methyl-4,5-dihydro-1H-imidazol-2-yl)thio)methyl)-(4aS,8aR)-4a,5,6,7,8,8a-hexahydrobenzo[4,5]imidazo[2,1-b]thiazole;
3-(3-((5,5-dimethyl-4,5-dihydro-1H-imidazol-2-yl)thio)propyl)-6,6-dimethyl-5,6-dihydroimidazo[2,1-b]thiazole;
3-(2-((5-benzyl-4,5-dihydro-1H-imidazol-2-yl)thio)ethyl)pyridine;
3-(((5,5-dimethyl-4,5-dihydro-1H-imidazol-2-yl)thio)methyl)-2-iodo-6,6-dimethyl-5,6-dihydroimidazo[2,1-b]thiazole;
5-benzyl-2-((1-benzylpyrrolidin-3-yl)thio)-4,5-dihydro-1H-imidazole;
7-(3-((5-benzyl-4,5-dihydro-1H-imidazol-2-yl)thio)propyl)-1,2,3,4-tetrahydro-1,8-naphthyridine;
6-benzyl-3-(((1-methyl-4,5-dihydro-1H-imidazol-2-yl)thio)methyl)-5,6-dihydroimidazo[2,1-b]thiazole;
3-((5,5-dimethyl-4,5-dihydro-1H-imidazol-2-yl)thio)-6,6-dimethyl-2,3,5,6-tetrahydroimidazo[2,1-b]thiazole;
   and pharmaceutically acceptable salts and solvates of any one of the aforementioned compounds.

The present invention also relates to each of the intermediates described further below in the examples section of this specification, including any one of these intermediates in non-salt form or in the form of a salt (e.g., a pharmaceutically acceptable salt) of the respective compound. Such intermediates can be used, in particular, in the synthesis of the compounds of formula (I).

In a first preferred embodiment, the compound of formula (I) is a compound of the following formula (Ia)

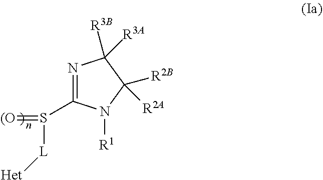

or a pharmaceutically acceptable salt or solvate thereof, wherein the groups/variables in formula (Ia), particularly R¹, R^{2A}, R^{2B}, R^{3A}, R^{3B}, n, L and Het, have the same meanings, including the same preferred meanings, as described and defined herein above for the compound of formula (I).

In a second preferred embodiment, the compound of formula (I) is a compound of the following formula (Ib)

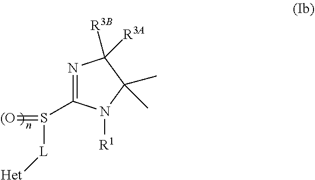

or a pharmaceutically acceptable salt or solvate thereof, wherein the groups/variables in formula (Ib), particularly R¹, R^{3A}, R^{3B}, n, L and Het, have the same meanings, including the same preferred meanings, as described and defined herein above for the compound of formula (I).

In a third preferred embodiment, the compound of formula (I) is a compound of the following formula (Ic)

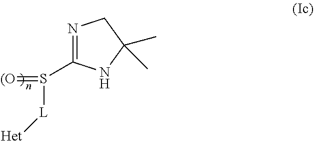

or a pharmaceutically acceptable salt or solvate thereof, wherein the groups/variables in formula (Ic), particularly n, L and Het, have the same meanings, including the same preferred meanings, as described and defined herein above for the compound of formula (I).

In a fourth preferred embodiment, the compound of formula (I) is a compound of the following formula (Id)

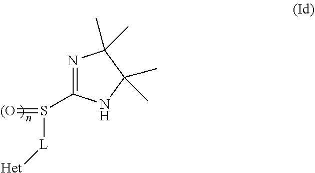

or a pharmaceutically acceptable salt or solvate thereof, wherein the groups/variables in formula (Id), particularly n, L and Het, have the same meanings, including the same preferred meanings, as described and defined herein above for the compound of formula (I).

In a fifth preferred embodiment, the compound of formula (I) is a compound of the following formula (Ie)

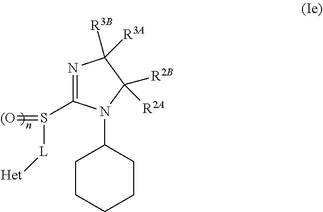

or a pharmaceutically acceptable salt or solvate thereof, wherein the groups/variables in formula (Ie), particularly R^{2A}, R^{2B}, R^{3A}, R^{3B}, n, L and Het, have the same meanings, including the same preferred meanings, as described and defined herein above for the compound of formula (I).

In a sixth preferred embodiment, the compound of formula (I) is a compound of the following formula (If)

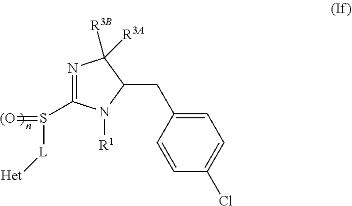

or a pharmaceutically acceptable salt or solvate thereof, wherein the groups/variables in formula (if), particularly R¹, R^{3A}, R^{3B}, n, L and Het, have the same meanings, including the same preferred meanings, as described and defined herein above for the compound of formula (I).

In a seventh preferred embodiment, the compound of formula (I) is a compound of the following formula (Ig)

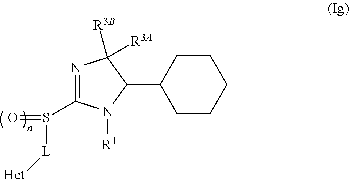

or a pharmaceutically acceptable salt or solvate thereof, wherein the groups/variables in formula (Ig), particularly R¹, R^{3A}, R^{3B}, n, L and Het, have the same meanings, including the same preferred meanings, as described and defined herein above for the compound of formula (I).

In an eighth preferred embodiment, the compound of formula (I) is a compound of the following formula (Ih)

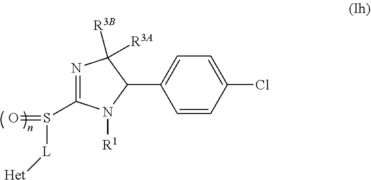

or a pharmaceutically acceptable salt or solvate thereof, wherein the groups/variables in formula (Ih), particularly R¹, R^{3A}, R^{3B}, n, L and Het, have the same meanings, including the same preferred meanings, as described and defined herein above for the compound of formula (l).

In a ninth preferred embodiment, the compound of formula (I) is a compound of the following formula (Ii)

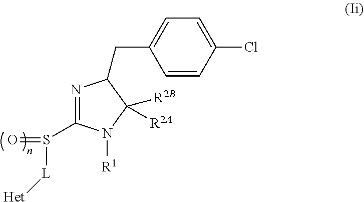

or a pharmaceutically acceptable salt or solvate thereof, wherein the groups/variables in formula (Ii), particularly R¹, R^{2A}, R^{2B}, n, L and Het, have the same meanings, including the same preferred meanings, as described and defined herein above for the compound of formula (I).

In a tenth preferred embodiment, the compound of formula (I) is a compound of the following formula (Ij)

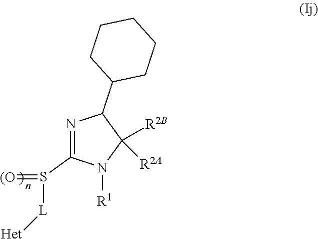

or a pharmaceutically acceptable salt or solvate thereof, wherein the groups/variables in formula (Ij), particularly R¹, R^{2A}, R^{2B}, n, L and Het, have the same meanings, including the same preferred meanings, as described and defined herein above for the compound of formula (I).

In an 11^{th} preferred embodiment, the compound of formula (I) is a compound of the following formula (Ik)

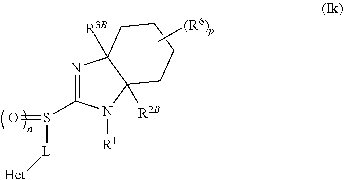

or a pharmaceutically acceptable salt or solvate thereof, wherein p is an integer of 0 to 6, and wherein the further groups/variables in formula (Ik), particularly R¹, R^{2B}, R^{3B}, R⁶, n, L and Het, have the same meanings, including the same preferred meanings, as described and defined herein above for the compound of formula (I). It will be understood that p indicates the number of substituents R⁶ that are bound to the cyclohexyl moiety comprised in the bicyclic ring system of the compound of formula (Ik); if p is 0, then this phenyl moiety is not substituted with any group R⁶, i.e. is substituted with hydrogen instead of R⁶. Preferably, p is 0, 1, 2 or 3, more preferably p is 0, 1 or 2, and even more preferably p is 0.

In a 12^{th} preferred embodiment, the compound of formula (I) is a compound of the following formula (Im)

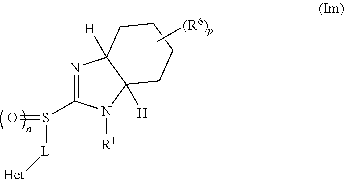

or a pharmaceutically acceptable salt or solvate thereof, wherein p is an integer of 0 to 6, and wherein the further groups/variables in formula (Im), particularly R¹, R⁶, n, L and Het, have the same meanings, including the same preferred meanings, as described and defined herein above for the compound of formula (I). It will be understood that p indicates the number of substituents R⁶ that are bound to the cyclohexyl moiety comprised in the bicyclic ring system of the compound of formula (Im); if p is 0, then this phenyl moiety is not substituted with any group R⁶. i.e. is substituted with hydrogen instead of R⁶. Preferably, p is 0, 1, 2 or 3, more preferably p is 0, 1 or 2, and even more preferably p is 0. The invention also specifically relates to each stereoisomer of the compound of formula (Im), including in particular:

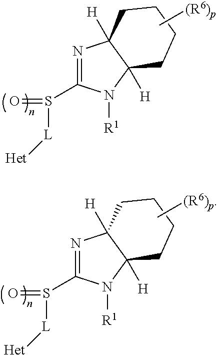

It is preferred that the above-depicted two stereocenters of the compound of formula (Im) have the trans-configuration. In this regard, the present invention also specifically relates to each individual enantiomer of the trans-isomer of the compound of formula (Im).

In a 13^{th} preferred embodiment, the compound of formula (I) is a compound of the following formula (In)

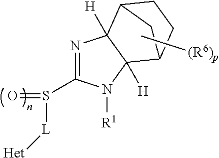

or a pharmaceutically acceptable salt or solvate thereof, wherein p is an integer of 0 to 6, and wherein the further groups/variables in formula (in), particularly R¹, R⁶, n, L and Het, have the same meanings, including the same preferred meanings, as described and defined herein above for the compound of formula (I). It will be understood that p indicates the number of substituents R⁶ that are bound to the norbornane ring comprised in the tricyclic ring system of the compound of formula (In); if p is 0, then this norbornane ring is not substituted with any group R⁶, i.e. is substituted with hydrogen instead of R⁶. Preferably, p is 0, 1, 2 or 3, more preferably p is 0, 1 or 2. In particular, p may be 0.

In a 14^{th} preferred embodiment, the compound of formula (I) is 3-((4,5-dihydro-1H-imidazol-2-ylthio)methyl)-5,6-dihydroimidazo[2,1-b]thiazole or a pharmaceutically acceptable salt or solvate thereof. Yet, the invention also relates to a compound of formula (I) which is different from the aforementioned compound, or a pharmaceutically acceptable salt or solvate thereof.

In a 15^{th} preferred embodiment, the compound of formula (I) is a compound of the following formula (Io)

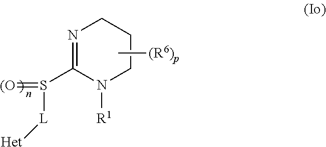

or a pharmaceutically acceptable salt or solvate thereof, wherein p is an integer of 0 to 6, and wherein the further groups/variables in formula (Io), particularly R¹, R⁶, n, L and Het, have the same meanings, including the same preferred meanings, as described and defined herein above for the compound of formula (I). It will be understood that p indicates the number of substituents R⁶ that are bound to the 1,4,5,6-tetrahydropyrimidinyl ring of the compound of formula (Io); if p is 0, then this 1,4,5,6-tetrahydropyrimidinyl ring is not substituted with any group R⁶, i.e. is substituted with hydrogen instead of R⁶. Preferably, p is 0, 1, 2 or 3, more preferably p is 0, 1 or 2. In particular, p may be 0.

In a 16^{th} preferred embodiment, the compound of formula (I) is a compound of the following formula (Ip)

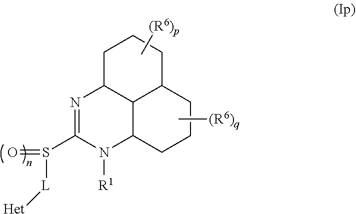

or a pharmaceutically acceptable salt or solvate thereof, wherein p and q are each independently an integer of 0 to 6, and wherein the further groups/variables in formula (Ip), particularly R¹, R⁶, n, L and Het, have the same meanings, including the same preferred meanings, as described and defined herein above for the compound of formula (I). It will be understood that p and q indicate the number of substituents R⁶ that are bound to the corresponding cyclohexyl moiety comprised in the tricyclic ring system of the compound of formula (Ip); if p or q is 0, then the corresponding cyclohexyl moiety is not substituted with any group R⁶, i.e. is substituted with hydrogen instead of R⁶. Preferably, p and q are each independently selected from 0, 1, 2 and 3, more preferably p and q are each independently selected from 0, 1 and 2. In particular, p and q may each be 0.

In a 17^{th} preferred embodiment, the compound of formula (I) is a compound of the following formula (Iq)

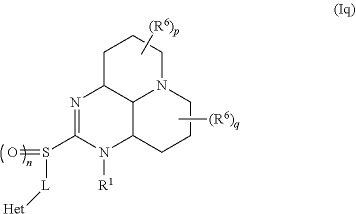

or a pharmaceutically acceptable salt or solvate thereof, wherein p and q are each independently an integer of 0 to 6, and wherein the further groups/variables in formula (Iq), particularly R¹, R⁶, n, L and Het, have the same meanings, including the same preferred meanings, as described and defined herein above for the compound of formula (I). It will be understood that p and q indicate the number of substituents R⁶ that are bound to the corresponding piperidine moiety comprised in the tricyclic ring system of the compound of formula (Iq); if p or q is 0, then the corresponding piperidine moiety is not substituted with any group R⁶, i.e. is substituted with hydrogen instead of R⁶. Preferably, p and q are each independently selected from 0, 1, 2 and 3, more preferably p and q are each independently selected from 0, 1 and 2. In particular, p and q may each be 0.

In an 18^{th} preferred embodiment, the compound of formula (I) is a compound of the following formula (Ir)

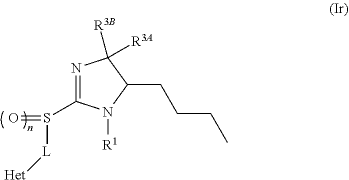

or a pharmaceutically acceptable salt or solvate thereof, wherein the groups/variables in formula (Ir), particularly R¹, R^{3A}, R^{3B}, n, L and Het, have the same meanings, including the same preferred meanings, as described herein above for the compound of formula (I).

In a 19^{th} preferred embodiment, the compound of formula (I) is a compound of the following formula (Is)

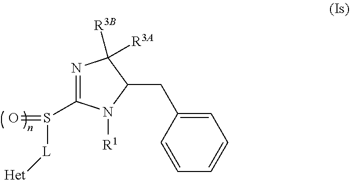

or a pharmaceutically acceptable salt or solvate thereof, wherein the groups/variables in formula (is), particularly R¹, R^{3A}, R^{3B}, n, L and Het, have the same meanings, including the same preferred meanings, as described herein above for the compound of formula (I).

In a 20^{th} preferred embodiment, the compound of formula (I) is a compound of the following formula (It)

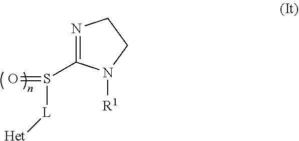

or a pharmaceutically acceptable salt or solvate thereof, wherein the groups/variables in formula (It), particularly R¹, n, L and Het, have the same meanings, including the same preferred meanings, as described and defined herein above for the compound of formula (I).

For a person skilled in the field of synthetic chemistry, various ways for the preparation of the compounds of formula (I) will be readily apparent. For example, the compounds of formula (I) can be prepared in accordance with or in analogy to the synthetic routes described in the examples section.

The following definitions apply throughout the present specification and the claims, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl. As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms; the term “C₀₋₅ alkylene” indicates that a covalent bond (corresponding to the option “C₀ alkylene”) or a C₁₋₅ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₅ alkenylene” denotes an alkenylene group having 2 to 5 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more (e.g., one or two) carbon-to-carbon double bonds. A “C₂₋₅ alkynylene” denotes an alkynylene group having 2 to 5 carbon atoms. Unless defined otherwise, the term “alkynylene” preferably refers to C₂₋₄ alkynylene (including, in particular, linear C₂₋₄ alkynylene).

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). If the aryl is a bridged and/or fused ring system which contains, besides one or more aromatic rings, at least one non-aromatic ring (e.g., a saturated ring or an unsaturated alicyclic ring), then one or more carbon ring atoms in each non-aromatic ring may optionally be oxidized (i.e., to form an oxo group). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 3H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “cycloalkylene” refers to a cycloalkyl group, as defined herein above, but having two points of attachment, i.e. a divalent saturated hydrocarbon ring group. “Cycloalkylene” may, e.g., refer to cyclopropylene (e.g., cyclopropan-1,1-diyl or cyclopropan-1,2-diyl), cyclobutylene (e.g., cyclobutan-1,1-diyl, cyclobutan-1,2-diyl, or cyclobutan-1,3-diyl), cyclopentylene (e.g., cyclopentan-1,1-diyl, cyclopentan-1,2-diyl, or cyclopentan-1,3-diyl), or cyclohexylene (e.g., cyclohexan-1,1-diyl, cyclohexan-1,2-diyl, cyclohexan-1,3-diyl, or cyclohexan-1,4-diyl). Unless defined otherwise, “cycloalkylene” preferably refers to a C₃₋₇ cycloalkylene, and more preferably refers to a C₃₋₅ cycloalkylene. Moreover, unless defined otherwise, a particularly preferred example of a “cycloalkylene” is cyclopropylene.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “heterocycloalkylene” refers to a heterocycloalkyl group, as defined herein above, but having two points of attachment. “Heterocycloalkylene” may, e.g., refer to aziridinylene, azetidinylene, pyrrolidinylene, imidazolidinylene, pyrazolidinylene, piperidinylene, piperazinylene, azepanylene, diazepanylene (e.g., 1,4-diazepanylene), oxazolidinylene, isoxazolidinylene, thiazolidinylene, isothiazolidinylene, morpholinylene, thiomorpholinylene, oxazepanylene, oxiranylene, oxetanylene, tetrahydrofuranylene, 1,3-dioxolanylene, tetrahydropyranylene, 1,4-dioxanylene, oxepanylene, thiiranylene, thietanylene, tetrahydrothiophenylene (i.e., thiolanylene), 1,3-dithiolanylene, thianylene, or thiepanylene. Unless defined otherwise, “heterocycloalkylene” preferably refers to a divalent 3 to 7 membered saturated monocyclic ring group, wherein said ring group contains one or more (e.g., one, two, three, or four) ring heteroatoms independently selected from O, S and N, wherein the remaining ring atoms are carbon atoms, wherein one or more S ring atoms (if present) and/or one or more N ring atoms (if present) are optionally oxidized, and wherein one or more carbon ring atoms are optionally oxidized; more preferably, “heterocycloalkylene” refers to a divalent 3 to 5 membered saturated monocyclic ring group containing one or two (preferably one) ring heteroatoms independently selected from O, S and N, wherein the remaining ring atoms are carbon atoms. Moreover, unless defined otherwise, particularly preferred examples of a “heterocycloalkylene” include aziridinylene, oxiranylene, thiiranylene, azetidinylene (e.g., azetidin-3,3-diyl), oxetanylene (e.g., oxetan-3,3-diyl), thietanylene (e.g., thietan-3,3-diyl), pyrrolidinylene, tetrahydrofuranylene, or tetrahydrothiophenylene.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the present invention embraces all pharmaceutically acceptable salt forms of the compounds of formula (I) which may be formed, e.g., by protonation of an atom carrying an electron lone pair which is susceptible to protonation; such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. A pharmaceutically acceptable salt of the compound of formula (I) is preferably not a hydroiodide salt. Preferred pharmaceutically acceptable salts of the compounds of formula (I) include a hydrochloride salt, a hydrobromide salt, a mesylate salt, a sulfate salt, a tartrate salt, a fumarate salt, an acetate salt, an oxalate salt, a citrate salt, and a phosphate salt. A particularly preferred pharmaceutically acceptable salt of the compound of formula (I) is a hydrochloride salt. Accordingly, if a compound of formula (I), including any one of the specific compounds of formula (I) described herein, is provided in the form of a pharmaceutically acceptable salt, it is preferred that the respective compound is in the form of a hydrochloride salt, a hydrobromide salt, a mesylate salt, a sulfate salt, a tartrate salt, a fumarate salt, an acetate salt, an oxalate salt, a citrate salt, or a phosphate salt, and it is particularly preferred that it is in the form of a hydrochloride salt.

The present invention also specifically relates to the compound of formula (I), including any one of the specific compounds of formula (I) described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I). It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., R²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (2H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds of formula (I) may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the above described pharmaceutical compositions comprising a compound of formula (I) may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Particularly preferred routes of administration are oral administration or parenteral administration.

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or a pharmaceutical composition comprising the compound of formula (I) can be administered in monotherapy (e.g., without concomitantly administering any further therapeutic agents, or without concomitantly administering any further therapeutic agents against the same disease that is to be treated or prevented with the compound of formula (I)). However, the compound of formula (I) or a pharmaceutical composition comprising the compound of formula (I) can also be administered in combination with one or more further therapeutic agents, preferably in combination with one or more further therapeutic agents selected from antimalarial agents, steroids, methotrexate, Janus kinase inhibitors, Toll-like receptors inhibitors and interferon inhibitors. If the compound of formula (I) is used in combination with a second therapeutic agent active against the same disease or condition, the dose of each compound may differ from that when the corresponding compound is used alone, in particular, a lower dose of each compound may be used. The combination of the compound of formula (I) with one or more further therapeutic agents may comprise the simultaneous/concomitant administration of the compound of formula (I) and the further therapeutic agent(s) (either in a single pharmaceutical formulation or in separate pharmaceutical formulations), or the sequential/separate administration of the compound of formula (I) and the further therapeutic agent(s). If administration is sequential, either the compound of formula (I) according to the invention or the one or more further therapeutic agents may be administered first. If administration is simultaneous, the one or more further therapeutic agents may be included in the same pharmaceutical formulation as the compound of formula (I), or they may be administered in two or more different (separate) pharmaceutical formulations.

The subject or patient to be treated in accordance with the present invention may be an animal (e.g., a non-human animal). Preferably, the subject/patient is a mammal. More preferably, the subject/patient is a human (e.g., a male human or a female human; particularly a female human) or a non-human mammal (such as, e.g, a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease, as used herein, is well-known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well-known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features described herein, including any combination of general and/or preferred features. In particular, the invention specifically relates to each combination of meanings (including general and/or preferred meanings) for the various groups and variables comprised in formula (I).

In this specification, a number of documents including patent applications, scientific literature and manufacturers' manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The present invention is also described by the appended illustrative figures:

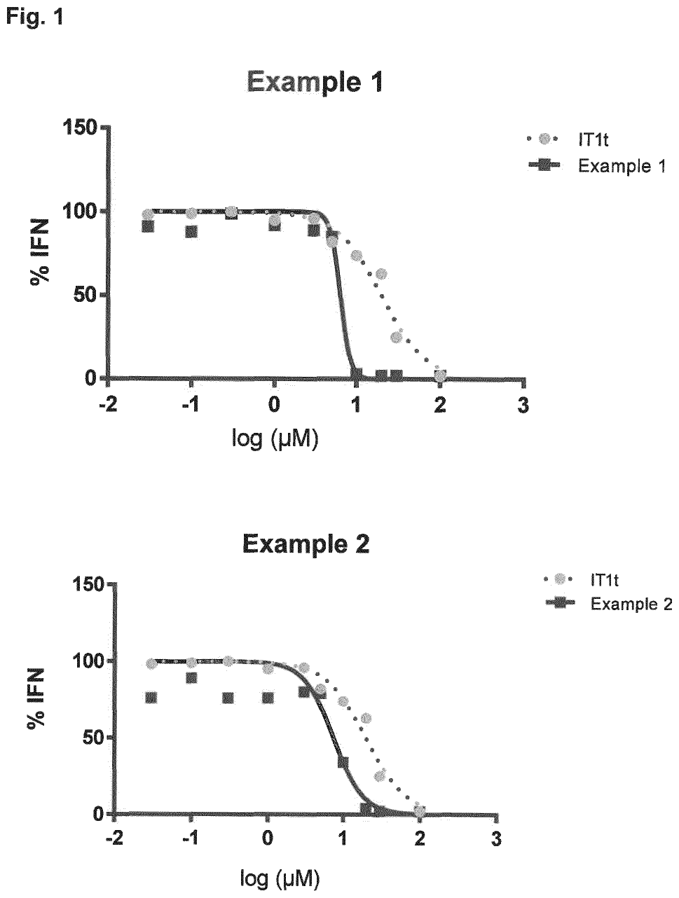

FIG. 1: Effect of examples 1 and 2 on IFN production by activated immune cells (PBMC) from 3 healthy donors. PBMC from 3 different healthy donors blood were isolated by Ficoll gradient and cells were incubated with different concentrations of example 1 or 2 or IT1t before activation with the TLR7 ligand R848 overnight. IFN secretion in the supernatant was quantified using STING-37 reporter cell line. Levels of IFN were measured by quantifying the luciferase activity induced by the presence of IFN. See example 71.

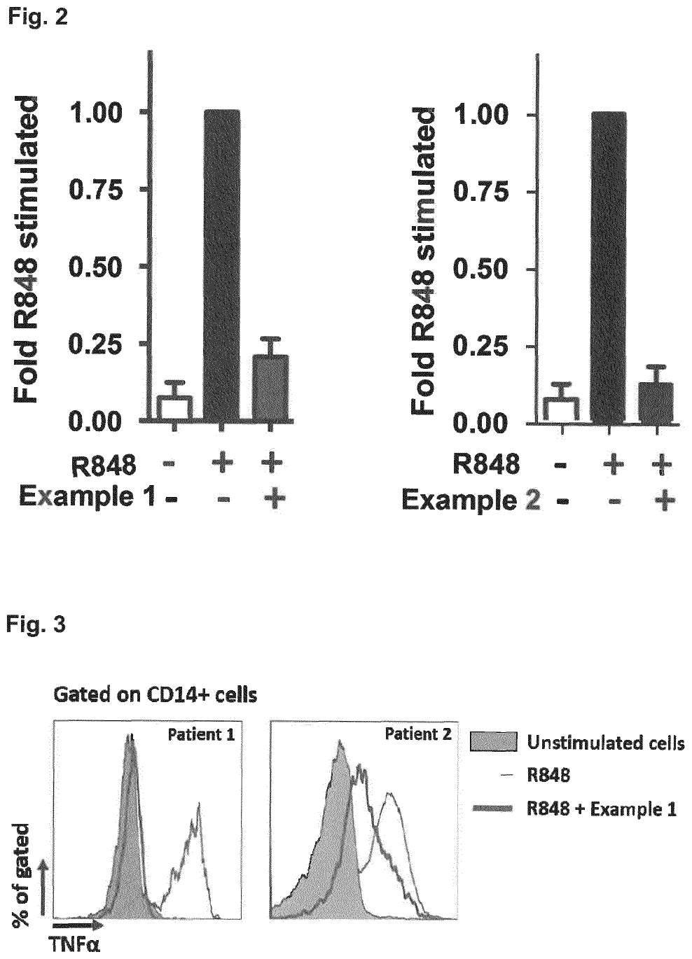

FIG. 2: Effect of examples 1 and 2 on IFN production by activated immune cells (PBMC) from 3 lupus patients. PBMC from 3 different lupus patients were isolated by Ficoll gradient and cells were incubated with example 1 (10 μM) or example 2 (10 μM) before activation with the TLR7 ligand R848 overnight. IFN in the supernatants was quantified using STING-37 reporter cell line. Levels of IFN were measured by quantifying the increased luciferase activity induced by the presence of IFN. See example 71.

FIG. 3: Effect of example 1 on TNFα production by monocytes from 2 juvenile dermatomyositis patients. PBMCs from 2 different patients were isolated by Ficoll gradient and cells were incubated with example 1 before activation with the TLR7 ligand R848 overnight. TNF production in monocytes (CD14+ cells) was quantified by intracellular staining by flow cytometry. See example 71.

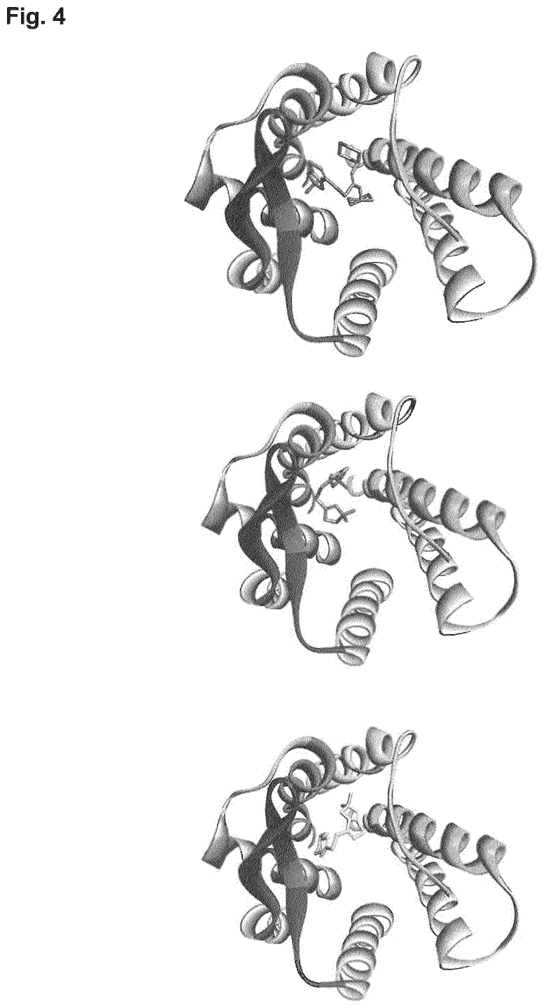

FIG. 4: Modeling of example 1 and 2 interaction with CXCR4. Modeling of IT1t (upper panel), example 2 (middle panel) and example 1 (lower panel) in the minor binding pocket of CXCR4. See example 71.

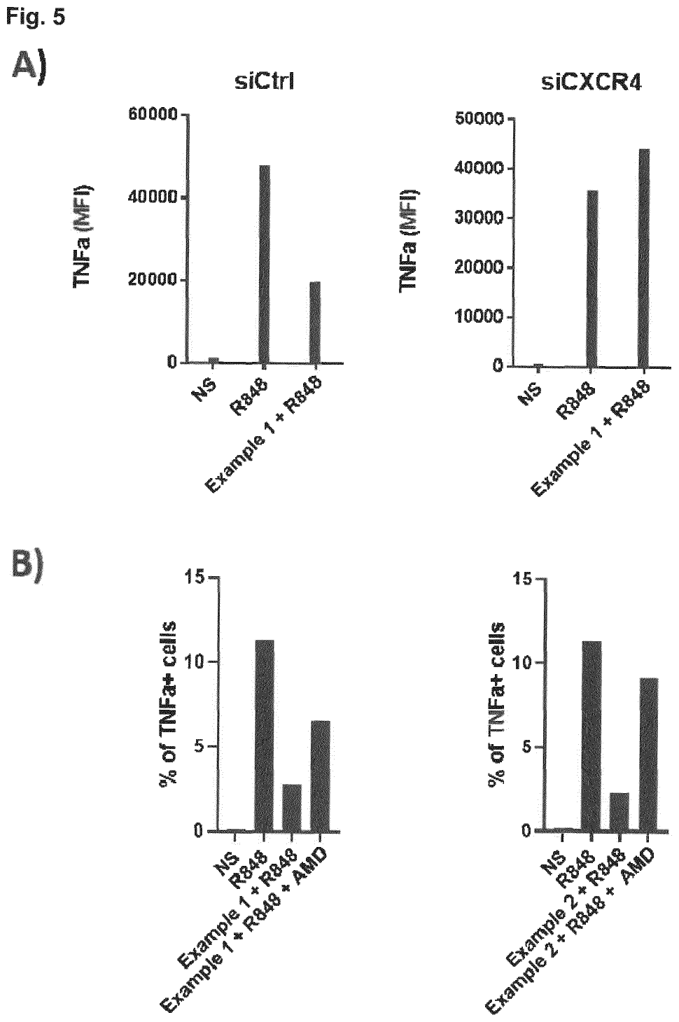

FIG. 5: Requirement of CXCR4 for the effect of examples 1 and 2 on TNFα production by monocytes from healthy donors. (A) Monocytes from healthy donors were isolated and treated for 24 h with a siRNA CXCR4 (siCXCR4) or a siRNA Control (siCtrl) at 160 nM and CXCR4 expression was assessed by flow cytometry (data not shown). (B) Monocytes from healthy donors were isolated and treated for 1 h with AMD3100 or buffer alone (negative control). In both (A) and (B), cells were then incubated with example 1 (5 μM) or example 2 (1 μM) or buffer alone before stimulation with the TLR7 ligand R848 overnight. TNFα production in monocytes (CD14+ cells) was quantified by intracellular staining by flow cytometry. NS: not stimulated cells; R848: cells stimulated with R848 alone; Example 1 or 2+R848: cells incubated with example 1 or 2 before stimulation with R848; Example 1 or 2+R848+AMD: cells incubated with AMD3100 and then example 1 or 2 before stimulation with R848; MFI: mean fluorescence intensity; % of TNFa+ cells: percentage of cells positive for TNFα staining. See example 71.

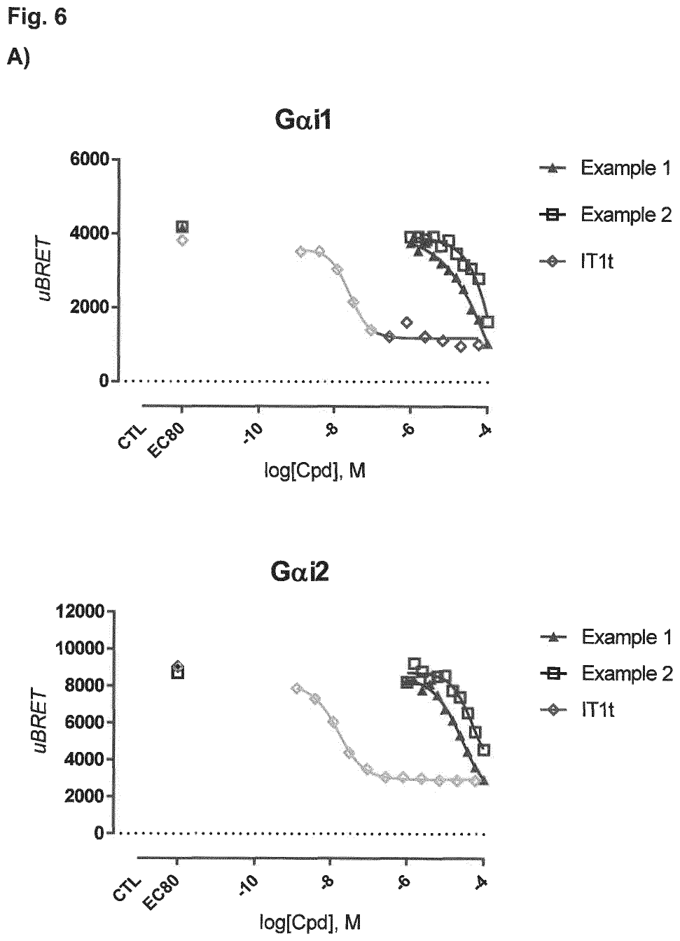

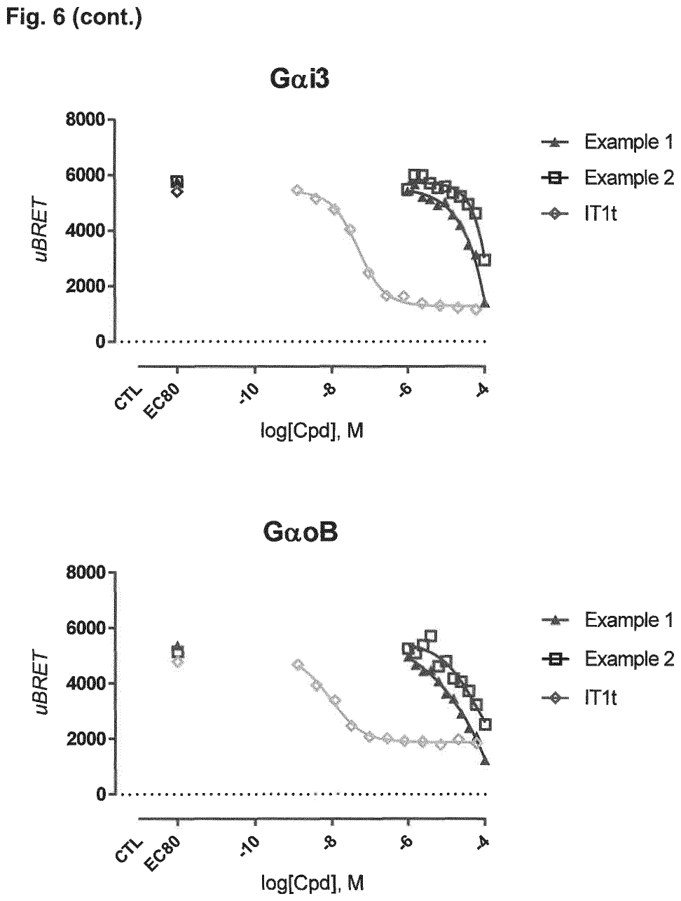

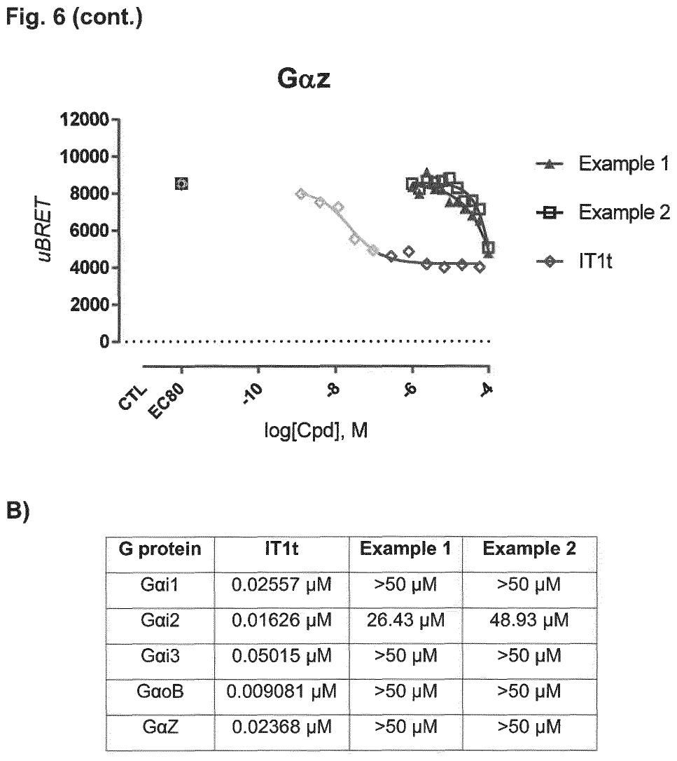

FIG. 6: Antagonist activity of examples 1 and 2 on the CXCR4-CXCL12 signaling pathway. HEK-293 T cells were co-transfected with several DNA plasmids encoding: hCXCR4; a G protein (Gαi1, Gαi2, Gαi3, GαoB, or Gαz); an intracellular effector fused to luciferase (BRET donor); a plasma membrane effector fused to GFP (BRET acceptor). Cells were then first incubated for 10 minutes with different concentrations of example 1 or 2 or IT1t alone before stimulation by an EC80 CXCL12 concentration and luminescence was recorded. (A) Dose-response curves are fitted using the sigmoïdal dose-response (variable slope) analysis in GraphPad Prism software (GraphPad Software). (B) Calculated IC₅₀ of antagonist activity. CTL: cells not treated with CXCL12 nor any compound (negative control); EC₈₀: cells stimulated by an EC80 CXCL12 concentration alone (positive control); uBRET: BRET signal; N.D.: Not Determined.

The invention will now be described by reference to the following examples which are merely illustrative and are not to be construed as a limitation of the scope of the present invention.

### EXAMPLES

The compounds/examples described in this section are defined by their chemical formulae and their corresponding chemical names. In case of conflict between any chemical formula and the corresponding chemical name indicated herein, the present invention relates to both the compound/example defined by the chemical formula and the compound/example defined by the chemical name, and particularly relates to the compound/example defined by the chemical formula.

General Experimental Procedures

1) General Synthetic Pathway

Exemplary compounds of general formula (I) and their pharmaceutically acceptable salts can be synthesized for example, but not only, according to a method adapted from the work of Gebhard Thoma and Emanuel Escher (Thoma G et al., J Med Chem, 2008, 51(24), 7915-20; Mona C E et al, Org Biomol Chem, 2016, 14(43), 10298-10311), as illustrated in the following schemes:

a) Preparation of Examples of General Formula (I) with n=0

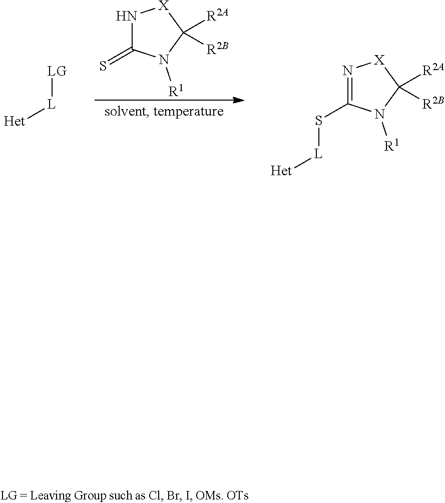

The electrophile (Het-L-LG) can be reacted with a cyclic thiourea in an appropriate solvent (such as MeCN, EtOH, DMF or DMA, or mixtures of these) at the suitable temperature (25 to 110° C., preferably 80° C.), optionally in the presence of sodium or potassium iodide until completion of the reaction (preferably overnight) to afford the desired alkylated thiourea.

For examples with Het=

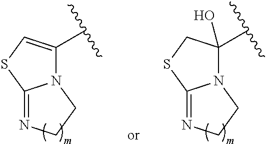

and L=(CH₂)ₘ, and LG=Cl, the electrophile (Het-L-LG) can be prepared as follows:

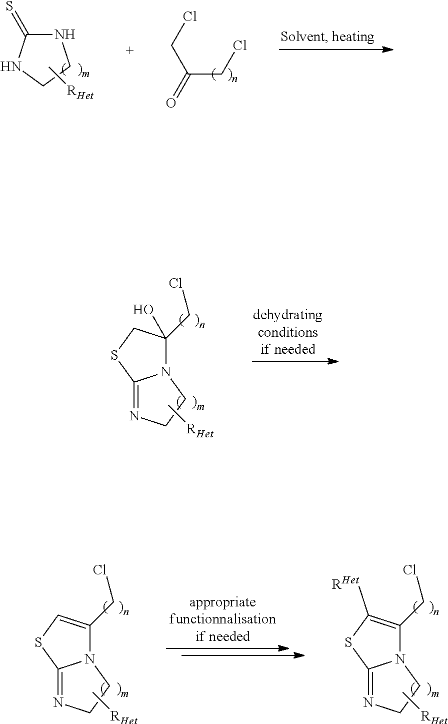

A thiourea can be reacted with a dichloro-ketone in an appropriate solvent such as MeCN, DMF or DMA at a suitable temperature (typically 50° C. to 80° C.). If the dehydration has not occurred yet (typically at low temperature, or when 6 or 7-membered ring cyclic thioureas are used) the hydrated intermediate can be isolated as such, or it can be further reacted under dehydrating conditions such as addition of molecular sieve and/or stronger heating (typically 110° C. in MeCN), or heating in an acidic medium such as HCl in dioxane and/or in DMF and/or in DMA. Finally, when necessary the dehydrated bicyclic electrophile can be further functionalized, for example by halogenation, optionally followed by further functionalization for example via pallado- or copper-catalyzed coupling such as Suzuki (Maluenda et al, (2015) Molecules, 20: 7528), Stille or Neigishi (Haas et al, (2016) ACS Catal., 6: 1540) coupling.

b) Preparation of Examples of General Formula (I) with n=1 or 2

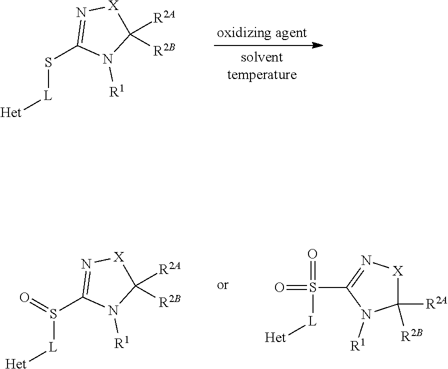

The examples of general formula (I) with n=1 or 2 can be prepared from the examples of general formula (I) with n=0 by reaction with the right amount (preferably 1 equivalent for n=1 and 2 equivalents for n=2) of an appropriate oxidizing agent in a suitable solvent (e.g. 3-chloroperbenzoic acid in dichloromethane or dihydrogen peroxide in water or methanol).

c) Preparation of the Starting Cyclic Thioureas

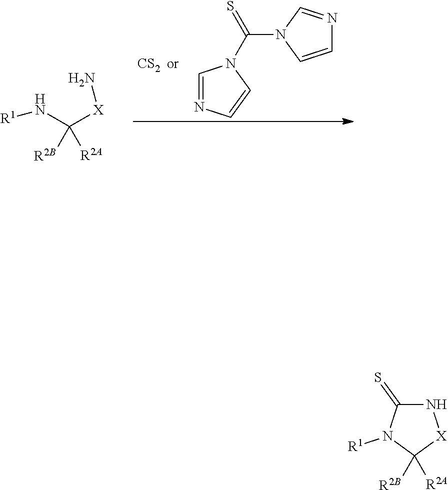

The starting cyclic thioureas can be cyclized from the appropriate diamine or its salt (typically the dihydrochloride) in the presence of di(1H-imidazol-1-yl)methanethione or carbon disulfide and optionally of a base such as triethylamine (preferably when the diamine salt is used) in the appropriate solvent (preferably dichloromethane).

2) General Conditions

All reagents were commercial grade and used without further purification. Reactions were typically run using commercial anhydrous solvents under argon atmosphere.

Column chromatography was generally performed with a Biotage Isolera Four apparatus using Interchim PURIFLASH jumbo pack silica HP cartridges pre-filed with 50 μm silica gel. Alternatively, Interchim® PURIFLASH jumbo pack silica HP cartridges pre-filed with 15 μM silica gel or Interchim® PURIFLASH jumbo pack silica SDT cartridges pre-filed with 20 μM silica gel or Biotage Sfär KP-Amino D cartridges pre-filed with 50 μM silica gel could be used when necessary.

Thin layer chromatography was carried out using pre-coated silica gel F-254 plates or Biotage KP-NH TLC plates.

Releasing of free bases from the corresponding salts was carried out using Biotage ISOLUTE® SCX-2 cation exchange cartridges.

¹H-NMR spectra were recorded on a Bruker AV-500 spectrometer or on a Bruker AMX-400 spectrometer. Proton chemical shifts are listed relative to residual CD₃OD (3.31 ppm), DMSO (2.50 ppm) or D₂O (4.78 ppm). Splitting patterns are designated as s (singlet), d (doublet), dd (doublet-doublet), t (triplet), tt (triplet-triplet), td (triplet-doublet), q (quartet), quint (quintuplet), sex (sextuplet), sept (septuplet), m (multiplet), b (broad).

UPLC-MS analyses were recorded with an UPLC Waters Aquity platform with a photodiode array detector (190-400 nm) using an Acquity CSH C₁₈ 1.7 μm (2.1×30 mm) column. The mobile phase consisted in a gradient of water with 0.025% of TFA and acetonitrile with 0.025% of TFA. The flow rate was 0.8 mL per min. All analyses were performed at 55° C. The UPLC system was coupled with a Waters SQD2 platform. All mass spectra were full-scan experiments (mass range 100-800 armu) and were obtained using electrospray ionization.

HPLC-MS were recorded using an HPLC Waters platform with a 2767 sample manager, a 2525 pump, a photodiode array detector (190-400 nm). This HPLC system was coupled with a Waters Acquity QDa detector. All mass spectra were full-scan experiments (mass range 110-850 amu) and were obtained using electro spray ionization. For analytical samples, the selected column was a C₁₈-AQ 5 μm (4.6×250 mm). For preparative purifications, the selected column was either column A an XSelect CSH prep C₁₈ 5 μm (19×100 mm) or column B a C₁₈-AQ 5 μm (21.2×250 mm). The mobile phase in all cases consisted in an appropriate gradient of water with 0.1% of formic acid and acetonitrile with 0.1% of formic acid. The flow rate was 1 mL/min in analytical mode, and in preparative mode 25 mL/min for column A and 21 mL/min for column B. All HPLC-MS were performed at room temperature.

Melting points were measured on a Barnstead Electrothermal 9100 and are not corrected.

Unless mentioned otherwise all compounds isolated by filtration or centrifugation were dried overnight in high vacuum at 50-70° C.

3 General Procedures and Methods:

General Procedure 1a: Dihydrothiazole Formation

A solution of thiourea (1.0 equiv) and di-chloroketone (1.0-1.5 equiv) in MeCN (0.2 M) was heated overnight at 80° C. The resulting mixture was allowed to cool down to rt, then Et₂O was optionally added to help the precipitation. The resulting precipitate was filtrated and triturated in MeCN. Optionally the filtrate was concentrated to dryness, triturated in cold MeCN and filtrated to recover more product. The product was further purified when necessary.

General Procedure 1b: Dihydrothiazole Formation

A solution of thiourea (1.0 equiv) and di-chloroketone (1.0-1.5 equiv) in MeCN (0.2 M) was heated overnight at 80° C. The resulting precipitate was filtrated and washed with MeCN to isolate the hydrate intermediate that was then suspended in HCl 4N in dioxane (0.8 M) and stirred at 80° C. for 1-18 h. The resulting suspension was allowed to cool down to rt, then Et₂O was optionally added to help the precipitation. The precipitate was filtrated and triturated in Et₂O. Optionally the filtrate was concentrated to dryness, triturated in cold MeCN and filtrated to recover more product. The product was further purified when necessary.

General Procedure 1c: Dihydrothiazole Formation

A solution of thiourea (1.0 equiv) and di-chloroketone (1.0-1.5 equiv) in MeCN (0.2 M) was heated overnight at 80° C. The resulting mixture was concentrated to dryness. The product was further purified when necessary.

General Procedure 1d: Dihydrothiazole Formation

A solution of thiourea (1.0 equiv) and di-chloroketone (1.0-1.5 equiv) in MeCN (0.2 M) was heated overnight at 80° C. The resulting precipitate was filtrated and washed with MeCN to isolate the hydrate intermediate that was then suspended in HCl 4N in dioxane (0.8 M) and stirred at 80° C. for 1-18 h. The reaction mixture was then concentrated to dryness. The product was further purified when necessary.

General Procedure 2a: Thiourea Formation

At 0° C., to a solution of diamine (1.0 equiv) in DCM (0.6 M) was added a solution of di(1H-imidazol-1-yl)methanethione (1.0 equiv) in DCM (0.4 M). The reaction mixture was stirred at 0° C. to rt for 1-4 h. Then the reaction mixture was diluted with DCM and washed once with a saturated solution of NaHCO₃. The aqueous phase was extracted several times with DCM and the combined organic extracts were washed with brine, filtered through a hydrophobic cartridge and concentrated to dryness. When necessary the crude was further purified.

General Procedure 2b: Thiourea Formation

At 0° C., to a solution of diamine (1.0 equiv) in DCM (0.6 M) was added a solution of di(1H-imidazol-1-yl)methanethione (1.0-1.1 equiv) in DCM (0.3 M). The reaction mixture was stirred at 0° C. to rt for 1-4 h. Then the suspension was filtrated and the solid was washed with cold DCM. Optionally the filtrate was concentrated to dryness, triturated in cold DCM and filtrated to recover more product. When necessary the crude was further purified.

General Procedure 2c: Thiourea Formation

At 0° C., to a suspension of diamine hydrochloride (1.0 equiv) in DCM (0.2 M) was added triethylamine (2.2 equiv) and the mixture was stirred at 0° C. for 15 min. Then, at 0° C., di(1H-imidazol-1-yl)methanethione (1.0 equiv) was added in one portion. The reaction mixture was stirred at 0° C. to rt for 1-4 h. Then the reaction mixture was diluted with DCM and washed once with a saturated solution of NaHCO₃. The aqueous phase was extracted several times with DCM and the combined organic extracts were washed with brine, filtered through a hydrophobic cartridge and concentrated to dryness. When necessary the crude was further purified.

General Procedure A: Alkylation of Thioureas to Afford Examples

A suspension of electrophile (1.0-3.0 equiv), thiourea (1.0-1.5 equiv) and optionally sodium iodide (1.0-2.0 equiv) in a solvent (C=0.2 M) was heated at 80-110° C. for 2 h to 5 days. The product was isolated as detailed hereinafter.

### Synthesis of Exemplary Compounds of the Invention

### Intermediates: Dihydrothiazole Electrophiles

### Intermediate 1: 3-(chloromethyl)-6,6-dimethyl-5,6-dihydroimidazo[2,1-b]thiazole hydrochloride

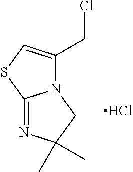

To a solution of 4,4-dimethylimidazolidine-2-thione (40.0 g, 1.0 equiv) in MeCN (600 mL) was added 1,3-dichloropropan-2-one (39.0 g, 1.0 equiv). The reaction mixture was stirred at 80° C. for 17 h and concentrated to dryness. The crude residue was triturated in ethylene glycol dimethyl ether at 100° C. for 2 h to afford 3-(chloromethyl)-6,6-dimethyl-5,6-dihydroimidazo[2,1-b]thiazole hydrochloride (26.5 g, 35%) as a grey solid. M/Z (M³⁵[Cl]+H)⁺: 203.1.

### Intermediate 2: 3-(chloromethyl)-5,6-dihydroimidazo[2,1-b]thiazole hydrochloride

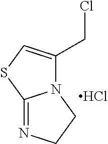

Intermediate 2 was obtained according to general procedure 1a, starting from imidazolidine-2-thione (2.0 g, 1.0 equiv) and 1,3-dichloropropan-2-one (3.7 g, 1.5 equiv) to afford 3-(chloromethyl)-5,6-dihydroimidazo[2,1-b]thiazole hydrochloride (4.1 g, 99%) as a white solid. M/Z (M[³⁵Cl]+H)⁺=174.5.

### Intermediate 3: trans-3-(chloromethyl)-4a,5,6,7,8,8a-hexahydrobenzo[4,5]imidazo[2,1-b]thiazole hydrochloride

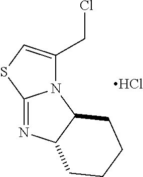

Intermediate 3 was obtained according to general procedure 1a, starting from intermediate 20 (60 mg, 1.0 equiv) and 1,3-dichloropropan-2-one (49 mg, 1.0 equiv) to afford trans-3-(chloromethyl)-4a,5,6,7,8,8a-hexahydrobenzo[4,5]imidazo[2,1-b]thiazole hydrochloride (45 mg, 45%) as a white solid. M/Z (M[³⁵Cl]+H)⁺=229.1.

### Intermediate 4: cis-3-(chloromethyl)-4a,5,6,7,8,8a-hexahydrobenzo[4,5]imidazo[2,1-b]thiazole hydrochloride

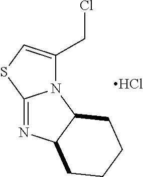

Intermediate 4 was obtained according to general procedure 1a, starting from intermediate 22 (60 mg, 1.0 equiv) and 1,3-dichloropropan-2-one (49 mg, 1.0 equiv) to afford cis-3-(chloromethyl)-4a,5,6,7,8,8a-hexahydrobenzo[4,5]imidazo[2,1-b]thiazole hydrochloride (105 mg, 69%) as a white solid. M/Z (M[³⁵Cl]+H)⁺=229.1.

### Intermediate 5: 3-(chloromethyl)-6,7-dihydro-5H-thiazolo[3,2-a]pyrimidine hydrochloride

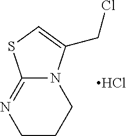 [SMILES: ClCC1=CSC2=NCCCN12 ; Cl]

Intermediate 5 was obtained according to general procedure 1b, starting from tetrahydropyrimidine-2(1H)-thione (250 mg, 1.0 equiv) and 1,3-dichloropropan-2-one (273 mg, 1.0 equiv) to afford 3-(chloromethyl)-6,7-dihydro-5H-thiazolo[3,2-a]pyrimidine hydrochloride as a white solid (67 mg, 69%). M/Z (M[³⁵Cl]+H)⁺=189.1.

### Intermediate 6: 3-(chloromethyl)-5,6,7,8-tetrahydrothiazolo[3,2-a][1,3]diazepine hydrochloride

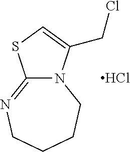 [SMILES: ClCC1=CSC2=NCCCCN12 ; Cl]

Intermediate 6 was obtained according to general procedure 1b, starting from 1,3-diazepane-2-thione (250 mg, 1.0 equiv) and 1,3-dichloropropan-2-one (244 mg, 1.0 equiv) to afford 3-(chloromethyl)-5,6,7,8-tetrahydrothiazolo[3,2-a][1,3]diazepine hydrochloride (351 mg, 76%) as a beige solid. M/Z (M[³⁵Cl]+H)⁺=203.1.

### Intermediate 7: 6-benzyl-3-(chloromethyl)-5,6-dihydroimidazo[2,1-b]thiazole hydrochloride

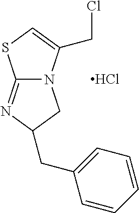 [SMILES: ClCC1=CSC2=NC(Cc3ccccc3)CN12 ; Cl]

Intermediate 7 was obtained according to general procedure 1a, starting from intermediate 25 (200 mg, 1.0 equiv) and 1,3-dichloropropan-2-one (132 mg, 1.0 equiv) to afford 6-benzyl-3-(chloromethyl)-5,6-dihydroimidazo[2,1-b]thiazole hydrochloride (268 mg, 86%) as a white solid. M/Z (M[³⁵Cl]+H)⁺=265.1.

### Intermediate 8: 6-butyl-3-(chloromethyl)-5,6-dihydroimidazo[2,1-b]thiazole hydrochloride

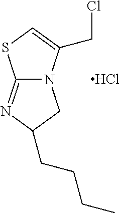

A solution of intermediate 23 (120 mg, 1.0 equiv) and 1,3-dichloroacetone (96 mg, 1.0 equiv) in MeCN (3 mL) was heated at 50° C. for 18 h and then at 100° C. for 6 h. It was then allowed to crystallize at rt overnight, cooled to 0° C. and the precipitate was filtered and rinsed with cold MeCN. The filtrate was concentrated to dryness, triturated in cold MeCN and filtrated. The solids were combined to afford 6-butyl-3-(chloromethyl)-5,6-dihydroimidazo[2,1-b]thiazole hydrochloride (125 mg, 62%) as a beige powder. M/Z (M+[³⁵Cl]H)⁺: 231.1.

### Intermediate 9: 3-(chloromethyl)-5,5,6,6-tetramethyl-5,6-dihydroimidazo[2,1-b]thiazole hydrochloride

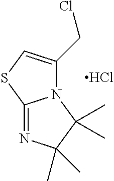

Intermediate 9 was obtained according to general procedure 1c, starting from intermediate 24 (200 mg, 1.0 equiv) and 1,3-dichloropropan-2-one (160 mg, 1.0 equiv) to afford crude 3-(chloromethyl)-5,5,6,6-tetramethyl-5,6-dihydroimidazo[2,1-b]thiazole hydrochloride (236 mg) as a brown oil that was used as such in the next step. M/Z (M[³⁵Cl]+H)⁺=231.1.

### Intermediate 10: cis-3-(chloromethyl)-6,7,8,9-tetrahydro-5H-5,9-methanothiazolo[3,2-a][1,3]diazocine hydrochloride

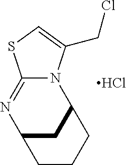

Intermediate 10 was obtained according to general procedure 1b, starting from intermediate 26 (200 mg, 1.0 equiv) and 1,3-dichloropropan-2-one (160 mg, 1.0 equiv) to afford a racemic mixture of cis-3-(chloromethyl)-6,7,8,9-tetrahydro-5H-5,9-methanothiazolo[3,2-a][1,3]diazocine hydrochloride (395 mg, quant.) as a brown solid. M/Z (M[³⁵Cl]+H)⁺: 229.1.

### Intermediate 11; 3′-(chloromethyl)-5′H-spiro[cyclopropane-1,6′-imidazo[2,1-b]thiazole]hydrochloride

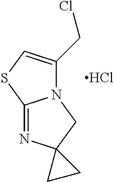 [SMILES: ClCC1=CSC2=NC3(CC3)CN12 ; Cl]

Intermediate 11 was obtained according to general procedure 1a, starting from intermediate 27 (142 mg, 1.0 equiv) and 1,3-dichloropropan-2-one (141 mg, 1.0 equiv) to afford 3′-(chloromethyl)-5′H-spiro[cyclopropane-1,6′-imidazo[2,1-b]thiazole] hydrochloride (143 mg, 54%) as a beige solid. M/Z (M[³⁵Cl]+H)⁺: 201.1.

### Intermediate 12: 3-(chloromethyl)-6,6-dimethyl-6,7-dihydro-5H-thiazolo[3,2-a]pyrimidine hydrochloride

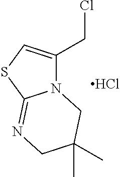

Intermediate 12 was obtained according to genera procedure 1d, starting from intermediate 29 (200 mg, 1.0 equiv) and 1,3-dichloropropan-2-one (264 mg, 1.5 equiv) to obtain 3-(chloromethyl)-6,6-dimethyl-6,7-dihydro-5H-thiazolo[3,2-a]pyrimidine hydrochloride (320 mg, 91%) as a white solid. M/Z (M[³⁵Cl]+H)⁺: 217.1.

### Intermediate 13: cis-3-(chloromethyl)-4a,5,7,7a-tetrahydrofuro[3′,4′:4,5]imidazo[2,1-b]thiazole hydrochloride

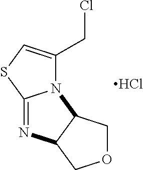

Intermediate 13 was obtained according to general procedure 1a, starting from intermediate 28 (200 mg, 1.0 equiv) and 1,3-dichloropropan-2-one (176 mg, 1.0 equiv) to obtain a racemic mixture of cis-3-(chloromethyl)-4a,5,7,7a-tetrahydrofuro[3′,4′:4,5]imidazo[2,1-b]thiazole hydrochloride in a mixture with 15 mol % of a racemic mixture of cis-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole dihydrochloride (289 mg) as a beige solid. M/Z (M[³⁵Cl]+H)⁺: 217.1.

### Intermediate 14: 3′-(chloromethyl)-5′H-spiro[cyclohexane-1,6′-imidazo[2,1-b]thiazole]hydrochloride

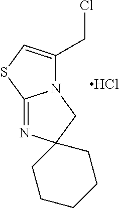 [SMILES: ClCC1=CSC2=NC3(CCCCC3)CN12 ; Cl]

Intermediate 14 was obtained according to general procedure 1c, starting from intermediate 30 (200 mg, 1.0 equiv) and 1,3-dichloropropan-2-one (224 mg, 1.5 equiv). The crude was dissolved in MeCN (1 mL), precipitated with Et₂O (5 mL) and filtrated to obtain 3′-(chloromethyl)-5′H-spiro[cyclohexane-1,6′-imidazo[2,1-b]thiazole] hydrochloride (273 mg, 83%) as a brown solid. M/Z (M[³⁵Cl]+H)⁺: 243.1.

### Intermediate 15: 3-(chloromethyl)-5a,6,7,8,9,9a-hexahydro-5H-thiazolo[2,3-b]quinazoline hydrochloride

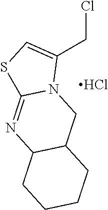 [SMILES: ClCC1=CSC2=NC3CCCCC3CN12 ; Cl]

Intermediate 15 was obtained according to general procedure 1d, starting from intermediate 31 (200 mg, 1.0 equiv) and 1,3-dichloropropan-2-one (160 mg, 1.0 equiv). The crude residue was dissolved in MeCN (1 mL) and precipitated by addition of Et₂O (5 mL). The solid was filtrated and washed with Et₂O to obtain 3-(chloromethyl)-5a,6,7,8,9,9a-hexahydro-5H-thiazolo[2,3-b]quinazoline hydrochloride (264 mg, 81%) as a beige solid. M/Z (M[³⁵Cl]+H)⁺: 243.1.

### Intermediate 16: 3-(chloromethyl)-6-phenyl-5,6-dihydroimidazo[2,1-b]thiazole hydrochloride

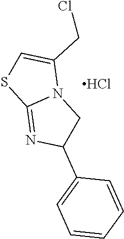 [SMILES: ClCC1=CSC2=NC(c3ccccc3)CN12 ; Cl]

Intermediate 16 was obtained according to general procedure 1a, starting from intermediate 33 (200 mg, 1.0 equiv) and 1,3-dichloropropan-2-one (214 mg, 1.5 equiv). The crude purified by trituration in MeCN (4 mL) and then recristallised in MeCN (12 mL) to obtain 3-(chloromethyl)-6-phenyl-5,6-dihydroimidazo[2,1-b]thiazole hydrochloride (100 mg, 31%) as a beige solid. M/Z (M[³⁵Cl]+H)⁺: 251.1

### Intermediate 17: 3-(chloromethyl)-6-isopropyl-5,6-dihydroimidazo[2,1-b]thiazole hydrochloride

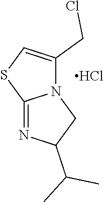

Intermediate 17 was obtained according to general procedure 1a, starting from intermediate 32 (150 mg, 1.0 equiv) and 1,3-dichloropropan-2-one (198 mg, 1.5 equiv) to obtain 3-(chloromethyl)-6-isopropyl-5,6-dihydroimidazo[2,1-b]thiazole hydrochloride (240 mg, 92%) as a beige solid. M/Z (M³⁵[Cl]+H)⁺: 217.1.

### Intermediate 18: trans-3-(chloromethyl)-5,6-diisopropyl-5,6-dihydroimidazo[2,1-b]thiazole hydrochloride

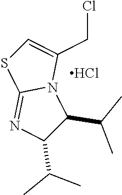

Intermediate 18 was obtained according to general procedure 1c, starting from intermediate 34 (180 mg, 1.0 equiv) and 1,3-dichloropropan-2-one (184 mg, 1.5 equiv). The crude was dissolved in MeCN (0.5 mL), precipitated with Et₂O (20 mL) and centrifuged. The resulting solid was purified by preparative HPLC (column A, H₂O+0.1% HCOOH/MeCN+0.1% HCOOH 95:5 to 55:45). The pure fractions were freeze-dried with 1N aqueous HCl to obtain a racemic mixture of trans-3-(chloromethyl)-5,6-diisopropyl-5,6-dihydroimidazo[2,1-b]thiazole hydrochloride (90 mg, 32%) as a beige solid. M/Z (M[³⁵Cl]+H)⁺: 259.1.

### Intermediate 19: 3-(3-chloropropyl)-6,6-dimethyl-,6-dihydroimidazo[2,1-b]thiazole hydrochloride

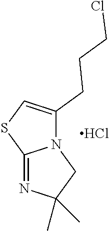

Intermediate 19 was obtained according to general procedure 1c, starting from 4,4-dimethylimidazolidine-2-thione (100 mg, 1.0 equiv) and 1,5-dichloropentan-2-one (135 mg, 95 wt %, 1.1 equiv). The crude was triturated in Et₂O (20 mL) until getting a white solid that was isolated by centrifugation to obtain 3-(3-chloropropyl)-6,6-dimethyl-5,6-dihydroimidazo[2,1-b]thiazole hydrochloride (186 mg, 91%) as a white solid. M/Z (M[³⁵Cl]+H)⁺: 231.1.

### Intermediates: Thioureas

### Intermediate 20: (3aR,7aR)-octahydro-2H-benzo[d]imidazole-2-thione

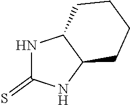

Intermediate 20 was obtained according to general procedure 2a, starting from (1R,2R)-cyclohexane-1,2-diamine (150 mg). The crude was purified by flash chromatography (Cyclohex 100% to Cyclohex/EtOAc 6:4) to obtain (3aR,7aR)-octahydro-2H-benzo[d]imidazole-2-thione (127 mg, 62%) as a yellow solid. M/Z (M+H)⁺: 157.1

### Intermediate 21: (3aS,7aS)-octahydro-2H-benzo[d]imidazole-2-thione

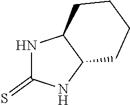

Intermediate 21 was obtained according to general procedure 2a, starting from (1S,2S)-cyclohexane-1,2-diamine (150 mg). The crude was purified by flash chromatography (Cyclohex 100% to Cyclohex/EtOAc 7:3) to obtain (3aS,7aS)-octahydro-2H-benzo[d]imidazole-2-thione (94 mg, 46%) as a yellow solid. M/Z (M+H)⁺: 157.2.

### Intermediate 22: (3aR,7aS)-octahydro-2H-benzo[d]imidazole-2-thione

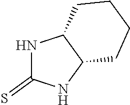

Intermediate 22 was obtained according to general procedure 2a, starting from (1R,2S)-cyclohexane-1,2-diamine (150 mg). The crude was purified by flash chromatography (Cyclohex 100% to Cyclohex/EtOAc 6:4) to obtain (3aR,7aS)-octahydro-2H-benzo[d]imidazole-2-thione (158 mg, 77%) as a yellow solid. M/Z (M+H)⁺: 157.2.

### Intermediate 23: 4-butylimidazolidine-2-thione

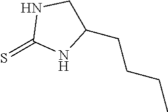

Intermediate 23 was obtained according to general procedure 2a, starting from hexane-1,2-diamine (250 mg). The crude was purified by flash chromatography (Cyclohex 100% to Cyclohex/EtOAc 6:4) to obtain 4-butylimidazolidine-2-thione (248 mg, 73%) as a yellow solid.

M/Z (M+H)⁺: 159.2.

### Intermediate 24: 4,4,5,5-tetramethylimidazolidine-2-thione

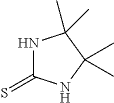

Intermediate 24 was obtained according to general procedure 2a, starting from 2,3-dimethylbutane-2,3-diamine (500 mg) to obtain 4,4,5,5-tetramethylimidazolidine-2-thione (651 mg, 96%) as a white solid. M/Z (M+H)⁺: 159.2.

### Intermediate 25: 4-benzylimidazolidine-2-thione

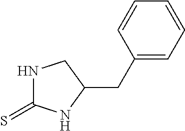 [SMILES: S=C1NCC(Cc2ccccc2)N1]

Intermediate 25 was obtained according to general procedure 2a, starting from 3-phenylpropane-1,2-diamine (500 mg). The crude was triturated in DCM and Et₂O to afford 4-benzylimidazolidine-2-thione (407 mg, 64%) as a pale yellow solid. M/Z (M+H)⁺: 193.1.

### Intermediate 26: (1R,5S)-2,4-diazabicyclo[3.3.1]nonane-3-thione

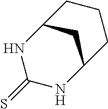

Intermediate 26 was obtained according to general procedure 2a, starting from (1R,3S)-cyclohexane-1,3-diamine (500 mg). The crude was purified by flash chromatography (Cyclohex/EtOAc 5:5 to EtOAc 100%) to obtain (1R,5S)-2,4-diazabicyclo[3.3.1]nonane-3-thione (357 mg, 52%) as a white solid. M/Z (M+H)⁺: 157.1.

### Intermediate 27: 4,6-diazaspiro[2.4]heptane-5-thione

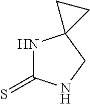

To a suspension of 1-(aminomethyl)cyclopropan-1-amine dihydrochloride (200 mg, 1.0 equiv) in DCM (5 mL) was added triethylamine (386 μL, 2.2 equiv). The reaction mixture was stirred at rt for 6 h. Then di(1H-imidazol-1-yl)methanethione (224 mg, 1.0 equiv) was added and the reaction mixture was stirred at rt for 2 h and was concentrated to dryness. The residue was purified by flash chromatography (CyHex 100% to EtOAc 100%) to obtain 4,6-diazaspiro[2.4]heptane-5-thione (105 mg, 65%) as a white solid. M/Z (M+H)⁺: 129.1.

### Intermediate 28: (3aR,6aS)-tetrahydro-1H-furo[3,4-d]imidazole-2(3H)-thione

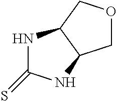

Intermediate 28 was obtained according to general procedure 2b, starting from (3R,4S)-tetrahydrofuran-3,4-diamine (500 mg) to obtain (3aR,6aS)-tetrahydro-1H-furo[3,4-d]imidazole-2(3H)-thione (560 mg, 79%) as a white solid. M/Z (M+H)⁺: 145.1.

### Intermediate 29: 5,5-dimethyltetrahydropyrimidine-2(1H)-thione

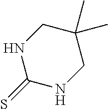

At 0° C., to a solution of 2,2-dimethylpropane-1,3-diamine (1.00 g, 1.0 equiv) in DCM (16 mL) was added a solution of di(1H-imidazol-1-yl)methanethione (1.74 g, 1.0 equiv) in DCM (32 mL). The reaction mixture was stirred at 0° C. for 4 h and then concentrated to dryness. The residue was triturated in MeCN (10 mL) at 25° C. for 2 h, then the solid was filtered and washed with MeCN to obtain 5,5-dimethyltetrahydropyrimidine-2(1H)-thione (500 mg, 35%) as a white solid. M/Z (M+H)⁺: 145.1.

### Intermediate 30: 1,3-diazaspiro[4.5]decane-2-thione

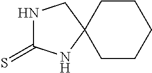 [SMILES: S=C1NCC2(CCCCC2)N1]

Intermediate 30 was obtained according to general procedure 2c, starting from 1-(aminomethyl)cyclohexan-1-amine dihydrochloride (500 mg). The crude was co-evaporated twice with cyclohexane to obtain 1,3-diazaspiro[4.5]decane-2-thione (355 mg, 84%) as a yellow solid. M/Z (M+H)⁺: 171.1.

### Intermediate 31: octahydroquinazoline-2(1H)-thione

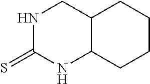 [SMILES: S=C1NCC2CCCCC2N1]

At 0° C., to a solution of 2-(aminomethyl)cyclohexan-1-amine (500 mg, 1.0 equiv) in DCM (7 mL) was added a solution of di(1H-imidazol-1-yl)methanethione (695 mg, 1.0 equiv) in DCM (14 mL). The reaction mixture was stirred at 0° C. for 4 h, then it was concentrated in vacuo. The residue was triturated in MeCN (10 mL) at 25° C. for 1 h. The solid was filtrated and washed with MeCN. The filtrate was concentrated to dryness, purified by flash chromatography (20 μm, DCM 100% to DCM/MeOH 90:10) and combined with the solid to obtain octahydroquinazoline-2(1H)-thione (380 mg, 57%) as a white solid. M/Z (M+H)⁺: 171.1.

### Intermediate 32: 4-isopropylimidazolidine-2-thione

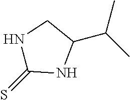

Intermediate 32 was obtained according to general procedure 2c but lowering the reaction mixture concentration to 0.02 M, starting from 3-methylbutane-1,2-diamine dihydrochloride (450 mg). The crude was purified by flash chromatography (20 μm, Cyclohexane 100% to EtOAc 100%) to obtain 4-isopropylimidazolidine-2-thione (198 mg, 53) as a pale yellow solid. M/Z (M+H)⁺: 145.2.

### Intermediate 3: 4-phenylimidazolidine-2-thione

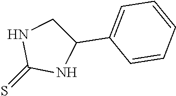 [SMILES: S=C1NCC(c2ccccc2)N1]

Intermediate 33 was obtained according to general procedure 2b, starting from 1-phenylethane-1,2-diamine (500 mg) to obtain 4-phenylimidazolidine-2-thione (441 mg, 67%) as a white solid. M/Z (M+H)⁺: 179.1.

### Intermediate 34: (4S,5S)-4,5-diisopropylimidazolidine-2-thione

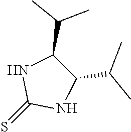

Intermediate 34 was obtained according to general procedure 2c but lowering the reaction mixture concentration to 0.02 M, starting from (3S,4S)-2,5-dimethylhexane-3,4-diamine dihydrochloride (500 mg). The crude was purified by flash chromatography (20 μm, CyHex 100% to EtOAc 100%) to obtain (4S,5S)-4,5-diisopropylimidazolidine-2-thione (270 mg, 63%) as a pale yellow solid. M/Z (M+H)⁺: 187.1.

### Intermediates: Miscellaneous

### Intermediate 35: 5-(2-iodoethyl)quinoline

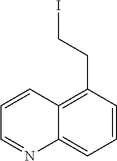 [SMILES: ICCc1cccc2ncccc12]

To a solution of 2-(quinolin-5-yl)ethan-1-ol (300 mg, 1.0 equiv) in DCM (17 mL) was added imidazole (165 mg, 1.4 equiv) and triphenylphosphine (545 mg, 1.2 equiv). At 0° C., iodine (571 mg, 1.3 equiv) was added in one portion and the reaction mixture was allowed to warm up slowly at rt and was for 1 h. It was then diluted with DCM (50 mL), washed with water (2×75 mL), a Na₂S₂O₃ saturated solution (2×75 mL), then with water (50 mL) and brine (60 mL), dried over magnesium sulfate and concentrated to dryness. The crude was purified by flash chromatography (DCM 100% to DCM/MeOH 90:10) to afford 5-(2-iodoethyl)quinoline (272 mg, 56%) as an orange solid. M/Z (M+H)⁺: 284.0.

### Intermediate 36: methyl 3-(5,6,7,8-tetrahydro-1,8-naphthyridin-2-yl)propanoate

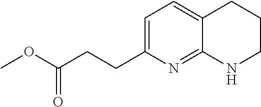 [SMILES: COC(=O)CCc1ccc2c(n1)NCCC2]

To a solution of 3-(5,6,7,8-tetrahydro-1,8-naphthyridin-2-yl)propanoic acid (500 mg, 1.0 equiv) in MeOH (12 mL) was added sulfuric acid (238 mg, 1.0 equiv). Then the reaction mixture was heated at 65° C. for 2 h and concentrated to dryness. The residue was purified by flash chromatography (DCM 100% to DCM/MeOH 90:10) to afford methyl 3-(5,6,7,8-tetrahydro-1,8-naphthyridin-2-yl)propanoate (328 mg, 61%) as a yellow solid. M/Z (M+H)⁺=221.1

### Intermediate 37: 3-(5,6,7,8-tetrahydro-1,8-naphthyridin-2-yl)propan-1-ol

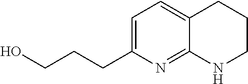 [SMILES: OCCCc1ccc2c(n1)NCCC2]

At 0° C., to a solution of intermediate 36 (328 mg, 1.0 equiv) in THF (12 mL) was added LiAlH₄ 1 M in THF (3.18 mL, 2.0 equiv). The reaction mixture was stirred at rt for 1 h, then cooled down to 0° C., diluted with Et₂O (25 mL) and hydrolyzed with water (0.12 mL), aq. 15% NaOH (0.12 mL) and additional water (0.36 mL). The resulting white precipitate was filtered-off and the filtrate was concentrated to dryness to obtain crude 3-(5,6,7,8-tetrahydro-1,8-naphthyridin-2-yl)propan-1-ol (88 mg) as an orange oil. M/Z (M+H)⁺: 193.1.

### Intermediate 38: 7-(3-chloropropyl)-1,2,3,4-tetrahydro-1,8-naphthyridine

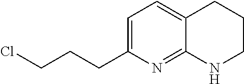 [SMILES: ClCCCc1ccc2c(n1)NCCC2]

At 0° C., to a solution of intermediate 37 (306 mg, 1.0 equiv) in DCM (4 mL) was added thionyl chloride (1.0 mL, 9.0 equiv) and DMF (12 μL, 0.1 equiv). The reaction mixture was stirred at 30° C. for 2 h and then poured dropwise at 0° C. into water (15 mL). Then this aqueous layer was washed with DCM (3×10 mL). The resulting aqueous layer was neutralized with a saturated solution of NaHCO₃ and extracted with DCM (3×10 mL). The combined organic layers were dried over magnesium sulfate and concentrated to dryness. The crude was purified by flash chromatography (15 μm, DCM 100% to DCM/MeOH 90:10) to afford 7-(3-chloropropyl)-1,2,3,4-tetrahydro-1,8-naphthyridine (72 mg, 86 wt % purity, 18% over 2 steps) as a yellow oil. M/Z (M+H)⁺=211.1.

### Intermediate 39: 3-(chloromethyl)-2-iodo-6,6-dimethyl-5,6-dihydroimidazo[2,1-b]thiazole hydrochloride

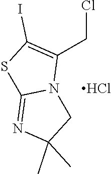

To a suspension of intermediate 1 (500 mg, 1.0 equiv) and iodine (796 mg, 1.5 equiv) in MeCN (10 mL) was added silver(I) sulfate (978 mg, 1.5 equiv). The reaction mixture was stirred at rt and in the dark for 3 h. The precipitate was filtered off, the filtrate was evaporated to dryness and purified by preparative HPLC (column A, H₂O+0.1% HCOOH/MeCN+0.1% HCOOH 95:5 to 55:45). The pure fractions were freeze-dried with 1N aqueous HCl to afford 3-(chloromethyl)-2-iodo-6,6-dimethyl-5,6-dihydroimidazo[2,1-b]thiazole hydrochloride (195 mg, 26%) as a light yellow solid. M/Z (M[³⁵Cl]+H)⁺: 328.9.

### Intermediate 40: 1-benzyl-3-iodopyrrolidine hydrochloride

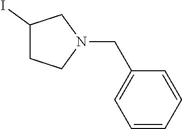 [SMILES: IC1CCN(Cc2ccccc2)C1]

To a solution of 1-benzylpyrrolidin-3-ol (100 mg, 1.0 equiv) in DCM (3 mL) was added 1H-imidazole (54 mg, 1.4 equiv) and triphenylphosphine (178 mg, 1.2 equiv). At 0° C., iodine (186 mg, 1.3 equiv) was added in one portion, then the reaction mixture was allowed to warm up slowly to rt and was stirred for 18 h. The reaction mixture was diluted with DCM (20 mL), washed with water (2×50 mL), with a Na₂S₂O₃ aqueous saturated solution (2×50 mL), with water (25 mL), with brine (50 mL), dried over magnesium sulfate and concentrated to dryness. The crude was purified by flash chromatography (20 μm, DCM 100% to DCM/MeOH 95:5) to afford 1-benzyl-3-iodopyrrolidine (62 mg, 38%) as a yellow oil. M/Z (M+H)⁺=288.0.

### Examples

Examples 1 to 69 were prepared according to general procedure A using the reaction conditions detailed in the following table, and isolated as described hereinafter.

| | | | Nal | | | |
|---|---|---|---|---|---|---|
| | Electrophile | Thiourea | (mass, | | | |
| Example | (mass, equiv) | (mass, equiv) | equiv) | Solvent | T ° C. | time |
| 1 | intermediate 1 | cis/trans-2- | — | MeCN | 110 | 4 h |
| | (714 mg, 3.0 equiv) | benzimidazolinethione | | | | |
| | | (156 mg, 1.0 equiv) | | | | |
| 2 | intermediate 1 | 4,4-dimethyl-2- | — | MeCN | 110 | 3 h |
| | (50 mg, 1.0 equiv) | imidazolidinethione (27 mg, | | | | |
| | | 1.0 equiv) | | | | |
| 3 | intermediate 1 | intermediate 20 | — | MeCN | 110 | 4 h |
| | (70 mg, 1.0 equiv) | (50 mg, 1.1 equiv) | | | | |
| 4 | intermediate 1 | intermediate 21 | — | MeCN | 110 | 5 h |
| | (70 mg, 1.0 equiv) | (50 mg, 1.1 equiv) | | | | |
| 5 | intermediate 1 | intermediate 22 | — | MeCN | 110 | 4 h |
| | (70 mg, 1.0 equiv) | (50 mg, 1.1 equiv) | | | | |
| 6 | intermediate 2 | imidazolidine-2-thione | — | MeCN | 110 | 2 h |
| | (24 mg, 1.0 equiv). | (50 mg, 1.0 equiv) | | | | |
| 7 | intermediate 3 | 4,4-dimethyl-2- | — | MeCN | 110 | 18 h |
| | (45 mg, 1.0 equiv) | imidazolidinethione (33 mg, | | | | |
| | | 1.5 equiv) | | | | |
| 8 | intermediate 1 | imidazolidine-2-thione | — | EtOH | 90 | 4 h |
| | (100 mg, 1.0 equiv) | (64 mg, 1.5 equiv) | | | | |
| 9 | intermediate 1 | tetrahydropyrimidine-2(1H)- | — | MeCN | 110 | 4 h |
| | (100 mg, 1.0 equiv) | thione (73 mg, 1.5 equiv) | | | | |
| 10 | intermediate 4 | tetrahydropyrimidine-2(1H)- | — | MeCN | 110 | 18 h |
| | (100 mg, 1.0 equiv) | thione (74 mg, 1.5 equiv) | | | | |
| 11 | intermediate 1 | 1-methylimidazolidine-2- | — | EtOH | 80 | 3 h |
| | (100 mg, 1.0 equiv) | thione | | | | |
| | | (73 mg, 1.5 equiv) | | | | |
| 12 | intermediate 1 | 1,3-diazepane-2-thione | — | MeCN | 80 | 18 h |
| | (100 mg, 1.0 equiv) | (60 mg, 1.1 equiv) | | | | |
| 13 | intermediate 1 | intermediate 23 | — | MeCN | 80 | 16 h |
| | (70 mg, 1.0 equiv) | (69 mg, 1.5 equiv) | | | | |
| 14 | intermediate 5 | 4,4-dimethyl-2- | — | MeCN | 80 | 18 h |
| | (95 mg, 1.0 equiv) | imidazolidinethione (51 mg, | | | | |
| | | 1.1 equiv) | | | | |
| 15 | intermediate 6 | 4,4-dimethyl-2- | — | MeCN | 80 | 18 h |
| | (100 mg, 1.0 equiv) | imidazolidinethione (60 mg, | | | | |
| | | 1.1 equiv) | | | | |
| 16 | intermediate 1 | intermediate 24 | — | MeCN | 80 | 18 h |
| | (100 mg, 1.0 equiv) | (73 mg, 1.1 equiv) | | | | |
| 17 | intermediate 1 | intermediate 25 | — | MeCN | 80 | 18 h |
| | (100 mg, 1.0 equiv) | (80 mg, 1.0 equiv) | | | | |
| 18 | intermediate 7 | 4,4-dimethyl-2- | — | MeCN | 80 | 18 h |
| | (100 mg, 1.0 equiv) | imidazolidinethione (48 mg, | | | | |
| | | 1.1 equiv) | | | | |
| 19 | intermediate 8 | 4,4-dimethyl-2- | — | MeCN | 80 | 18 h |
| | (120 mg, 1.0 equiv) | imidazolidinethione (64 mg, | | | | |
| | | 1.1 equiv) | | | | |
| 20 | intermediate 9 | 4,4-dimethyl-2- | — | MeCN | 80 | 18 h |
| | (236 mg, 1.0 equiv) | imidazolidinethione | | | | |
| | | (127 mg, 1.1 equiv) | | | | |
| 21 | intermediate 2 | 4,4-dimethyl-2- | — | EtOH | 80 | 18 h |
| | (100 mg, 1.0 equiv) | imidazolidinethione | | | | |
| | | (93 mg, 1.5 equiv) | | | | |
| 22 | intermediate 1 | intermediate 26 | — | MeCN | 80 | 20 h |
| | (100 mg, 1.0 equiv) | (72 mg, 1.1 equiv) | | | | |
| 23 | intermediate 10 | 4,4-dimethyl-2- | — | MeCN | 80 | 4 h |
| | (170 mg, 1.0 equiv) | imidazolidinethione | | | | |
| | | (91 mg, 1.1 equiv) | | | | |
| 24 | intermediate 1 | intermediate 27 | — | MeCN | 80 | 18 h |
| | (100 mg, 1.0 equiv) | (63 mg, 1.2 equiv) | | | | |
| 25 | intermediate 1 | intermediate 28 | — | MeCN | 80 | 24 h |
| | (100 mg, 1.0 equiv) | (66 mg, 1.1 equiv) | | | | |
| 26 | intermediate 5 | imidazolidine-2-thione | — | MeCN | 80 | 16 h |
| | (95 mg, 1.0 equiv) | (50 mg, 1.1 equiv) | | | | |
| 27 | intermediate 6 | imidazolidine-2-thione | — | MeCN | 80 | 16 h |
| | (100 mg, 1.0 equiv) | (47 mg, 1.1 equiv) | | | | |
| 28 | intermediate 1 | intermediate 29 | — | MeCN | 80 | 24 h |
| | (100 mg, 1.0 equiv) | (66 mg, 1.1 equiv) | | | | |
| 29 | intermediate 5 | tetrahydropyrimidine-2(1H)- | — | MeCN | 80 | 16 h |
| | (100 mg, 1.0 equiv) | thione | | | | |
| | | (59 mg, 1.1 equiv) | | | | |
| 30 | intermediate 2 | intermediate 23 | — | MeCN | 80 | 3 d |
| | (100 mg, 1.0 equiv) | (83 mg, 1.1 equiv) | | | | |
| 31 | 3-(3-chloropropyl)pyridine | 4,4-dimethyl-2- | — | MeCN | 80 | 5 d |
| | hydrochloride | imidazolidinethione | | | | |
| | (150 mg, 2.0 equiv) | (50 mg, 1.0 equiv) | | | | |
| 32 | 3-(chloromethyl)pyridine | 4,4-dimethyl-2- | — | MeCN | 80 | 18 h |
| | hydrochloride | imidazolidinethione | | | | |
| | (170 mg, 2.0 equiv) | (70 mg, 1.0 equiv) | | | | |
| 33 | intermediate 11 | 4,4-dimethyl-2- | — | MeCN | 80 | 18 h |
| | (100 mg, 1.0 equiv) | imidazolidinethione | | | | |
| | | (60 mg, 1.1 equiv) | | | | |
| 34 | intermediate 12 | 4,4-dimethyl-2- | — | MeCN | 80 | 20 h |
| | (100 mg, 1.0 equiv) | imidazolidinethione | | | | |
| | | (57 mg, 1.1 equiv) | | | | |
| 35 | intermediate 2 | tetrahydropyrimidine-2(1H)- | — | MeCN | 80 | 16 d |
| | (100 mg, 1.0 equiv) | thione (61 mg, 1.1 equiv) | | | | |
| 36 | intermediate 13 | intermediate 28 | — | MeCN | 80 | 23 h |
| | (100 mg, 1.0 equiv) | (57 mg, 1.0 equiv) | | | | |
| 37 | intermediate 6 | tetrahydropyrimidine-2(1H)- | — | MeCN | 80 | 16 h |
| | (100 mg, 1.0 equiv) | thione | | | | |
| | | (53 mg, 1.1 equiv) | | | | |
| 38 | 1-(3-chloropropyl)-1H- | 4,4-dimethyl-2- | — | MeCN | 80 | 40 h |
| | imidazole hydrochloride | imidazolidinethione | | | | |
| | (167 mg, 2.0 equiv) | (60 mg, 1.0 equiv) | | | | |
| 39 | intermediate 1 | intermediate 30 | — | MeCN | 80 | 22 h |
| | (100 mg, 1.0 equiv) | (78 mg, 1.1 equiv) | | | | |
| 40 | intermediate 1 | intermediate 31 | — | MeCN | 80 | 22 h |
| | (100 mg, 1.0 equiv) | (78 mg, 1.1 equiv) | | | | |
| 41 | intermediate 35 | 4,4-dimethyl-2- | — | MeCN | 80 | 18 h |
| | (100 mg, 1.0 equiv) | imidazolidinethione | | | | |
| | | (60 mg, 1.1 equiv) | | | | |
| 42 | intermediate 14 | 4,4-dimethyl-2- | — | MeCN | 80 | 20 h |
| | (100 mg, 1.0 equiv) | imidazolidinethione | | | | |
| | | (51 mg, 1.1 equiv) | | | | |
| 43 | intermediate 15 | 4,4-dimethyl-2- | — | MeCN | 80 | 20 h |
| | (100 mg, 1.0 equiv) | imidazolidinethione | | | | |
| | | (51 mg, 1.1 equiv) | | | | |
| 44 | intermediate 2 | intermediate 20 | — | MeCN | 80 | 16 h |
| | (75 mg, 1.0 equiv) | 4 (61 mg, 1.1 equiv) | | | | |
| 45 | intermediate 5 | intermediate 20 | — | MeCN | 80 | 16 h |
| | (75 mg, 1.0 equiv) | (57 mg, 1.1 equiv) | | | | |
| 46 | intermediate 6 | intermediate 20 | — | MeCN | 80 | 16 h |
| | (75 mg, 1.0 equiv) | (54 mg, 1.1 equiv) | | | | |
| 47 | 1-(2-chloroethyl)-1H- | 4,4-dimethyl-2- | 230 mg, | EtOH | 80 | 18 h |
| | imidazole hydrochloride | imidazolidinethione | 2.0 | | | |
| | (257 mg, 2.0 equiv) | (100 mg, 1.0 equiv) | equiv | | | |
| 48 | intermediate 5 | intermediate 25 | — | MeCN | 80 | 16 h |
| | (75 mg, 1.0 equiv) | (71 mg, 1.1 equiv) | | | | |
| 49 | intermediate 1 | intermediate 32 | — | MeCN | 80 | 22 h |
| | (100 mg, 1.0 equiv) | (66 mg, 1.1 equiv) | | | | |
| 50 | intermediate 16 | 4,4-dimethyl-2- | — | MeCN | 80 | 38 h |
| | (100 mg, 1.0 equiv) | imidazolidinethione | | | | |
| | | (50 mg, 1.1 equiv) | | | | |
| 51 | intermediate 7 | intermediate 23 | — | EtOH | 50 | 4 h |
| | (100 mg, 1.0 equiv) | (58 mg, 1.1 equiv) | | | | |
| 52 | intermediate 17 | 4,4-dimethyl-2- | — | MeCN | 80 | 20 h |
| | (100 mg, 1.0 equiv) | imidazolidinethione | | | | |
| | | (57 mg, 1.1 equiv) | | | | |
| 53 | intermediate 1 | intermediate 33 | — | EtOH | 50 | 24 h |
| | (100 mg, 1.0 equiv) | (75 mg, 1.1 equiv) | | | | |
| 54 | intermediate 3 | intermediate 23 | — | MeCN | 80 | 16 h |
| | (100 mg, 1.0 equiv) | (66 mg, 1.1 equiv) | | | | |
| 55 | intermediate 3 | intermediate 25 | — | MeCN | 80 | 16 h |
| | (100 mg, 1.0 equiv) | (80 mg, 1.1 equiv) | | | | |
| 56 | intermediate 3 | intermediate 20 | — | MeCN | 80 | 16 h |
| | (100 mg, 1.0 equiv) | (65 mg, 1.1 equiv) | | | | |
| 57 | intermediate 1 | 1- | — | MeCN | 80 | 21 h |
| | (100 mg, 1.0 equiv) | methyltetrahydropyrimidine- | | | | |
| | | 2(1H)-thione (60 mg, 1.1 | | | | |
| | | equiv) | | | | |
| 58 | intermediate 1 | intermediate 34 | — | MeCN | 80 | 21 h |
| | (100 mg, 1.0 equiv) | (86 mg, 1.1 equiv) | | | | |
| 59 | intermediate 3 | imidazolidinethione | — | MeCN | 80 | 16 h |
| | (100 mg, 1.0 equiv) | (42 mg, 1.1 equiv) | | | | |
| 60 | intermediate 7 | intermediate 25 | — | EtOH | 50 | 16 h |
| | (100 mg, 1.0 equiv) | (70 mg, 1.0 equiv) | | | | |
| 61 | intermediate 7 | intermediate 20 | — | EtOH | 50 | 16 h |
| | (100 mg, 1.0 equiv) | (57 mg, 1.1 equiv) | | | | |
| 62 | intermediate 18 | 4,4-dimethyl-2- | — | MeCN | 80 | 20 h |
| | (90 mg, 1.0 equiv) | imidazolidinethione | | | | |
| | | (44 mg, 1.1 equiv) | | | | |
| 63 | intermediate 3 | 1-methylimidazolidine-2- | — | MeCN | 80 | 2 d |
| | (100 mg, 1.0 equiv) | thione (48 mg, 1.1 equiv) | | | | |
| 64 | intermediate 19 | 4,4-dimethyl-2- | — | MeCN | 80-110 | 61 h |
| | (90 mg, 1.0 equiv) | imidazolidinethione | | | | |
| | | (48 mg, 1.1 equiv) | | | | |
| 65 | 3-(2-bromoethyl)pyridine | intermediate 25 | 156 mg, | EtOH | 80 | 18 h |
| | hydrobromide (278 mg, | (100 mg, 1.0 equiv) | 2.0 | | | |
| | 2.0 equiv) | | equiv | | | |
| 66 | intermediate 39 | 4,4-dimethyl-2- | — | EtOH | 40 | 16 h |
| | (50 mg, 1.0 equiv) | imidazolidinethione | | | | |
| | | (18 mg, 1.0 equiv) | | | | |
| 67 | intermediate 40 | intermediate 25 | — | EtOH | 80 | 40 h |
| | (62 mg, 1.0 equiv) | (42 mg, 1.0 equiv) | | | | |
| 68 | intermediate 38 | intermediate 25 | 59 mg, | EtOH | 0 | 30 h |
| | (72 mg, 1.3 equiv) | (50 mg, 1.0 equiv) | 1.5 | | | |
| | | | equiv | | | |
| 69 | Intermediate 7 | 1-methylimidazolidine-2- | — | MeCN | 80 | 2 d |
| | (100 mg, 1.0 equiv) | thione | | | | |
| | | (58 mg, 1.5 equiv) | | | | |

### Example 1: 3-(((3a,4,5,6,7,7a-hexahydro-1H-benzo[d]imidazol-2-yl)thio)methyl)-6,6-dimethyl-5,6-dihydroimidazo[2,1-b]thiazole dihydrochloride (Cis/Trans Mixture)

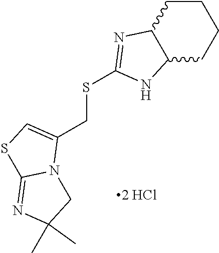 [SMILES: CC1(C)CN2C(CSC3=NC4CCCCC4N3)=CSC2=N1]

Example 1 was isolated by filtration of the reaction mixture followed by recrystallization of the solid twice from EtOAc/EtOH to afford 3-(((3a,4,5,6,7,7a-hexahydro-1H-benzo[d]imidazol-2-yl)thio)methyl)-6,6-dimethyl-5,6-dihydroimidazo[2,1-b]thiazole dihydrochloride (40%, cis/trans mixture) as a white solid.

¹H-NMR (D₂O, 500 MHz) δ: 1.40-1.60 (m, 3H, CHₐH_{b}+CH₂); 1.64 (s, 6H, 2 CH₃); 1.66-1.78 (m, 2H, CH₂); 1.86-1.97 (m, 2H, CH₂); 2.29 (d, J 11.7 Hz, 1H, CHₐH_{b}); 3.55-3.60 (m, 0.7H, one diastereoisomer of CH); 4.27-4.33 (m, 1.3H, other diastereoisomer of CH+CH); 4.33 (s, 0.7H, one diastereoisomer of N—CH₂); 4.35 (m, 1.3H, other diastereoisomer of N—CH₂); 4.58 (s, 2H, S—CH₂); 6.99 (s, 1H, Ar). M/Z (M+H)⁺: 323.0.

### Example 2: 3-(((5,5-dimethyl-4,5-dihydro-1H-imidazol-2-yl)thio)methyl)-6,6-dimethyl-5,6-dihydroimidazo[2,1-b]thiazole dihydrochloride

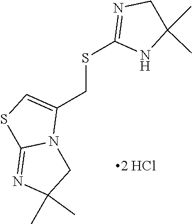 [SMILES: CC1(C)CN2C(CSC3=NCC(C)(C)N3)=CSC2=N1]

Example 2 was isolated by filtration of the reaction mixture followed by trituration of the solid in MeCN to afford 3-(((5,5-dimethyl-4,5-dihydro-1H-imidazol-2-yl)thio)methyl)-6,6-dimethyl-5,6-dihydroimidazo[2,1-b]thiazole dihydrochloride (53 mg, 69%) as a white powder.

¹H-NMR (DMSO-d₆, 400 MHz) δ:1.36 (s, 6H, 2 CH₃); 1.50 (s, 6H, 2 CH₃); 3.60 (s, 2H, S—CH₂); 4.24 (s, 2H, N—CH₂); 4.77 (s, 2H, N—CH₂); 7.11 (s, 1H, S—CH); 10.44 (bs, 1H, HCl salt); 10.76 (bs, 1H, HCl salt); 11.37 (bs, 1H, NH). M/Z (M+H)⁺: 297.2. Mp>250° C.

### Example 3: 3-((((3aR,7aR)-3a,4,5,6,7a-hexahydro-1H-benzo[d]imidazol-2-yl)thio)methyl)-6,6-dimethyl-5,6-dihydroimidazo[2,1-b]thiazole dihydrochloride

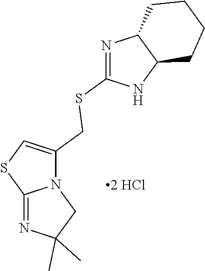 [SMILES: CC1(C)CN2C(CSC3=N[C@@H]4CCCC[C@H]4N3)=CSC2=N1]

Example 3 was isolated by trituration of the reaction mixture to allow the formation of a precipitate that was filtrated and further triturated in MeCN to afford 3-((((3aR,7aR)-3a,4,5,6,7,7a-hexahydro-1H-benzo[d]imidazol-2-yl)thio)methyl)-6,6-dimethyl-5,6-dihydroimidazo[2,1-b]thiazole dihydrochloride (85 mg, 73%) as a grey powder.

¹H-NMR (DMSO-d₆, 400 MHz) δ: 1.28-1.36 (m, 2H, CH₂—CH₂); 1.50 (bs, 8H, CH₂—CH₂+2CH₃); 1.76-1.78 (m, 2H, CH₂—CH₂); 2.15 (d, J 11.1 Hz, 2H, CH₂—H₂); 3.43-3.45 (m, 2H, 2N—CH); 4.23 (s, 2H, S—CH₂); 4.79 (dd, 2H, J 26.8 Hz, 15.7 Hz, N—CH₂); 7.13 (s, 1H, S—CH); 10.43 (bs, 1H, HCl salt); 11.18 (bs, 2H, NH+HCl salt). M/Z (M+H)⁺: 323.1. Mp: 65-75° C.

### Example 4: 3-((((3aS,7aS)-3a,4,5,6,7,7a-hexahydro-1H-benzo[d]imidazol-2-yl)thio)methyl)-6,6-dimethyl-5,6-dihydroimidazo[2,1-b]thiazole dihydrochloride

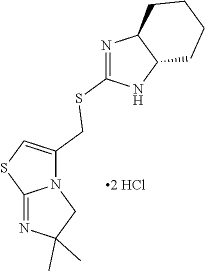 [SMILES: CC1(C)CN2C(CSC3=N[C@H]4CCCC[C@@H]4N3)=CSC2=N1]

Example 4 was isolated by trituration of the reaction mixture to allow the formation of a precipitate that was filtrated and further triturated in MeCN to afford 3-((((3aS,7aS)-3a,4,5,6,7,7a-hexahydro-1H-benzo[d]imidazol-2-yl)thio)methyl)-6,6-dimethyl-5,6-dihydroimidazo[2,1-b]thiazole dihydrochloride (80 mg, 69%) as a white powder.

¹H-NMR (DMSO-d₆, 400 MHz) δ: 1.28-1.36 (m, 2H, CH₂CH₂); 1.50 (bs, 8H, CH₂—CH₂+2 CH₃); 1.76-1.78 (m, 2H, CH₂—CH₂); 2.15 (d, J 11.2 Hz, 2H, CH₂—CH₂); 3.43-3.45 (m, 2H, 2N—CH); 4.23 (s, 2H, S—CH₂); 4.79 (dd, 2H, J 26.9 Hz, 15.7 Hz, N—CH₂); 7.14 (s, 1H, S—CH); 10.41 (bs, 1H, HCl salt); 11.29 (bs, 2H, NH+HCl salt). M/Z (M+H)⁺: 323.1. Mp: 167-175° C.

### Example 5: 3-((((3R,7aS)-3a,4,5,6,7,7a-hexahydro-1H-benzo[d]imidazol-2-yl)thio)methyl)-6,6-dimethyl-5,6-dihydroimidazo[2,1-b]thiazole dihydrochloride

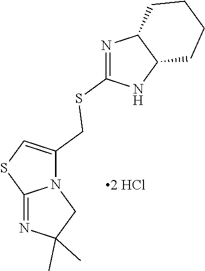 [SMILES: CC1(C)CN2C(CSC3=N[C@@H]4CCCC[C@@H]4N3)=CSC2=N1]

Example 5 was isolated by trituration of the reaction mixture to get a precipitate that was filtrated. The resulting solid was dissolved in hot MeOH (0.5 mL), then nBuOH (2 mL) was added and MeOH was removed in vacuo. The resulting white precipitate was filtered, triturated once in nBuOH, 3 times in Et₂O and freeze-dried in a mixture of water and 1N aqueous HCl to afford 3-((((3aR,7aS)-3a,4,5,6,7,7a-hexahydro-1H-benzo[d]imidazol-2-yl)thio)methyl)-6,6-dimethyl-5,6-dihydroimidazo[2,1-b]thiazole dihydrochloride (59 mg, 51%) as a white powder.

¹H-NMR (DMSO-d₆+D₂O, 400 MHz) δ: 1.28-1.39 (m, 4H, 2 CH₂—CH₂); 1.47 (s, 6H, 2 CH₃); 1.47-1.55 (m, 2H, CH₂—CH₂); 1.68-1.80 (m, 2H, CH₂—CH₂); 3.13-4.21 (m, 2H, 2N—CH+S—CH₂); 4.47 (s, 2H, N—CH₂); 6.89 (s, 1H, S—CH). M/Z (M+H)⁺: 323.2. Mp>250° C.

### Example 6: 3-(((4,5-dihydro-1H-imidazol-2-yl)thio)methyl)-5,6-dihydroimidazo[2,1-b]thiazole dihydrochloride

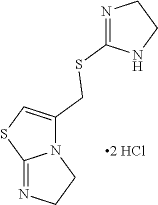 [SMILES: C1=C(CSC2=NCCN2)N2CCN=C2S1]

Example 6 was isolated by filtration of the reaction mixture followed by washing of the solid with MeCN to afford 3-(((4,5-dihydro-1H-imidazol-2-yl)thio)methyl)-5,6-dihydroimidazo[2,1-b]thiazole dihydrochloride (56 mg, 75%) as a white solid.

¹H-NMR (DMSO-d₆, 400 MHz) δ: 3.85 (s, 4H, S—CH₂+N—CH₂); 4.27-4.32 (m, 2H, N—CH₂); 4.41-4.46 (m, 2H, N—CH₂); 4.79 (s, 2H, N—CH₂); 7.09 (s, 1H, S—CH); 10.12 (bs, 1H, HCl salt); 10.79 (bs, 2H, NH+HCl salt). M/Z (M+H)⁺: 313.1. Mp: 245-250° C.

### Example 7: trans-3-(((4,4-dimethyl-4,5-dihydro-1H-imidazol-2-yl)thio)methyl)-4a,5,6,7,8,8a-hexahydrobenzo[4,5]imidazo[2,1-b]thiazole dihydrochloride

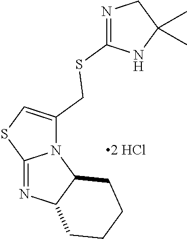

Example 7 was isolated by centrifugation of the reaction mixture followed by trituration of the solid in MeCN and in Et₂O to afford a racemic mixture of trans-3-(((4,4-dimethyl-4,5-dihydro-1H-imidazol-2-yl)thio)methyl)-4a,5,6,7,8,8a-hexahydrobenzo[4,5]imidazo[2,1-b]thiazole dihydrochloride (61 mg, 91%) as a white powder.

¹H-NMR (DMSO-d₆, 400 MHz) δ: 1.36 (d, J 4.0 Hz, 6H, 2 CH₃); 1.37-1.44 (m, 2H, CH₂—CHₐH_{b}); 1.60-1.70 (m, 1H, CH₂—CHₐH_{b}); 1.79-1.90 (m, 3H, CH₂—CHₐH_{b}); 2.21-2.25 (m, 1H, CH₂—CHₐH_{b}); 2.55-2.58 (m, 1H, CH₂—CHₐH_{b}); 3.60 (s, 2H, S—CH₂); 4.01 (ddd, J 14.1, 11.3, 3.2 Hz, 1H, N—CH); 4.18 (ddd, J 14.1, 11.3, 3.2 Hz, 1H, N—CH); 4.83 (dd, 2H, J 41.5, 15.9 Hz, N—CH₂); 7.24 (s, 1H, S—CH); 10.41 (bs, 1H, HCl salt); 10.74 (bs, 1H, HCl salt); 11.39 (bs, 1H, NH). M/Z (M+H)⁺: 323.1. Mp: 248-252° C.

### Example 8: 3-(((4,5-dihydro-1H-imidazol-2-yl)thio)methyl)-6,6-dimethyl-5,6-dihydroimidazo[2,1-b]thiazole dihydrochloride

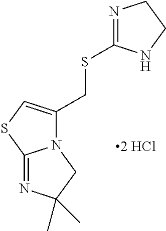 [SMILES: CC1(C)CN2C(CSC3=NCCN3)=CSC2=N1]

Example 8 was isolated by addition of Et₂O in the reaction mixture to precipitate the product that was filtrated. The resulting solid was then purified by precipitation with Et₂O from a solution in EtOH (once), and in MeOH (twice) and freeze-dried in a mixture of water and 1N aqueous HCl to afford 3-(((4,5-dihydro-1H-imidazol-2-yl)thio)methyl)-6,6-dimethyl-5,6-dihydroimidazo[2,1-b]thiazole dihydrochloride (112 mg, 79%) as a white solid.

¹H-NMR (DMSO-d₆, 400 MHz) δ: 1.50 (s, 6H, 2 CH₃); 3.85 (s, 4H, 2N—CH₂—CH₂); 4.23 (s, 2H, S—CH₂); 4.79 (s, 2H, N—CH₂); 7.12 (s, 1H, S—CH); 10.48 (bs, 1H, HCl salt); 10.90 (bs, 2H, HCl salt+NH). M/Z (M+H)⁺: 269.1. Mp: 140-144° C.

### Example 9: 6,6-dimethyl-3-(((1,4,5,6-tetrahydropyrimidin-2-yl)thio)methyl)-5,6-dihydroimidazo[2,1-b]thiazole dihydrochloride

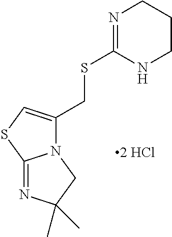 [SMILES: CC1(C)CN2C(CSC3=NCCCN3)=CSC2=N1]

Example 9 was isolated by filtration of the reaction mixture. The solid was washed with MeCN and freeze-dried in a mixture of water and 1N aqueous to afford 6,6-dimethyl-3-(((1,4,5,6-tetrahydropyrimidin-2-yl)thio)methyl)-5,6-dihydroimidazo[2,1-b]thiazole dihydrochloride (138 mg, 93%) as a beige solid.

¹H-NMR (DMSO-d₆, 400 MHz) δ: 1.51 (s, 6H, 2 CH₃); 1.82 (q, J 5.5 Hz, 2H, N—CH₂—CH₂); 3.35 (bs, 4H, 2N—CH₂—CH₂); 4.26 (s, 2H, S—CH₂); 4.71 (s, 2H, N—CH₂); 6.96 (s, 1H, S—CH); 10.55 (bs, 1H, NH); 10.62 (bs, 2H, 2 HCl salts). M/Z (M+H)⁺: 283.2.

### Example 10: cis-3-(((4,4-dimethyl-4,5-dihydro-1H-imidazol-2-yl)thio)methyl)-4a,5,6,7,8,8a-hexahydrobenzo[4,5]imidazo[2,1-b]thiazole dihydrochloride

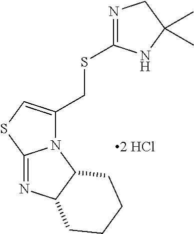

Example 10 was isolated by centrifugation of the reaction mixture followed by trituration of the solid in MeCN and in Et₂O to afford a racemic mixture of cis-3-(((4,4-dimethyl-4,5-dihydro-1H-imidazol-2-yl)thio)methyl)-4a,5,6,7,8,8a-hexahydrobenzo[4,5]imidazo[2,1-b]thiazole dihydrochloride (134 mg, 90%) as a white powder.

¹H-NMR (DMSO-d₆, 400 MHz) δ:1.19-1.30 (m, 1H, CH₂—CHₐH_{b}); 1.37 (d, J 8.5 Hz, 6H, 2 CH₃); 1.52-1.62 (m, 2H, CH₂—CHₐH_{b}); 1.39-1.45 (m, 2H, CH₂—CHₐH_{b}); 1.74-1.83 (m, 1H, CH₂—CHₐH_{b}); 1.98-2.05 (m, 1H, CH₂—CHₐH_{b}); 2.17-2.26 (m, 1H, CH₂—CHₐH_{b}); 3.61 (s, 2H, S—CH₂); 4.65-4.69 (m, 2H, 2N—CH); 4.85-4.94 (m, 2H, N—CH₂); 7.18 (s, 1H, S—CH); 10.22 (bs, 1H, HCl salt); 10.73 (bs, 1H, HCl salt); 11.32 (bs, 1H, NH). M/Z (M+H)⁺: 323.1. Mp: 210-215° C.

### Example 11: 6,6-dimethyl-3-(((1-methyl-4,5-dihydro-1H-imidazol-2-yl)thio)methyl)-5,6-dihydroimidazo[2,1-b]thiazole dihydrochloride

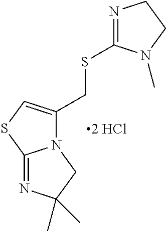 [SMILES: CN1CCN=C1SCC1=CSC2=NC(C)(C)CN12]

Example 11 was isolated by concentration to dryness of the reaction mixture followed by solubilization in iPrOH and precipitation with Et₂O. The resulting precipitate was centrifuged and triturated in twice in Et₂O to afford 6,6-dimethyl-3-(((1-methyl-4,5-dihydro-1H-imidazol-2-yl)thio)methyl)-5,6-dihydroimidazo[2,1-b]thiazole dihydrochloride (110 mg, 74%) as a white solid.

¹H-NMR (DMSO-d₆, 400 MHz) δ: 1.50 (s, 6H, 2 CH₃); 3.02 (s, 3H, N—CH₃); 3.78-3.83 (m, 2H, N—CH₂—CH₂); 3.87-3.92 (in, 2H, N—CH₂—CH₂); 4.24 (s, 2H, S—CH₂); 4.86 (s, 2H, N—CH₂); 7.16 (s, 1H, S—CH); 10.54 (bs, 1H, HCl salt); 11.12 (bs, 1H, HCl salt). M/Z (M+H)⁺: 283.2. Mp: 128-132° C.

### Example 12: 6,6-dimethyl-3-(((4,5,6,7-tetrahydro-1H-1,3-diazepin-2-yl)thio)methyl)-5,6-dihydroimidazo[2,1-b]thiazole dihydrochloride

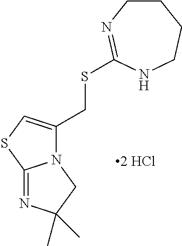 [SMILES: CC1(C)CN2C(CSC3=NCCCCN3)=CSC2=N1]

Example 12 was isolated by trituration of the reaction mixture followed by filtration and trituration of the solid in MeCN and in Et₂O to afford 6,6-dimethyl-3-(((4,5,6,7-tetrahydro-1H-1,3-diazepin-2-yl)thio)methyl)-5,6-dihydroimidazo[2,1-b]thiazole dihydrochloride (144 mg, 93%) as a white solid.

¹H-NMR (DMSO-d₆, 400 MHz) δ: 1.51 (s, 6H, 2 CH₃); 1.71 (bs, 4H, 2N—CH₂—CH₂); 3.41 (bs, 4H, 2N—CH₂—CH₂); 4.27 (s, 2H, S—CH₂); 4.72 (s, 2H, N—CH₂); 6.91 (s, 1H, S—CH); 10.42 (m, 3H, NH+2 HCl salts). M/Z (M+H)⁺: 297.0. Mp: >250° C.

### Example 13: 3-(((5-butyl-4,5-dihydro-1H-imidazol-2-yl)thio)methyl)-6,6-dimethyl-5,6-dihydroimidazo[2,1-b]thiazole dihydrochloride

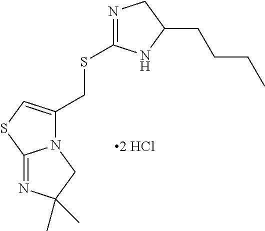 [SMILES: CCCCC1CN=C(SCC2=CSC3=NC(C)(C)CN23)N1]

Example 13 was isolated by evaporation to dryness of the reaction mixture. The resulting oil was solubilized in MeOH and precipitated with Et₂O (5 times). The resulting solid was triturated twice in Et₂O and freeze-dried in a mixture of water and 1N aqueous HCl to afford 3-(((5-butyl-4,5-dihydro-1H-imidazol-2-yl)thio)methyl)-6,6-dimethyl-5,6-dihydroimidazo[2,1-b]thiazole dihydrochloride (104 mg, 89%) as a white powder.

¹H-NMR (DMSO-d₆, 400 MHz) δ: 0.87 (t, J 7.0 Hz, 3H, CH₂—CH₂—CH₂—CH₃); 1.16-1.36 (m, 4H, CH₂—CH₂—CH₂—CH₃); 1.43-1.66 (m, 8H, 2 CH₃+CH₂—CH₂—CH₂—CH₃); 3.51 (dd, J 11.0, 7.4 Hz, 1H, N—CHₐH_{b}); 3.94 (t, J 11.0 Hz, 1H, N—CHₐH_{b}); 4.20-4.28 (m, 3H, S—CH₂+N—CH—CH₂); 4.72 (d, 1H, J 15.8 Hz, N—CHₐH_{b}); 4.86 (d, 1H, J 15.8 Hz, N—CHₐH_{b}); 7.09 (s, 1H, S—CH); 10.44 (s, 1H, HCl salt); 10.86 (s, 1H, HCl salt); 11.08 (s, 1H, NH). M/Z (M+H)⁺: 325.2. Mp: 95-104° C.

### Example 14: 3-(((5,5-dimethyl-4,5-dihydro-1H-imidazol-2-yl)thio)methyl)-6,7-dihydro-5H-thiazolo[3,2-a]pyrimidine dihydrochloride

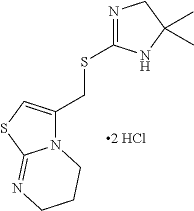 [SMILES: CC1(C)CN=C(SCC2=CSC3=NCCCN23)N1]

Example 14 was isolated by trituration of the reaction mixture to get an off-white precipitate that was filtered and washed with MeCN. The solid was then solubilized in MeOH and precipitated with Et₂O (twice). The resulting solid was triturated twice in Et₂O to afford 3-(((5,5-dimethyl-4,5-dihydro-1H-imidazol-2-yl)thio)methyl)-6,7-dihydro-5H-thiazolo[3,2-a]pyrimidine dihydrochloride as a white solid (106 mg, quant.).

¹H-NMR (DMSO-d₆, 400 MHz) δ: 1.36 (s, 6H, 2 CH₃); 2.08 (quint, J 5.1 Hz, 2H, N—CH₂—CH₂); 3.48 (t, J 5.1 Hz, 2H, N—CH₂—CH₂); 3.59 (s, 2H, S—CH₂); 4.09 (t, J 5.1 Hz, 2H, N—CH₂—CH₂); 4.81 (s, 2H, N—CH₂); 7.20 (s, 1H, S—CH); 10.78 (bs, 2H, 2 HCl salt); 11.35 (bs, 1H, NH). M/Z (M+H)⁺: 283.1. Mp: 242-245° C.

### Example 15: 3-(((5,5-dimethyl-4,5-dihydro-1H-imidazol-2-yl)thio)methyl)-5,6,7,8-tetrahydrothiazolo[3,2-a][1,3]diazepine dihydrochloride

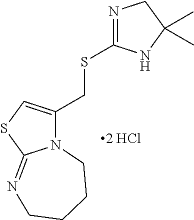 [SMILES: CC1(C)CN=C(SCC2=CSC3=NCCCCN23)N1]

Example 15 was isolated by trituration of the reaction mixture to get an off-white precipitate that was filtered and washed with MeCN to afford 3-(((5,5-dimethyl-4,5-dihydro-1H-imidazol-2-yl)thio)methyl)-5,6,7,8-tetrahydrothiazolo[3,2-a][1,3]diazepine dihydrochloride (145 mg, 94%) as a white solid.

¹H-NMR (DMSO-d₆, 400 MHz) δ: 1.35 (s, 6H, 2 CH₃); 1.93-1.99 (m, 2H, N—CH₂—CH₂); 2.03-2.09 (m, 2H, N—CH₂—CH₂); 3.59 (bs, 4H, N—CH₂—CH₂+S—CH₂); 4.24-4.27 (m, 2H, N—CH₂—H₂); 4.83 (s, 2H, N—CH₂); 7.31 (s, 1H, S—CH); 10.49 (bs, 1H, HCl salt); 10.74 (bs, 1H, HCl salt); 11.34 (bs, 1H, NH). M/Z (M+H)⁺: 297.0. Mp: 244-247° C.

### Example 16: 6,6-dimethyl-3-(((4,4,5,5-tetramethyl-4,5-dihydro-1H-imidazol-2-yl)thio)methyl)-5,6-dihydroimidazo[2,1-b]thiazole dihydrochloride

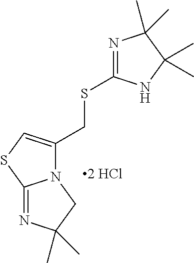 [SMILES: CC1(C)CN2C(CSC3=NC(C)(C)C(C)(C)N3)=CSC2=N1]

Example 16 was isolated by trituration of the reaction mixture to get an off-white precipitate that was filtered and washed with MeCN. The solid was then solubilized in MeOH and precipitated with Et₂O (twice). The resulting solid was triturated twice in Et₂O and freeze-dried in a mixture of water and 1N aqueous HCl (2 equiv) to afford 6,6-dimethyl-3-(((4,4,5,5-tetramethyl-4,5-dihydro-1H-imidazol-2-yl)thio)methyl)-5,6-dihydroimidazo[2,1-b]thiazole dihydrochloride (125 mg, 75%) as a yellow solid.

¹H-NMR (DMSO-d₆, 400 MHz) δ: 1.22 (s, 12H, 4 CH₃); 1.50 (s, 6H, 2 CH₃); 4.24 (s, 2H, S—CH₂); 4.77 (s, 2H, N—CH₂); 7.10 (s, 1H, S—CH); 10.43 (bs, 1H, HCl salt); 11.16 (bs, 2H, HCl salt+NH). M/Z (M+H)⁺: 325.2. Mp: 185-205° C.

### Example 17: 3-(((5-benzyl-4,5-dihydro-1H-imidazol-2-yl)thio)methyl)-6,6-dimethyl-5,6-dihydroimidazo[2,1-b]thiazole dihydrochloride

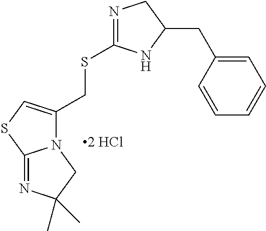 [SMILES: CC1(C)CN2C(CSC3=NCC(Cc4ccccc4)N3)=CSC2=N1]

Example 17 was isolated by filtration of the reaction mixture. The solid was then solubilized in MeOH and precipitated with Et₂O (4 times). The resulting solid was triturated in Et₂O and freeze-dried in a mixture of water and 1N aqueous HCl (2 equiv) to afford 3-(((5-benzyl-4,5-dihydro-1H-imidazol-2-yl)thio)methyl)-6,6-dimethyl-5,6-dihydroimidazo[2,1-b]thiazole dihydrochloride (148 mg, 82%) as a beige solid.

¹H-NMR (DMSO-d₆, 400 MHz) δ: 1.50 (s, 6H, 2 CH₃); 2.87-2.97 (m, 2H, Ph-CH₂); 3.56-3.61 (m, 1H, N—CHₐH_{b}); 3.88 (t, J 10.8 Hz, 1H, N—CHₐH_{b}); 4.20 (s, 2H, S—CH₂); 4.55-4.63 (m, 1H, N—CH—CH₂); 4.66-4.75 (m, 2H, N—CH₂); 7.03 (s, 1H, S—CH); 7.25-7.35 (m, 5H, Ar); 10.35 (bs, 1H, HCl salt); 10.68 (bs, 1H, HCl salt); 11.05 (bs, 1H, NH). M/Z (M+H)⁺: 359.1. Mp: 83-103° C.

### Example 18: 6-benzyl-3-(((5,5-dimethyl-4,5-dihydro-1H-imidazol-2-yl)thio)methyl)-5,6-dihydroimidazo[2,1-b]thiazole dihydrochloride

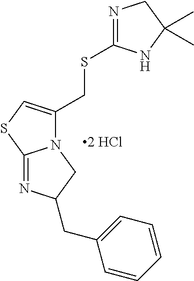 [SMILES: CC1(C)CN=C(SCC2=CSC3=NC(Cc4ccccc4)CN23)N1]

Example 18 was isolated by precipitation of the reaction mixture with Et₂O, followed by filtration. The resulting solid was freeze-dried in a mixture of water and 1N aqueous HCl (2 equiv) to afford 6-benzyl-3-(((5,5-dimethyl-4,5-dihydro-1H-imidazol-2-yl)thio)methyl)-5,6-dihydroimidazo[2,1-b]thiazole dihydrochloride (132 mg, 92%) as a white solid.

¹H-NMR (DMSO-d₆, 400 MHz) δ: 1.34 (d, J 3.0 Hz 6H, 2 CH₃); 3.03-3.12 (m, 2H, Ph-CH₂); 3.58 (s, 2H, S—CH₂); 4.22-4.27 (m, 1H, N—CHₐH_{b}); 4.45 (t, J 10.7 Hz, 1H, N—CHₐH_{b}); 4.69-4.78 (m, 2H, N—CH₂); 4.99-5.07 (m, 1H, N—CH); 7.10 (s, 1H, S—CH); 7.24-7.37 (m, 5H, Ar); 10.19 (bs, 1H, HCl salt); 10.73 (bs, 1H, HCl salt); 11.34 (bs, 1H, NH). M/Z (M+H)⁺: 359.1. Mp: 63-74° C.

### Example 19: 6-butyl-3-(((4,4-dimethyl-4,5-dihydro-1H-imidazol-2-yl)thio)methyl)-5,6-dihydroinidazo[2,1-b]thiazole dihydrochloride

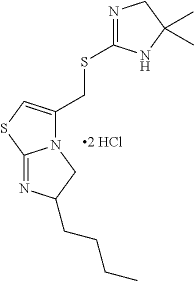 [SMILES: CCCCC1CN2C(CSC3=NCC(C)(C)N3)=CSC2=N1]

Example 19 was isolated by trituration of the reaction mixture in MeCN to get a white precipitate that was filtered and washed with MeCN. The resulting solid was then triturated twice in Et₂O to obtain 6-butyl-3-(((4,4-dimethyl-4,5-dihydro-1H-imidazol-2-yl)thio)methyl)-5,6-dihydroimidazo[2,1-b]thiazole dihydrochloride (158 mg, 89%) as a white powder.

¹H-NMR (DMSO-d₆, 400 MHz) δ: 0.90 (t, J 6.9 Hz, 3H, CH₂—CH₂—CH₂—CH₃); 1.24-1.35 (m, 4H, H₂—CH₂—CH₂—CH₃); 1.36 (d, J 2.8 Hz, 6H, 2 CH₃); 1.66-1.79 (m, 2H, CH₂—H₂—CH₂—CH₃); 3.60 (s, 2H, S—CH₂); 4.15 (dd, J 10.6, 7.6 Hz, 1H, N—CHₐH_{b}); 4.54 (t, J 10.6 Hz, 1H, N—CHₐH_{b}); 4.66-4.83 (m, 3H, N—CH—CH₂+N—CH₂); 7.11 (s, 1H, S—CH); 10.43 (bs, 1H, HCl salt); 10.74 (bs, 1H, HCl salt); 11.36 (bs, 1H, NH). M/Z (M+H)⁺: 325.2. Mp: 210-218° C.

### Example 20: 3-(((5,5-dimethyl-4,5-dihydro-1H-imidazol-2-yl)thio)methyl)-5,5,6,6-tetramethyl-5,6-dihydroimidazo[2,1-b]thiazole dihydrochloride

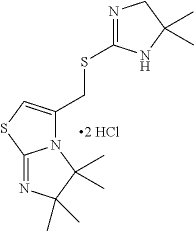 [SMILES: CC1(C)CN=C(SCC2=CSC3=NC(C)(C)C(C)(C)N23)N1]

Example 20 was isolated by filtration of the reaction mixture followed by trituration of the solid in isopropanol. The resulting solid was freeze-dried in a mixture of water and 1N aqueous HCl (2 equiv) to afford 3-(((5,5-dimethyl-4,5-dihydro-1H-imidazol-2-yl)thio)methyl)-5,5,6,6-tetramethyl-5,6-dihydroimidazo[2,1-b]thiazole dihydrochloride (92 mg, 18% yield over 2 steps) as a white solid.

¹H-NMR (DMSO-d₆, 400 MHz) δ: 1.35 (d, J 7.3 Hz, 12H, 4 CH₃); 1.54 (s, 6H, 2 CH₃); 3.62 (s, 2H, S—CH₂); 4.86 (s, 2H, N—CH₂); 7.07 (s, 1H, S—CH); 10.60 (bs, 1H, HCl salt); 10.82 (bs, 1H, HCl salt); 11.42 (bs, 1H, NH). M/Z (M+H)⁺: 325.2. Mp: 140-160° C.

### Example 21: 3-(((5,5-dimethyl-4,5-dihydro-1H-imidazol-2-yl)thio)methyl)-5,6-dihydroimidazo[2,1-b]thiazole dihydrochloride

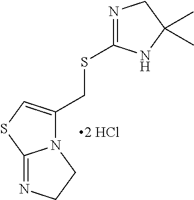 [SMILES: CC1(C)CN=C(SCC2=CSC3=NCCN23)N1]

Example 21 was isolated by concentration to dryness of the reaction mixture. The residue was then solubilized in MeOH and precipitated with Et₂O. The resulting solid was triturated in Et₂O to afford 3-(((5,5-dimethyl-4,5-dihydro-1H-imidazol-2-yl)thio)methyl)-5,6-dihydroimidazo[2,1-b]thiazole dihydrochloride (155 mg, 96%) as a white solid.

¹H-NMR (DMSO-d₆, 400 MHz) δ: 1.35 (s, 6H, 2 CH₃); 3.59 (s, 2H, S—CH₂); 4.27-4.35 (m, 2H, N—CH₂—CH₂); 4.41-4.46 (m, 2H, N—CH₂—CH₂); 4.77 (s, 2H, N—CH₂); 7.09 (s, 1H, S—CH); 9.97 (bs, 1H, HCl salt); 10.74 (bs, 1H, HCl salt); 11.34 (bs, 1H, NH). M/Z (M+H)⁺: 269.1. Mp: 220-230° C.

### Example 22: 3-((((1R,5S)-2,4-diazabicyclo[3.3.1]non-2-en-3-yl)thio)methyl)-6,6-dimethyl-5,6-dihydroimidazo[2,1-b]thiazole dihydrochloride

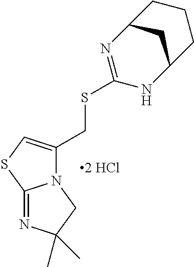 [SMILES: CC1(C)CN2C(CSC3=N[C@@H]4CCC[C@@H](C4)N3)=CSC2=N1]

Example 22 was isolated by centrifugation of the reaction mixture. The resulting solid was triturated in MeCN (2×2 mL), in Et₂O (2×2 mL) and then solubilized in hot MeOH (4 mL) and precipitated with Et₂O (40 mL). The resulting solid was freeze-dried in water to obtain 3-((((1R,5S)-2,4-diazabicyclo[3.3.1]non-2-en-3-yl)thio)methyl)-6,6-dimethyl-5,6-dihydroimidazo[2,1-b]thiazole dihydrochloride (100 mg, 50%) as a white solid.

¹H-NMR (DMSO-d₆, 400 MHz) δ: 0.88-0.97 (m, 1H, CHₐH_{b}); 1.50 (s, 6H, 2 CH₃); 1.56-1.67 (m, 6H, 3 CH₂); 1.94 (d, J 13.0 Hz, 1H, CHₐH_{b}); 3.87 (s, 2H, 2N—CH); 4.22 (s, 2H, S—CH₂); 4.62 (s, 2H, N—CH₂); 6.92 (s, 1H, S—CH); 10.18 (bs, 1H, HCl salt); 10.49 (bs, 2H, NH+HCl salt). M/Z (M+H)⁺: 323.1. Mp>250° C.

### Example 23: cis-3-(((5,5-dimethyl-4,5-dihydro-1H-imidazol-2-yl)thio)methyl)-6,7,8,9-tetrahydro-5H-5,9-methanothiazolo[3,2-a][1,3]diazocine dihydrochloride

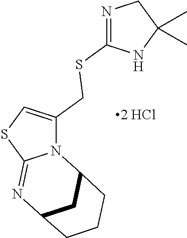

Example 23 was isolated by centrifugation of the reaction mixture. The resulting solid was triturated in MeCN (2×2 mL), in Et₂O (2×2 mL) to obtain a racemic mixture of cis-3-(((5,5-dimethyl-4,5-dihydro-1H-imidazol-2-yl)thio)methyl)-6,7,8,9-tetrahydro-5H-5,9-methanothiazolo[3,2-a][1,3]diazocine dihydrochloride (182 mg, 72% over 2 steps) as a white solid.

¹H-NMR (DMSO-d₆, 400 MHz) δ: 1.08-1.20 (m, 1H, CHₐH_{b}); 1.36 (s, 6H, 2 CH₃); 1.62-1.86 (m, 5H, 2 CH₂+CHₐH_{b}); 2.05-209 (m, 1H, CHₐH_{b}); 2.17 (d, J 13.4 Hz, 1H, CHₐH_{b}); 3.60 (s, 2H, S—CH₂); 4.04 (bs, 1H, N—CH); 4.73-4.86 (m, 3H, N—CH+N—CH₂); 7.18 (s, 1H, S—CH); 10.80 (bs, 1H, HCl salt); 11.12 (bs, 1H, HCl salt); 11.42 (bs, 1H, NH). M/Z (M+H)⁺: 324.1. Mp: 240-245° C.

### Example 24: 3-(((4,6-diazaspiro[2.4]hept-5-en-5-yl)thio)methyl)-6,6-dimethyl-5,6-dihydroimidazo[2,1-b]thiazole dihydrochloride

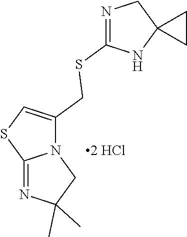 [SMILES: CC1(C)CN2C(CSC3=NCC4(CC4)N3)=CSC2=N1]

Example 24 was isolated by centrifugation of the reaction mixture. The solid was triturated in MeCN (2×2 mL), then solubilized in MeOH and precipitated with Et₂O (twice). The resulting solid was freeze-dried in a mixture of water and 1N aqueous HCl (2 equiv) to afford 3-(((4,6-diazaspiro[2.4]hept-5-en-5-yl)thio)methyl)-6,6-dimethyl-5,6-dihydroimidazo[2,1-b]thiazole dihydrochloride (147 mg, 96%) as a light brown solid.

¹H-NMR (DMSO-ds, 400 MHz) δ: 0.89-0.93 (m, 2H, N—O—CH₂); 1.15-1.19 (m, 2H, N—O—CH₂); 1.50 (s, 6H, 2 CH₃); 3.90 (s, 2H, N—CH₂); 4.22 (s, 2H, S—CH₂); 4.80 (s, 2H, N—CH₂); 7.17 (s, 1H, S—CH); 10.43 (bs, 1H, HCl salt); 10.82 (bs, 1H, HCl salt); 11.67 (bs, 1H, NH). M/Z (M+H)⁺: 295.2.

### Example 25: (3aR,6aS)-2-(((6,6-dimethyl-5,6-dihydroinidazo[2,1-b]thiazol-3-yl)methyl)thio)-3a,4,6,6a-tetrahydro-1H-furo[3,4-d]imidazole dihydrochloride

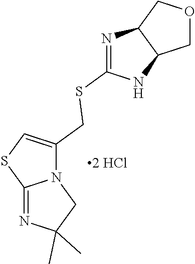 [SMILES: CC1(C)CN2C(CSC3=N[C@H]4COC[C@H]4N3)=CSC2=N1]

Example 25 was isolated by centrifugation of the reaction mixture. The solid was triturated in MeCN (2×2 mL) and in Et₂O (2×2 mL) to obtain (3aR,6aS)-2-(((6,6-dimethyl-5,6-dihydroimidazo[2,1-b]thiazol-3-yl)methyl)thio)-3a,4,6,6a-tetrahydro-1H-furo[3,4-d]imidazole dihydrochloride (110 mg, 55%) as a white solid.

¹H-NMR (DMSO-d₆+D₂O, 400 MHz) δ: 1.46 (s, 6H, 2 CH₃); 3.51-3.53 (m, 2H, 2N—CH); 3.89 (d, 2H, J 10.9 Hz, 2 O—CHₐH_{b}); 4.14 (s, 2H, S—CH₂); 4.44 (s, 2H, N—CH₂); 4.79-4.83 (m, 2H, 2 O—CHₐH_{b}); 6.87 (s, 1H, S—CH). M/Z (M+H)⁺: 311.2. Mp>250° C.

### Example 26: 3-(((4,5-dihydro-1H-imidazol-2-yl)thio)methyl)-6,7-dihydro-5H-thiazolo[3,2-a]pyrimidine dihydrochloride

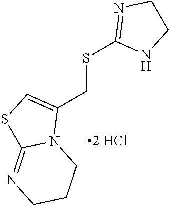 [SMILES: C1=C(CSC2=NCCN2)N2CCCN=C2S1]

Example 26 was isolated by centrifugation of the reaction mixture. The solid was triturated in MeCN (2×2 mL) and Et₂O (3 mL) to afford 3-(((4,5-dihydro-1H-imidazol-2-yl)thio)methyl)-6,7-dihydro-5H-thiazolo[3,2-a]pyrimidine dihydrochloride (133 mg, 92%) as a white solid.

¹H-NMR (DMSO-ds, 400 MHz) δ: 2.02-2.11 (m, 2H, CH₂); 3.42-3.49 (m, 2H, N—CH₂); 3.84 (s, 4H, 2N—CH₂); 3.98-4.05 (m, 2H, N—CH₂); 4.52 (s, 2H, S—CH₂); 6.98 (s, 1H, S—CH); NH and HCl salt signal not observed. M/Z (M+H)⁺: 256.4. Mp>250° C.

### Example 7: 3-(((4,5-dihydro-1H-imidazol-2-yl)thio)methyl)-5,6,7,8-tetrahydrothiazolo[3,2-a][1,3]diazepine dihydrochloride

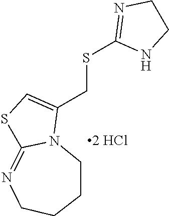 [SMILES: C1=C(CSC2=NCCN2)N2CCCCN=C2S1]

Example 27 was isolated by centrifugation of the reaction mixture. The solid was triturated in MeCN (2×2 mL) and Et₂O (3 mL) to afford 3-(((4,5-dihydro-1H-imidazol-2-yl)thio)methyl)-5,6,7,8-tetrahydrothiazolo[3,2-a][1,3]diazepine dihydrochloride (128 mg, 89%) as a white solid.

¹H-NMR (DMSO-d₆, 400 MHz) δ: 1.90-1.98 (m, 2H, CH₂); 1.99-2.07 (m, 2H, CH₂); 3.54-3.57 (m, 2H, N—CH₂); 3.83 (s, 4H, 2N—CH₂); 4.15-4.18 (m, 2H, N—CH₂); 4.52 (s, 2H, S—CH₂); 7.06 (s, 1H, S—CH); NH and HCl salt signals not observed. M/Z (M+H)⁺: 269.4. Mp>250° C.

### Example 28: 3-(((5,5-dimethyl-1,4,5,6-tetrahydropyrimidin-2-yl)thio)methyl)-6,6-dimethyl-5,6-dihydroimidazo[2,1-b]thiazole dihydrochloride

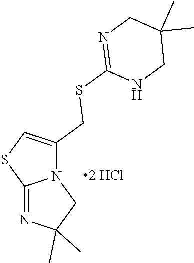 [SMILES: CC1(C)CN=C(SCC2=CSC3=NC(C)(C)CN23)NC1]

Example 28 was isolated by centrifugation of the reaction mixture. The solid was triturated in MeCN (2×2 mL) and Et₂O (2×2 mL) to obtain 3-(((5,5-dimethyl-1,4,5,6-tetrahydropyrimidin-2-yl)thio)methyl)-6,6-dimethyl-5,6-dihydroimidazo[2,1-b]thiazole dihydrochloride (145 mg, 91%) as a white solid.

¹H-NMR (DMSO-d₆+D₂O, 400 MHz) δ: 0.89 (s, 6H, 2 CH₃); 1.48 (s, 6H, 2 CH₃); 3.07 (s, 4H, 2N—CH₂); 4.20 (s, 2H, S—CH₂); 4.51 (s, 2H, N—CH₂); 6.85 (s, 1H, S—CH). M/Z (M+H)⁺: 311.1. Mp>250° C.

### Example 29: 3-(((1,4,5,6-tetrahydropyrimidin-2-yl)thio)methyl)-6,7-dihydro-5H-thiazolo[3,2-a]pyrimidine dihydrochloride

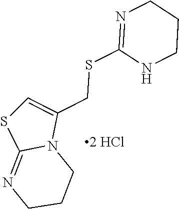 [SMILES: C1=C(CSC2=NCCCN2)N2CCCN=C2S1]

Example 29 was isolated by centrifugation of the reaction mixture. The solid was triturated in MeCN (3 mL) and Et₂O (3 mL) to obtain 3-(((1,4,5,6-tetrahydropyrimidin-2-yl)thio)methyl)-6,7-dihydro-5H-thiazolo[3,2-a]pyrimidine dihydrochloride (129 mg, 85%) as a white solid.

¹H-NMR (DMSO-d₆, 400 MHz) δ: 1.81 (quint, J 5.8 Hz, 2H, CH₂); 2.09 (quint, J 5.6 Hz, 2H, CH₂); 2.35 (t, J 5.8 Hz, 4H, 2N—CH₂); 3.49 (t, J 5.6 Hz, 2H, N—CH₂); 4.13 (t, J 5.6 Hz, 2H, N—CH₂); 4.70 (s, 2H, S—CH₂); 7.01 (s, 1H, S—CH); 10.57 (bs, 2H, NH+HCl salt); 10.81 (bs, 1H, HCl salt). M/Z (M+H)⁺: 269.2. Mp>250° C.

### Example 30: 3-(((5-butyl-4,5-dihydro-1H-imidazol-2-yl)thio)methyl)-5,6-dihydroimidazo[2,1-b]thiazole dihydrochloride

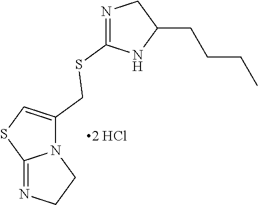 [SMILES: CCCCC1CN=C(SCC2=CSC3=NCCN23)N1]

Example 30 was isolated by centrifugation of the reaction mixture. The solid was triturated in MeCN (2×2 mL) and Et₂O (3 mL) to obtain 3-(((5-butyl-4,5-dihydro-1H-imidazol-2-yl)thio)methyl)-5,6-dihydroimidazo[2,1-b]thiazole dihydrochloride (78 mg, 45%) as a white solid.

¹H-NMR (DMSO-d₆, 400 MHz) δ: 0.88 (t, J 7.0 Hz, 3H, CH₃); 1.17-1.34 (m, 4H, CH₂—CH₂—CH₂—CH₃); 1.48-1.64 (m, 2H, CH₂—CH₂—CH₂—CH₃); 3.51 (dd, J 11.0, 7.6 Hz, 1H, N—CHₐH_{b}); 3.94 (t, J 11.0 Hz, 1H, N—CHₐH_{b}); 4.20-4.33 (m, 3H, N—CH—H₂+N—CH₂); 4.39-4.50 (m, 2H, N—CH₂); 4.68-4.77 (m, 1H, S—CHₐH_{b}); 4.81-4.94 (m, 1H, S—CHₐH_{b}); 7.08 (s, 1H, S—CH); 9.95 (bs, 1H, HCl salt); 10.83 (bs, 1H, NH); 11.07 (bs, 1H, HCl salt). M/Z (M+H)⁺: 297.1. Mp: 176-178° C.

### Example 31: 3-(3-((5,5-dimethyl-4,5-dihydro-1H-imidazol-2-yl)thio)propyl)pyridine dihydrochloride

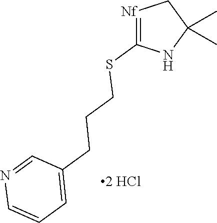

Example 31 was isolated by concentration to dryness of the reaction mixture. Then the residue was purified by flash chromatography (KPNH, DCM 100% to DCM/MeOH 90:10). The resulting yellow oil was dissolved in DCM, then HCl 2 M in Et₂O (5 equiv) was added. The supernatant was removed to afford 3-(3-((5,5-dimethyl-4,5-dihydro-1H-imidazol-2-yl)thio)propyl)pyridine dihydrochloride (58 mg, 47%) as a yellow sticky solid.

¹H-NMR (DMSO-d₆+D₂O, 400 MHz) δ: 1.34 (s, 6H, 2 CH₃); 1.96-2.03 (m, 2H, CH₂—CH₂); 2.88 (t, J 7.6 Hz, 2H, Ar—CH₂); 3.20 (t, J 7.6 Hz, 2H, S—CH₂); 3.60 (s, 2H, N—CH₂); 7.88-7.91 (m, 1H, Ar); 8.35-8.37 (m, 1H, Ar); 8.69-8.70 (m, 1H, Ar); 8.74 (bs, 1H, Ar). M/Z (M+H)⁺: 250.2.

### Example 32: 3-(((5,5-dimethyl-4,5-dihydro-1H-imidazol-2-yl)thio)methyl)pyridine dihydrochloride

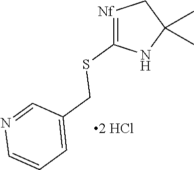

Example 32 was isolated by filtration of the reaction mixture and washing of the solid with MeCN to afford 3-(((5,5-dimethyl-4,5-dihydro-1H-imidazol-2-yl)thio)methyl)pyridine dihydrochloride (171 mg, quant.) as a white hygroscopic solid.

¹H-NMR (DMSO-d₆, 400 MHz) δ: 1.32 (s, 6H, 2 CH₃); 3.58 (s, 2H, S—CH₂); 4.82 (s, 2H, N—CH₂); 7.80-7.83 (m, 1H, Ar); 8.40-8.42 (m, 1H, Ar); 8.74-8.76 (in, 1H, Ar); 8.98-8.99 (m, 1H, Ar); 10.68 (bs, 1H, HCl salt); 11.18 (bs, 1H, NH); HCl salt not observed. M/Z (M+H)⁺: 222.1,

### Example 33: 3′-(((5,5-dimethyl-4,5-dihydro-1H-imidazol-2-yl)thio)ethyl)-5′H-spiro[cyclopropane-1,6′-imidazo[2,1-b]thiazole] dihydrochloride

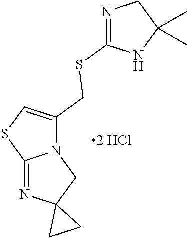 [SMILES: CC1(C)CN=C(SCC2=CSC3=NC4(CC4)CN23)N1]

Example 33 was isolated by filtration of the reaction mixture followed by washing with MeCN. The solid was then solubilized in MeOH and precipitated with Et₂O (twice). The resulting solid was freeze-dried in a mixture of water and 1N aqueous HOI (2 equiv) to afford 3′-(((5,5-dimethyl-4,5-dihydro-1H-imidazol-2-yl)thio)methyl)-5′H-spiro[cyclopropane-1,6′-imidazo[2,1-b]thiazole] dihydrochloride (139 mg, 90%) as a sticky brown solid.

¹H-NMR (DMSO-d₆, 400 MHz) δ: 1.00-1.03 (m, 4H, 2 C—CH₂); 1.36 (s, 6H, 2 CH₃); 3.60 (s, 2H, S—CH₂); 4.53 (s, 2H, N—CH₂); 4.78 (s, 2H, N—CH₂); 7.16 (s, 1H, S—CH); 10.57 (bs, 1H, HCl salt); 10.73 (bs, 1H, HCl salt); 11.32 (bs, 1H, NH). M/Z (M+H)⁺: 295.1.

### Example 34: 3-(((5,5-dimethyl-4,5-dihydro-1H-imidazol-2-yl)thio)methyl)-6,6-dimethyl-6,7-dihydro-5H-thiazolo[3,2-a]pyrimidine dihydrochloride

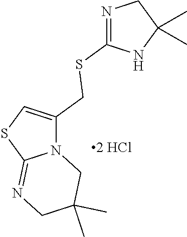 [SMILES: CC1(C)CN=C2SC=C(CSC3=NCC(C)(C)N3)N2C1]

Example 34 was isolated by centrifugation of the reaction mixture. The solid was triturated in MeCN (2×2 mL) and Et₂O (3 mL) to obtain 3-(((5,5-dimethyl-4,5-dihydro-1H-imidazol-2-yl)thio)methyl)-6,6-dimethyl-6,7-dihydro-5H-thiazolo[3,2-a]pyrimidine dihydrochloride (140 mg, 93%) as a white solid.

¹H-NMR (DMSO-d₆, 400 MHz) δ: 1.03 (s, 6H, 2 CH₃); 1.36 (s, 6H, 2 CH₃); 3.24 (s, 2H, N—CH₂); 3.60 (s, 2H, S—CH₂); 3.88 (s, 2H, N—CH₂); 4.83 (s, 2H, N—CH₂); 7.28 (s, 1H, S—CH); 10.74 (bs, 1H, HCl salt); 10.92 (bs, 1H, HCl salt); 11.36 (bs, 1H, NH). M/Z (M+H)⁺: 311.2. Mp: 169-175° C.

### Example 35: 3-(((1,4,5,6-tetrahydropyrimidin-2-yl)thio)methyl)-5,6-dihydroimidazo[2,1-b]thiazole dihydrochloride

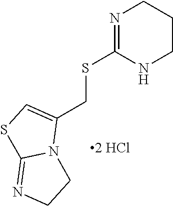 [SMILES: C1=C(CSC2=NCCCN2)N2CCN=C2S1]

Example 35 was isolated by centrifugation of the reaction mixture. The solid was solubilized in MeOH (3 mL) and then poured into Et₂O (5 mL). The resulting precipitate was isolated by centrifugation to afford 3-(((1,4,5,6-tetrahydropyrimidin-2-yl)thio)methyl)-5,6-dihydroimidazo[2,1-b]thiazole dihydrochloride (50 mg, 32%) as a beige solid.

¹H-NMR (DMSO-d₆, 400 MHz) δ: 1.82 (quint, J 5.6 Hz, 2H, CH₂); 3.35 (bs, 4H, 2N—CH₂); 4.29-4.34 (in, 2H, N—CH₂); 4.44-4.49 (m, 2H, N—CH₂); 4.66 (bs, 2H, S—CH₂); 6.91 (s, 1H, S—CH); 9.98 (bs, 1H, HCl salt); 10.55 (bs, 2H, NH+HCl salt). M/Z (M+H)⁺: 255.1. Mp: 100-102° C.

### Example 36: mixture of (4aS,7aR)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole dihydrochloride and (4aR,7aS)-3-((((3aR,6aS)-3a,4,6,6a-tetrahydro-1H-furo[3,4-d]imidazol-2-yl)thio)methyl)-4a,5,7,7a-tetrahydrofuro[3′,4′: 4,5]imidazo[2,1-b]thiazole dihydrochloride

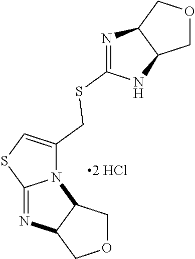 [SMILES: C1=C(CSC2=N[C@H]3COC[C@H]3N2)N2C(=N[C@@H]3COC[C@@H]32)S1]

Example 36 was isolated by centrifugation of the reaction mixture. The solid was triturated in MeCN (2×2 mL) and Et₂O (3 mL) to obtain a mixture of (4aS,7aR)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole dihydrochloride and (4aR,7aS)-3-((((3aR,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole dihydrochloride (138 mg, 72% over 2 steps) as a white solid.

The product was isolated as a mixture of diastereoisomers (ratio 6:4).

¹H-NMR (DMSO-d₆, 400 MHz) δ: 3.53 (m, 2H, 2N—CH); 3.66 (ddd, J 16.0, 11.0, 4.5 Hz, 2H, 2N—CH); 3.98 (dd, J 10.2, 3.9 Hz, 2H, 2 O—CHₐH_{b}); 4.07 (d, J 11.0 Hz, 1H, O—CHₐH_{b}); 4.39 (d, J 11.0 Hz, 1H, O—CHₐH_{b}); 4.77 (d, J 16.0 Hz, 1H, O—CHₐH_{b}); 4.86 (m, 2H, 2 O—CHₐH_{b}); 4.94 (d, J 16.0 Hz, 1H, O—CHₐH_{b}); 5.33 (d, J 4.0 Hz, 0.4H, S—CHₐH_{b} of one diastereoisomer); 5.35 (d, J 4.0 Hz, 0.6H, S—CHₐH_{b} of other diastereoisomer); 5.48 (d, J 4.0 Hz, 0.6H, S—CHₐH_{b} of other diastereoisomer); 5.50 (d, J 4.0 Hz, 0.4H, S—CHₐH_{b} of one diastereoisomer); 7.10 (s, 1H, S—CH); 10.45 (bs, 1H, HCl salt); 11.31 (bs, 2H, HCl salt+NH). M/Z (M+H)⁺: 325.1. Mp>250° C.

### Example 37: 3-(((1,4,5,6-tetrahydropyrimidin-2-yl)thio)methyl)-5,6,7,8-tetrahydrothiazolo[3,2-a][1,3]diazepine dihydrochloride

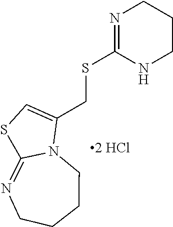 [SMILES: C1=C(CSC2=NCCCN2)N2CCCCN=C2S1]

Example 37 was isolated by centrifugation of the reaction mixture. The solid was triturated in MeCN (2 mL) and Et₂O (3 mL) to afford 3-(((1,4,5,6-tetrahydropyrimidin-2-yl)thio)methyl)-5,6,7,8-tetrahydrothiazolo[3,2-a][1,3]diazepine dihydrochloride (144 mg, 97%) as a white solid.

¹H-NMR (DMSO-d₆, 400 MHz) δ: 1.82 (quint, J 5.8 Hz, 2H, CH₂); 1.93-1.99 (m, 2H, CH₂); 2.06-2.12 (in, 2H, CH₂); 3.35 (t, J 5.8 Hz, 4H, 2N—CH₂); 3.61-3.64 (m, 2H, N—CH₂); 4.27-4.30 (m, 2H, N—CH₂); 4.77 (s, 2H, S—CH₂); 7.12 (s, 1H, S—CH); 10.57 (bs, 3H, NH+2 HCl salts). M/Z (M+H)⁺: 283.2. Mp>250° C.

### Example 38: 1-(3-((5,5-dimethyl-4,5-dihydro-1H-imidazol-2-yl)thio)propyl)-1H-imidazole dihydrochloride

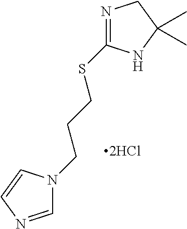 [SMILES: CC1(C)CN=C(SCCCn2ccnc2)N1]

Example 38 was isolated by concentration to dryness of the reaction mixture. The residue was purified by flash chromatography (KPNH, DCM 100% to DCM/MeOH 90:10). The resulting brown oil was dissolved in DCM, then HCl 2 M in Et₂O (5 equiv) was added. The supernatant was removed, then the residue was solubilized in MeOH and precipitated with Et₂O (twice). The resulting solid was freeze-dried in a mixture of water and 1N aqueous HCl (2 equiv) to afford 1-(3-((5,5-dimethyl-4,5-dihydro-1H-imidazol-2-yl)thio)propyl)-1H-imidazole dihydrochloride (96 mg, 67%) as a yellow hydroscopic solid.

¹H-NMR (DMSO-d₆, 400 MHz) δ: 1.37 (s, 6H, 2 CH₃); 2.19-2.26 (m, 2H, CH₂—CH₂); 3.29-3.36 (in, 2H, Ar—CH₂); 3.60 (s; 2H, N—CH₂); 4.34 (t, J 6.9 Hz, 2H, S—CH₂); 7.70 (t, J 1.6 Hz, 1H, N—CH); 7.85 (t, J 1.6 Hz, 1H, N—CH); 9.24-9.25 (m, 1H, N—CH); 10.56 (bs, 1H, HCl salt); 10.95 (bs, 1H, HCl salt); 14.73 (bs, 1H, NH). M/Z (M+H)⁺: 239.3.

### Example 39: 3-(((1,3-diazaspiro[4.5]dec-2-en-2-yl)thio)methyl)-6,6-dimethyl-5,6-dihydroimidazo[2,1-b]thiazole dihydrochloride

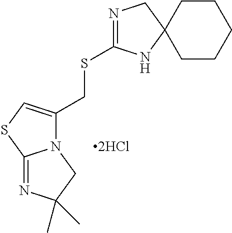 [SMILES: CC1(C)CN2C(CSC3=NCC4(CCCCC4)N3)=CSC2=N1]

Example 39 was isolated by centrifugation of the reaction mixture. The solid was triturated in MeCN (2×2 mL) and Et₂O (2×2 mL) to obtain 3-(((1,3-diazaspiro[4.5]dec-2-en-2-yl)thio)methyl)-6,6-dimethyl-5,6-dihydroimidazo[2,1-b]thiazole dihydrochloride (146 mg, 85%) as a white solid.

¹H-NMR (DMSO-d₆, 400 MHz) δ: 1.24-1.46 (m, 4H, 2 CH₂); 1.50 (s, 6H, 2 CH₃); 1.64-1.68 (m, 6H, 3 CH₂); 3.64 (s, 2H, N—CH₂); 4.23 (s, 2H, S—CH₂); 4.77 (s, 2H, N—CH₂); 7.06 (s, 1H, S—CH); 10.34 (bs, 1H, HCl salt); 10.78 (bs, 1H, HCl salt); 11.19 (s, 1H, NH). M/Z (M+H)⁺: 337.1. Mp: 225-231° C.

### Example 40: 6,6-dimethyl-3-(((1,4,4a,5,6,7,8,8a-octahydroquinazolin-2-yl)thio)methyl)-5,6-dihydroimidazo[2,1-b]thiazole dihydrochloride

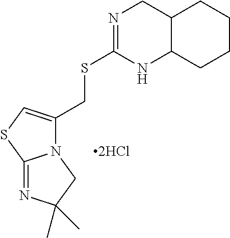 [SMILES: CC1(C)CN2C(CSC3=NCC4CCCCC4N3)=CSC2=N1]

Example 40 was isolated by centrifugation of the reaction mixture. The solid was triturated in MeCN (2×2 mL) and Et₂O (2×2 mL) to obtain 6,6-dimethyl-3-(((1,4,4a,5,6,7,8,8a-octahydroquinazolin-2-yl)thio)methyl)-5,6-dihydroimidazo[2,1-b]thiazole dihydrochloride (146 mg, 85%) as a white solid.

The product was isolated as a mixture of 2 couples of enantiomers (ratio 0.6:0.4):

¹H-NMR (DMSO-d₆, 400 MHz) δ: 0.96-1.38 (in, 5H, CH₂ of mixture of the 2 couples of enantiomers); 1.51 (s, 6H, 2 CH₃ of one couple of enantiomers+2 CH₃ of other couple of enatiomers); 1.56-1.86 (m, 3H, CH₂ of mixture of the 2 couples of enantiomers); 1.98-2.04 (m, 0.6H, OH of one couple of enantiomers); 2.14-2.18 (m, 0.4H, CH of other couple of enantiomers); 3.00 (dd, J 11.0, 1.6 Hz, 0.4H, OH of other couple of enantiomers); 3.13 (dt, J 11.0, 3.0 Hz, 0.4H, CHₐH_{b} of other couple of enantiomers); 3.22 (dd, J 13.5, 4.2 Hz, 0.6H, CHₐH_{b} of one couple of enantiomers); 3.39 (dd, J 13.5, 4.2 Hz, 1H, CHₐH_{b} of one couple of enantiomers+CHₐH_{b} of other couple of enantiomers); 3.67-3.71 (m, 0.6H, CH of one couple of enantiomers); 4.26-4.34 (m, 2H, S—CH₂ of one couple of enantiomers+S—CH₂ of other couple of enantiomers); 4.71 (d, J 3.0 Hz, 0.8H, N—CH₂ of other couple of enantiomers); 4.76 (s, 1.2H, N—CH₂ of one couple of enantiomers); 6.92 (s, 0.4H, S—CH of other couple of enantiomers); 6.94 (s, 0.6H, S—CH of one couple of enantiomers); 10.45-10.52 (m, 2H, 2 HCl salt of one couple of enantiomers+2 HCl salt of other couple of enantiomers); 10.65 (bs, 1H, NH of one couple of enantiomers+NH of other couple of enantiomers). M/Z (M+H)⁺: 337.1. Mp: 245-249° C.

### Example 41: 5-(2-((5,5-dimethyl-4,5-dihydro-1H-imidazol-2-yl)thio)ethyl)quinoline dihydrochloride

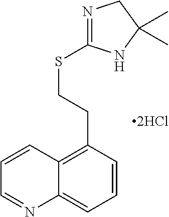 [SMILES: CC1(C)CN=C(SCCc2cccc3ncccc23)N1]

Example 41 was isolated by concentration to dryness of the reaction mixture. Then the residue was passed through an ISOLUTE® SCX-2 cartridge (DOM and MeOH, then NH₃ 7 M in MeOH) and then purified by flash chromatography (15 μm, DCM 100% to DCM/MeOH 80:20). The resulting white solid was dissolved in DOM and precipitated with HCl 2 M in Et₂O. The solid was filtrated to afford 5-(2-((5,5-dimethyl-4,5-dihydro-1H-imidazol-2-yl)thio)ethyl)quinoline dihydrochloride (46 mg, 36%) as a white solid.

¹H-NMR (D₂O, 400 MHz) δ: 1.22 (s, 6H, 2 CH₃); 3.44 (s, 2H, N—CH₂); 3.63-3.66 (m, 2H, S—CH₂); 3.71-3.75 (m, 2H, Ar—CH₂); 7.93-7.94 (m, 1H, Ar); 8.11-8.17 (m, 2H, Ar); 8.22-8.24 (m, 1H, Ar); 9.16-9.17 (m, 1H, Ar); 9.34-9.37 (m, 1H, Ar). M/Z (M+H)⁺: 286.1. Mp: 240-245° C.

### Example 42: 3′-(((5,5-dimethyl-4,5-dihydro-1H-imidazol-2-yl)thio)methyl)-5′H-spiro[cyclohexane-1,6′-imidazo[2,1-b]thiazole] dihydrochloride

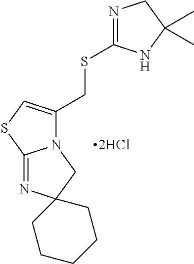 [SMILES: CC1(C)CN=C(SCC2=CSC3=NC4(CCCCC4)CN23)N1]

Example 42 was isolated by centrifugation of the reaction mixture. The solid was triturated in MeCN (2×2 mL) and Et₂O (2×2 mL) to obtain 3′-(((5,5-dimethyl-4,5-dihydro-1H-imidazol-2-yl)thio)methyl)-5′H-spiro[cyclohexane-1,6′-imidazo[2,1-b]thiazole] dihydrochloride (130 mg, 89%) as a white solid.

¹H-NMR (DMSO-d₆, 400 MHz) δ: 1.36 (s, 6H, 2 CH₃); 1.42-1.52 (m, 4H, 2 CH₂); 1.61-1.68 (m, 2H, CH₂); 1.81-1.83 (m, 4H, 2 CH₂); 3.60 (s, 2H, S—CH₂); 4.27 (s, 2H, N—CH₂); 4.76 (s, 2H, N—CH₂); 7.09 (s, 1H, S—CH); 10.74-10.86 (m, 2H, 2 HCl salt); 11.35 (bs, 1H, NH). M/Z (M+H)⁺: 337.1. Mp: 190-198° C.

### Example 43: 3-(((5,5-dimethyl-4,5-dihydro-1H-imidazol-2-yl)thio)methyl)-5a,6,7,8,9,9a-hexahydro-5H-thiazolo[2,3-b]quinazoline dihydrochloride

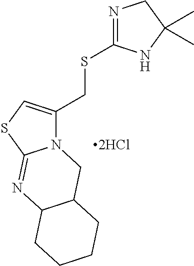 [SMILES: CC1(C)CN=C(SCC2=CSC3=NC4CCCCC4CN23)N1]

Example 43 was isolated by centrifugation of the reaction mixture. The solid was triturated in MeCN (2×2 mL) and Et₂O (2×2 mL) to obtain 3-(((5,5-dimethyl-4,5-dihydro-1H-imidazol-2-yl)thio)methyl)-5a,6,7,8,9,9a-hexahydro-5H-thiazolo[2,3-b]quinazoline dihydrochloride (140 mg, 96%) as a white solid.

The product was isolated as a mixture of 2 couples of enantiomers (ratio 1:1): ¹H-NMR (DMSO-d₆, 400 MHz) δ: 1.08-1.29 (m, 2H, 2 CHₐH_{b}); 1.36 (dd, J 7.5, 3.4 Hz, 6H, 2 CH₃); 1.42-1.86 (m, 6H, 2 CHₐH_{b}+4 CH H); 2.03-2.08 (m, 0.5H, CH of one couple of enantiomers); 2.28-2.31 (m, 0.5H, CH of other couple of enantiomers); 3.21-3.26 (m, 0.5H, N—CHₐH_{b} of one couple of enantiomers); 3.54-3.62 (m; 0.5H, N—CHₐH_{b} of other couple of enantiomers); 3.60 (s, 2H, N—CH₂); 3.82-3.86 (m, 0.5H, N—CHₐH_{b} of one couple of enantiomers); 4.02-4.12 (m, 1H, N—CH); 4.32 (dd, J 12.0, 4.5 Hz, 0.5H, N—CHₐH_{b} of other couple of enantiomers); 4.67 (d, J 15.5 Hz, 0.5H, S—CHₐH_{b} of other couple of enantiomers); 4.77-4.85 (m, 1H, S—CH₂ of one couple of enantiomers); 4.91 (d, J 15.5 Hz, 0.5H, S—CHₐH_{b} of other coupler of enantiomers); 7.21 (s, 1H, S—CH); 10.71-10.82 (m, 2H, 2 HCl salt); 11.31 (bs, 1H, NH). M/Z (M+H)⁺: 337.1. Mp: 241-245° C.

### Example 44: 3-((((3aR,7aR)-3a,4,5,6,7,7a-hexahydro-1H-benzo[d]imidazol-2-yl)thio)methyl)-5,6-dihydroimidazo[2,1-b]thiazole dihydrochloride

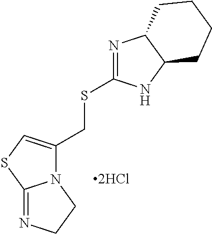 [SMILES: C1=C(CSC2=N[C@@H]3CCCC[C@H]3N2)N2CCN=C2S1]

Example 44 was isolated by centrifugation of the reaction mixture. The solid was triturated in MeCN (3 mL), EtOH (2×1.5 mL) and Et₂O (3 mL) to afford 3-((((3aR,7aR)-3a,4,5,6,7,7a-hexahydro-1H-benzo[d]imidazol-2-yl)thio)methyl)-5,6-dihydroimidazo[2,1-b]thiazole dihydrochloride (39 mg, 30%) as a white solid.

¹H-NMR (DMSO-d₆, 400 MHz) δ: 1.24-1.36 (m, 2H, CH₂); 1.47-1.54 (m, 2H, CH₂); 1.76-1.78 (m, 2H, CH₂); 2.14 (d, J 11.2 Hz, 2H, CH₂); 3.40-3.48 (m, 2H, 2N—CH); 4.27-4.33 (m, 2H, N—CH₂); 4.41-4.46 (m, 2H. N—CH₂); 4.77 (d, J 15.6 Hz, 1H. S—CHₐH_{b}); 4.85 (d, J 15.6 Hz, 1H, S—CHₐH_{b}); 7.13 (s, 1H, S—CH); 9.96 (bs, 1H, HCl salt); 11.31 (bs, 2H, NH+HCl salt). M/Z (M+H)⁺: 295.5. Mp: 230-233° C.

### Example 45: 3-((((3aR,7aR)-3a,4,5,6,7,7a-hexahydro-1H-benzo[d]imidazol-2-yl)thio)methyl)-6,7-dihydro-5H-thiazolo[3,2-a]pyrimidine dihydrochloride

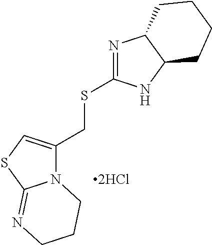 [SMILES: C1=C(CSC2=N[C@@H]3CCCC[C@H]3N2)N2CCCN=C2S1]

Example 45 was isolated by centrifugation of the reaction mixture. The solid was triturated in MeCN (2×3 mL) and Et₂O (3 mL) to afford 3-((((3aR,7aR)-3a,4,5,6,7,7a-hexahydro-1H-benzo[d]imidazol-2-yl)thio)methyl)-6,7-dihydro-5H-thiazolo[3,2-a]pyrimidine dihydrochloride (93 mg, 73%) as a white solid.

¹H-NMR (DMSO-d₆, 400 MHz) δ: 1.24-1.36 (m, 2H, CH₂); 1.46-1.55 (m, 2H, CH₂); 1.77-1.75 (m, 2H, CH₂); 2.05-2.10 (m, 2H, CH₂); 2.14 (d, J 11.2 Hz, 2H, CH₂); 3.42-3.45 (m, 2H, 2N—CH); 3.48 (t, J 5.6 Hz, 2H, N—CH₂); 4.09 (t, J 5.6 Hz 2H, N—CH₂); 4.79 (d, J 15.6 Hz, 1H, S—CHₐH_{b}); 4.89 (d, J 15.6 Hz, 1H, S—CHₐH_{b}); 7.22 (s, 1H, S—CH); 10.81 (bs, 1H, HCl salt); 11.35 (bs, 2H, NH+HCl salt). M/Z (M+H)⁺: 309.1. Mp>250° C.

### Example 46: 3-((((3aR,7aR)-3a,4,5,6,7,7a-hexahydro-1H-benzo[d]imidazol-2-yl)thio)methyl)-5,6,7,8-tetrahydrothiazolo[3,2-a][1,3]diazepine dihydrochloride

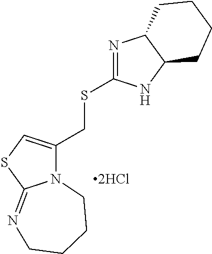 [SMILES: C1=C(CSC2=N[C@@H]3CCCC[C@H]3N2)N2CCCCN=C2S1]

Example 46 was isolated by centrifugation of the reaction mixture. The solid was triturated in MeCN (2×3 mL) and Et₂O (3 mL) to afford 3-((((3aR,7aR)-3a,4,5,6,7,7a-hexahydro-1H-benzo[d]imidazol-2-yl)thio)methyl)-5,6,7,8-tetrahydrothiazolo[3,2-a][1,3]diazepine dihydrochloride (106 mg, 86%) as a white solid.

¹H-NMR (DMSO-d₆, 400 MHz) δ: 1.24-1.36 (m, 2H, CH₂); 1.46-1.55 (m, 2H, CH₂); 1.77-1.75 (m, 2H, CH₂); 1.93-2.00 (m, 2H, CH₂); 2.02-2.09 (m, 2H, CH₂); 2.14 (d, J 11.2 Hz, 2H, CH₂); 3.43-3.45 (m, 2H, 2N—CH); 3.60-3.61 (m, 2H, N—CH₂); 4.25-4.27 (m, 2H, N—CH₂); 4.82 (d, J 15.4 Hz, 1H, S—CHₐH_{b}); 4.93 (d, J 15.4 Hz, 1H, S—CHₐH_{b}); 7.35 (s, 1H, S—CH); 10.56 (bs, 1H, HOI salt); 11.36 (bs, 2H, NH+HCl salt). M/Z (M+H)⁺: 323.1. Mp>250° C.

### Example 47: 1-(2-((5,5-dimethyl-4,5-dihydro-1H-imidazol-2-yl)thio)ethyl)-1H-imidazole dihydrochloride

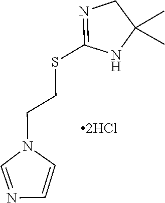

Example 47 was isolated by concentration to dryness of the reaction mixture. Then the residue was passed through an ISOLUTE® SCX-2 cartridge (DCM and MeOH, then NH₃ 7 M in MeOH) and then purified twice by flash chromatography (KPNH, DCM 100% to DCM/MeOH 95:05 then 15 μm, DCM 100% to DCM/MeOH 90:10). The resulting colorless oil was dissolved in DCM and then HCl 2 M in Et₂O (5.0 equiv) was added. The supernatant was removed and the residue was freeze-dried in a mixture of water and 1N aqueous HCl to afford 1-(2-((5,5-dimethyl-4,5-dihydro-1H-imidazol-2-yl)thio)ethyl)-1H-imidazole dihydrochloride (78 mg, 34%) as a beige solid.

¹H-NMR (DMSO-d₆, 400 MHz) δ: 1.37 (s, 6H, 2 CH₃); 3.60 (s, 2H, N—CH₂); 3.87 (t, J 6.9 Hz, 2H, S—CH₂); 4.56 (t, J 6.9 Hz, 2H, Ar—CH₂); 7.72 (t, J 1.7 Hz, 1H, N—CH); 7.90 (t, J 1.7 Hz, 1H, N—CH); 9.33 (t, J 1.4 Hz, 1H, N—CH); 10.64 (bs, 1H, HCl salt); 11.01 (bs, 1H, NH); HCl salt not observed. M/Z (M+H)⁺: 225.2.

### Example 48: 3-(((5-benzyl-4,5-dihydro-1H-imidazol-2-yl)thio)methyl)-6,7-dihydro-5H-thiazolo[3,2-a]pyrimidine dihydrochloride

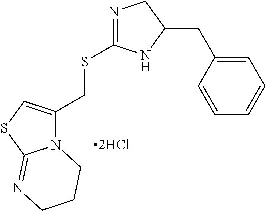 [SMILES: C1=C(CSC2=NCC(Cc3ccccc3)N2)N2CCCN=C2S1]

Example 48 was isolated by centrifugation of the reaction mixture. The solid was triturated in MeCN (2×3 mL) and Et₂O (3 mL). The resulting solid was solubilized in water (10 mL) and this aqueous layer was washed with DCM (10 mL) and freeze-dried to give 3-(((5-benzyl-4,5-dihydro-1H-imidazol-2-yl)thio)methyl)-6,7-dihydro-5H-thiazolo[3,2-a]pyrimidine dihydrochloride (36 mg, 26%) as a white solid.

¹H-NMR (DMSO-d₆, 400 MHz) δ: 2.05-2.11 (m, 2H, CH₂); 2.87-2.97 (m, 2H, CH—CH₂—Ar); 3.50 (t, J 5.6 Hz, 2H, N—CH₂); 3.58 (dd, J 11.2, 6.8 Hz, 1H, N—CHₐH_{b}—CH); 3.87 (t, J 11.2 Hz, 1H, N—CHₐ—VCH); 4.06 (t, J 5.6 Hz, 2H, N—CH₂); 4.55-4.63 (m, 1H, N—CH—CH₂); 4.72-4.81 (m, 2H, S—CH₂); 7.12 (s, 1H, S—CH); 7.25-7.35 (m, 5H, Ar); 10.77 (bs, 2H, NH+HCl salt); 11.12 (bs, 1H, HCl salt). M/Z (M+H)⁺: 345.2. Mp: 50-53° C.

### Example 49: 3-(((5-isopropyl-4,5-dihydro-1H-imidazol-2-yl)thio)methyl)-6,6-dimethyl-5,6-dihydroimidazo[2,1-b]thiazole dihydrochloride

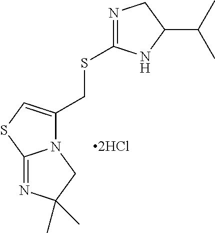 [SMILES: CC(C)C1CN=C(SCC2=CSC3=NC(C)(C)CN23)N1]

Example 49 was isolated by centrifugation of the reaction mixture. The solid was triturated in MeCN (2×2 mL) and Et₂O (2×2 mL) to obtain 3-(((5-isopropyl-4,5-dihydro-1H-imidazol-2-yl)thio)methyl)-6,6-dimethyl-5,6-dihydroimidazo[2,1-b]thiazole dihydrochloride (147 mg, 92%) as a white solid.

¹H-NMR (DMSO-d₆, 400 MHz) δ: 0.84 (dd, J 6.7, 2.6 Hz, 6H, 2 CH₃); 1.50 (d, J 2.1 Hz, 6H, 2 CH₃); 1.80 (sept, J 6.7 Hz, 1H, CH); 3.60 (dd, J 11.4, 7.4 Hz, 1H, N—CHₐH_{b}); 3.89 (t, J 11.4 Hz, 1H, N—CHₐH_{b}); 4.07-4.13 (in, 1H, N—CH); 4.21 (d, J 10.7 Hz, 1H, S—CHₐH_{b}); 4.29 (d, J 10.7 Hz, 1H, S—CHₐH_{b}); 4.75 (d, J 15.7 Hz, 1H, N—CHₐH_{b}); 4.92 (d, J 15.7 Hz, 1H, N—CHₐH_{b}); 7.09 (s, 1H, S—CH); 10.55 (bs, 1H, HCl salt); 11.01 (bs, 2H, HCl salt+NH). M/Z (M+H)⁺: 311.1. Mp: 234-237° C.

### Example 50: 3-(((5,5-dimethyl-4,5-dihydro-1H-imidazol-2-yl)thio)methyl)-6-phenyl-5,6-dihydroimidazo[2,1-b]thiazole dihydrochloride

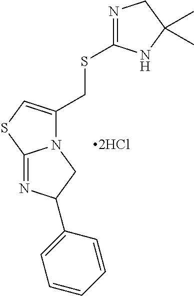 [SMILES: CC1(C)CN=C(SCC2=CSC3=NC(c4ccccc4)CN23)N1]

Example 50 was isolated by centrifugation of the reaction mixture. The solid was triturated in MeCN (2×2 mL) and Et₂O (2×2 mL) to obtain 3-(((5,5-dimethyl-4,5-dihydro-1H-imidazol-2-yl)thio)methyl)-6-phenyl-5,6-dihydroimidazo[2,1-b]thiazole dihydrochloride (136 mg, 94%) as a white solid.

¹H-NMR (DMSO-d₆, 400 MHz) δ: 1.37 (d, J 4.0 Hz, 6H, 2 CH₃); 3.61 (s, 2H, S—CH₂); 4.31 (dd, J 10.9, 8.2 Hz, 1H, N—CHₐH_{b}); 4.71 (d, J 15.6 Hz, 1H, N—CHₐH_{b}); 4.82 (d, J 15.6 Hz, 1H, N—CHₐH_{b}); 4.91 (t, J 10.9 Hz, 1H, N—CH—Ar); 5.89 (dd, J 10.9, 8.2 Hz, 1H, N—CHₐH); 7.19 (s, 1H, S—CH); 7.40-7.51 (m, 5H, Ar); 10.69 (bs, 2H, 2 HCl salt); 11.29 (bs, 1H, NH). M/Z (M+H)⁺: 345.1. Mp: 245-247° C.

### Example 51: 6-benzyl-3-(((5-butyl-4,5-dihydro-1H-imidazol-2-yl)thio)methyl)-5,6-dihydroinidazo[2,1-b]thiazole dihydrochloride

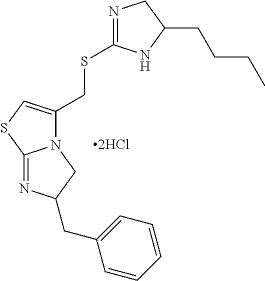 [SMILES: CCCCC1CN=C(SCC2=CSC3=NC(Cc4ccccc4)CN23)N1]

Example 51 was isolated by addition of Et₂O (3 mL) to the reaction mixture. The resulting precipitate was isolated by centrifugation and triturated in Et₂O (3 mL) to afford 6-benzyl-3-(((5-butyl-4,5-dihydro-1H-imidazol-2-yl)thio)methyl)-5,6-dihydroimidazo[2,1-b]thiazole dihydrochloride (102 mg, 67%) as a white solid.

¹H-NMR (DMSO-d₆+D₂O, 400 MHz) δ: 0.86 (t, J 7.0 Hz, 3H, CH₃); 1.13-1.31 (m, 4H, CH₂—CH₂—CH₂—CH₃); 1.7-1.58 (m, 2H, CH₂—CH₂—CH₂—CH₃); 3.06 (d, J 6.4 Hz, 2H, CH—CH₂—Ar); 3.48-3.54 (m, 1H, N—CHₐH_{b}—CH); 3.94 (t, 1H, N—CHₐH_{b}—CH); 4.16-4.27 (m, 2H, N—CHₐH_{b}—CH+N—CH—CH₂); 4.40-4.60 (m, 3H, N—CHₐH_{b}—CH+S—CH₂); 4.97-5.04 (m, 1H, N—CH—CH₂); 6.92 (s, 1H, S—CH); 7.24-7.36 (m, 5H, Ar). M/Z (M+H)⁺: 387.2. Mp: 150-154° C.

### Example 52: 3-(((5,5-dimethyl-4,5-dihydro-1H-imidazol-2-yl)thio)methyl)-6-isopropyl-5,6-dihydroimidazo[2,1-b]thiazole dihydrochloride

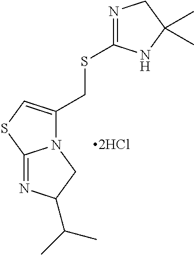 [SMILES: CC(C)C1CN2C(CSC3=NCC(C)(C)N3)=CSC2=N1]

Example 52 was isolated by centrifugation of the reaction mixture. The solid was triturated in MeCN (2×2 mL) and Et₂O (2×2 mL) to obtain 3-(((5,5-dimethyl-4,5-dihydro-1H-imidazol-2-yl)thio)methyl)-6-isopropyl-5,6-dihydroimidazo[2,1-b]thiazole dihydrochloride (137 mg, 91%) as a white solid.

¹H-NMR (DMSO-d₆, 400 MHz) δ: 0.92 (dd, J 10.5 6.8 Hz, 6H, 2 CH₃); 1.36 (d, J 2.5 Hz, 6H, 2 CH₃); 1.95 (m, 1H, CH); 3.60 (s, 2H, S—CH₂); 4.27 (dd, J 10.5, 6.8 Hz, 1H, N—CH); 4.46-4.59 (m, 2H, N—CH₂); 4.71-4.81 (m, 2H, N—CH₂); 7.10 (s, 1H, S—CH); 10.52-10.67 (m, 2H, 2 HCl salt); 11.25 (bs, 1H, NH). M/Z (M+H)⁺: 311.3. Mp: 227-232° C.

### Example 53: 6,6-dimethyl-3-(((4-phenyl-4,5-dihydro-1H-imidazol-2-yl)thio)methyl)-5,6-dihydroimidazo[2,1-b]thiazole dihydrochloride

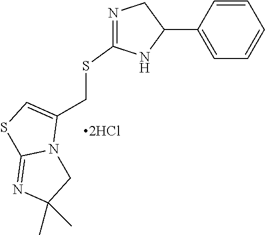 [SMILES: CC1(C)CN2C(CSC3=NCC(c4ccccc4)N3)=CSC2=N1]

Example 53 was isolated by addition of Et₂O (4 mL) to the reaction mixture. The resulting precipitated was isolated by centrifugation, triturated in Et₂O (2×2 mL) and freeze-dried in water to obtain 6,6-dimethyl-3-(((4-phenyl-4,5-dihydro-1H-imidazol-2-yl)thio)methyl)-5,6-dihydroimidazo[2,1-b]thiazole dihydrochloride (80 mg, 46%) as a white solid.

¹H-NMR (DMSO-d₆, 400 MHz) δ: 1.50 (d, J 10.0 Hz, 6H, 2 CH₃); 3.71 (dd, J 11.4, 8.0 Hz, 1H, N—CHₐH_{b}); 4.25 (q, J 23.4, 10.6 Hz, 2H, N—CH₂); 4.33 (t, J 11.4 Hz, 1H, N—CH—Ar); 4.72 (d, J 15.9 Hz, 1H, S—CHₐH_{b}); 4.86 (d, J 15.9 Hz, 1H, S—CHₐH_{b}); 5.41 (dd, J 11.4, 8.0 Hz, 1H, N—CHₐH_{b}); 7.08 (s, 1H, S—CH); 7.31-7.46 (in, 5H, Ar); 10.41 (bs, 1H, HCl salt); 11.04-11.20 (m, 2H, HCl salt+NH). M/Z (M+H)⁺: 345.0. Mp: 110-120° C.

### Example 54: trans-3-(((5-butyl-4,5-dihydro-1H-imidazol-2-yl)thio)methyl)-4a,5,6,7,8,8a-hexahydrobenzo[4,5]imidazo[2,1-b]thiazole dihydrochloride

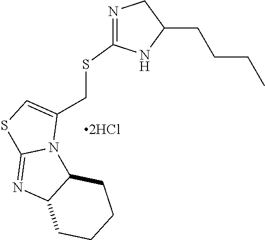

Example 54 was isolated by centrifugation of the reaction mixture. The solid was triturated in MeCN (2×2 mL) and Et₂O (3 mL) to obtain a racemic mixture of trans-3-(((5-butyl-4,5-dihydro-1H-imidazol-2-yl)thio)methyl)-4a,5,6,7,8,8a-hexahydrobenzo[4,5]imidazo[2,1-b]thiazole dihydrochloride (129 mg, 81%) as a white solid.

The product was isolated as a mixture of 2 couples of enantiomers (ratio 0.4:0.6):

¹H-NMR (DMSO-d₆, 400 MHz) δ: 0.86-0.89 (m, 3H, CH₃); 1.17-1.42 (m, 6H, 3 CH₂); 1.48-1.69 (m, 3H, CH₂+CHₐH_{b}); 1.79-1.89 (m, 3H, CHₐH_{b}+CH₂); 2.23 (d, J 12.0 Hz, 1H, CHₐH_{b}); 2.55-2.60 (m, 1H, CHₐH_{b}); 3.51 (dd, J 11.0 Hz, 1H, N—HₐH_{b}); 3.94 (t, J 11.0 Hz, 1H, N—CHₐH_{b}); 3.98-4.05 (m, 1H, N—CH); 4.14-4.27 (m, 2H, 2N—CH); 4.75 (d, J 15.8 Hz, 0.6H, S—CHₐH_{b} of one couple of enantiomers); 4.81-4.91 (m, 0.8H, S—CH₂ of other couple of enantiomers); 4.99 (d, J 15.8 Hz, 0.6H, S—CHₐH_{b} of one couple of enantiomers); 7.22 (s, 0.6H, S—CH of one couple of enantiomers); 7.23 (s, 0.4H, S—CH of other couple of enantiomers); 10.46 (bs, 1H, HCl salt); 10.84 (bs, 1H, HCl salt); 11.15 (bs, 1H, NH). M/Z (M+H)⁺: 351.1. Mp: 226-230° C.

### Example 55: trans-3-(((5-benzyl-4,5-dihydro-1H-imidazol-2-yl)thio)methyl)-4a,5,6,7,8,8a-hexahydrobenzo[4,5]imidazo[2,1-b]thiazole dihydrochloride

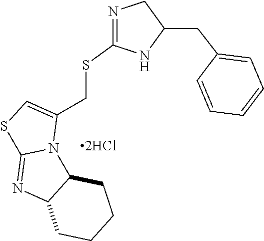

Example 55 was isolated by centrifugation of the reaction mixture. The solid was triturated in MeCN (3 mL) and Et₂O (3 mL). The resulting solid was solubilized in water (6 mL) and this aqueous layer was washed with DCM (10 mL) and freeze-dried to afford a racemic mixture of trans-3-(((5-benzyl-4,5-dihydro-1H-imidazol-2-yl)thio)methyl)-4a,5,6,7,8,8a-hexahydrobenzo[4,5]imidazo[2,1-b]thiazole dihydrochloride (113 mg, 66%) as a yellow solid.

¹H-NMR (DMSO-d₆, 400 MHz) δ: 1.29-1.42 (m, 2H, CH₂); 1.61-1.70 (m, 1H, CHₐH_{b}); 1.79-1.90 (m, 3H, CH₂+CHₐH_{b}); 2.23-2.25 (m, 1H, CHₐH_{b}); 2.54-2.58 (m, 1H, CHₐH_{b}); 2.87-2.97 (m, 2H, CH₂—Ar); 3.58 (dd, J 11.2, 6.8 Hz, 1H, N—CHₐH_{b}); 3.88 (t, J 11.2 Hz, 1H, N—CHₐH_{b}); 4.01 (ddd, J 14.2, 11.2, 3.0 Hz, 1H, N—CHₐH_{b}); 4.11-4.17 (m, 1H, N—CHₐH_{b}); 4.56-4.63 (m, 1H, N—CH); 4.72-4.76 (m, 1H, S—CHₐH_{b}); 4.86-4.90 (m, 1H, S—CHₐH_{b}); 7.19 (s, 1H, S—CH); 7.25-7.35 (m, 5H, Ar); 10.44 (bs, 1H, HCl salt); 10.79 (bs, 1H, HCl salt); 11.18 (bs, 1H, NH). M/Z (M+H)⁺: 385.1. Mp: 137-142° C.

### Example trans-3-((((3aS,7aS)-3a,4,5,6,7,7a-hexahydro-1H-benzo[d]imidazol-2-yl)thio)methyl)-4a,5,6,7,8,8a-hexahydrobenzo[4,5]imidazo[2,1-b]thiazole dihydrochloride

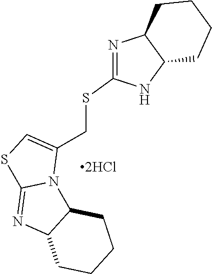

Example 56 was isolated by centrifugation of the reaction mixture. The solid was triturated in MeCN (2×2 mL) and Et₂O (3 mL) to afford a diastereoisometric mixture of trans-3-((((3aS,7aS)-3a,4,5,6,7,7a-hexahydro-1H-benzo[d]imidazol-2-yl)thio)methyl)-4a,5,6,7,8,8a-hexahydrobenzo[4,5]imidazo[2,1-b]thiazole dihydrochloride (116 mg, 73%) as a white solid.

¹H-NMR (DMSO-d₆, 400 MHz) δ: 1.23-1.42 (m, 4H, 2 CH₂); 1.45-1.57 (m, 2H, CH₂); 1.60-1.90 (m, 6H, CH₂+2 CH₂); 2.14 (d, J 11.4 Hz, 2H, CH₂); 2.23 (d, J 11.4 Hz, 1H, CHₐH_{b}); 2.53-2.61 (m, 1H, CHₐH_{b}); 3.94-3.49 (m, 2H, 2N—CH); 4.01 (ddd, J 14.4, 11.2, 3.2 Hz, 1H, N—CH); 4.17 (t, J 12.2 Hz, 1H, N—CH); 4.80-4.92 (m, 2H, S—CH₂); 7.26 (s, 1H, S—CH); 10.39 (bs, 1H, HCl salt); 11.31 (bs, 2H, NH+HCl salt). M/Z (M+H)⁺: 349.1. Mp: 244-246° C.

### Example 57: 6,6-dimethyl-3-(((1-methyl-1,4,5,6-tetrahydropyrimidin-2-yl)thio)methyl)-5,6-dihydroimidazo[2,1-b]thiazole dihydrochloride

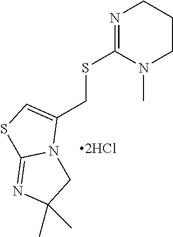 [SMILES: CN1CCCN=C1SCC1=CSC2=NC(C)(C)CN12]

Example 57 was isolated by centrifugation of the reaction mixture. The solid was triturated in MeCN (2×2 mL) and Et₂O (3 mL) to obtain 6,6-dimethyl-3-(((1-methyl-1,4,5,6-tetrahydropyrimidin-2-yl)thio)methyl)-5,6-dihydroimidazo[2,1-b]thiazole dihydrochloride (102 mg, 66%) as a white solid.

¹H-NMR (DMSO-d₆, 400 MHz) δ: 1.51 (s, 6H, 2 CH₃); 1.92 (q, J 17.2, 11.5, 5.8 Hz, 2H, CH₂); 3.25 (s, 3H, N—CH₃); 3.33 (t, J 5.8 Hz, 2H, N—CH₂); 3.50 (t, J 5.8 Hz, 2H, N—CH₂); 4.26 (s, 2H, S—CH₂); 4.71 (s, 2H, N—CH₂); 7.03 (s, 1H, S—CH); 10.51 (bs, 2H, 2 HCl salt). M/Z (M+H)⁺: 297.1. Mp: 245-249° C.

### Example 58: 3-((((4S,5S)-4,5-diisopropyl-4,5-dihydro-1H-imidazol-2-yl)thio)methyl)-6,6-dimethyl-5,6-dihydroimidazo[2,1-b]thiazole dihydrochloride

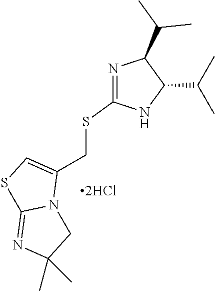 [SMILES: CC(C)[C@@H]1N=C(SCC2=CSC3=NC(C)(C)CN23)N[C@H]1C(C)C]

Example 58 was isolated by centrifugation of the reaction mixture. The solid was triturated in MeCN (2×2 mL) and Et₂O (3 mL) to obtain 3-((((4S,5S)-4,5-diisopropyl-4,5-dihydro-1H-imidazol-2-yl)thio)methyl)-6,6-dimethyl-5,6-dihydroimidazo[2,1-b]thiazole dihydrochloride (162 mg, 91%) as a white solid.

¹H-NMR (DMSO-d₆, 400 MHz) δ: 0.82 (d, J 6.8 Hz, 6H, 2 CH₃); 0.85 (d, J 6.8 Hz, 6H, 2 CH₃); 1.50 (d, J 1.9 Hz, 6H, 2 CH₃); 1.69-1.77 (m, 2H, 2 OH); 3.76-3.79 (m, 2H, 2N—CH); 4.20 (d, J 10.7 Hz, 1H, S—CHₐH_{b}); 4.35 (d, J 10.7 Hz, 1H, S—CHₐH_{b}); 4.72 (d, J 15.8 Hz, 1H, N—CHₐH); 5.06 (d, J 15.8 Hz, 1H, N—CHₐH_{b}); 7.08 (s, 1H, S—CH); 10.50 (bs, 1H, HCl salt); 11.09 (bs, 2H, HCl salt+NH). M/Z (M+H)⁺: 353.2. Mp: 154-160° C.

### Example 59: trans-3-(((4,5-dihydro-1H-imidazol-2-yl)thio)methyl)-4a,5,6,7,8,8a-hexahydrobenzo[4,5]imidazo[2,1-b]thiazole dihydrochloride

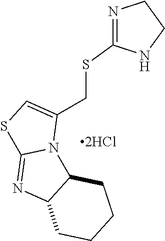

Example 59 was isolated by centrifugation of the reaction mixture. The residue was triturated in MeCN (2 mL) and EtOH (2 mL), then solubilized in MeOH (0.8 mL) and precipitated with Et₂O (2.5 mL). The resulting solid was isolated by centrifugation, triturated in Et₂O (3 mL) and freeze-dried in water to afford a racemic mixture of trans-3-(((4,5-dihydro-1H-imidazol-2-yl)thio)methyl)-4a,5,6,7,8,8a-hexahydrobenzo[4,5]imidazo[2,1-b]thiazole dihydrochloride (90 mg, 65%) as a yellow solid.

¹H-NMR (DMSO-d_{b}, 400 MHz) δ: 1.29-1.43 (m, 2H, CH₂); 1.60-1.69 (m, 1H, CHₐH_{b}); 1.75-1.93 (m, 3H, CH₂+CHₐH_{b}); 2.21-2.25 (m, 1H, CHₐH_{b}); 2.56-2.59 (m, 1H, CHₐH_{b}); 3.85 (s, 4H, 2N—CH₂); 4.01 (ddd, J 14.4, 11.6, 2.8 Hz, 1H, N—CHₐH_{b}); 4.17 (ddd, J 14.4, 11.6, 2.8 Hz, 1H, N—CHₐH_{b}); 4.79 (d, J 15.8 Hz, 1H, S—CHₐH_{b}); 4.90 (d, J 15.8 Hz, 1H, S—CHₐH_{b}); 7.23 (s, 1H, S—CH); 10.35-11.03 (m, 3H, NH+2 HCl salts). M/Z (M+H)⁺: 295.3. Mp: 115-120° C.

### Example 60: 6-benzyl-3-(((5-benzyl-4,5-dihydro-1H-imidazol-2-yl)thio)methyl)-5,6-dihydroimidazo[2,1-b]thiazole dihydrochloride

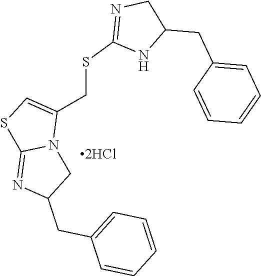 [SMILES: C1=C(CSC2=NCC(Cc3ccccc3)N2)N2CC(Cc3ccccc3)N=C2S1]

Example 60 was isolated by addition of Et₂O (4 mL) to the reaction mixture. The resulting precipitate was isolated by centrifugation, triturated in Et₂O (3 mL) and then solubilized in water (8 mL). This aqueous layer was washed with DCM (10 mL) and freeze-dried to afford 6-benzyl-3-(((5-benzyl-4,5-dihydro-1H-imidazol-2-yl)thio)methyl)-5,6-dihydroimidazo[2,1-b]thiazole dihydrochloride (122 mg, 75%) as a white solid.

¹H-NMR (DMSO-d₆, 400 MHz) δ: 2.87-2.98 (m, 2H, CH₂—Ar); 3.04-3.14 (m, 2H, CH₂—Ar); 3.56 (dd, J 11.2, 6.8 Hz, 1H, N—CHₐH_{b}); 3.86 (t, J 11.2 Hz, 1H, N—CHₐH_{b}); 4.22-4.26 (m, 1H, —CHₐH_{b}); 4.43 (t, J 10.6 Hz, 1H, N—CHₐH_{b}); 4.54-4.62 (m, 1H, N—CH); 4.61-4.78 (m, 2H, S—CH₂); 4.99-5.07 (m, 1H, N—CH); 7.05 (s, 1H, S—CH); 7.24-7.38 (in, 10H, Ar); 10.20 (bs, 1H, HCl salt); 10.76 (bs, 1H, HCl salt); 11.14 (bs, 1H, NH). M/Z (M+H)⁺: 421.2. Mp: 117-121° C.

### Example 61: 6-benzyl-3-((((3aR,7aR)-3a,4,5,6,7,7a-hexahydro-1H-benzo[d]imidazol-2-yl)thio)methyl)-5,6-dihydroimidazo[2,1-b]thiazole dihydrochloride

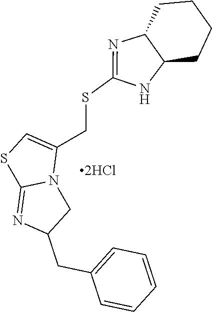 [SMILES: C1=C(CSC2=N[C@@H]3CCCC[C@H]3N2)N2CC(Cc3ccccc3)N=C2S1]

Example 61 was isolated by addition of Et₂O (4 mL) to the reaction mixture. The resulting precipitate was isolated by centrifugation, triturated in Et₂O (3 mL) and then solubilized in water (8 mL). This aqueous layer was washed with DCM (10 mL) and freeze-dried to afford 6-benzyl-3-((((3aR,7aR)-3a,4,5,6,7,7a-hexahydro-1H-benzo[d]imidazol-2-yl)thio)methyl)-5,6-dihydroimidazo[2,1-b]thiazole dihydrochloride (93 mg, 61%) as a white solid.

¹H-NMR (DMSO-d₆, 400 MHz) δ: 1.25-1.36 (m, 2H, CH₂); 1.44-1.55 (m, 2H, CH₂); 1.72-1.82 (m, 2H, CH₂); 2.15 (d, J 11.0 Hz, 2H, CH₂); 3.03-3.13 (m, 2H, CH₂—Ar); 3.38-3.46 (in, 2H, N—CH); 4.24 (dd, J 10.6, 7.0 Hz, 1H, N—CHₐH_{b}); 4.43 (t, J 10.6 Hz, 1H, N—CHₐH_{b}); 4.69-4.77 (m, 2H, S—CH₂); 4.99-5.07 (m, 1H, N—CH); 7.10 (s, 1H, S—OH); 7.26-7.38 (m, 5H, Ar); 10.12 (bs, 1H, HCl salt); 11.22 (bs, 2H, NH+HCl salt). M/Z (M+H)⁺: 385.2. Mp: 125-130° C.

### Example 62 trans-3-(((5,5-dimethyl-4,5-dihydro-1H-imidazol-2-yl)thio)methyl)-5,6-diisopropyl-5,6-dihydroimidazo[2,1-b]thiazole dihydrochloride

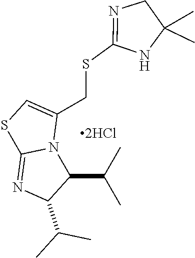 [SMILES: CC(C)[C@@H]1N=C2SC=C(CSC3=NCC(C)(C)N3)N2[C@H]1C(C)C]

Example 62 was isolated by centrifugation of the reaction mixture. The solid was triturated in MeCN (2×2 mL) and Et₂O (2×2 mL) to obtain a racemic mixture of trans-3-(((5,5-dimethyl-4,5-dihydro-1H-imidazol-2-yl)thio)methyl)-5,6-diisopropyl-5,6-dihydroimidazo[2,1-b]thiazole dihydrochloride (105 mg, 81%) as a white solid.

¹H-NMR (DMSO-d₆, 400 MHz) δ: 0.69 (d, J 6.8 Hz, 3H, CH₃); 0.90 (dd, J 6.8, 2.0 Hz, 6H, 2 CH₃); 0.96 (d, J 6.8 Hz, 3H, CH₃); 1.34 (d, J 6.8 Hz, 6H, 2 CH₃); 1.82-1.90 (m, 1H, OH); 2.34-2.39 (m, 1H, CH); 3.60 (s, 2H, S—CH₂); 4.34 (dd, J 5.4, 3.1 Hz, 1H, N—CH); 4.73 (d, J 16.0 Hz, 1H, N—CHₐH_{b}); 4.79 (t, J 3.1 Hz, 1H, N—CH); 4.97 (d, J 16.0 Hz, 1H, N—CHₐH_{b}); 7.11 (s, 1H, S—CH); 10.71 (bs, 2H, 2 HCl salts); 11.35 (bs, 1H, NH). M/Z (M+H)⁺: 353.1. Mp: 237-241° C.

### Example 63: trans-3-(((1-methyl-4,5-dihydro-1H-imidazol-2-yl)thio)methyl)-4a,5,6,7,8,8a-hexahydrobenzo[4,5]imidazo[2,1-b]thiazole dihydrochloride

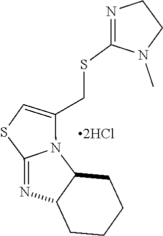

Example 63 was isolated by centrifugation of the reaction mixture. The solid was triturated in MeCN (2×2 mL) and Et₂O (2×2.5 mL) to obtain a racemic mixture of trans-3-(((1-methyl-4,5-dihydro-1H-imidazol-2-yl)thio)methyl)-4a,5,6,7,8,8a-hexahydrobenzo[4,5]imidazo[2,1-b]thiazole dihydrochloride (50 mg, 35%) as a white solid.

¹H-NMR (DMSO-d₆, 400 MHz) δ: 1.31-1.44 (m, 2H, CH₂); 1.61-1.70 (m, 1H, CHₐH_{b}); 1.79-1.88 (m, 3H, CH₂+CHₐH_{b}); 2.22-2.25 (m, 1H, CHₐH_{b}); 2.57-2.60 (m, 1H, CHₐH_{b}); 3.04 (s, 3H, N—CH₃); 3.78-3.93 (m, 4H, 2N—CH₂); 4.01 (ddd, J 14.4, 11.2, 3.2 Hz, 1H, N—CH); 4.14-4.21 (m, 1H, N—CH); 4.87-5.01 (m, 2H, S—CH₂); 7.28 (s, 1H, S—CH); 10.49 (bs, 1H, HCl salt); 11.18 (bs, 1H, HCl salt). M/Z (M+H)⁺: 309.1. Mp: 204-207° C.

### Example 64: 3-(3-((5,5-dimethyl-4,5-dihydro-1H-imidazol-2-yl)thio)propyl)-6,6-dimethyl-5,6-dihydroimidazo[2,1-b]thiazole dihydrochloride

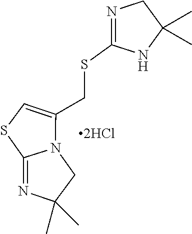 [SMILES: CC1(C)CN2C(CSC3=NCC(C)(C)N3)=CSC2=N1]

Example 64 was isolated by removal of the supernatant. Then the oily residue was triturated in Et₂O (5 mL) until getting a precipitate that was isolated by centrifugation and triturated again in Et₂O (2×2 mL). The resulting solid was then taken up in water and the resulting aqueous layer was washed with DCM (3×20 mL) and freeze-dried to obtain 3-(3-((5,5-dimethyl-4,5-dihydro-1H-imidazol-2-yl)thio)propyl)-6,6-dimethyl-5,6-dihydroimidazo[2,1-b]thiazole dihydrochloride (42 mg, 31%) as a yellow solid.

¹H-NMR (DMSO-d₆, 400 MHz) δ:1.36 (s, 6H, 2 CH₃); 1.49 (s, 6H, 2 CH₃); 1.94 (quint, J 7.5 Hz, 2H, CH₂); 2.70 (t, J 7.5 Hz, 2H, S—CH₂); 3.34 (t, J 7.5 Hz, 2H, S—CH₂); 3.61 (s, 2H, N—CH₂); 4.17 (s, 2H, N—CH₂); 6.62 (s, 1H, S—CH); 10.40 (bs, 2H, 2 HCl salts); 10.85 (bs, 1H, NH). M/Z (M+H)⁺]: 325.1. Mp: 85-95° C.

### Example 65: 3-(2-((5-benzyl-4,5-dihydro-1H-imidazol-2-yl)thio)ethyl)pyridine dihydrochloride

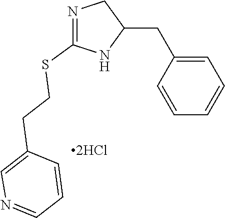 [SMILES: c1ccc(CC2CN=C(SCCc3cccnc3)N2)cc1 ; "·2HCl"]

Example 65 was isolated by concentration to dryness of the reaction mixture. The residue was passed through an ISOLUTE® SCX-2 cartridge (DCM and MeOH, then NH₃ 7N in MeOH) and then purified by flash chromatography (20 μm, DCM 100% to DCM/MeOH 80:20). The resulting yellow oil was freeze-dried in a mixture of water and 1N aqueous HCl (5 equiv) to afford 3-(2-((5-benzyl-4,5-dihydro-1H-imidazol-2-yl)thio)ethyl)pyridine dihydrochloride (103 mg, 54%) as a colorless sticky oil.

¹H-NMR (DMSO-d₆, 400 MHz) δ: 2.93-2.95 (m, 2H, Ar—CH₂); 3.02-3.10 (m, 2H, Ar—CH₂); 3.56-3.62 (m, 3H, S—CH₂+N—CHₐH_{b}—CH); 3.86-3.91 (t, J 10.9 Hz, 1H, N—CHₐH_{b}—CH); 4.55-4.62 (m, 1H, N—CH); 7.22-7.37 (m, 5H, Ar); 7.70-7.73 (dd, J 7.8, 5.2 Hz, 1H, Ar); 8.16-8.18 (in, 1H, Ar); 8.65-8.67 (dd, J 5.2, 1.4 Hz 1H, Ar); 8.75-8.76 (d, J 1.4 Hz 1H, Ar); 10.38 (bs, 1H, HCl salt); 10.71 (bs, 1H, HCl salt); NH signal not observed. M/Z (M+H)⁺: 298.1.

### Example 66: 3-(((5,5-dimethyl-4,5-dihydro-1H-imidazol-2-yl)thio)methyl)-2-iodo-6,6-dimethyl-5,6-dihydroimidazo[2,1-b]thiazole dihydrochloride

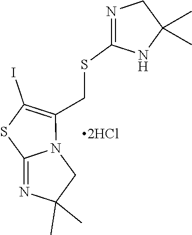 [SMILES: CC1(C)CN2C(=N1)SC(I)=C2CSC1=NCC(C)(C)N1]

Example 66 was isolated by precipitation of the reaction mixture by dropwise addition of Et₂O (50 mL). Then the supernatant was removed and the resulting solid was triturated in Et₂O (2×2 mL) and freeze-dried in water to afford 3-(((5,5-dimethyl-4,5-dihydro-1H-imidazol-2-yl)thio)methyl)-2-iodo-6,6-dimethyl-5,6-dihydroimidazo[2,1-b]thiazole dihydrochloride (51 mg, 70%) as a beige solid.

¹H-NMR (DMSO-d₆, 400 MHz) δ: 1.40 (s, 6H, 2 CH₃); 1.49 (s, 6H, 2 CH₃); 3.67 (s, 2H, S—CH₂); 4.23 (s, 2H, N—CH₂); 4.59 (s, 2H, N—CH₂); 10.23 (bs, 1H, HCl salt); 10.44 (bs, 1H, HCl salt); 10.82 (bs, 1H, NH). M/Z (M+H)⁺: 423.1. Mp: 169-180° C. (decomp.).

### Example 67: 5-benzyl-2-((1-benzylpyrrolidin-3-yl)thio)-4,5-dihydro-1H-imidazole dihydrochloride

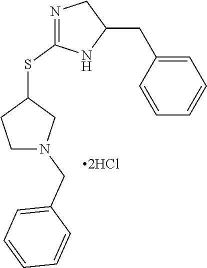 [SMILES: c1ccc(CC2CN=C(SC3CCN(Cc4ccccc4)C3)N2)cc1]

Example 67 was isolated by concentration to dryness of the reaction mixture. The crude was purified by flash chromatography (DCM 100% to DCM/MeOH 90:10). The resulting yellow oil was passed through an ISOLUTE® SCX-2 cartridge (DCM and MeOH, then NH₃ 2N in MeOH) and freeze-dried in a mixture of water and 1N aqueous HCl (5 equiv). The resulting pale yellow solid was purified by preparative HPLC (column B, H₂O+0.1% HCOOH/MeCN+0.1% HCOOH 95:5 to 55:45) and freeze-dried with HCl 1N (5 equiv) to obtain 5-benzyl-2-((1-benzylpyrrolidin-3-yl)thio)-4,5-dihydro-1H-imidazole dihydrochloride (6 mg, 6%) as a pale yellow solid.

¹H-NMR (DMSO-d₆, 400 MHz) δ: 1.97-2.17 (m, 1H, CHₐH_{b}); 2.54-2.61 (m, 1H, CHₐH_{b}); 2.71-2.80 (m, 1H, S—CH); 2.92-2.93 (d, J 5.9 Hz, 2H, Ar—CH₂); 3.11-3.34 (in, 2H, N—CH₂); 3.58-3.62 (dd, J 6.7, 11.0 Hz, 1H, N—CHₐH_{b}—CH); 3.69-3.79 (m, 1H, N—CHₐH_{b}); 3.86-3.91 (t, J 11.0 Hz, 1H, N—CHₐH_{b}—CH); 4.30-4.45 (m, 3H, Ar—CH₂+N—CHₐH_{b}); 4.55-4.64 (m, 1H, N—CH); 7.20-7.35 (m, 5H, Ar); 7.44-7.48 (m, 3H, Ar); 7.60-7.67 (m, 2H, Ar); 10.38-10.47 (m, 1H, HCl salt); 10.74-10.85 (m, 1H, HCl salt); 11.79 (bs, 1H, NH). M/Z (M+H)⁺: 352.1.

### Example 68: 7-(3-((5-benzyl-4,5-dihydro-1H-imidazol-2-yl)thio)propyl)-1,2,3,4-tetrahydro-1,8-naphthyridine dihydrochloride

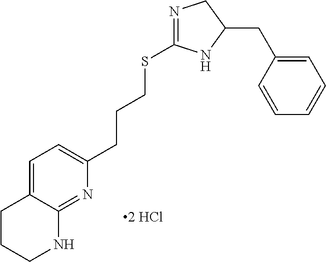 [SMILES: c1ccc(CC2CN=C(SCCCc3ccc4c(n3)NCCC4)N2)cc1]

Example 68 was isolated by filtration of the reaction mixture. The filtrate was concentrated to dryness, passed through an ISOLUTE® SCX-2 cartridge (DCM and MeOH, then NH₃ 7N in MeOH) and then purified by flash chromatography (20 μm, DCM 100% to DCM/MeOH 80:20). The resulting colorless oil was freeze-dried in a mixture of water and 1N aqueous HCl (5 equiv) to afford 7-(3-((5-benzyl-4,5-dihydro-1H-imidazol-2-yl)thio)propyl)-1,2,3,4-tetrahydro-1,8-naphthyridine dihydrochloride (35 mg, 31%) as a white solid.

¹H-NMR (DMSO-dₑ+D₂O, 400 MHz) δ: 1.78-1.84 (in, 2H, CH₂); 1.88-1.96 (m, 2H, CH₂); 2.71-2.74 (m, 4H, Ar—CH₂); 2.89-2.91 (m, 2H, Ar—CH₂); 3.13-3.16 (m, 2H, NH—CH₂); 3.38-3.41 (m, 2H, S—CH₂); 3.57-3.60 (m, 1H, N—CHₐH_{b}—CH); 3.85-3.90 (t, J 10.9 Hz, 1H, N—CHₐH_{b}—CH); 4.53-4.60 (m, 1H, N—CH); 6.57-6.59 (d, J 7.3 Hz, 1H, Ar); 7.20-7.32 (m, 5H, Ar); 7.60-7.61 (d, J 7.3 Hz, 1H, Ar). M/Z (M+H)⁺: 367.2.

### Example 69: 6-benzyl-3-(((1-methyl-4,5-dihydro-1H-imidazol-2-yl)thio)methyl)-5,6-dihydroimidazo[2,1-b]thiazole dihydrochloride

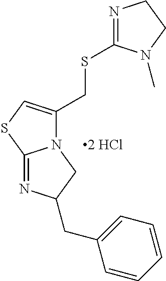 [SMILES: CN1CCN=C1SCC1=CSC2=NC(Cc3ccccc3)CN12]

Example 69 was isolated by addition of Et₂O (3 mL) to the reaction mixture. The resulting precipitate was isolated by centrifugation, triturated in Et₂O (2×2 mL), purified by preparative HPLC (column B, H₂O+0.1% HCOOH/MeCN+0.1% HCOOH 95:5 to 55:45). The combined clean fractions were freeze-dried with HCl 1N (2 equiv) and dissolved in water (5 mL). The resulting aqueous layer was washed with DCM (2×10 mL) and freeze-dried to obtain 6-benzyl-3-(((1-methyl-4,5-dihydro-1H-imidazol-2-yl)thio)methyl)-5,6-dihydroimidazo[2,1-b]thiazole dihydrochloride (20 mg, 14%) as a white solid.

¹H-NMR (DMSO-d₆, 400 MHz) δ: 3.03 (s, 3H, N—CH₃); 3.06-3.14 (m, 2H, Ph-CH₂); 3.77-3.93 (m, 4H, 2N—CH₂); 4.24 (dd, J 10.7, 6.7 Hz, 1H, N—CHₐH_{b}); 4.48 (t, J 10.7 Hz, 1H, N—CHₐH_{b}); 4.73 (d, J 15.6 Hz, 1H, S—CHₐH_{b}); 4.81 (d, J 15.6 Hz, 1H, S—CHₐH_{b}); 4.99-5.06 (m, 1H, N—CH); 7.09 (s, 1H, S—CH); 7.26-7.37 (m, 5H, Ar); 10.07 (bs, 1H, HCl salt); 10.85 (bs, 1H, HCl salt). M/Z (M+H)⁺: 345.1.

### Example 70: 3-((5,5-dimethyl-4,5-dihydro-1H-imidazol-2-yl)thio)-6,6-dimethyl-2,3,5,6-tetrahydroimidazo[2,1-b]thiazole dihydrochloride

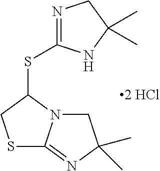 [SMILES: CC1(C)CN2C(=N1)SCC2SC1=NCC(C)(C)N1]

To a solution of 4,4-dimethylimidazolidine-2-thione (200 mg, 1.0 equiv) in EtOH (1.5 mL) was added 1,2-dichloro-1-ethoxyethane (221 μL, 85% purity, 1.0 equiv) and HCl aq. 6N (3 μL, 0.01 equiv). The reaction mixture was heated at 40° C. for 21 h. The resulting precipitate was isolated by centrifugation, triturated in EtOH (2×0.5 mL) and in Et₂O (2×2 mL) to afford 3-((5,5-dimethyl-4,5-dihydro-1H-imidazol-2-yl)thio)-6,6-dimethyl-2,3,5,6-tetrahydroimidazo[2,1-b]thiazole dihydrochloride (45 mg, 16%) as a white solid.

¹H-NMR (DMSO-d₆, 400 MHz) δ: 1.27 (s, 3H, CH₃); 1.28 (s, 3H, CH₃); 1.45 (s, 6H, 2 CH₃); 3.17 (d, J 9.3 Hz, 1H, N—CHₐH_{b}); 3.31 (d, J 10.2 Hz, 1H, N—CHₐH_{b}); 3.57 (d, J 9.3 Hz, 1H, N—CHₐH_{b}); 3.61 (d, J 10.2 Hz, 1H, N—CHₐH_{b}); 3.93 (dd, J 12.4, 2.8 Hz, 1H, S—CHₐH_{b}); 4.36 (dd, J 12.4, 8.2 Hz, 1H, S—CHₐH_{b}); 6.58 (dd, J 8.2, 2.8 Hz, 1H, S—CH—N); 9.19 (bs, 1H, HCl salt); 11.25 (bs, 1H, HCl salt); NH signal not observed. M/Z (M+H)⁺: 285.1. Mp: 240-243° C.

### Example 71: Biological Evaluation

Materials and Methods:

A. Immune Cells Preparation

1) The blood from healthy donors was obtained from two sources. Either from “Etablissement Français du Sang” (convention #07/CABANEL/106), Paris, France or from the Apheresis and Blood Donation Unit at the Institute of Transfusion Medicine and Immunogenetics (IKT), Ulm, Germany after being approved by the Institutional Review Board at Ulm University.

Material from Juvenile Dermatomyositis and Lupus patients were obtained from Necker Hospital, Paris, France or from Imagine Institute, Paris, France. The study was approved by the Conité des Protection des Personnes (ID-RCB/EUDRACT: 2014-A01017-40 and 2018-A01358-47) in France.

Experimental procedures with human blood have been approved by Necker Hospital Ethical Committees for human research and were done according to the European Union guidelines and the Declaration of Helsinki and informed consent was obtained from all donors.

In vitro experiments were performed using human peripheral blood mononuclear cells (PBMCs) isolated by density centrifugation from peripheral blood leukocyte separation medium (Cambrex, Gaithersburg, Md.).

PBMCs were cultured in RPMI 1640 (Invitrogen, Gaithersburg, Md.) (R10) containing 10% heat-inactivated fetal bovine serum, 100 U/mL Penicilium and 100 μg/mL Streptomycin (1% Pen-Strep) and 1 mM glutamine (Hyclone, Logan, Utah).

Monocytes were purified from PBMCs by positive selection with the CD14 microbeads (Miltenyi Biotec) and MACS column.

2) The blood from healthy donors was also obtained from “Etablissement Français du Sang” (convention #19/EFS/029), Paris, France.

In vitro experiments were performed using human peripheral blood mononuclear cells (PBMCs) isolated by density centrifugation from peripheral blood leukocyte separation medium, lymphoprep (Stemcell Technologies).

PBMCs were cultured in RPMI 1640 (Sigma-Aldrich, MO, USA) containing 10% heat-inactivated fetal bovine serum (Sigma-Aldrich, MO, USA) at 37° C./5% CO₂.

B. Immune Cells Stimulation

1) The PBMCs used herein were prepared as described in part A “immune cells preparation”, section 1) above. PBMCs were seeded at 2.10⁶/mL. Cells were pre-treated with different concentration of example 1 or 2 or IT1t (Tocris). Cells were then stimulated with the TLR7/8 agonist resiquimod—R848 (Invivogen) at 5 μg/mL. When stated, AMD (20 μM) (Sigma-Aldrich, MO, USA) was added to the cells 1 h before incubation with example 1.

2) The PBMCs used herein were prepared as described in part A “immune cells preparation”, section 2) above. PBMCs were seeded at 4.10⁶/mL. Cells were pre-treated with different concentration of examples according to the invention or IT1t. Cells were then stimulated with the TLR7/8 agonist resiquimod—R848 (Sigma-Aldrich, MO, USA) at 5 μg/mL.

C. CXR4 Knockdown Experiments

Isolated monocytes were seeded at 10⁵ cells/100 μL in 96-well plates and incubated at 37° C. Cyclophilin B (control) and CXCR4 small interfering RNA (siRNA) (Smart Pool, Dharmarcon) was diluted in DOTAP (Roche Applied Sciences). The mix was gently mixed and incubated at room temperature for 15 minutes. After incubation, the mix was added to cells in culture at a final concentration of 160 nM. Finally, cells were incubated at 37° C. for 24 hours before stimulation.

D. Quantification of Interferon Secretion

1) To quantify the secretion of functional IFN, a biological assay based on a stable cell line where luciferase reporter gene is controlled by five Interferon-Stimulated Response Elements (STING37 cell line) was used. First, supernatants of R848-stimulated PBMCs (see part B, section 1) above) were harvested after 24 h of stimulation and frozen at −20° C. for storage. Then, supernatants were dispensed in culture wells of a 96-well plate containing 35.103 STING37 cells per well. After 24 h of culture, luciferase activity was determined by adding 50 μL of Bright-Glo reagents (Promega) to culture wells and measuring bioluminescence with a luminometer.

2) To quantify the secretion of functional IFN, a biological assay based on a stable cell line where luciferase reporter gene is controlled by five Interferon-Stimulated Response Elements (twINNE cell line) was used. It is important to note that the twINNE cell line is the same as the above mentioned STING-37 cell line but with constitutively expressed nanoluciferase. First, supernatants of R848-stimulated PBMCs (see part B, section 2) above) were harvested after 24 h of stimulation and frozen at −20° C. for storage. Then, supernatants were dispensed in culture wells of a 96-well plate containing 35.103 twINNE cells per well in Dulbecco's Modified Eagle's Medium supplemented with 10% Foetal Bovine Serum, 100 U/mL Penicillin and 100 μg/mL Streptomycin (1% Pen-Strep) and 1 mM glutamine (Sigma-Aldrich, MO, USA) at 37° C./5% C₂. After 24 h of culture, luciferase activity was determined by adding 60 μL of Bright-Go reagents (Promega, Wisconsin, USA) to culture wells and measuring bioluminescence with EnSpire Multimode Plate Reader (PerkinElmer, Massachusetts, USA). When stated, for IC₅₀ calculation, dose-response curves fitted using the nonlin fit (variable slope) analysis in GraphPad Prism software (GraphPad Software, California, USA).

E. Quantification of TNFα Production

R848-stimulated PBMCs were washed in PBS and then incubated with a viability stain (Zombie-Aqua, Biolegend) for 30 min at 4° C. After washing, the cells were resuspended in PBS containing 2% FCS and 2 mM EDTA and stained with the extracellular mix using APC-anti-BDCA4 (clone 12C₂), FITC-anti-CD123 (clone 6H6), PerCP.cy5.5-anti-CD56 (clone 5.1H11), BV421-anti-HLADR (clone L243), PE.cy7-anti-CD19 (clone 4G7), APC-cy.7-anti-CD14 (clone M5E2) all from Biolegend and used at 1/200 and V500-anti-CD3 (BD Bioscience, clone SP34-2, 1/200). For the TNFα intracellular staining a Fixation/Permeabilization Solution Kit (BD Cytofix/Cytoperm) was used according to the manufacturer's protocol. Briefly, the cells were fixed for 10 min at 4° C. with 100 μL of the Fixation/Permeabilization solution then washed and stained in 100 μL of the BD Perm/Wash Buffer containing TNFα-APC (Miltenyi, 1/500) antibody for 1 h at 4° C. Data acquisition was performed on a Canto II flow cytometer using Diva software (BD Biosciences, San Jose, Calif.). FlowJo software (Treestar, Ashland, Oreg.) was used to analyze data.

F. Computational Details

The molecular docking program cDOCKER was used for automated molecular docking simulations and various scoring function were used to rank poses: Jain, cDocker Interaction optimized, Ludi. PDB files were cleaned using the prepare protein protocol of Discovery Studio 2016 (Biovia, Dassault System), membrane was added according to Im. W algorithm Ligands and their conformer were prepared using prepare ligand protocol after conformation generation. Complexes were selected on the basis of criteria of interacting energy combined with geometrical matching quality as well as compromise of scoring function. Figures were generated with Discovery studio 2016 (Biovia, Dassault System) graphics system. An interaction was considered a hydrophobic interaction if the Van der Waals fraction was 0.7 and was considered a hydrogen bond if it was between a listed donor and acceptor and the angles and distances formed by the atoms surrounding the hydrogen bond lay within the default criteria. RMSD were calculated using Discovery studio 2016 (Biovia, Dassault System) and with IT1t in CXCR4/I1t co-crystal as reference (PDB code 3ODU).

G. BRET Assays

Examples of the present invention were tested for their antagonist activity on human CXCR4 (hCXCR4) receptor transiently over-expressed in HEK-293 T cells. Compounds exert antagonist activity if they decrease the action of CXCL12 on the receptor.

The assay used to measure compound activity is based on BRET (Bioluminescence Resonance Energy Transfer) biosensors and is designed to monitor the plasma membrane translocation of protein that interacts with specific Ga subunit. The specific effector (luciferase tagged: BRET donor) recruited at the membrane will be in close proximity to a plasma membrane anchor (GFP tagged: BRET acceptor) to induce a BRET signal (Hamdan et al, 2006, Chapter 5, Current Protocols in Neuroscience).

HEK-293 T cells are maintained in Dulbecco's Modified Eagle's Medium supplemented with 10% Foetal Calf Serum, 1% Penicillin/Streptomycin at 37° C./5% CO₂. Cells are co-transfected using polyethylenimine (25 kDa linear) with several DNA plasmids encoding: hCXCR4; Gαi1, Gαi2, Gαi3, GαoB, or Gαz; an intracellular effector fused to luciferase (BRET donor); a plasma membrane effector fused to GFP (BRET acceptor). After transfection, cells are cultured for 48 h at 37° C./5% CO₂.

On the day of the assay, cells are detached using trypsin 0.05%, resuspended in assay buffer (1.8 mM CaCl₂, 1 mM MgCl₂, 2.7 mM KCl, 137 mM NaCl, 0.4 mM NaH₂PO₄, 5.5 mM D-Glucose, 11.9 mM NaHCO₃, 25 mM Hepes) and seeded in 384 well plate at a density of 20,000 cells per well. Then, plates are equilibrated 3.5 hours at 37° C. before adding compounds. Compounds and luciferase substrate are added to the cells using an automated device (Freedom Evo®, Tecan) and BRET readings are collected on EnVision (PerkinEImer) with specific filters (410 nm BW 80 nm, 515 nm BW 30 nm).

Cells are first incubated for 10 minutes with the compound alone. Then, cells are stimulated by an EC80 CXCL12 concentration for additional 10 minutes and luminescence is recorded. EC80 CXCL12 concentration is the concentration giving 80% of the maximal CXCL12 response. Antagonist activity is evaluated in comparison to basal signals evoked by EC80 CXCL12 alone.

For IC₅₀ determination, a dose-response test is performed using 20 concentrations (ranging over 6 logs) of each compound. Dose-response curves are fitted using the sigmoidal dose-response (variable slope) analysis in GraphPad Prism software (GraphPad Software) and IC₅₀ of antagonist activity is calculated. Dose-response experiments are performed in duplicate, in two independent experiments.

H. Quantification of Cytokine Production

Cytokine expression levels were assessed using LEGENDplex Human inflammation panel 1 Kit (BioLegend, California, USA), which is a bead-based multiplex assay, using fluorescence-encoded beads. This panel allows simultaneous quantification into the same supernatant of activated PBMCs treated with different concentrations of examples of formula (I) according to the invention or IT1t on a set of 13 human inflammatory cytokines/chemokines, including IL-1β, IFN-α2, IFN-γ, TNF-α, MCP-1 (CCL2), IL-6, IL-8 (CXCL8), IL-10, IL-12p70, IL-17A, IL-18, IL-23, and IL-33. First, supernatants of R848-stimulated PBMCs were harvested after 24 h of stimulation and frozen at −20° C. for storage. Supernatants were dispensed into a 96-well V-bottom plate. Protocol pursued as per manufacturer recommendations (Manual for BioLegend LEGENDplex, Multi-Analyte Flow Assay Kit, Human Inflammation Panel 1, version 750000393_R01).

Results:

Effect of Exam Les 1 and 2 on Interferon Production by Peripheral Blood Mononuclear Cells (PBMC) from Healthy Donors

PBMC from healthy donors were cultured (as specified in Materials and Methods, part B “immune cells stimulation”, section 1)) in the presence of different concentrations of examples 1 and 2 or IT1t (positive control) and activated by R848. IFN production was quantified using STING-37 reporter cell line. Both examples 1 and 2 showed a high potency to reduce 100% of IFN production by activated PBMCs (see FIG. 1). This anti-inflammatory activity was more pronounced than the one of IT1t. This validates that examples 1 and 2 have immunomodulatory activity in a mixed culture system containing various immune cells populations.

Effect of Examples 1 and 2 on Interferon Production by PBMCs from Lupus Patients

PBMC from three SLE patients were pre-treated with 10 μM of examples 1 and 2 before being activated by R848. Both molecules were able to reduce IFN production by activated PBMCs to a level similar to the one of unstimulated negative controls (see FIG. 2). These results clearly demonstrate the therapeutic potential of the compounds of formula (I), including in particular examples 1 and 2, to treat type I interferonopathies and inflammatory or autoimmune diseases.

Effect of Example 1 on TNF-Alpha Production by Monocytes from Juvenile Dermatomyositis Patients

PBMCs from 2 juvenile dermatomyositis patients were cultured in the presence of example 1 and the TLR7 activator R848 overnight. TNF-α production in CD14+ monocytes was strongly inhibited by example 1 (see FIG. 3). This result demonstrates that the compounds of formula (1), including example 1, can inhibit the production of several inflammatory cytokines (e.g. IFN, TNFα) in several immune cells (e.g., PBMCs, monocytes). It highlights the high potency of these compounds to reduce inflammation from cells directly involved in the pathology of autoimmune and inflammatory diseases.

Modeling of Examples 1 and 2 Binding to CXCR4

Modeling of IT1t and examples 1 and 2 interactions with CXCR4 was performed. Examples 1 and 2 are found to interact in the same minor binding pocket as iT1t (FIG. 4) with affinity of the same order of magnitude. Calculated arbitrary values for the maximal affinity score are −68.1 kcal/mol for IT1t, −78.6 kcal/mol for example 1 and −63.4 kcal/mol for example 2. The similarity of interactions with CXCR4 between IT1t and examples 1 and 2 indicates common downstream effects on inflammation.

Role of CXCR4 in the Effect of Examples 1 and 2 on TNF-Alpha Production by Monocytes from Healthy Donors

The anti-inflammatory effect of examples 1 and 2 on monocytes from healthy donors was evaluated in a context where their interaction with CXCR4 was inhibited. Either CXCR4 gene was silenced using small interfering RNA (siRNA) or cells were pre-incubated with the well-known CXCR4 antagonist AMD3100 (plerixafor) prior to incubation with example 1 or 2 and R848 (FIG. 5). Both CXCR4 siRNA and AMD3100 restored TNFα production by activated monocytes in the presence of example 1. AMD3100 also restored TNFα production by activated monocytes in the presence of example 2. These results unambiguously demonstrate that CXCR4 is required for the inhibitory activity of examples 1 and 2.

Antagonist Activity of Examples 1 and 2 on the CXCR4-CXCL12 Signaling Pathway

HEK-293 T cells were transfected to allow measuring the recruitment of G proteins involved in intracellular signaling (Gαi1, Gαi2, Gαi3, GαoB, or Gαz) by human CXCR4 receptor (hCXCR4) via the BRET technology. Cells were then incubated with various concentrations of example 1 or 2 or IT1t before stimulation with an EC80 concentration of CXCL12 (FIG. 6). As expected, IT1t inhibited the recruitment of G proteins induced by the hCXCR4-CXCL12 interaction in a dose dependent manner. However, surprisingly, although examples 1 and 2 were found to have greater CXCR4-dependent anti-inflammatory activity than IT1t (FIGS. 1 and 5), their antagonist activity was significantly lower than IT1t in all the conditions tested (FIG. 6). The reduction of antagonist activity is advantageous as it avoids undesirable toxic side effects, and thus allows the long-term administration of the respective compounds. Altogether, the results presented here clearly demonstrate that the compounds of the present invention, including examples 1 and 2, are potent inhibitors of the production of interferons and inflammatory cytokines by specifically targeting CXCR4 while having minimal impact on the CXCR4-CXCL12 signaling pathway.

Effect of Compounds of Formula I on Interferon Production by Peripheral Blood Mononuclear Cells PBMCs from Health Donors

PBMCs from two healthy donors were cultured (as specified in Materials and Methods, part B “immune cells stimulation”, section 2)) in the presence of different concentrations of examples of formula (I) according to the invention (see Table 1 below) or IT1t (positive control) and activated by 5 μg/mL of R848. IFN production was quantified (as specified in Materials and Methods, part D “quantification of interferon secretion”, section 2)) using twINNE reporter cell line. The examples with IC₅₀<31 μM according to the invention showed a higher potency than IT1t to reduce IFN production by activated PBMCs, as detailed in Table 1.

TABLE 1
Effect of various examples according to the invention on IFN production by activated
PBMCs. PBMCs from 2 healthy donors' blood were isolated by Ficoll gradient and
cells were incubated with different concentrations of the respective example or IT1t
before activation with the TLR7 ligand R848 overnight. IFN secretion in the
supernatant was quantified using twINNE reporter cell line. Levels of IFN were
measured by quantifying the luciferase activity induced by the presence of IFN and
IC₅₀ of anti-inflammatory activity were calculated using GraphPad prism software.
The IC₅₀ values of all the examples presented are the means of two results over 2
healthy donors. For IT1t, the IC₅₀ value presented is the mean calculated over all the
experiments performed to determine the IC₅₀ of the examples.

| Examples | IC₅₀ |
|---|---|
| 3, 4, 7, 11, 13, 17, 30, 44, 48, 51, 53, 54, 55, 56, 59, 60, | IC₅₀ ≤ 10 μM |
| 61, 63, 66 | |
| 2, 5, 6, 8, 10, 18, 19, 20, 21, 24, 39, 42, 43, 49, 50, 52, 62, | 10 μM < IC₅₀ < 31 μM |
| 68 | |
| IT1t | 31 μM |
| 9, 12, 14, 15, 16, 22, 23, 25, 26, 27, 28, 29, 31, 32, 33, 34 | IC₅₀ ≥ 31 μM |
| 35, 36, 37, 38, 40 41, 45, 46, 47, 57, 58, 64, 65, 70 | |

Effect of Compounds of Formula I on Cytokine Production by PBMCs from Healthy Donors

PBMCs from two healthy donors were pre-treated with different concentrations of examples of formula (I) according to the invention (see Table 2 below) before being activated by 5 μg/mL R848. IFN production was quantified using twINNE reporter cell line. Cytokine expression levels were assessed in the same supernatants using LEGENDplex Human Inflammation panel 1 Kit. The examples according to the invention were potent in reducing the expression of inflammatory cytokines such as IL-1β, IFN-γ, TNF-α, IL-10, IL-12p70, IL-18 and IL-23. Interestingly, all the tested examples exhibited the same profile of inhibition of the production of inflammatory cytokines (IL-1β, IFN-γ, TNF-α, IL-10, IL-12p70, IL-18 and IL-23) and demonstrated no or poor inhibition on other inflammatory cytokines such as MCP-1, IL-6, IL-8 and IL-33, similarly to IT1t. This indicates that these anti-inflammatory effects are specific and share the same mechanism of action as that of IT1t. In addition, examples 3, 7, 17, and 18 were more potent that i1t in reducing the expression of inflammatory cytokines such as IL-1β, IFN-γ, TNF-α, IL-10, IL-12p70, IL-18 and IL-23, with IC₅₀ on the expression of cytokines equal or below 20 μM on all such cytokines.

TABLE 2
Effect of various examples according to the invention on cytokine production by
activated immune cells (PBMCs) from 2 healthy donors represented by IC₅₀ values.

| | IC₅₀ (μM) | | | | | | |
|---|---|---|---|---|---|---|---|
| Example | 1L-1β | IFN-γ | TNF-α | IL-10 | IL-12p70 | IL-18 | IL-23 |
| 2 | 32 | 26 | 46 | 9.1 | 14 | 13 | 4.7 |
| 3 | 12 | 7.2 | 11 | 11 | 2.6 | 6.9 | 1.7 |
| 7 | 9.9 | 9.8 | 20 | 5.6 | 6.7 | 4.2 | 2.9 |
| 17 | 3.6 | 3.2 | 4.6 | 2.7 | 0.34 | 0.54 | 2.2 |
| 18 | 5.6 | 6.1 | 7.9 | 3.7 | 3.6 | 0.2 | 2.9 |
| 63 | 2.8 | 2.7 | NA | NA | 1.2 | 1.3 | 0.69 |
| IT1t | 38 | 24 | 48 | 20 | 16 | 9.7 | 14 |

PBMCs from 2 different healthy donors were isolated by Ficoll gradient and cells were incubated with the respective example or IT1t at different concentrations before activation with the TLR7 ligand R848 overnight.
Levels of cytokines were measured by LEGENDplex Human Inflammation panel 1 Kit.
“NA” = no inhibitory activity detected.

1. A compound of formula (I) 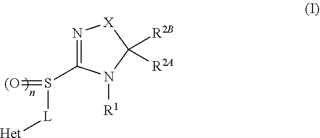
or a pharmaceutically acceptable salt or solvate thereof, wherein:
X is selected from 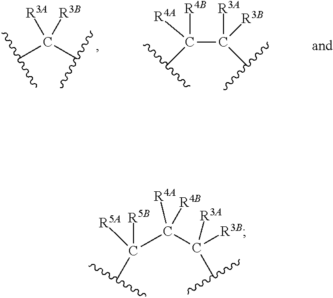
R¹ is selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C₁₋₅ alkyl), and the alkyl moiety in said —COO(C₁₋₅ alkyl) are each optionally substituted with one or more groups R^{Alk}, and further wherein said carbocyclyl and said heterocyclyl are each optionally substituted with one or more groups R^{Cyc};
R^{2A}, R^{2A}, R^{3A}, R^{3B}, R^{4A}, R^{4B}, R^{5A} and R^{5B} are each independently a group -L²⁰-R²⁰, or alternatively:
R^{2A} and R^{3A} may be mutually joined to form, together with the carbon atoms that they are attached to, a cycloalkyl or heterocycloalkyl, wherein said cycloalkyl or said heterocycloalkyl is optionally substituted with one or more groups R⁶, and/or
R^{3A} and R^{4A} may be mutually joined to form, together with the carbon atoms that they are attached to, a cycloalkyl or heterocycloalkyl, wherein said cycloalkyl or said heterocycloalkyl is optionally substituted with one or more groups R⁶, and/or
R^{4A} and R^{5A} may be mutually joined to form, together with the carbon atoms that they are attached to, a cycloalkyl or heterocycloalkyl, wherein said cycloalkyl or said heterocycloalkyl is optionally substituted with one or more groups R⁶; and
if R^{2A} is not mutually joined with R^{3A}, then R^{2A} and R^{2B} may be mutually joined to form, together with the carbon atom that they are attached to, a cycloalkyl or heterocycloalkyl, wherein said cycloalkyl or said heterocycloalkyl is optionally substituted with one or more groups R⁶, and/or
if R^{3A} is not mutually joined with R^{2A} or R^{4A}, then R^{3A} and R^{3B} may be mutually joined to form, together with the carbon atom that they are attached to, a cycloalkyl or heterocycloalkyl, wherein said cycloalkyl or said heterocycloalkyl is optionally substituted with one or more groups R⁶, and/or
if R^{4A} is not mutually joined with R^{3A} or R^{5A}, then R^{4A} and R^{4B} may be mutually joined to form, together with the carbon atom that they are attached to, a cycloalkyl or heterocycloalkyl, wherein said cycloalkyl or said heterocycloalkyl is optionally substituted with one or more groups R⁶, and/or
if R^{5A} is not mutually joined with R^{4A}, then R^{5A} and R^{5B} may be mutually joined to form, together with the carbon atom that they are attached to, a cycloalkyl or heterocycloalkyl, wherein said cycloalkyl or said heterocycloalkyl is optionally substituted with one or more groups R⁶; and
if R^{2B} is not mutually joined with R^{2A}, and if R^{4B} is not mutually joined with R^{4A} then R^{2B} and R^{4B} may be mutually joined to form a C₁₋₃ alkylene, wherein said alkylene is optionally substituted with one or more groups R⁶, and wherein one —CH₂— unit comprised in said alkylene is optionally replaced by a group selected from —O—, —NH—, —N(C₁₋₅ alkyl)-, —CO—, —S—, —SO—, and —SO₂, or
if R^{2B} is not mutually joined with R^{2A}, and if R^{5B} is not mutually joined with R^{5A} then R^{2B} and R^{5B} may be mutually joined to form a C₁₋₃ alkylene, wherein said alkylene is optionally substituted with one or more groups R⁶, and wherein one —CH₂— unit comprised in said alkylene is optionally replaced by a group selected from —O—, —NH—, —N(C₁₋₅ alkyl)-, —CO—, —S—, —SO—, and —SO₂, or
if R^{3B} is not mutually joined with R^{3A}, and if R^{5B} is not mutually joined with R^{5A}, then R^{3B} and R^{5B} may be mutually joined to form a C₁₋₃ alkylene, wherein said alkylene is optionally substituted with one or more groups R⁶, and wherein one —CH₂— unit comprised in said alkylene is optionally replaced by a group selected from —O—, —NH—, —N(C₁₋₅ alkyl)-, —CO—, —S—, —SO—, and —SO₂; and
any remaining groups among R^{2A}, R^{2A}, R^{3A}, R^{3B}, R^{4A}, R^{4A}, R^{5A} and R^{5B}, which are not mutually joined, are each independently a group -L²⁰-R²⁰;
each R⁶ is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{AC}-R^{AC};
each L²⁰ is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —O(C₁₋₅ haloalkyl), —CN, —OR²¹, —NR²¹R²¹, —NR²¹OR²¹, —COR²¹, —COOR²¹, —OCOR²¹, —CONR²¹R²¹, —NR²¹COR², —NR²¹COR²¹, —OCONR²¹R²¹, —SR²¹, —SOR²¹, —SO₂R²¹, —SO₂NR²¹R²¹, —NR²¹SO₂R²¹, —SO₃R²¹, and —NO₂, and further wherein one or more —CH₂— units comprised in said alkylene, said alkenylene or said alkynylene are each optionally replaced by a group independently selected from —O—, —NR²¹—, —CO—, —S—, —SO—, and —SO₂—;
each R²⁰ is independently selected from hydrogen, C₁₋₅ alkyl, C₂₋₅ alkenyl, C₂₋₅ alkynyl, halogen, C₁₋₅ haloalkyl, —O(C₁₋₅ haloalkyl), —CN, —OR²², —NR²²R²², —NR²²OR²², —COR²², —COOR²², —OCR²², —CONR²²R²², —NR²²R²², —NR²²COOR²², —OCONR²²R²², —SR²², —SOR²², —SO₂R²², —SO₂NR²²R²², —NR²²SO₂R²², —SO₃R²², —NO₂, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
each R²¹ and each R²² is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
each R^{Alk} is independently selected from —OH, —O(C₁₋₅ alkyl), —O(C₁₋₅ alkylene)-OH, —(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{AC}-R^{AC};
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, heterocycloalkyl, and -L^{AC}-RAC;
each L^{AC} is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—;
each R^{AC} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O(C₁₋₅ haloalkyl), —CN, —NO₂, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl);
n is 0, 1 or 2;
L is a covalent bond or C₁₋₅ alkylene, wherein said alkylene is optionally substituted with one or more groups R^{L}, and further wherein one or more —CH₂— units comprised in said alkylene are each optionally replaced by a group independently selected from cycloalkylene and heterocycloalkylene;
each R^{L} is independently selected from —OH, —O(C₁₋₅ alkyl), ═O, —SH, —S(C₁₋₅ alkyl), —NH₂, —NH(C₁₋₅ alkyl), —N(C₁₋₅ alkyl)(C₁₋₅ alkyl), halogen, —CF₃, —CN, cycloalkyl, and heterocycloalkyl;
Het is a cyclic group selected from any one of the following groups: 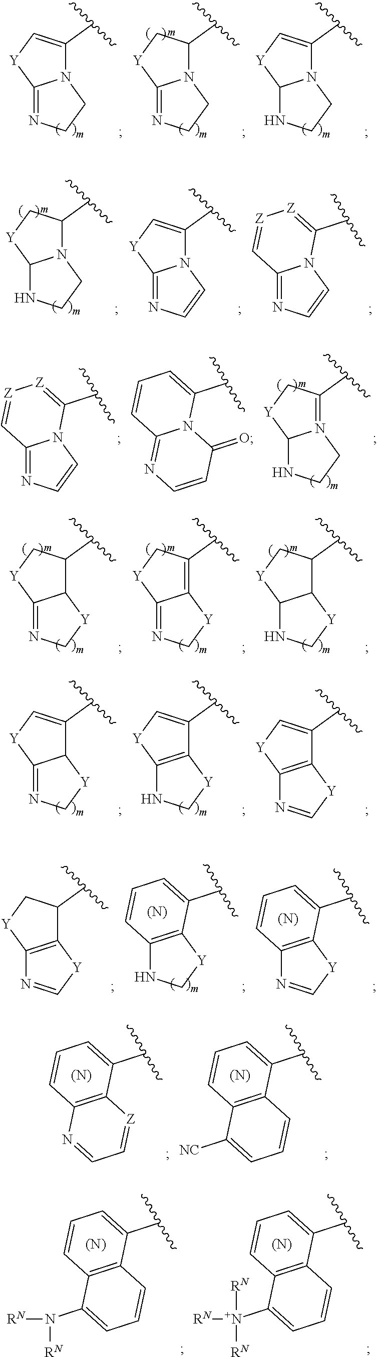 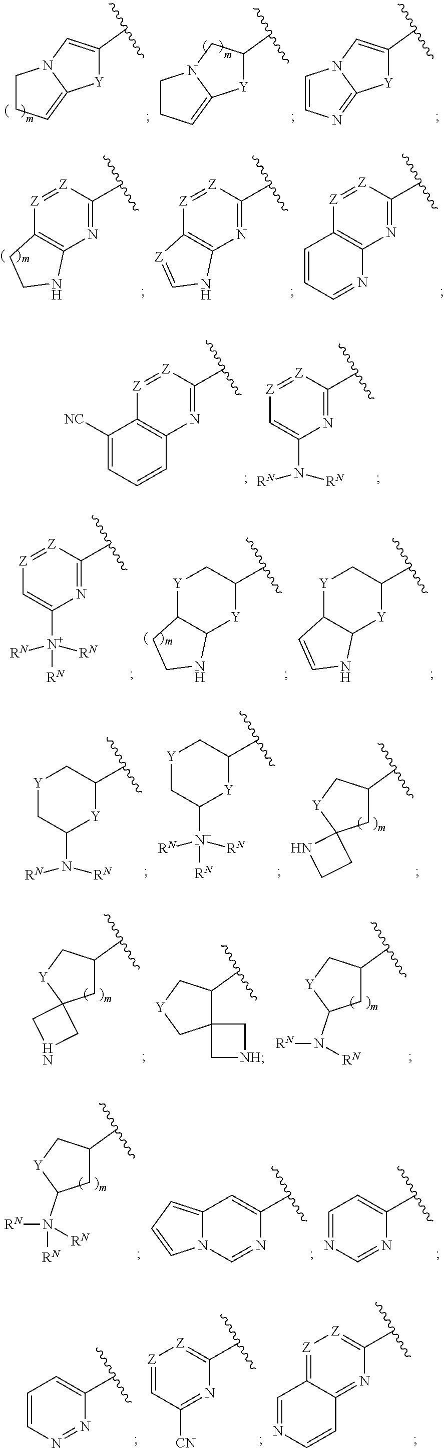 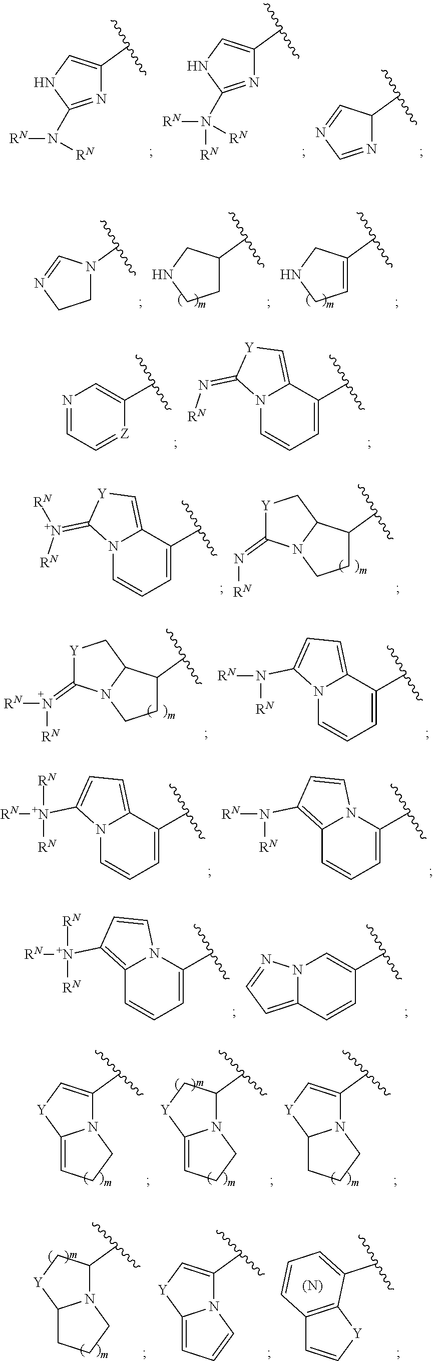 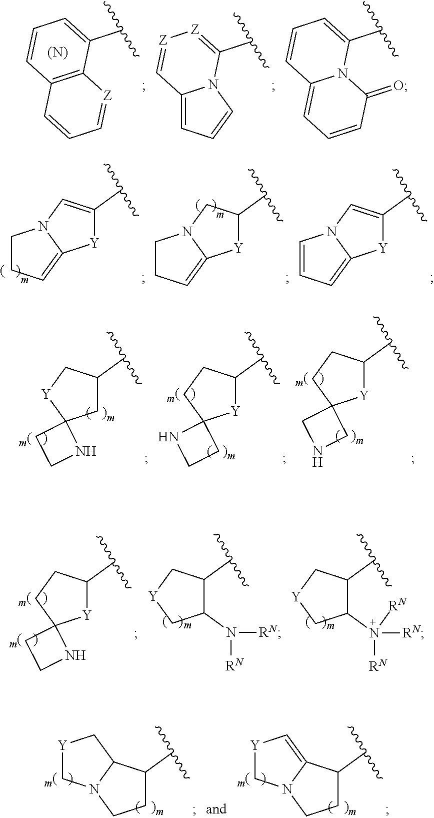
wherein each of the above-depicted groups is optionally substituted with one or more groups R^{Het};
wherein each m is independently 1, 2 or 3;
wherein each ring atom Y is independently selected from S, O, SO₂, NH and CH₂;
wherein each ring atom Z is independently C or N; and
wherein the symbol “(N)” depicted inside a ring indicates that 0, 1, 2 or 3 ring atom(s) of the respective ring is/are nitrogen ring atom(s);
each R^{N} is independently selected from hydrogen, C₁₋₅ alkyl, C₂₋₅ alkenyl, C₂₋₅ alkynyl, —O(C₁₋₅ alkyl), —CO(C₁₋₅ alkyl), —COO(C₁₋₅ alkyl), carbocyclyl, and heterocyclyl, wherein said alkyl, said alkenyl, said alkynyl, the alkyl moiety in said —O(C₁₋₅ alkyl), the alkyl moiety in said —CO(C₁₋₅ alkyl), and the alkyl moiety in said —COO(C₁₋₅ alkyl) are each optionally substituted with one or more groups R^{Alk}, wherein said carbocyclyl and said heterocyclyl are each optionally substituted with one or more groups R^{Cyc}, and further wherein any two groups R^{N} that are attached to the same nitrogen atom may also be mutually joined to form, together with the nitrogen atom that they are attached to, a heterocyclyl which is optionally substituted with one or more groups R^{Cyc};
each R^{Het} is independently a group -L^{H1}-R^{H1}; any two groups R^{Het}, which are attached to the same carbon ring atom of Het, may also be mutually joined to form, together with the carbon atom that they are attached to, a cycloalkyl or a heterocycloalkyl, wherein said cycloalkyl or said heterocycloalkyl is optionally substituted with one or more groups R^{Cyc}; and any two groups R^{Het}, which are attached to different ring atoms of Het, may also be mutually joined to form a C₁₋₅ alkylene, wherein said alkylene is optionally substituted with one or more groups R^{Cyc}, and wherein one —CH₂— unit comprised in said alkylene is optionally replaced by a group selected from —O—, —NH—, —N(C₁₋₅ alkyl)-, —CO—, —S—, —SO—, and —SO₂—;
each L^{H1} is independently selected from a bond, C₁₋₅ alkylene, C₂₋₅ alkenylene, and C₂₋₅ alkynylene, wherein said alkylene, said alkenylene and said alkynylene are each optionally substituted with one or more groups independently selected from halogen, C₁₋₅ haloalkyl, —O(C₁₋₅ haloalkyl), —CN, —OR^{H2}, —NR^{H2}R^{H2}, —N⁺R^{H2}R^{H2}R^{H2}, —NR^{H2}OR^{H2}, —COR^{H2}, —COOR^{H2}, —OCOR^{H2}, —CONR^{H2}R^{H2}, —NR^{H2}COR^{H2}, —NR^{H2}COOR^{H2}, —OCONR^{H2}R^{H2}, —SR^{H2}, —SOR^{H2}, —SO₂R^{H2}, —SO₂NR^{H2}R^{H2}, —NR^{H2}SO₂R^{H2}, —SO₃R^{H2}, and —NO₂, and further wherein one or more —CH₂— units comprised in said alkylene, said alkenylene or said alkynylene are each optionally replaced by a group independently selected from —O—, —NR^{H2}—, —CO—, —S—, —SO—, and —SO₂—;
each R^{H1} is independently selected from C₁₋₅ alkyl, C₂₋₅ alkenyl, C₂₋₅ alkynyl, halogen, C₁₋₅ haloalkyl, —O(C₁₋₅ haloalkyl), —CN, —OR^{H3}, —NR^{H3}R^{H3}, —N⁺R^{H3}R^{H3}R^{H3}, —NR^{H3}OR^{H3}, —COR^{H3}, —COOR^{H3}, —OCOR^{H3}, —CONR^{H3}R^{H3}, —NR^{H3}COR^{H3}, —NR^{H3}COOR^{H3}, —OCONR^{H3}R^{H3}, —SR^{H3}, —SOR^{H3}, SO₂R^{H3}, SO₂NR^{H3}R^{H3}, NR^{H3}SO₂R^{H3}, —SO₃R^{H3}, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
each R^{H2} and each R^{H3} is independently selected from hydrogen, C₁₋₅ alkyl, C₂₋₅ alkenyl, C₂₋₅ alkynyl, carbocyclyl, and heterocyclyl, wherein said alkyl, said alkenyl and said alkynyl are each optionally substituted with one or more groups R^{Alk}, and further wherein said carbocyclyl and said heterocyclyl are each optionally substituted with one or more groups R^{Cyc};
and further wherein the following compounds are excluded from formula (I):
3-((4,5-dihydro-1H-imidazol-2-ylthio)methyl)-2,3,5,6-tetrahydroimidazo[2,1-b]thiazol-3-ol;
2-(4,5-dihydro-1H-imidazol-2-ylthio)-2,3,5,6-tetrahydroimidazo[2,1-b]thiazole;
3-(4,5-dihydro-1H-imidazol-2-ylthio)-2,3,5,6-tetrahydroimidazo[2,1-b]thiazole;
2-(4,5-dihydro-1H-imidazol-2-ylthio)-1-(2-mercapto-4,5-dihydro-1H-imidazol-1-yl)ethanone;
5-chloro-6-((4,5-dihydro-1H-imidazol-2-ylthio)methyl)pyrimidine-2,4-diol;
5-methyl-6-((4,5-dihydro-1H-imidazol-2-ylthio)methyl)pyrimidine-2,4-diol;
6-(4,5-dihydro-1H-imidazol-2-ylthio)-9H-purine;
2-chloro-6-(4,5-dihydro-1H-imidazol-2-ylthio)-9H-purine;
2-amino-6-(4,5-dihydro-1H-imidazol-2-ylthio)-9H-purine;
8-isopropoxy-7-(1-methyl-1H-imidazol-2-ylthio)-5-(1-methyl-4,5-dihydro-1H-imidazol-2-ylthio)quinoline; and
2-(4,5-dihydro-1H-imidazol-2-ylthio)-1-(pyridin-3-yl)ethanone.

2. The compound of claim 1, wherein R¹ is selected from hydrogen, C₁₋₅ alkyl, and cycloalkyl, wherein said cycloalkyl is optionally substituted with one or more groups R^{Cyc}.

3. The compound of claim 1 or 2, wherein X is

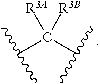

4. The compound of claim 3, wherein R^{2A}, R^{2B}, R^{3A} and R^{3B} are each independently a group -L²⁰-R²⁰.

5. The compound of claim 3, wherein R^{2A} and R^{3A} are mutually joined to form, together with the carbon atoms that they are attached to, a cycloalkyl or heterocycloalkyl, wherein said cycloalkyl or said heterocycloalkyl is optionally substituted with one or more groups R⁶, and further wherein R^{2B} and R^{3B} are each independently a group -L²⁰-R²⁰.

6. The compound of any one of claims 1 to 5, wherein each group -L²⁰-R²⁰ is independently selected from hydrogen, C₁₋₅ alkyl, C₂₋₅ alkenyl, C₂₋₅ alkynyl, halogen, C₁₋₅ haloalkyl, —(C₀₋₅ alkylene)-O(C₁₋₅ haloalkyl), —(C₀₋₅ alkylene)-CN, —(C₀₋₅ alkylene)-OH, —(C₀₋₅ alkylene)-O(C₁₋₅ alkyl), —(C₀₋₅ alkylene)-O(C₁₋₅ alkylene)-OH, —(C₀₋₅ alkylene)-O(C₁₋₅ alkylene)-O(C₁₋₅ alkyl), —(C₀₋₅ alkylene)-NH₂, —(C₀₋₅ alkylene)-NH(C₁₋₅ alkyl), —(C₀₋₅ alkylene)-N(C₁₋₅ alkyl)(C₁₋₅ alkyl), —(C₀₋₅ alkylene)-CHO, —(C₀₋₅ alkylene)-CO(C₁₋₅ alkyl), —(C₀₋₅ alkylene)-COOH, —(C₀₋₅ alkylene)-COO(C₁₋₅ alkyl), —(C₀₋₅ alkylene)-O—CO(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₀₋₅ alkylene)-NH—CO(C₁₋₅ alkyl), —(C₀₋₅ alkylene)-N(C₁₋₅ alkyl)-CO(C₁₋₅ alkyl), —(C₀₋₅ alkylene)-SH, —(C₀₋₅ alkylene)-S(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₀₋₅ alkylene)-NH—SO₂—(C₁₋₅ alkyl), —(C₀₋₅ alkylene)-N(C₁₋₅ alkyl)-SO₂—(C₁₋₅ alkyl), —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein the cycloalkyl moiety in said —(C₀₋₅ alkylene)-cycloalkyl, the aryl moiety in said —(C₀₋₅ alkylene)-aryl, the heterocycloalkyl moiety in said —(C₀₋₅ alkylene)-heterocycloalkyl, and the heteroaryl moiety in said —(C₀₋₅ alkylene)-heteroaryl are each optionally substituted with one or more groups R^{Cyc}.

7. The compound of any one of claims 1 to 6, wherein L is a covalent bond, —CH₂—, or —C(═O)CH₂—, wherein said —C(═O)CH₂— is attached via its C(═O) carbon atom to the group Het and via its CH₂ carbon atom to the group —S(═O)—; and further wherein n is 0.

8. The compound of any one of claims 1 to 7, wherein Het is a cyclic group selected from any one of the following groups: 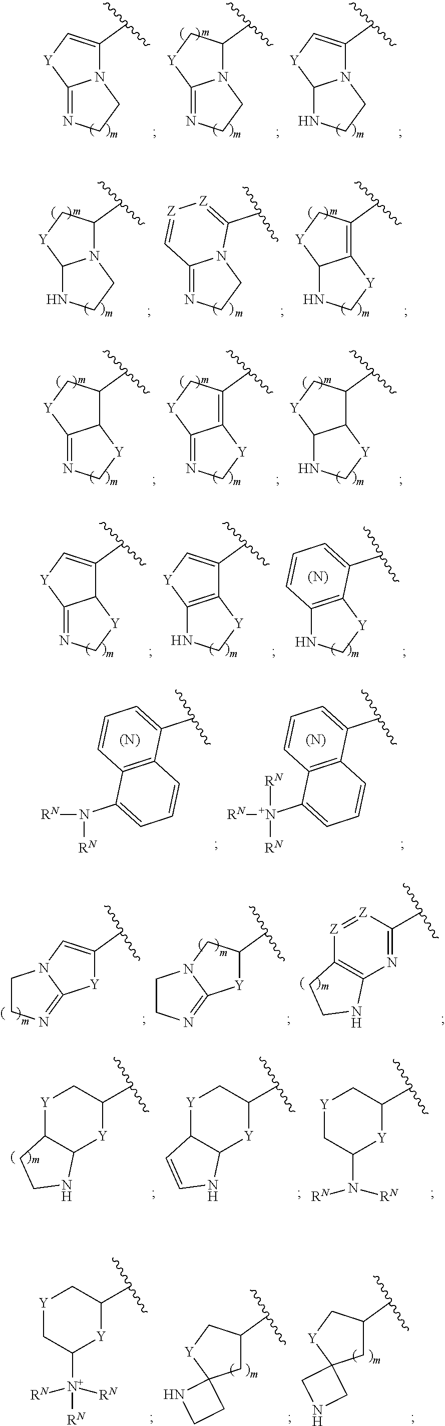 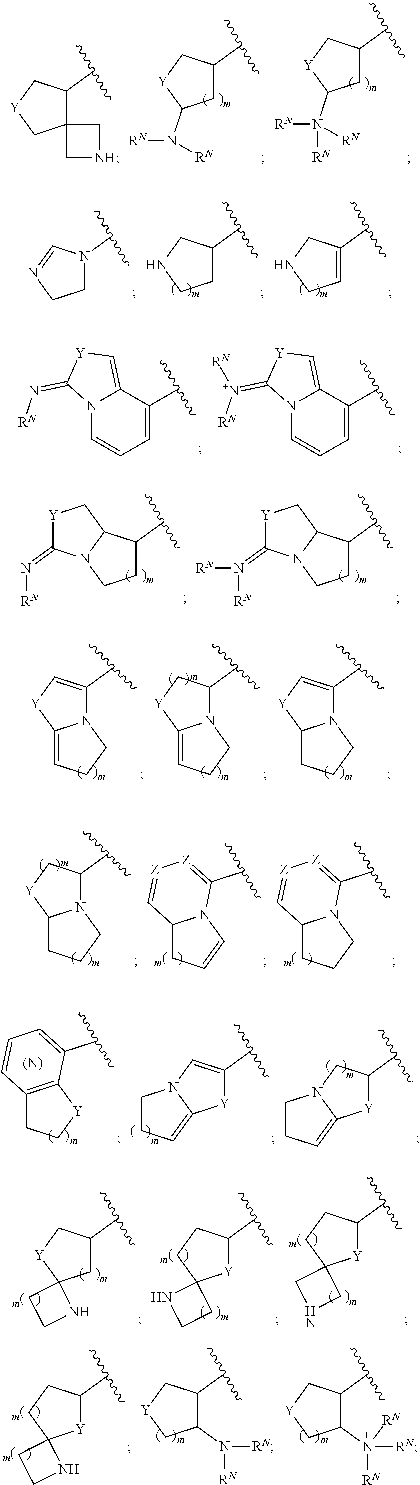 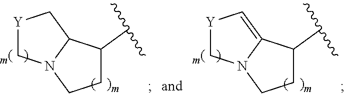
wherein each of the above-depicted groups is optionally substituted with one or more groups R^{Het};
wherein each m is independently 1, 2 or 3;
wherein each ring atom Y is independently selected from S, O, SO₂, NH and CH₂;
wherein each ring atom Z is independently C or N; and
wherein the symbol “(N)” depicted inside a ring indicates that 0, 1, 2 or 3 ring atom(s) of the respective ring is/are nitrogen ring atom(s).

9. The compound of any one of claims 1 to 8, wherein Het is a group 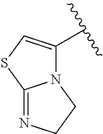 [SMILES: *C1=CSC2=NCCN12]
which is optionally substituted with one or more groups R^{Het}.

10. The compound of any one of claims 1 to 9, wherein:
X is 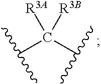
L is —CH₂—;
n is 0; and
Het is a group 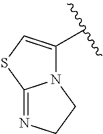 [SMILES: *C1=CSC2=NCCN12]
which is optionally substituted with one or more groups R^{Het}.

11. The compound of claim 1, wherein said compound is selected from:
3-(((3a,4,5,6,7,7a-hexahydro-1H-benzo[d]imidazol-2-yl)thio)methyl)-6,6-dimethyl-5,6-dihydroimidazo[2,1-b]thiazole;
trans-3-(((3a,4,5,6,7,7a-hexahydro-1H-benzo[d]imidazol-2-yl)thio)methyl)-6,6-dimethyl-5,6-dihydroimidazo[2,1-b]thiazole;
cis-3-(((3a,4,5,6,7,7a-hexahydro-1H-benzo[d]imidazol-2-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-6,6-dimethyl-5,6-dihydroimidazo[2,1-b]thiazole;
3-((((3aR,7aR)-3a,4,5,6,7,7a-hexahydro-1H-benzo[d]imidazol-2-yl)thio)methyl)-6,6-dimethyl-5,6-dihydroimidazo[2,1-b]thiazole;
3-((((3aS,7aS)-3a,4,5,6,7,7a-hexahydro-1H-benzo[d]imidazol-2-yl)thio)methyl)-6,6-dimethyl-5,6-dihydroimidazo[2,1-b]thiazole;
3-((((3aR,7aS)-3a,5,6,7,7a-hexahydro-1H-benzo[d]imidazol-2-yl)thio)methyl)-6,6-dimethyl-5,6-dihydroimidazo[2,1-b]thiazole;
3-((((3aS,7aR)-3a,4,5,6,7,7a-hexahydro-1H-benzo[d]imidazol-2-yl)thio)methyl)-6,6-dimethyl-5,6-dihydroimidazo[2,1-b]thiazole;
3-(((4,5-dihydro-1H-imidazol-2-yl)thio)methyl)-5,6-dihydroimidazo[2,1-b]thiazole;
3-(((4,4-dimethyl-4,5-dihydro-1H-imidazol-2-yl)thio)methyl)-4a,5,6,7,8,8a-hexahydrobenzo[4,5]imidazo[2,1-b]thiazole;
trans-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
3-(((4,5-dihydro-1H-imidazol-2-yl)thio)methyl)-6,6-dimethyl-5,6-dihydroimidazo[2,1-b]thiazole;
6,6-dimethyl-3-(((1,4,5,6-tetrahydropyrimidin-2-yl)thio)methyl)-5,6-dihydroimidazo[2,1-b]thiazole;
cis-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
(4aR,8aS)-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
(4aR,8aR)-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
(4aS,8aR)-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
(4aS,8aS)-3-(((4,4-dimethyl-4,5-dihydro-1H-imidazol-2-yl)thio)methyl)-4a,5,6,7,8,8a-hexahydrobenzo[4,5]imidazo[2,1-b]thiazole;
6,6-dimethyl-3-(((1-methyl-4,5-dihydro-1H-imidazol-2-yl)thio)methyl)-5,6-dihydroimidazo[2,1-b]thiazole;
6,6-dimethyl-3-(((4,5,6,7-tetrahydro-1H-1,3-diazepin-2-yl)thio)methyl)-5,6-dihydroimidazo[2,1-b]thiazole;
3-(((5-butyl-4,5-dihydro-1H-imidazol-2-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-6,7-dihydro-5H-thiazolo[3,2-a]pyrimidine;
3-(((5,5-dimethyl-4,5-dihydro-1H-imidazol-2-yl)thio)methyl)-5,6,7,8-tetrahydrothiazolo[3,2-a][1,3]diazepine;
6,6-dimethyl-3-(((4,4,5,5-tetramethyl-4,5-dihydro-1H-imidazol-2-yl)thio)methyl)-5,6-dihydroimidazo[2,1-b]thiazole;
3-(((5-benzyl-4,5-dihydro-1H-imidazol-2-yl)thio)methyl)-6,6-dimethyl-5,6-dihydroimidazo[2,1-b]thiazole;
6-benzyl-3-(((5,5-dimethyl-4,5-dihydro-1H-imidazol-2-yl)thio)methyl)-5,6-dihydroimidazo[2,1-b]thiazole;
6-butyl-3-(((4,4-dimethyl-4,5-dihydro-1H-imidazol-2-yl)thio)methyl)-5,6-dihydroimidazo[2,1-b]thiazole;
3-(((5,5-dimethyl-4,5-dihydro-1H-imidazol-2-yl)thio)methyl)-5,5,6,6-tetramethyl-5,6-dihydroimidazo[2,1-b]thiazole;
3-(((5,5-dimethyl-4,5-dihydro-1H-imidazol-2-yl)thio)methyl)-5,6-dihydroimidazo[2,1-b]thiazole;
3-(((2,4-diazabicyclo[3.3.1]non-2-en-3-yl)thio)methyl)-6,6-dimethyl-5,6-dihydroimidazo[2,1-b]thiazole;
cis-3-(((2,4-diazabicyclo[3.3.1]non-2-en-3-yl)thio)methyl)-6,6-dimethyl-5,6-dihydroimidazo[2,1-b]thiazole;
trans-3-(((2,4-diazabicyclo[3.3.1]non-2-en-3-yl)thio)methyl)-6,6-dimethyl-5,6-dihydroimidazo[2,1-b]thiazole;
3-((((1R,5S)-2,4-diazabicyclo[3.3.1]non-2-en-3-yl)thio)methyl)-6,6-dimethyl-5,6-dihydroimidazo[2,1-b]thiazole;
3-((((1S,5R)-2,4-diazabicyclo[3.3.1]non-2-en-3-yl)thio)methyl)-6,6-dimethyl-5,6-dihydroimidazo[2,1-b]thiazole;
3-((((1R,5R)-2,4-diazabicyclo[3.3.1]non-2-en-3-yl)thio)methyl)-6,6-dimethyl-5,6-dihydroimidazo[2,1-b]thiazole;
3-((((1S,5S)-2,4-diazabicyclo[3.3.1]non-2-en-3-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-6,7,8,9-tetrahydro-5H-5,9-methanothiazolo[3,2-a][1,3]diazocine;
cis-3-(((5,5-dimethyl-4,5-dihydro-1H-imidazol-2-yl)thio)methyl)-6,7,8,9-tetrahydro-5H-5,9-methanothiazolo[3,2-a][1,3]diazocine;
trans-3-(((5,5-dimethyl-4,5-dihydro-1H-imidazol-2-yl)thio)methyl)-6,7,8,9-tetrahydro-5H-5,9-methanothiazolo[3,2-a][1,3]diazocine;
3-(((5,5-dimethyl-4,5-dihydro-1H-imidazol-2-yl)thio)methyl)-(5S,9R)-6,7,8,9-tetrahydro-5H-5,9-methanothiazolo[3,2-a][1,3]diazocine;
3-(((5,5-dimethyl-4,5-dihydro-1H-imidazol-2-yl)thio)methyl)-(5R,9S)-6,7,8,9-tetrahydro-5H-5,9-methanothiazolo[3,2-a][1,3]diazocine;
3-(((5,5-dimethyl-4,5-dihydro-1H-imidazol-2-yl)thio)methyl)-(5S,9S)-6,7,8,9-tetrahydro-5H-5,9-methanothiazolo[3,2-a][1,3]diazocine;
3-(((5,5-dimethyl-4,5-dihydro-1H-imidazol-2-yl)thio)methyl)-(5R,9R)-6,7,8,9-tetrahydro-5H-5,9-methanothiazolo[3,2-a][1,3]diazocine;
3-(((4,6-diazaspiro[2.4]hept-5-en-5-yl)thio)methyl)-6,6-dimethyl-5,6-dihydroimidazo[2,1-b]thiazole;
2-(((6,6-dimethyl-5,6-dihydroimidazo[2,1-b]thiazol-3-yl)methyl)thio)-3a,4,6,6a-tetrahydro-1H-furo[3,4-d]imidazole;
cis-2-(((6,6-dimethyl-5,6-dihydroimidazo[2,1-b]thiazol-3-yl)methyl)thio)-3a,4,6,6a-tetrahydro-1H-furo[3,4-d]imidazole;
trans-2-(((6,6-dimethyl-5,6-dihydroimidazo[2,1-b]thiazol-3-yl)methyl)thio)-3a,4,6,6a-tetrahydro-1H-furo[3,4-d]imidazole;
(3aR,6aS)-2-(((6,6-dimethyl-5,6-dihydroimidazo[2,1-b]thiazol-3-yl)methyl)thio)-3a,4,6,6a-tetrahydro-1H-furo[3,4-d]imidazole;
(3aS,6aR)-2-(((6,6-dimethyl-5,6-dihydroimidazo[2,1-b]thiazol-3-yl)methyl)thio)-3a,4,6,6a-tetrahydro-1H-furo[3,4-d]imidazole;
(3aR,6aR)-2-(((6,6-dimethyl-5,6-dihydroimidazo[2,1-b]thiazol-3-yl)methyl)thio)-3a,4,6,6a-tetrahydro-1H-furo[3,4-d]imidazole;
(3aS,6aS)-2-(((6,6-dimethyl-5,6-dihydroimidazo[2,1-b]thiazol-3-yl)methyl)thio)-3a,4,6,6a-tetrahydro-1H-furo[3,4-d]imidazole;
3-(((4,5-dihydro-1H-imidazol-2-yl)thio)methyl)-6,7-dihydro-5H-thiazolo[3,2-a]pyrimidine;
3-(((4,5-dihydro-1H-imidazol-2-yl)thio)methyl)-5,6,7,8-tetrahydrothiazolo[3,2-a][1,3]diazepine;
3-(((5,5-dimethyl-1,4,5,6-tetrahydropyrimidin-2-yl)thio)methyl)-6,6-dimethyl-5,6-dihydroimidazo[2,1-b]thiazole;
3-(((1,4,5,6-tetrahydropyrimidin-2-yl)thio)methyl)-6,7-dihydro-5H-thiazolo[3,2-a]pyrimidine;
3-(((5-butyl-4,5-dihydro-1H-imidazol-2-yl)thio)methyl)-5,6-dihydroimidazo[2,1-b]thiazole;
3-(3-((5,5-dimethyl-4,5-dihydro-1H-imidazol-2-yl)thio)propyl)pyridine;
3-(((5,5-dimethyl-,5-dihydro-1H-imidazol-2-yl)thio)methyl)pyridine;
3′-(((5,5-dimethyl-4,5-dihydro-1H-imidazol-2-yl)thio)methyl)-5′H-spiro[cyclopropane-1,6′-imidazo[2,1-b]thiazole];
3-(((5,5-dimethyl-4,5-dihydro-1H-imidazol-2-yl)thio)methyl)-6,6-dimethyl-6,7-dihydro-5H-thiazolo[3,2-a]pyrimidine;
3-(((1,4,5,6-tetrahydropyrimidin-2-yl)thio)methyl)-5,6-dihydroimidazo[2,1-b]thiazole;
3-(((3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
cis-3-((((cis)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
cis-3-((((trans)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
trans-3-((((cis)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
trans-3-((((trans)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aR)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aS)-3-((((3aR,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aR)-3-((((3aR,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aR)-3-((((3aR,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aR)-3-((((3aS,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aS)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aS)-3-((((3aR,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aS)-3-((((3aS,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aR)-3-((((3aR,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aR)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aR)-3-((((3aR,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aR,7aR)-3-((((3aS,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aS)-3-((((3aR,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aS)-3-((((3aS,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aS)-3-((((3aR,6aR)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
(4aS,7aS)-3-((((3aS,6aS)-3a,4,6,6a-tetrahydro-1H-furo[3,4-d]imidazol-2-yl)thio)methyl)-4a,5,7,7a-tetrahydrofuro[3′,4′:4,5]imidazo[2,1-b]thiazole;
3-(((1,4,5,6-tetrahydropyrimidin-2-yl)thio)methyl)-5,6,7,8-tetrahydrothiazolo[3,2-a][1,3]diazepine;
1-(3-((5,5-dimethyl-4,5-dihydro-1H-imidazol-2-yl)thio)propyl)-1H-imidazole;
3-(((1,3-diazaspiro[4.5]dec-2-en-2-yl)thio)methyl)-6,6-dimethyl-5,6-dihydroimidazo[2,1-b]thiazole;
6,6-dimethyl-3-(((1,4,4a,5,6,7,8,8a-octahydroquinazolin-2-yl)thio)methyl)-5,6-dihydroimidazo[2,1-b]thiazole;
trans-6,6-dimethyl-3-(((1,4,4a,5,6,7,8,8a-octahydroquinazolin-2-yl)thio)methyl)-5,6-dihydroimidazo[2,1-b]thiazole;
cis-6,6-dimethyl-3-(((1,4,4a,5,6,7,8,8a-octahydroquinazolin-2-yl)thio)methyl)-5,6-dihydroimidazo[2,1-b]thiazole;
6,6-dimethyl-3-((((4aR,8aR)-1,4,4a,5,6,7,8,8a-octahydroquinazolin-2-yl)thio)methyl)-5,6-dihydroimidazo[2,1-b]thiazole;
6,6-dimethyl-3-((((4aS,8aS)-1,4,a,5,6,7,8,8a-octahydroquinazolin-2-yl)thio)methyl)-5,6-dihydroimidazo[2,1-b]thiazole;
6,6-dimethyl-3-((((4aR,8aS)-1,4,4a,5,6,7,8,8a-octahydroquinazolin-2-yl)thio)methyl)-5,6-dihydroimidazo[2,1-b]thiazole;
6,6-dimethyl-3-((((4aS,8aR)-1,4,4a,5,6,7,8,8a-octahydroquinazolin-2-yl)thio)methyl)-5,6-dihydroimidazo[2,1-b]thiazole;
5-(2-((5,5-dimethyl-4,5-dihydro-1H-imidazol-2-yl)thio)ethyl)quinoline;
3′-(((5,5-dimethyl-4,5-dihydro-1H-imidazol-2-yl)thio)methyl)-5′H-spiro[cyclohexane-1,6′-imidazo[2,1-b]thiazole];
3-(((5,5-dimethyl-4,5-dihydro-1H-imidazol-2-yl)thio)methyl)-5a,6,7,8,9,9a-hexahydro-5H-thiazolo[2,3-b]quinazoline;
trans-3-(((5,5-dimethyl-4,5-dihydro-1H-imidazol-2-yl)thio)methyl)-5a,6,7,8,9,9a-hexahydro-5H-thiazolo[2,3-b]quinazoline;
cis-3-(((5,5-dimethyl-4,5-dihydro-1H-imidazol-2-yl)thio)methyl)-5a,6,7,8,9,9a-hexahydro-5H-thiazolo[2,3-b]quinazoline;
3-(((5,5-dimethyl-4,5-dihydro-1H-imidazol-2-yl)thio)methyl)-(5aR,9aR)5a,6,7,8,9,9a-hexahydro-5H-thiazolo[2,3-b]quinazoline;
3-(((5,5-dimethyl-4,5-dihydro-1H-imidazol-2-yl)thio)methyl)-(5aS,9aS)5a,6,7,8,9,9a-hexahydro-5H-thiazolo[2,3-b]quinazoline;
3-(((5,5-dimethyl-4,5-dihydro-1H-imidazol-2-yl)thio)methyl)-(5aR,9aS)5a,6,7,8,9,9a-hexahydro-5H-thiazolo[2,3-b]quinazoline;
3-(((5,5-dimethyl-4,5-dihydro-1H-imidazol-2-yl)thio)methyl)-(5aS,9aR)5a,6,7,8,9,9a-hexahydro-5H-thiazolo[2,3-b]quinazoline;
3-(((3a,4,5,6,7,7a-hexahydro-1H-benzo[d]imidazol-2-yl)thio)methyl)-5,6-dihydroimidazo[2,1-b]thiazole;
trans-3-(((3a,4,5,6,7,7a-hexahydro-1H-benzo[d]imidazol-2-yl)thio)methyl)-5,6-dihydroimidazo[2,1-b]thiazole;
cis-3-(((3a,4,5,6,7,7a-hexahydro-1H-benzo[d]imidazol-2-yl)thio)methyl)-5,6-dihydroimidazo[2,1-b]thiazole;
3-((((3aR,7aR)-3a,4,5,6,7,7a-hexahydro-1H-benzo[d]imidazol-2-yl)thio)methyl)-5,6-dihydroimidazo[2,1-b]thiazole;
3-((((3aS,7aS)-3a,4,5,6,7,7a-hexahydro-1H-benzo[d]imidazol-2-yl)thio)methyl)-5,6-dihydroimidazo[2,1-b]thiazole;
3-((((3aR,7aS)-3a,4,5,6,7,7a-hexahydro-1H-benzo[d]imidazol-2-yl)thio)methyl)-5,6-dihydroimidazo[2,1-b]thiazole;
3-((((3aS,7aR)-3a,4,5,6,7,7a-hexahydro-1H-benzo[d]imidazol-2-yl)thio)methyl)-5,6-dihydroimidazo[2,1-b]thiazole;
3-(((3a,4,5,6,7,7a-hexahydro-1H-benzo[d]imidazol-2-yl)thio)methyl)-6,7-dihydro-5H-thiazolo[3,2-a]pyrimidine;
trans-3-(((3a,4,5,6,7,7a-hexahydro-1H-benzo[d]imidazol-2-yl)thio)methyl)-6,7-dihydro-5H-thiazolo[3,2-a]pyrimidine;
cis-3-(((3a4,5,6,7,7a-hexahydro-1H-benzo[ ]imidazol-2-yl)thio)methyl)-6,7-dihydro-5H-thiazolo[3,2-a]pyrimidine;
3-((((3aR,7aR)-3a,4,5,6,7a-hexahydro-1H-benzo[d]imidazol-2-yl)thio)methyl)-6,7-dihydro-5H-thiazolo[3,2-a]pyrimidine;
3-((((3aS,7aS)-3a,4,5,6,7a-hexahydro-1H-benzo[d]imidazol-2-yl)thio)methyl)-6,7-dihydro-5H-thiazolo[3,2-a]pyrimidine;
3-((((3aR,7aS)-3a,4,5,6,7,7a-hexahydro-1H-benzo[d]imidazol-2-yl)thio)methyl)-6,7-dihydro-5H-thiazolo[3,2-a]pyrimidine;
3-((((3aS,7aR)-3a,4,5,6,7,7a-hexahydro-1H-benzo[d]imidazol-2-yl)thio)methyl)-6,7-dihydro-5H-thiazolo[3,2-a]pyrimidine;
3-(((3a,4,5,6,7,7a-hexahydro-1H-benzo[d]imidazol-2-yl)thio)methyl)-5,6,7,8-tetrahydrothiazolo[3,2-a][1,3]diazepine;
trans-3-(((3a,4,5,6,7,7a-hexahydro-1H-benzo[d]imidazol-2-yl)thio)methyl)-5,6,7,8-tetrahydrothiazolo[3,2-a][1,3]diazepine;
cis-3-(((3a,4,5,6,7,7a-hexahydro-1H-benzo[d]imidazol-2-yl)thio)methyl)-5,6,7,8-tetrahydrothiazolo[3,2-a][1,3]diazepine;
3-((((3aR,7aR)-3a,4,5,6,7,7a-hexahydro-1H-benzo[d]imidazol-2-yl)thio)methyl)-5,6,7,8-tetrahydrothiazolo[3,2-a][1,3]diazepine;
3-((((3aS,7aS)-3a,4,5,6,7,7a-hexahydro-1H-benzo[d]imidazol-2-yl)thio)methyl)-5,6,7,8-tetrahydrothiazolo[3,2-a][1,3]diazepine;
3-((((3aR,7aS)-3a,4,5,6,7,7a-hexahydro-1H-benzo[d]imidazol-2-yl)thio)methyl)-5,6,7,8-tetrahydrothiazolo[3,2-a][1,3]diazepine;
3-((((3aS,7aR)-3a,4,5,6,7,7a-hexahydro-1H-benzo[d]imidazol-2-yl)thio)methyl)-5,6,7,8-tetrahydrothiazolo[3,2-a][1,3]diazepine;
1-(2-((5,5-dimethyl-4,5-dihydro-1H-imidazol-2-yl)thio)ethyl)-1H-imidazole;
3-(((5-benzyl-4,5-dihydro-1H-imidazol-2-yl)thio)methyl)-6,7-dihydro-5H-thiazolo[3,2-a]pyrimidine;
3-(((5-isopropyl-4,5-dihydro-1H-imidazol-2-yl)thio)methyl)-6,6-dimethyl-5,6-dihydroimidazo[2,1-b]thiazole;
3-(((5,5-dimethyl-4,5-dihydro-1H-imidazol-2-yl)thio)methyl)-6-phenyl-5,6-dihydroimidazo[2,1-b]thiazole;
6-benzyl-3-(((5-butyl-4,5-dihydro-1H-imidazol-2-yl)thio)methyl)-5,6-dihydroimidazo[2,1-b]thiazole;
3-(((5,5-dimethyl-4,5-dihydro-1H-imidazol-2-yl)thio)methyl)-6-isopropyl-5,6-dihydroimidazo[2,1-b]thiazole;
6,6-dimethyl-3-(((4-phenyl-4,5-dihydro-1H-imidazol-2-yl)thio)methyl)-5,6-dihydroimidazo[2,1-b]thiazole;
3-(((5-butyl-4,5-dihydro-1H-imidazol-2-yl)thio)methyl)-4a,5,6,7,8,8a-hexahydrobenzo[4,5]imidazo[2,1-b]thiazole;
trans-3-(((5-butyl-4,5-dihydro-1H-imidazol-2-yl)thio)methyl)-4a,5,6,7,8,8a-hexahydrobenzo[4,5]imidazo[2,1-b]thiazole;
cis-3-(((5-butyl-4,5-dihydro-1H-imidazol-2-yl)thio)methyl)-4a,5,6,7,8,8a-hexahydrobenzo[4,5]imidazo[2,1-b]thiazole;
3-(((5-butyl-4,5-dihydro-1H-imidazol-2-yl)thio)methyl)-(4aR,8aR)-4a,5,6,7,8,8a-hexahydrobenzo[4,5]imidazo[2,1-b]thiazole;
3-(((5-butyl-4,5-dihydro-1H-imidazol-2-yl)thio)methyl)-(4aS,8aS)-4a,5,6,7,8,8a-hexahydrobenzo[4,5]imidazo[2,1-b]thiazole;
3-(((5-butyl-4,5-dihydro-1H-imidazol-2-yl)thio)methyl)-(4aR,8aS)-4a,5,6,7,8,8a-hexahydrobenzo[4,5]imidazo[2,1-b]thiazole;
3-(((5-butyl-4,5-dihydro-1H-imidazol-2-yl)thio)methyl)-(4aS,8aR)-4a,5,6,7,8,8a-hexahydrobenzo[4,5]imidazo[2,1-b]thiazole;
3-(((5-benzyl-4,5-dihydro-1H-imidazol-2-yl)thio)methyl)-4a,5,6,7,8,8a-hexahydrobenzo[4,5]imidazo[2,1-b]thiazole;
trans-3-(((5-benzyl-4,5-dihydro-1H-imidazol-2-yl)thio)methyl)-4a,5,6,7,8,8a-hexahydrobenzo[4,5]imidazo[2,1-b]thiazole;
cis-3-(((5-benzyl-4,5-dihydro-1H-imidazol-2-yl)thio)methyl)-4a,5,6,7,8,8a-hexahydrobenzo[4,5]imidazo[2,1-b]thiazole;
3-(((5-benzyl-4,5-dihydro-1H-imidazol-2-yl)thio)methyl)-(4aS,8aR)-4a,5,6,7,8,8a-hexahydrobenzo[4,5]imidazo[2,1-b]thiazole;
3-(((5-benzyl-4,5-dihydro-1H-imidazol-2-yl)thio)methyl)-(4aR,8aS)-4a,5,6,7,8,8a-hexahydrobenzo[4,5]imidazo[2,1-b]thiazole;
3-(((5-benzyl-4,5-dihydro-1H-imidazol-2-yl)thio)methyl)-(4aR,8aR)-4a,5,6,7,8,8a-hexahydrobenzo[4,5]imidazo[2,1-b]thiazole;
3-(((5-benzyl-4,5-dihydro-1H-imidazol-2-yl)thio)methyl)-(4aS,8aS)-4a,5,6,7,8,8a-hexahydrobenzo[4,5]imidazo[2,1-b]thiazole;
3-(((3a,4,5,6,7,7a-hexahydro-1H-benzo[d]imidazol-2-yl)thio)methyl)-4a,5,6,7,8,8a-hexahydrobenzo[4,5]imidazo[2,1-b]thiazole;
trans-3-((((3aS,7aS)-3a,4,5,6,7,7a-hexahydro-1H-benzo[d]imidazol-2-yl)thio)methyl)-4a,5,6,7,8,8a-hexahydrobenzo[4,5]imidazo[2,1-b]thiazole;
cis-3-((((3aS,7aS)-3a,4,5,6,7,7a-hexahydro-1H-benzo[d]imidazol-2-yl)thio)methyl)-4a,5,6,7,8,8a-hexahydrobenzo[4,5]imidazo[2,1-b]thiazole;
3-((((3aS,7aS)-3a,4,5,6,7,7a-hexahydro-1H-benzo[d]imidazol-2-yl)thio)methyl)-(4aR,8aS)-4a,5,6,7,8,8a-hexahydrobenzo[4,5]imidazo[2,1-b]thiazole;
3-((((3aS,7aS)-3a,4,5,6,7,7a-hexahydro-1H-benzo[d]imidazol-2-yl)thio)methyl)-(4aS,8aR)-4a,5,6,7,8,8a-hexahydrobenzo[4,5]imidazo[2,1-b]thiazole;
3-((((3aS,7aS)-3a,4,5,6,7,7a-hexahydro-1H-benzo[d]imidazol-2-yl)thio)methyl)-(4aR,8aR)-4a,5,6,7,8,8a-hexahydrobenzo[4,5]imidazo[2,1-b]thiazole;
3-((((3aS,7aS)-3a,4,5,6,7,7a-hexahydro-1H-benzo[d]imidazol-2-yl)thio)methyl)-(4aS,8aS)-4a,5,6,7,8,8a-hexahydrobenzo[4,5]imidazo[2,1-b]thiazole;
3-((((3aR,7aR)-3a,4,5,6,7,7a-hexahydro-1H-benzo[d]imidazol-2-yl)thio)methy)-(4aR,8aS)-4a,5,6,7,8,8a-hexahydrobenzo[4,5]imidazo[2,1-b]thiazole;
3-((((3aR,7aR)-3a,4,5,6,7,7a-hexahydro-1H-benzo[d]imidazol-2-yl)thio)methyl)-(4aS,8aR)-4a,5,6,7,8,8a-hexahydrobenzo[4,5]imidazo[2,1-b]thiazole;
3-((((3aR,7aR)-3a,4,5,6,7,7a-hexahydro-1H-benzo[d]imidazol-2-yl)thio)methyl)-(4aR,8aR)-4a,5,6,7,8,8a-hexahydrobenzo[4,5]imidazo[2,1-b]thiazole;
3-((((3aR,7aR)-3a,4,5,6,7,7a-hexahydro-1H-benzo[d]imidazol-2-yl)thio)methyl)-(4aS,8aS)-4a,5,6,7,8,8a-hexahydrobenzo[4,5]imidazo[2,1-b]thiazole;
3-((((3aR,7aS)-3a,4,5,6,7,7a-hexahydro-1H-benzo[d]imidazol-2-yl)thio)methyl)-(4aR,8aS)-4a,5,6,7,8,8a-hexahydrobenzo[4,5]imidazo[2,1-b]thiazole;
3-((((3aR,7aS)-3a,4,5,6,7,7a-hexahydro-1H-benzo[d]imidazol-2-yl)thio)methyl)-(4aS,8aR)-4a,5,6,7,8,8a-hexahydrobenzo[4,5]imidazo[2,1-b]thiazole;
3-((((3aR,7aS)-3a,4,5,6,7,7a-hexahydro-1H-benzo[d]imidazol-2-yl)thio)methyl)-(4aR,8aR)-4a,5,6,7,8,8a-hexahydrobenzo[4,5]imidazo[2,1-b]thiazole;
3-((((3aR,7aS)-3a,4,5,6,7,7a-hexahydro-1H-benzo[d]imidazol-2-yl)thio)methyl)-(4aS,8aS)-4a,5,6,7,8,8a-hexahydrobenzo[4,5]imidazo[2,1-b]thiazole;
3-((((3aS,7aR)-3a,4,5,6,7,7a-hexahydro-1H-benzo[d]imidazol-2-yl)thio)methyl)-(4aR,8aS)-4a,5,6,7,8,8a-hexahydrobenzo[4,5]imidazo[2,1-b]thiazole;
3-((((3aS,7aR)-3a,4,5,6,7,7a-hexahydro-1H-benzo[d]imidazol-2-yl)thio)methyl)-(4aS,8aR)-4a,5,6,7,8,8a-hexahydrobenzo[4,5]imidazo[2,1-b]thiazole;
3-((((3aS,7aR)-3a,4,5,6,7,7a-hexahydro-1H-benzo[d]imidazol-2-yl)thio)methyl)-(4aR,8aR)-4a,5,6,7,8,8a-hexahydrobenzo[4,5]imidazo[2,1-b]thiazole;
3-((((3aS,7aR)-3a,4,5,6,7,7a-hexahydro-1H-benzo[d]imidazol-2-yl)thio)methyl)-(4aS,8aS)-4a,5,6,7,8,8a-hexahydrobenzo[4,5]imidazo[2,1-b]thiazole;
6,6-dimethyl-3-(((1-methyl-1,4,5,6-tetrahydropyrimidin-2-yl)thio)methyl)-5,6-dihydroimidazo[2,1-b]thiazole;
3-(((4,5-diisopropyl-4,5-dihydro-1H-imidazol-2-yl)thio)methyl)-6,6-dimethyl-5,6-dihydroimidazo[2,1-b]thiazole;
trans-3-(((4,5-diisopropyl-4,5-dihydro-1H-imidazol-2-yl)thio)methyl)-6,6-dimethyl-5,6-dihydroimidazo[2,1-b]thiazole;
cis-3-(((4,5-diisopropyl-4,5-dihydro-1H-imidazol-2-yl)thio)methyl)-6,6-dimethyl-5,6-dihydroimidazo[2,1-b]thiazole;
3-((((4S,5S)-4,5-diisopropyl-4,5-dihydro-1H-imidazol-2-yl)thio)methyl)-6,6-dimethyl-5,6-dihydroimidazo[2,1-b]thiazole;
3-((((4R,5R)-4,5-diisopropyl-4,5-dihydro-1H-imidazol-2-yl)thio)methyl)-6,6-dimethyl-5,6-dihydroimidazo[2,1-b]thiazole;
3-((((4S,5R)-4,5-diisopropyl-4,5-dihydro-1H-imidazol-2-yl)thio)methyl)-6,6-dimethyl-5,6-dihydroimidazo[2,1-b]thiazole;
3-((((4R,5S)-4,5-diisopropyl-4,5-dihydro-1H-imidazol-2-yl)thio)methyl)-6,6-dimethyl-5,6-dihydroimidazo[2,1-b]thiazole;
3-(((4,5-dihydro-1H-imidazol-2-yl)thio)methyl)-4a,5,6,7,8,8a-hexahydrobenzo[4,5]imidazo[2,1-b]thiazole;
trans-3-(((4,5-dihydro-1H-imidazol-2-yl)thio)methyl)-4a,5,6,7,8,8a-hexahydrobenzo[4,5]imidazo[2,1-b]thiazole;
cis-3-(((4,5-dihydro-1H-imidazol-2-yl)thio)methyl)-4a,5,6,7,8,8a-hexahydrobenzo[4,5]imidazo[2,1-b]thiazole;
3-(((4,5-dihydro-1H-imidazol-2-yl)thio)methyl)-(4aR,8aS)-4a,5,6,7,8,8a-hexahydrobenzo[4,5]imidazo[2,1-b]thiazole;
3-(((4,5-dihydro-1H-imidazol-2-yl)thio)methyl)-(4aS,8aR)-4a,5,6,7,8,8a-hexahydrobenzo[4,5]imidazo[2,1-b]thiazole;
3-(((4,5-dihydro-1H-imidazol-2-yl)thio)methyl)-(4aR,8aR)-4a,5,6,7,8,8a-hexahydrobenzo[4,5]imidazo[2,1-b]thiazole;
3-(((4,5-dihydro-1H-imidazol-2-yl)thio)methyl)-(4aS,8aS)-4a,5,6,7,8,8a-hexahydrobenzo[4,5]imidazo[2,1-b]thiazole;
6-benzyl-3-(((5-benzyl-4,5-dihydro-1H-imidazol-2-yl)thio)methyl)-5,6-dihydroimidazo[2,1-b]thiazole;
6-benzyl-3-(((3a,4,5,6,7,7a-hexahydro-1H-benzo[d]imidazol-2-yl)thio)methyl)-5,6-dihydroimidazo[2,1-b]thiazole;
trans-6-benzyl-3-(((3a,4,5,6,7,7a-hexahydro-1H-benzo[d]imidazol-2-yl)thio)methyl)-5,6-dihydroimidazo[2,1-b]thiazole;
cis-6-benzyl-3-(((3a,4,5,6,7,7a-hexahydro-1H-benzo[d]imidazol-2-yl)thio)methyl)-5,6-dihydroimidazo[2,1-b]thiazole;
6-benzyl-3-((((3aR,7aR)-3a,4,5,6,7,7a-hexahydro-1H-benzo[d]imidazol-2-yl)thio)methyl)-5,6-dihydroimidazo[2,1-b]thiazole;
6-benzyl-3-((((3aS,7aS)-3a,4,5,6,7,7a-hexahydro-1H-benzo[d]imidazol-2-yl)thio)methyl)-5,6-dihydroimidazo[2,1-b]thiazole;
6-benzyl-3-((((3aR,7aS)-3a,4,5,6,7,7a-hexahydro-1H-benzo[d]imidazol-2-yl)thio)methyl)-5,6-dihydroimidazo[2,1-b]thiazole;
6-benzyl-3-((((3aS,7aR)-3a,4,5,6,7,7a-hexahydro-1H-benzo[d]imidazol-2-yl)thio)methyl)-5,6-dihydroimidazo[2,1-b]thiazole;
3-(((5,5-dimethyl-4,5-dihydro-1H-imidazol-2-yl)thio)methyl)-5,6-diisopropyl-5,6-dihydroimidazo[2,1-b]thiazole;
trans-3-(((5,5-dimethyl-4,5-dihydro-1H-imidazol-2-yl)thio)methyl)-5,6-diisopropyl-5,6-dihydroimidazo[2,1-b]thiazole;
cis-3-(((5,5-dimethyl-4,5-dihydro-1H-imidazol-2-yl)thio)methyl)-5,6-diisopropyl-5,6-dihydroimidazo[2,1-b]thiazole;
3-(((5,5-dimethyl-4,5-dihydro-1H-imidazol-2-yl)thio)methyl)-(5R,6R)-5,6-diisopropyl-5,6-dihydroimidazo[2,1-b]thiazole;
3-(((5,5-dimethyl-4,5-dihydro-1H-imidazol-2-yl)thio)methyl)-(5S,6S)-5,6-diisopropyl-5,6-dihydroimidazo[2,1-b]thiazole;
3-(((5,5-dimethyl-4,5-dihydro-1H-imidazol-2-yl)thio)methyl)-(5R,6S)-5,6-diisopropyl-5,6-dihydroimidazo[2,1-b]thiazole;
3-(((5,5-dimethyl-4,5-dihydro-1H-imidazol-2-yl)thio)methyl)-(5S,6R)-5,6-diisopropyl-5,6-dihydroimidazo[2,1-b]thiazole;
3-(((1-methyl-4,5-dihydro-1H-imidazol-2-yl)thio)methyl)-4a,5,6,7,8,8a-hexahydrobenzo[4,5]imidazo[2,1-b]thiazole;
trans-3-(((1-methyl-4,5-dihydro-1H-imidazol-2-yl)thio)methyl)-4a,5,6,7,8,8a-hexahydrobenzo[4,5]imidazo[2,1-b]thiazole;
cis-3-(((1-methyl-4,5-dihydro-1H-imidazol-2-yl)thio)methyl)-4a,5,6,7,8,8a-hexahydrobenzo[4,5]imidazo[2,1-b]thiazole;
3-(((1-methyl-4,5-dihydro-1H-imidazol-2-yl)thio)methyl)-(4aR,8aR)-4a,5,6,7,8,8a-hexahydrobenzo[4,5]imidazo[2,1-b]thiazole;
3-(((1-methyl-4,5-dihydro-1H-imidazol-2-yl)thio)methyl)-(4aS,8aS)-4a,5,6,7,8,8a-hexahydrobenzo[4,5]imidazo[2,1-b]thiazole;
3-(((1-methyl-4,5-dihydro-1H-imidazol-2-yl)thio)methyl)-(4aR,8aS)-4a,5,6,7,8,8a-hexahydrobenzo[4,5]imidazo[2,1-b]thiazole;
3-(((1-methyl-4,5-dihydro-1H-imidazol-2-yl)thio)methyl)-(4aS,8aR)-4a,5,6,7,8,8a-hexahydrobenzo[4,5]imidazo[2,1-b]thiazole;
3-(3-((5,5-dimethyl-4,5-dihydro-1H-imidazol-2-yl)thio)propyl)-6,6-dimethyl-5,6-dihydroimidazo[2,1-b]thiazole;
3-(2-((5-benzyl-4,5-dihydro-1H-imidazol-2-yl)thio)ethyl)pyridine;
3-(((5,5-dimethyl-4,5-dihydro-1H-imidazol-2-yl)thio)methyl)-2-iodo-6,6-dimethyl-5,6-dihydroimidazo[2,1-b]thiazole;
5-benzyl-2-((1-benzylpyrrolidin-3-yl)thio)-4,5-dihydro-1H-imidazole;
7-(3-((5-benzyl-4,5-dihydro-1H-imidazol-2-yl)thio)propyl)-1,2,3,4-tetrahydro-1,8-naphthyridine;
6-benzyl-3-(((1-methyl-4,5-dihydro-1H-imidazol-2-yl)thio)methyl)-5,6-dihydroimidazo[2,1-b]thiazole;
3-((5,5-dimethyl-4,5-dihydro-1H-imidazol-2-yl)thio)-6,6-dimethyl-2,3,5,6-tetrahydroimidazo[2,1-b]thiazole;
or a pharmaceutically acceptable salt or solvate of any one of these compounds.

12. A pharmaceutical composition comprising the compound of any one of claims 1 to 11 and a pharmaceutically acceptable excipient.

13. The compound of any one of claims 1 to 11 or the pharmaceutical composition of claim 12 for use in the treatment or prevention of an inflammatory disorder, an autoimmune disorder, an autoinflammatory disorder, or an interferonopathy.

14. The compound for use according to claim 13 or the pharmaceutical composition for use according to claim 13, wherein the inflammatory disorder, autoimmune disorder, autoinflammatory disorder or interferonopathy to be treated or prevented is selected from Aicardi-Goutières syndrome, familial chilblain lupus, Singleton-Merten syndrome, proteasome-associated autoinflammatory syndrome, deficiency of adenosine deaminase 2, retinal vasculopathy with cerebral leukodystrophy, STING-associated vasculopathy with onset in infancy, spondyloenchondrodysplasia, ISG15 deficiency, an interferonopathy associated with genetic dysfunction, familial Mediterranean fever, TNF receptor associated periodic fever syndrome, periodic fever, aphthous stomatitis, pharyngitis, cervical adenitis, pyogenic arthritis, pyoderma gangrenosum, acne, Blau syndrome, neonatal onset multisystem inflammatory disease, familial cold autoinflammatory syndrome, hyperimmunoglobulinemia D with periodic fever syndrome, Muckle-Wells syndrome, chronic infantile neurological cutaneous and articular syndrome, deficiency of interleukin-1 receptor antagonist, haploinsufficiency of A20, deficiency of IL-36 receptor antagonist, CARD14-mediated psoriasis, inflammatory bowel disease, PLCG2-associated autoinflammation, antibody deficiency and immune dysregulation, an inflammatory disorder associated with genetic dysfunction, rheumatoid arthritis, spondyloarthritis, osteoarthritis, gout, idiopathic juvenile arthritis, psoriatic arthritis, eczema, psoriasis, scleroderma, systemic lupus erythematosus, Sjögren's syndrome, dermatomyositis, overlapping myositis, mixed connective tissue disease, undifferentiated connective tissue disease, chronic obstructive pulmonary disease, bowel inflammation, Crohn disease, Behçet's disease, ulcerative colitis, sepsis, macrophages activation syndrome, acute respiratory distress syndrome, type it diabetes, asthma, chronic wounds, autism, multiple sclerosis, Alzheimer's disease, Parkinson's disease, chronic inflammatory demyelinating polyneuropathy, juvenile dermatomyositis, and an inflammatory complication associated with a viral infection.

15. The compound of any one of claims 1 to 11 or the pharmaceutical composition of claim 12 for use in the treatment or prevention of rheumatoid arthritis, dermatomyositis or systemic lupus erythematosus.

16. In vitro use of a compound as defined in any one of claims 1 to 11 as a CXCR4 modulator.
